(19) United States
(12) Patent Application Publication
(10) Pub. No.: US 2026/0284174 A1
(43) Pub. Date: Sep. 24, 2026

(54) INFLUENZA VIRUS VACCINES

(71) Applicant: GLAXOSMITHKLINE BIOLOGICALS SA, Rixensart (BE)

(72) Inventors: Philip R. DORMITZER, Waltham, MA (US); Clarisse Marie-Madeleine LORIN, Rixensart (BE); Brian MOLDT, Wavre (BE); Pascal PEETERS, Wavre (BE)

(73) Assignee: GLAXOSMITHKLINE BIOLOGICALS SA, Rixensart (BE)

(21) Appl. No.: 19/478,656

(22) PCT Filed: Apr. 25, 2024
(86) PCT No.: PCT/EP2024/061349

§ 371 Date: Oct. 24, 2025

Related U.S. Application Data
(60) Provisional application No. 63/498,550, filed on Apr. 27, 2023.
(60) Provisional application No. 63/584,940, filed on Sep. 25, 2023.

(51) Int. Cl. A61K 39/145; A61K 9/127; A61K 9/51; A61K 39/00; A61P 31/16; C12N 7/00

(52) U.S. Cl. CPC A61K 39/145; A61K 9/127; A61K 9/5123; A61P 31/16; C12N 7/00; A61K 2039/53; A61K 2039/55555; A61K 2039/572; A61K 2039/575; A61K 2039/70; C12N 2760/16134; C12N 2760/16234

(57) ABSTRACT

The present invention is inter alia directed to immunogenic compositions comprising: (a) a first hemagglutinin (HA) antigen or a first nucleic acid, suitably mRNA, encoding the first HA antigen wherein the first HA antigen is derived from a strain of subtype H3 of Influenza A virus; and (b) a second HA antigen or a second nucleic acid, suitably mRNA, encoding the second HA antigen wherein the second HA antigen is derived from a strain of subtype H1 of Influenza A virus, wherein the ratio of (a):(b) is comprised between 1.5:1 and 20:1. The present invention is also directed to vaccines and kits or kits-of-parts comprising such. Immunogenic compositions, vaccines and kits-of-parts provided herein are suitable for use as a medicament, in particular, for use in the treatment or prophylaxis of an infection with an Influenza virus, suitably an Influenza A and/or Influenza B.

### TECHNICAL FIELD

The present invention is inter alia directed to immunogenic compositions comprising hemagglutinin (HA) antigens or nucleic acids, suitably mRNA, encoding the HA antigens wherein the HA antigens are derived from Influenza virus strains. The present invention is also directed to vaccines and kits or kits of parts comprising such. Immunogenic compositions, vaccines and kits-of-parts provided herein are suitable for use as a medicament, in particular, for use in the treatment or prophylaxis of an infection with an Influenza virus, suitably an Influenza A and/or Influenza B.

### BACKGROUND

Influenza viruses are RNA viruses belonging to the family Orthomyxoviridae (NCBI Taxonomy ID: 11308), being sub-divided into e.g. AlphaInfluenzavirus (the genus that includes Influenza A viruses) and BetaInfluenzavirus (the genus that includes Influenza B viruses), that circulate in all parts of the world. Influenza viruses cause acute respiratory illness often during local outbreaks or seasonal epidemics and occasionally during pandemics. Typical Influenza epidemics cause increases in incidence of pneumonia and lower respiratory disease by increased rates of hospitalization or mortality. The elderly or those with underlying chronic diseases are most likely to experience such complications, but young infants also may suffer severe disease.

Influenza viruses (mainly Influenza A and B viruses) have a significant impact on global public health, causing millions of cases of severe illness each year, thousands of deaths, and considerable economic losses.

Influenza viruses, such as Influenza A and B viruses, are enveloped viruses comprising eight pieces of segmented negative-sense RNA, which encode 11 proteins (HA, NA, NP, M1, M2, NS1, NEP, PA, PB1, PB1-F2, PB2). The best-characterized of these viral proteins are hemagglutinin (HA) and neuraminidase (NA), two large glycoproteins found on the outside of the viral particles. NA is an enzyme involved in the release of progeny virus from infected cells. HA is a lectin that mediates binding of the virus to target cells and entry of the viral genome into the target cell.

Currently, there are 18 described HA (H1-H18) subtypes and 11 described NA (N1-N11) subtypes of Influenza A viruses that potentially form 144 HA and NA combinations. Unlike Influenza A viruses that have a wide range of host, the Influenza B viruses almost exclusively infect humans. The Influenza B viruses are categorized into two distinct lineages: B/Victoria/2/1987-like (B/Victoria lineage) and B/Yamagata/16/1988-like (B/Yamagata lineage) viruses that have been circulating worldwide since 1983. Influenza virus B mutates at a rate 2 to 3 times slower than type A; however, it significantly impacts children and young adults annually.

Vaccination is currently the most widely used method to prevent Influenza outbreaks, particularly in high-risk population. Constant emergence of new strains of Influenza virus through antigenic drift is the virological basis for seasonal epidemics. Due to its constant evolving nature, the periodic update of viruses contained in Influenza (flu) vaccines is necessary for the vaccines to be effective. Public health authorities monitor the Influenza viruses circulating in humans and update the recommended composition of flu vaccines twice a year. The recommendations issued (usually, three or four different strains of Influenza virus) are used by the national vaccine regulatory agencies and pharmaceutical companies to develop, produce, and license Influenza vaccines for the following Influenza season. Vaccination is effective in reducing the risk of influenza illness by 40% to 60% in the overall population when vaccine strains and circulating viruses are similar.

Multivalent live attenuated (FLUMIST, AstraZeneca), inactivated (AFLURIA, FLUAD and FLUCELVAX, Seqirus; FLUARIX and FLULAVAL, GlaxoSmithKline; FLUZONE, Sanofi), or recombinant (FLUBLOK, Sanofi) flu vaccines are already available on the market for active immunization against disease caused by Influenza subtype A viruses and Influenza type B viruses contained in the vaccines.

Because HA is the major Influenza virus antigen recognized by neutralizing antibodies, this glycoprotein has been the focus of currently inactivated and recombinant approved flu vaccines. Most of those flu vaccines are quadrivalent vaccines, based on 4 HA derived from each of the four strains of Influenza virus specified by health authorities for inclusion in the annual seasonal vaccine (typically two Influenza subtype A strains and two Influenza type B strains), meaning designed to protect against those four different flu virus strains. Each of the 4 HA is present in the vaccine in an equimolar proportion. The standard dose of 1 HA (i.e. per strain) is 15 μg/0.5 ml, leading to a total (i.e. for the 4 HA) standard dose of 60 μg/0.5 ml. Some available flu vaccines are further approved for higher doses, e.g. 45 μg/0.5 ml of HA per strain (FLUBLOK, Sanofi) or 60 μg/0.7 ml of HA per strain (FLUZONE HIGH-DOSE, Sanofi).

Currently circulating in humans are subtype A (H1N1) and A (H3N2) influenza viruses. In the past ten years, H3N2 has tended to dominate in prevalence over H1N1, H1N2, and influenza B. It has been reported that in years in which H3N2 is the predominant strain, there are more hospitalizations. In the 2014-2015 vaccines, circulating A/H3N2 influenza viruses drifted significantly after strain selection, contributing to a drastic decrease of vaccine efficacy to only 19% during that season. Similarly, measured resistance to the standard antiviral drugs amantadine and rimantadine in H3N2 has increased from 1% in 1994 to 12% in 2003 to 91% in 2005.

Therefore, there remains a need to provide immunogenic compositions capable of eliciting a broad, rapid and robust immune response against Influenza virus.

### SUMMARY OF THE INVENTION

In a first aspect, the invention provides an immunogenic composition comprising:
(a) a first hemagglutinin (HA) antigen or a first nucleic acid, suitably mRNA, encoding the first HA antigen wherein the first HA antigen is derived from a strain of subtype H3 of Influenza A virus, suitably H3N2; and
(b) a second HA antigen or a second nucleic acid, suitably mRNA, encoding the second HA antigen wherein the second HA antigen is derived from a strain of subtype H1 of Influenza A virus, suitably H1N1, wherein the ratio of (a):(b) is comprised between 1.5:1 and 20:1, optionally between 1.5:1 and 5:1.

Also provided herein is an immunogenic composition comprising:
(a) a first mRNA encoding a HA of a strain of subtype H3 of Influenza A virus, suitably H3N2;
(b) a second mRNA encoding a HA of a strain of subtype H1 of Influenza A virus, suitably H1N1;
(c¹) a third mRNA encoding a HA of a first strain of Influenza B virus, suitably from B/Victoria lineage; and
(c²) optionally, a fourth mRNA encoding a HA of a second strain of Influenza B virus, suitably from B/Yamagata lineage,
   wherein the ratio of (a):(b): (c¹): (c²) is comprised between 1.5:1:1:1 and 20:1:1:1, optionally between 1.5:1:1:1 and 5:1:1:1.

In a second aspect, the invention provides a vaccine comprising the immunogenic composition as defined herein.

In a third aspect, the invention provides a kit or kit of parts comprising the antigens or the nucleic acids and/or the mRNAs, suitably the mRNAs of (a), (b), (c¹), (c²), (c³), (c⁴), (c⁵) and/or (c⁶), as in defined herein, optionally comprising a liquid vehicle for solubilizing, and, optionally, technical instructions providing information on administration and dosage of the components.

In a fourth aspect, the invention relates to the immunogenic composition, the vaccine, or the kit or kit of parts as described herein, for use as a medicament.

In a fifth aspect, the invention relates to the immunogenic composition, the vaccine, or the kit or kit of parts as described herein, for use in the treatment or prophylaxis of an infection with an Influenza virus, suitably an Influenza A and/or Influenza B.

In a sixth aspect, the invention relates to a method of treating or preventing a disorder or disease caused by an Influenza virus, suitably an Influenza A and/or Influenza B, wherein the method comprises applying or administering to a subject in need thereof the immunogenic composition, the vaccine or the kit or kit of parts as described herein.

In a seventh aspect, the invention relates to a method of eliciting an immune response, wherein the method comprises applying or administering to a subject in need thereof the immunogenic composition, the vaccine or the kit or kit of parts as described herein.

### BRIEF DESCRIPTION OF THE SEQUENCES

SEQ ID NO: 1 Amino acid sequence of HA from A/Michigan/45/2015 (H1N1).

SEQ ID NO: 2 Amino acid sequence of NA from A/Michigan/45/2015 (H1N1).

SEQ ID NO: 3 Amino acid sequence of HA from A/Switzerland/8060/2017 (H3N2).

SEQ ID NO: 4 Amino acid sequence of NA from A/Switzerland/8060/2017 (H3N2).

SEQ ID NO: 5 Amino acid sequence of HA from B/Colorado/06/2017.

SEQ ID NO: 6 Amino acid sequence of NA from B/Colorado/06/2017.

SEQ ID NO: 7 Amino acid sequence of HA from B/Phuket/3073/2013.

SEQ ID NO: 8 Amino acid sequence of NA from B/Phuket/3073/2013.

SEQ ID NO: 9 Amino acid sequence of HA from A/Singapore/INFIMH-16-0019/2016 (H3N2).

SEQ ID NO: 10 Amino acid sequence of NA from A/Singapore/INFIMH-16-0019/2016 (H3N2).

SEQ ID NO: 11 Amino acid sequence of HA from A/Brisbane/02/2018 (H1N1).

SEQ ID NO: 12 Amino acid sequence of NA from A/Brisbane/02/2018 (H1N1).

SEQ ID NO: 13 Amino acid sequence of HA from A/Kansas/14/2017 (H3N2).

SEQ ID NO: 14 Amino acid sequence of NA from A/Kansas/14/2017 (H3N2).

SEQ ID NO: 15 Amino acid sequence of HA from A/South Australia/34/2019 (H3N2).

SEQ ID NO: 16 Amino acid sequence of NA from A/South Australia/34/2019 (H3N2).

SEQ ID NO: 17 Amino acid sequence of HA from B/Washington/02/2019.

SEQ ID NO: 18 Amino acid sequence of NA from B/Washington/02/2019.

SEQ ID NO: 19 Amino acid sequence of HA from A/Guangdong-Maonan/SWL1536/2019 (H1N1).

SEQ ID NO: 20 Amino acid sequence of NA from A/Guangdong-Maonan/SWL1536/2019 (H1N1).

SEQ ID NO: 21 Amino acid sequence of HA from A/Hong Kong/2671/2019 (H3N2).

SEQ ID NO: 22 Amino acid sequence of NA from A/Hong Kong/2671/2019 (H3N2).

SEQ ID NO: 23 Amino acid sequence of HA from A/Hawaii/70/2019 (H1N1).

SEQ ID NO: 24 Amino acid sequence of NA from A/Hawaii/70/2019 (H1N1).

SEQ ID NO: 25 Amino acid sequence of HA from A/Hong Kong/45/2019 (H3N2).

SEQ ID NO: 26 Amino acid sequence of NA from A/Hong Kong/45/2019 (H3N2).

SEQ ID NO: 27 Amino acid sequence of HA from A/Victoria/2570/2019 (H1N1).

SEQ ID NO: 28 Amino acid sequence of NA from A/Victoria/2570/2019 (H1N1).

SEQ ID NO: 29 Amino acid sequence of HA from A/Wisconsin/588/2019 (H1N1).

SEQ ID NO: 30 Amino acid sequence of NA from A/Wisconsin/588/2019 (H1N1).

SEQ ID NO: 31 Amino acid sequence of HA from A/Cambodia/e0826360/2020 (H3N2).

SEQ ID NO: 32 Amino acid sequence of NA from A/Cambodia/e0826360/2020 (H3N2).

SEQ ID NO: 33 Amino acid sequence of HA from A/Darwin/9/2021 (H3N2).

SEQ ID NO: 34 Amino acid sequence of NA from A/Darwin/9/2021 (H3N2).

SEQ ID NO: 35 Amino acid sequence of HA from B/Austria/1359417/2021.

SEQ ID NO: 36 Amino acid sequence of NA from B/Austria/1359417/2021.

SEQ ID NO: 37 Amino acid sequence of HA from A/Darwin/6/2021 (H3N2).

SEQ ID NO: 38 Amino acid sequence of NA from A/Darwin/6/2021 (H3N2).

SEQ ID NO: 39 Amino acid sequence of HA from A/Victoria/4897/2022 (H1N1)

SEQ ID NO: 40 Amino acid sequence of NA from A/Victoria/4897/2022 (H1N1)

SEQ ID NO: 41 Amino acid sequence of HA from A/Wisconsin/67/2022 (H1N1)

SEQ ID NO: 42 Amino acid sequence of NA from A/Wisconsin/67/2022 (H1N1)

SEQ ID NO: 43 Amino acid sequence of HA from A/Sydney/5/2021 (H1N1).

SEQ ID NO: 44 Amino acid sequence of NA from A/Sydney/5/2021 (H1N1).

SEQ ID NO: 45 Amino acid sequence of HA from A/Thailand/8/2022 (H3N2).

SEQ ID NO: 46 Amino acid sequence of NA from A/Thailand/8/2022 (H3N2).

SEQ ID NO: 47 Amino acid sequence of HA from A/Massachusetts/18/2022 (H3N2).

SEQ ID NO: 48 Amino acid sequence of NA from A/Massachusetts/18/2022 (H3N2).

### DESCRIPTION OF THE FIGURES

FIG. 1: Domain structure of the Influenza A virus (IAV) HA protein. Domains in HA1 include fusion (F1), vestigial esterase (VE), and receptor-binding domain (RBD). Domains in HA2 include the HA2 ectodomain, transmembrane region©, and cytoplasmic tail (CT). The HA head includes the receptor-binding and vestigial esterase subdomains. The stalk (also known as “stem”) contains the HA1 fusion domains and the HA2 ectodomain.

FIG. 2A-C: Reactogenicity assessment of subjects in the CVSQIV Phase I influenza vaccination trial. FIG. 2A, Solicited Adverse Events in subjects at the indicated mRNA dose levels shown at the bottom of the chart. FIG. 2B, Solicited Adverse Events in subjects at the indicated mRNA dose levels separated between younger and older adults. FIG. 2A-B, Grade 0 events at the bottom of the chart above the dose level indication and percentages for increasing grade events arranged vertically. FIG. 2C, Solicited Adverse Events in subjects at the indicated mRNA dose levels separated for younger and older adults and separated between local and systemic events. Grade 0-1 events at the bottom of the chart above the dose level indication. The percentages for Grade 0-1 versus Grade≥2 are shown.

FIG. 3A-D: Graphs show the Geometric Mean Titer (95% CI) for Hemagglutinin Inhibition Assay (HAI) Assay in the Per Protocol Immunogenicity Set. Left panels show the HAI titers for all subjects at Day 1, Day 22 and Day 183 at the indicated vaccine mRNA dose levels. Data in the right panels are separated between younger adults (YA) and older adults (OA) at the indicated mRNA dose levels. Data are shown separately for each of the HA components encoded by the vaccine mRNA: H1N1 (FIG. 3A); H3N2 (FIG. 3B); B/Phuket (FIG. 3C); and B/Washington (FIG. 3D).

FIG. 4: Seroconversion rates (SCR) from HAI assay. The table in upper-left panel shows SCR (is defined as <1:10 pre-vaccination titers, the post-vaccination titers should be ≥1:40; if ≥1:10 pre-vaccination titers, the post-vaccination titers should be >four-fold increase from baseline). Data are shown for each encoded HA, at each dose level and either for all subjects or separated between younger and older adults. The graph in the lower left panel shows overall SCR for each encoded HA, at each dose level. The graph in the upper right panel shows SCR for each encoded HA, at each dose level, in younger adults. The graph in the lower right panel shows SCR for each encoded HA, at each dose level, in older adults.

FIG. 5: Shows the percentage of study subjects that exhibited a≥four-fold increase in anti-HA titer by microneutralization (MN) assay. The table in upper-left panel shows the percentage of subjects with >four-fold increase in anti-HA titer by MN assay. Data are shown for each encoded HA, at each dose level, and either for all subjects or separated between younger and older adults. The graph in the lower left panel shows overall 4-fold anti-HA increase by MN assay for each encoded HA, at each dose level. The graph in the upper right panel shows 4-fold anti-HA increase by MN assay for each encoded HA, at each dose level, in younger adults. The graph in the lower right panel shows 4-fold anti-HA increase by MN assay for each encoded HA, at each dose level, in older adults.

FIG. 6: Shows the percentage of study subjects that exhibited a≥four-fold increase in anti-NA titer by enzyme linked lectin assay (ELLA) assay. The table in upper-left panel shows the percentage of subjects with >four-fold increase anti-NA titer by ELLA assay. Data are shown for each encoded NA, at each dose level, and either for all subjects or separated between younger and older adults. The graph in the lower left panel shows overall 4-fold anti-NA increase by ELLA assay for each encoded NA, at each dose level. The graph in the upper right panel shows 4-fold anti-NA increase by ELLA assay for each encoded NA, at each dose level, in younger adults. The graph in the lower right panel shows 4-fold anti-NA increase by ELLA assay for each encoded NA, at each dose level, in older adults.

FIG. 7A-D: Shows HI titers induced by 4- or 7-component mRNA vaccines containing unmodified or modified (ψ and N1-mψ) nucleosides with equimolar proportions between the mRNA sequences. Female Balb/c mice (n=10/group) were vaccinated with 0.56 μg or 2.84 μg of the 4-component (4HA; unmodified, ψ and N1-mψ) and 1 μg or 2.84 μg of 7-component (4HA+3NA; unmodified, ψ and N1-mψ) mRNA-LNP vaccines. Control animals (n=5/group) received either physiological saline (NaCl) or one tenth of the human dose of the licensed QIVs FLUARIX Tetra NH21-22 or FLUZONE HD NH21-22. HI titers against influenza A/Wisconsin/588/2019 (H1N1pdm09) (A), A/Cambodia/e0826360/2020 (H3N2) (B) B/Washington/02/2019 (C) and B/Phuket/3073/2013 (D) were measured in serum of the mice two weeks post second immunization.

FIG. 8A-C: Shows NI titers induced by 7-component mRNA vaccines containing unmodified or modified (ψ and N1-mψ) nucleosides with equimolar proportions between the mRNA sequences. Female Balb/c mice (n=10/group) were vaccinated with 1 μg or 2.84 μg of 7-component (4HA+3NA; unmodified, ψ and N1-mψ) mRNA vaccines. Control animals (n=5/group) received either physiological saline (NaCl) or one tenth of the human dose of the licensed QIVs FLUARIX Tetra (NH21-22) or FLUZONE HD (NH21-22). NI titers against influenza A/Wisconsin/588/2019 (H1N1pdm09) (A), A/Cambodia/e0826360/2020 (H3N2) (B) and B/Washington/02/2019 (C) were measured in serum two weeks post second immunization.

FIG. 9A-D: Shows HI responses induced upon i.m. immunization of naive ferrets with 4-component and 8-component Flu Seasonal mRNA vaccine formulations. Female ferrets were immunized via i.m. route on Day 0 and 28 with 12.5 μg and 25 μg of the 4-component and 25 μg and 50 μg of the 8-component Flu Seasonal N1my mRNA vaccines (n=6). Control animals received either physiological saline (NaCl) (n=6/group) or full human dose of the licensed split-inactivated QIVs FLUARIX Tetra (NH22-23) (n=6/group) via i.m. route on Day 0 and 28. HI titers against influenza A/Wisconsin/588/2019 (H1N1pdm09) (A), A/Darwin/6/2021 (H3N2) (B), B/Austria/1359417/2021 (C) and B/Phuket/3073/2013 (D) were measured in serum of vaccinated animals collected on Day 55.

FIG. 10A-D: Shows microneutralization (MN) titers induced upon i.m. immunization of naive ferrets with 4-component and 8-component Flu Seasonal mRNA vaccine formulations. Female ferrets were immunized via i.m. route on Day 0 and 28 with 12.5 μg and 25 μg of the 4-component and 25 μg and 50 μg of the 8-component Flu Seasonal N1my mRNA vaccines (n=6). Control animals received either physiological saline (NaCl) (n=6/group) or full human dose of the licensed split-inactivated QIVs FLUARIX Tetra (NH22-23) (n=6/group) via i.m. route on Day 0 and 28. MN titers against influenza A/Wisconsin/588/2019 (H1N1pdm09) (A), A/Darwin/6/2021 (H3N2) (B), B/Austria/1359417/2021 (C) and B/Phuket/3073/2013 (D) were measured in serum of vaccinated animals collected on Day 55.

FIG. 11A-D: Shows neuraminidase inhibition (NI) titers determined using the enzyme linked lectin assay (ELLA) upon i.m. immunization of naive ferrets with 4-component and 8-component Flu Seasonal mRNA vaccine formulations. Female ferrets were immunized via i.m. route on Day 0 and 28 with 12.5 μg and 25 μg of the 4-component and 25 μg and 50 μg of the 8-component Flu Seasonal N1mw mRNA vaccines (n=6). Control animals received either physiological saline (NaCl) (n=6/group) or full human dose of the licensed split-inactivated QIVs FLUARIX Tetra (NH22-23) (n=6/group) via i.m. route on Day 0 and 28. NI titers against influenza A/Wisconsin/588/2019 (H1N1pdm09) (A), A/Darwin/6/2021 (H3N2) (B), B/Austria/1359417/2021 (C) and B/Phuket/3073/2013 (D) were measured in serum of vaccinated animals collected on Day 55.

FIG. 12A-D Shows HI titers induced upon immunization of human healthy adults (18-50 years old) with 1-component, 4-component and 8-component Flu Seasonal mRNA vaccine formulations. The control is a Flu D-QIV (FLUARIX, NH 2022-23). HI titers against influenza A/Victoria/2570/2019 (H1N1pdm09) (A), A/Darwin/6/2021 (H3N2) (B), B/Connecticut/01/2021 (C) and B/Phuket/3073/2013 (D) were measured on Day 29.

FIG. 13A-D Shows NI titers induced upon immunization of human healthy adults (18-50 years old) with 1-component, 4-component and 8-component Flu Seasonal mRNA vaccine formulations. The control is a Flu D-QIV (FLUARIX, NH 2022-23). NI titers against influenza A/Wisconsin/588/2019 (H1N1pdm09) (A), Flu A/Cambodia/e0826360/2020 (H3N2) (B), Flu B/Austria/1359417/2021 (C) and B/Phuket/3073/2013 (D) were measured on Day 29. FIG. 14A-D

Shows the percentage of human healthy adults (18-50 years old) with solicited events (any; A), local events (B) and systemic events (C) within 7 days of immunization with 1-component, 4-component and 8-component Flu Seasonal mRNA vaccine formulations. The control is a Flu D-QIV (FLUARIX, NH 2022-23). (D) shows the overall summary by event including grade 3 events.

FIG. 15 Shows the percentage of human healthy adults (18-50 years old) with related unsolicited events within 7 days of immunization with 1-component, 4-component and 8-component Flu Seasonal mRNA vaccine formulations. The control is a Flu D-QIV (FLUARIX, NH 2022-23).

### DETAILED DESCRIPTION OF THE INVENTION

The present application is filed together with a sequence listing in electronic format, which is part of the description (WIPO standard ST.26). The information contained in the sequence listing is incorporated herein by reference in its entirety. Where reference is made herein to a “SEQ ID NO”, the corresponding nucleic acid (n.a.) sequence or amino acid (aa) sequence in the sequence listing having the respective identifier is referred to. For many sequences, the sequence listing also provides additional detailed information, e.g. regarding certain structural features, sequence optimizations, GenBank (NCBI) or GISAID (epi) identifiers, or additional detailed information regarding its coding capacity. Where reference is made to “SEQ ID NOs” of other published patent applications or patents, said sequences, e.g. amino acid sequences or nucleic acid sequences, are explicitly incorporated herein by reference. Accordingly, these sequences constitute an integral part of the underlying description.

### Immunogenic Composition

Protective immune responses induced by vaccination against Influenza viruses are primarily directed to the viral HA protein, which is a glycoprotein on the surface of the virus responsible for interaction of the virus with host cell receptors.

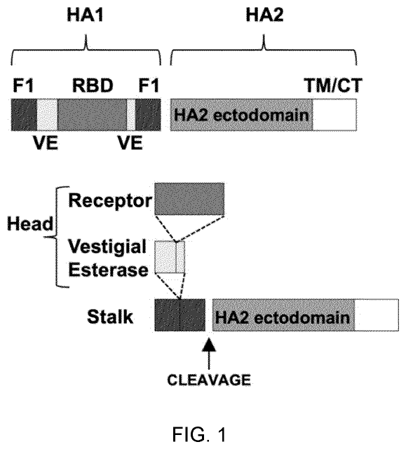

HA proteins on the virus surface are homotrimers of HA protein monomers that are enzymatically cleaved to yield amino-terminal HA1 and carboxy-terminal HA2 polypeptides. Structurally, hemagglutinin proteins are comprised of several domains: a globular head domain, a stalk domain (also referred to as a stem domain), a transmembrane domain, and a cytoplasmic domain (see FIG. 1, Russell et al., 2021).

It is generally thought that during infection of a host cell (e.g., a eukaryotic cell such as a human cell) with an Influenza virus, the hemagglutinin protein recognizes and binds to sialic acid of a receptor on the surface of a host cell facilitating attachment of the virus to the host cell. Following endocytosis of the virus and acidification of the endosome, the hemagglutinin protein undergoes a pH-dependent conformational change that allows for the hemagglutinin protein to facilitate fusion of the viral envelope with the endosome membrane of host cell and entry of the viral nucleic acid into the host cell.

The globular head consists exclusively of the major portion of the HA1 polypeptide, whereas the stem that anchors the HA protein into the viral lipid envelope is comprised of HA2 and part of HA1. The globular head of a HA protein includes two domains: the receptor binding domain (RBD), a domain that includes the sialic acid-binding site, and the vestigial esterase domain, a smaller region just below the RBD. In general, Influenza viruses are classified based on the amino acid sequences of the viral hemagglutinin protein and/or the amino acid sequence of the viral neuraminidase (NA). The differences in amino acid sequence between HA proteins of different subtypes are largely found within the sequence of the head domain of the protein. The amino acid sequence of the stalk domain is considered to be more conserved between HA subtypes compared to sequences of the head domain. Domains of the HA protein may be predicted using conventional methods known in the art.

Many naturally occurring and experimentally derived antibodies that bind and neutralize the HA protein are thought to bind epitopes within the head domain of HA and prevent or reduce interaction of HA with sialic acid on receptors of host cells, thereby preventing or reducing infection of the cell. Alternatively, or in addition, neutralizing antibodies may prevent or reduce fusion of the virus membrane with the membrane of the endosome. Such antibodies may bind epitopes within the stalk domain, thereby inhibiting the conformations change of the protein. Antibodies against Influenza often target variable antigenic sites in the globular head of HA and thus, neutralize only antigenically closely related viruses. The variability of the HA head is due to the constant antigenic drift (i.e., changes in the protein sequence) of Influenza viruses and is responsible for seasonal endemics of Influenza.

The inventors overcame the drawbacks of the prior art by administering an immunogenic composition comprising:
(a) a first hemagglutinin (HA) antigen or a first nucleic acid, suitably mRNA, encoding the first HA antigen wherein the first HA antigen is derived from a strain of subtype H3 of Influenza A virus suitably H3N2; and
(b) a second HA antigen or a second nucleic acid, suitably mRNA, encoding the second HA antigen wherein the second HA antigen is derived from a strain of subtype H1 of Influenza A virus, suitably H1N1,
   wherein the ratio of (a):(b) is comprised between 1.5:1 and 20:1, optionally between 1.5:1 and 5:1.

It has been found that the immunogenic compositions of the invention induce a broad, rapid, and robust immune response against Influenza virus, such as Influenza A and/or B.

In particular, or in addition, it has been found that the efficacy against different strains of Influenza virus of immunogenic compositions comprising (a) a first HA antigen or a first nucleic acid, suitably mRNA, encoding the first HA antigen which is derived from a strain of subtype H3 of Influenza A virus and (b) a second HA antigen or a second nucleic acid, suitably mRNA, encoding the second HA antigen which is derived from a strain of subtype H1 of Influenza A virus is enhanced when the ratio of (a):(b) is comprised between 1.5:1 and 20:1, optionally between 1.5:1 and 5:1.

In particular, or in addition, it has been found that the immunogenicity associated with the first HA antigen and/or the second HA antigen forming the immunogenic compositions of the invention is enhanced when the ratio of (a):(b) is comprised between 1.5:1 and 20:1, optionally between 1.5:1 and 5:1.

Suitably, the immunogenic compositions of the invention have at least some of the following advantageous features:
Translation of the nucleic acid, suitably mRNAs, encoding the first and second HA antigens at the site of injection/vaccination (e.g. muscle);
Induction of antigen-specific immune responses, suitably at a low dosage and dosing regimen;
Suitability for vaccination of infants and/or newborns or the elderly, in particular the elderly;
Suitability of the composition/vaccine for intramuscular administration;
Induction of specific and functional humoral immune response against Influenza virus, suitably Influenza A and/or B virus;
Induction of broad, functional cellular T-cell responses against Influenza virus, suitably Influenza A and/or B virus;
Induction of specific B-cell memory against Influenza virus, suitably Influenza A and/or B virus;
Induction of functional antibodies that can effectively neutralize the Influenza virus, suitably Influenza A and/or B virus;
Induction of functional antibodies that can effectively neutralize emerging variants of Influenza virus, suitably Influenza A and/or B virus;
Induction of protective immunity against Influenza virus infection, e.g. against Influenza A virus and/or Influenza B virus, or emerging variants thereof;
Fast onset of immune protection against Influenza virus, suitably Influenza A virus and/or Influenza B virus;
Longevity of the induced immune responses against Influenza virus, suitably Influenza A virus and/or Influenza B virus;
No enhancement of a virus infection (e.g. Influenza virus infection) due to vaccination or immunopathological effects;
No antibody dependent enhancement (ADE) caused by the nucleic acid-based composition/vaccine;
No excessive induction of systemic cytokine or chemokine response after application of the composition/vaccine, which could lead to an undesired high reactogenicity upon injection/vaccination;
Well tolerability, no side-effects, non-toxicity of the composition/vaccine;
Advantageous stability characteristics of the nucleic acid-based composition/vaccine;
Speed, adaptability, simplicity and scalability of the nucleic acid-based composition/vaccine production;
Advantageous injection/vaccination regimen that only requires a low dose of the composition/vaccine for sufficient protection.

Therefore, in a first aspect, the invention relates to an immunogenic composition comprising:
(a) a first hemagglutinin (HA) antigen or a first nucleic acid, suitably mRNA, encoding the first HA antigen wherein the first HA antigen is derived from a strain of subtype H3 of Influenza A virus; and
(b) a second HA antigen or a second nucleic acid, suitably mRNA, encoding the second HA antigen wherein the second HA antigen is derived from a strain of subtype H1 of Influenza A virus,
   wherein the ratio of (a):(b) is comprised between 1.5:1 and 20:1, optionally between 1.5:1 and 5:1.

In some embodiments, the ratio of (a):(b) is greater than 1:1.

In some embodiments, the ratio of (a):(b) is greater than 1:1 and lower or equal to 20:1.

In some embodiments, the ratio of (a):(b) is greater than 1:1 and lower or equal to 5:1.

In some embodiments, the ratio is a weight/weight ratio or a molar ratio. Suitably, the ratio is a weight/weight ratio.

As used herein, a “weight/weight ratio” or wt/wt ratio or wt: wt ratio refers to the ratio between the weights (masses) of the different components. A “molar ratio” refers to the ratio between different components (e.g., the number of mRNA encoding each antigen).

The terms “hemagglutinin”, “hemagglutinin protein”, and “HA” may be used interchangeably throughout and refer to a hemagglutinin protein that may be present on the surface of an Influenza virus.

In some embodiments, said strain of subtype H3 of Influenza A virus is selected from Influenza A viruses characterized by a neuraminidase (NA) selected from the group consisting of N1, N2, and N8, suitably N2.

In some embodiments, said strain of subtype H1 of Influenza A virus is selected from Influenza A viruses characterized by a neuraminidase (NA) selected from the group consisting of N1 and N2, more suitably N1.

The terms “neuraminidase”, “neuraminidase protein”, and “NA” may be used interchangeably throughout and refer to a neuraminidase protein that may be present on the surface of an Influenza virus.

In some embodiments, said strain of subtype H3 of Influenza A virus is selected from the group consisting of H3N1, H3N2 and H3N8, suitably H3N2.

In some embodiments, said strain of subtype H1 of Influenza A virus is selected from the group consisting of H1N1 and H1N2, suitably H1N1.

In some embodiments, said strain of Influenza A H1N1 virus is selected from the group consisting of A/Beijing/262/95 (H1N1)-like virus, A/New Caledonia/20/99 (H1N1)-like virus, A/Solomon Islands/3/2006 (H1N1)-like virus, A/Brisbane/59/2007 (H1N1)-like virus, A/California/7/2009 (H1N1)-like virus, A/California/7/2009 (H1N1) pdm09-like virus, A/Michigan/45/2015 (H1N1) pdm09-like virus, A/Victoria/2570/2019 (H1N1) pdm09-like virus, A/Wisconsin/588/2019 (H1N1) pdm09-like virus, A/Guangdong-Maonan/SWL1536/2019

(H1N1) pdm09-like virus, A/Hawaii/70/2019 (H1N1) pdm09-like virus, A/Brisbane/02/2018 (H1N1) pdm09-like virus, A/Christchurch/and 16/2010 A/South Dakota/6/2007, A/Victoria/4897/2022 (H1N1) pdm09-like virus, A/Wisconsin/67/2022 (H1N1) pdm09-like virus and A/Sydney/5/2021 (H1N1) pdm09-like virus.

In some embodiments, said strain of Influenza A H1N1 virus is selected from an Influenza A virus as listed in Table 1 and/or Table 2.

In some embodiments, said strain of Influenza A H1N1 virus is selected from an Influenza A virus which is recommended for Influenza virus vaccine composition by the WHO (https://www.who.int/teams/global-influenza-programme/vaccines/who-recommendations).

In some embodiments, said strain of Influenza A H3N2 virus is selected from the group consisting of A/Thailand/8/2022 (H3N2)-like virus, A/Massachusetts/18/2022 (H3N2)-like virus, A/Sydney/5/97 (H3N2)-like virus, A/Moscow/10/99 (H3N2)-like virus, A/Panama/2007/99, A/Fujian/411/2002 (H3N2)-like virus, A/Wyoming/3/2003, A/Kumamoto/102/2002, A/Wellington/1/2004 (H3N2)-like virus, A/California/7/2004 (H3N2)-like virus, A/New York/55/2004, A/Wisconsin/67/2005 (H3N2)-like virus, A/Hiroshima/52/2005, A/Brisbane/10/2007 (H3N2)-like virus, A/Uruguay/716/2007, A/Perth/16/2009 (H3N2)-like virus, A/Wisconsin/15/2009, A/Victoria/210/2009, A/Victoria/361/2011 (H3N2)-like virus, A/Ohio/2/2012, A/Maryland/2/2012, A/South Australia/30/2012, A/Brisbane/1/2012, A/Brisbane/6/2012, A (H3N2) virus antigenically like the cell-propagated prototype virus A/Victoria/361/2011, A/Texas/50/2012 (H3N2)-like virus, A/Darwin/9/2021 (H3N2)-like virus, A/Darwin/6/2021 (H3N2)-like virus, A/Cambodia/e0826360/2020 (H3N2)-like virus, A/Hong Kong/2671/2019 (H3N2)-like virus,

A/Hong Kong/45/2019 (H3N2)-like virus, A/Switzerland/9715293/2013 (H3N2)-like virus, A/South Australia/55/2014, A/Norway/466/2014, A/Stockholm/6/2014, A/Hong Kong/4801/2014 (H3N2)-like virus, A/Singapore/INFIMH-16-0019/2016 (H3N2)-like virus, A/Switzerland/8060/2017 (H3N2)-like virus, A/Kansas/14/2017 (H3N2)-like virus and A/South Australia/34/2019 (H3N2)-like virus.

In some embodiments, said strain of Influenza A H3N2 virus is selected from an Influenza A virus as listed in Table 1 and/or Table 2.

In some embodiments, said strain of Influenza A H3N2 virus is selected from an Influenza A virus which is recommended for Influenza virus vaccine composition by the WHO (https://www.who.int/teams/global-influenza-programme/vaccines/who-recommendations).

In some embodiments, the composition does not comprise a HA antigen or a nucleic acid, suitably mRNA, encoding a HA antigen from an influenza strain which is not recommended by WHO.

In some embodiments, the composition does not comprise a HA antigen or a nucleic acid, suitably mRNA, encoding a HA antigen identified or designed by machine learning.

TABLE 1
Recommended composition of Influenza virus vaccines for
use in the 1998-2025 northern hemisphere influenza season

| | | | | B strain (for |
|---|---|---|---|---|
| | | | | quadrivalent |
| Year | H1N1 strain | H3N2 strain | B strain | vaccines) |
| 1998- | A/Beijing/262/95 | A/Sydney/5/97(H3N2)- | B/Beijing/184/93-like | / |
| 1999 | (H1N1)-like virus | like virus | virus (The most widely | |
| | | | used vaccine virus is | |
| | | | B/Harbin/7/94) | |
| 1999- | A/Beijing/262/95 | A/Sydney/5/97(H3N2)- | B/Beijing/184/93-like | / |
| 2000 | (H1N1)-like virus | like virus | virus (The most widely | |
| | | | used vaccine virus is | |
| | | | B/Harbin/7/94) or | |
| | | | B/Shangdong/7/97-like | |
| | | | virus | |
| 2000- | A/New | A/Moscow/10/99 | B/Beijing/184/93-like | / |
| 2001 | Caledonia/20/99 | (H3N2)-like virus | virus (The most widely | |
| | (H1N1)-like virus | (A/Panama/2007/99 is | used vaccine strain is | |
| | | an A/Moscow/10/99 | B/Yamanashi/166/98) | |
| | | (H3N2)-like virus) | | |
| 2001- | A/New | A/Moscow/10/99 | B/Sichuan/379/99-like | / |
| 2002 | Caledonia/20/99 | (H3N2)-like virus | virus | |
| | (H1N1)-like virus | (A/Panama/2007/99 is | (B/Johannesburg/5/99 | |
| | | an A/Moscow/10/99 | and B/Victoria/504/ | |
| | | (H3N2)-like virus) | 2000 are | |
| | | | B/Sichuan/379/99-like | |
| | | | viruses) | |
| 2002- | A/New | A/Moscow/10/99 | B/Hong | / |
| 2003 | Caledonia/20/99 | (H3N2)-like virus | Kong/330/2001-like | |
| | (H1N1)-like virus | (A/Panama/2007/99 is | virus | |
| | | an A/Moscow/10/99 | | |
| | | (H3N2)-like virus) | | |
| 2003- | A/New | A/Moscow/10/99 | B/Hong | / |
| 2004 | Caledonia/20/99 | (H3N2)-like virus | Kong/330/2001-like | |
| | (H1N1)-like virus | (A/Panama/2007/99 is | virus (Used vaccine | |
| | | an A/Moscow/10/99 | strains include | |
| | | (H3N2)-like virus) | B/Shandong/7/97, | |
| | | | B/Hong | |
| | | | Kong/330/2001, | |
| | | | B/Hong | |
| | | | Kong/1434/2002) | |
| 2004- | A/New | A/Fujian/411/2002(H3N2)- | B/Shanghai/361/ | / |
| 2005 | Caledonia/20/99 | like virus (Used | 2002-like virus | |
| | (H1N1)-like virus | vaccine virus is | (Candidate vaccine | |
| | | A/Wyoming/3/2003. | viruses include | |
| | | A/Kumamoto/102/ | B/Shanghai/361/2002 | |
| | | 2002 is also available | and B/Jilin/20/2003/ | |
| | | as a vaccine virus) | which is a | |
| | | | B/Shanghai/361/ | |
| | | | 2002-like virus) | |
| 2005- | A/New | A/California/7/ | B/Shanghai/361/ | / |
| 2006 | Caledonia/20/99 | 2004(H3N2)-like virus | 2002-like virus | |
| | (H1N1)-like virus | (A/New York/55/2004 | (Candidate vaccine | |
| | | is available as a | viruses include | |
| | | vaccine virus) | B/Shanghai/361/2002 | |
| | | | and B/Jilin/20/2003 | |
| | | | which is a | |
| | | | B/Shanghai/361/ | |
| | | | 2002-like virus) | |
| 2006- | A/New | A/Wisconsin/67/2005 | B/Malaysia/2506/ | / |
| 2007 | Caledonia/20/99 | (H3N2)-like virus | 2004-like virus | |
| | (H1N1)-like virus | (A/Wisconsin/67/2005 | (B/Malaysia/2506/2004 | |
| | | (H3N2) and | virus and | |
| | | A/Hiroshima/52/2005) | B/Ohio/1/2005) | |
| 2007- | A/Solomon | A/Wisconsin/67/2005 | B/Malaysia/2506/ | / |
| 2008 | Islands/3/2006 | (H3N2)-like virus | 2004-like virus | |
| | (H1N1)-like virus | (A/Wisconsin/67/2005 | | |
| | | (H3N2) and | | |
| | | A/Hiroshima/52/2005) | | |
| 2008- | A/Brisbane/59/2007 | A/Brisbane/10/2007 | B/Florida/4/2006-like | / |
| 2009 | (H1N1)-like virus | (H3N2)-like virus | virus (B/Florida/4/2006 | |
| | | | and B/Brisbane/3/ | |
| | | | 2007) | |
| 2009- | A/Brisbane/59/2007 | A/Brisbane/10/2007 | B/Brisbane/60/2008- | / |
| 2010 | (H1N1)-like virus | (H3N2)-like virus | like virus | |
| | | (A/Brisbane/10/2007 | (B/Brisbane/33/2008 | |
| | | and A/Uruguay/716/ | is a B/Brisbane/60/ | |
| | | 2007) | 2008-like virus) | |
| 2010- | A/California/7/2009 | A/Perth/16/2009 | B/Brisbane/60/2008- | / |
| 2011 | (H1N1)-like virus | (H3N2)-like virus | like virus | |
| | | (A/Wisconsin/15/2009 | | |
| | | is an A/Perth/16/2009 | | |
| | | (H3N2)-like virus and | | |
| | | is a 2010 southern | | |
| | | hemisphere vaccine | | |
| | | virus) | | |
| 2011- | A/California/7/2009 | A/Perth/16/2009 | B/Brisbane/60/2008- | / |
| 2012 | (H1N1)-like virus | (H3N2)-like virus | like virus | |
| 2012- | A/California/7/2009 | A/Victoria/361/2011 | B/Wisconsin/1/2010- | / |
| 2013 | (H1N1)pdm09-like | (H3N2)-like virus | like virus | |
| | virus | | | |
| 2013- | A/California/7/2009 | A(H3N2) virus | B/Massachusetts/2/2012- | B/Brisbane/60/2008- |
| 2014 | (H1N1)pdm09-like | antigenically like the | like virus | like virus |
| | virus (A/Christchurch/ | cell-propagated | | (B/Brisbane/33/2008 |
| | 16/2010 is an | prototype virus | | is a B/Brisbane/60/ |
| | A/California/7/2009- | A/Victoria/361/2011 | | 2008-like virus) |
| | like virus) | (A/Texas/50/2012 is | | |
| | | an A(H3N2) virus | | |
| | | antigenically like the | | |
| | | cell-propagated | | |
| | | prototype virus | | |
| | | A/Victoria/361/2011) | | |
| 2014- | A/California/7/2009 | A/Texas/50/2012 | B/Massachusetts/2/2012- | B/Brisbane/60/2008- |
| 2015 | (H1N1)pdm09-like | (H3N2)-like virus | like virus | like virus |
| | virus | | | |
| 2015- | A/California/7/2009 | A/Switzerland/9715293/ | B/Phuket/3073/2013- | B/Brisbane/60/2008- |
| 2016 | (H1N1)pdm09-like | 2013 (H3N2)-like | like virus | like virus |
| | virus | virus | | |
| 2016- | A/California/7/2009 | A/Hong | B/Brisbane/60/2008- | B/Phuket/3073/2013- |
| 2017 | (H1N1)pdm09-like | Kong/4801/2014 | like virus | like virus |
| | virus | (H3N2)-like virus | | |
| 2017- | A/Michigan/45/2015 | A/Hong | B/Brisbane/60/2008- | B/Phuket/3073/2013- |
| 2018 | (H1N1)pdm09-like | Kong/4801/2014 | like virus | like virus |
| | virus | (H3N2)-like virus | | |
| 2018- | A/Michigan/45/2015 | A/Singapore/INFIMH- | B/Colorado/06/2017- | B/Phuket/3073/2013- |
| 2019 | (H1N1)pdm09-like | 16-0019/2016 (H3N2)- | like virus | like virus |
| | virus | like virus | (B/Victoria/2/87 | (B/Yamagata/16/88 |
| | | | lineage) | lineage) |
| 2019- | A/Brisbane/02/2018 | A/Kansas/14/2017 | B/Colorado/06/2017- | B/Phuket/3073/2013- |
| 2020 | (H1N1)pdm09-like | (H3N2)-like virus | like virus | like virus |
| | virus | | (B/Victoria/2/87 | (B/Yamagata/16/88 |
| | | | lineage) | lineage) |
| 2020- | A/Guangdong- | A/Hong | B/Washington/02/2019 | B/Phuket/3073/2013 |
| 2021 | Maonan/SWL1536/2019 | Kong/2671/2019 | (B/Victoria lineage)- | (B/Yamagata lineage)- |
| | (H1N1)pdm09-like | (H3N2)-like virus or | like virus | like virus |
| | virus or | A/Hong Kong/45/2019 | | |
| | A/Hawaii/70/2019 | (H3N2)-like virus | | |
| | (H1N1)pdm09-like | | | |
| | virus | | | |
| 2021- | A/Victoria/2570/2019 | A/Cambodia/e0826360/ | B/Washington/02/2019 | B/Phuket/3073/2013 |
| 2022 | (H1N1)pdm09-like | 2020 (H3N2)-like | (B/Victoria lineage)- | (B/Yamagata lineage)- |
| | virus or | virus | like virus | like virus |
| | A/Wisconsin/588/ | | | |
| | 2019 (H1N1)pdm09- | | | |
| | like virus | | | |
| 2022- | A/Victoria/2570/2019 | A/Darwin/9/2021 | B/Austria/1359417/2021 | B/Phuket/3073/2013 |
| 2023 | (H1N1)pdm09-like | (H3N2)-like virus or | (B/Victoria | (B/Yamagata lineage)- |
| | virus or | A/Darwin/6/2021 | lineage)-like virus | like virus |
| | A/Wisconsin/588/ | (H3N2)-like virus | | |
| | 2019 (H1N1)pdm09- | | | |
| | like virus | | | |
| 2023- | A/Victoria/4897/2022 | A/Darwin/9/2021 | B/Austria/1359417/2021 | B/Phuket/3073/2013 |
| 2024 | (H1N1) pdm09-like | (H3N2)-like virus or | (B/Victoria | (B/Yamagata lineage)- |
| | virus or | A/Darwin/6/2021 | lineage)-like virus | like virus |
| | A/Wisconsin/67/ | (H3N2)-like virus | | |
| | 2022(H1N1) pdm09- | | | |
| | like virus | | | |
| 2024- | A/Victoria/4897/2022 | A/Thailand/8/2022 | B/Austria/1359417/ | B/Phuket/3073/2013 |
| 2025 | (H1N1)pdm09-like | (H3N2)-like virus or | 2021 (B/Victoria | (B/Yamagata lineage)- |
| | virus or | A/Massachusetts/18/ | lineage)-like virus | like virus |
| | A/Wisconsin/67/2022 | 2022 (H3N2)-like virus | | |
| | (H1N1)pdm09-like | | | |
| | virus | | | |

TABLE 2
Recommended composition of Influenza virus vaccines for
use in the 1999-2024 southern hemisphere influenza season

| | | | | B strain (for |
|---|---|---|---|---|
| | | | | quadrivalent |
| Year | H1N1 strain | H3N2 strain | B strain | vaccines) |
| 1999 | A/Beijing/262/95(H1N1)- | A/Sydney/5/97(H3N2)- | B/Beijing/184/93-like | / |
| | like virus | like virus | virus (The most widely | |
| | | | used vaccine virus is | |
| | | | B/Harbin/7/94) | |
| 2000 | A/New | A/Moscow/10/99 | B/Beijing/184/93-like | / |
| | Caledonia/20/99 | (H3N2)-like virus | virus (The most widely | |
| | (H1N1)-like virus | | used vaccine strain is | |
| | | | B/Yamanashi/166/98) | |
| | | | or B/Shangdong/7/97- | |
| | | | like virus | |
| 2001 | A/New | A/Moscow/10/99 | B/Sichuan/379/99-like | / |
| | Caledonia/20/99 | (H3N2)-like virus | virus | |
| | (H1N1)-like virus | (A/Panama/2007/99 is | | |
| | | an A/Moscow/10/99 | | |
| | | (H3N2)-like virus) | | |
| 2002 | A/New | A/Moscow/10/99 | B/Sichuan/379/99-like | / |
| | Caledonia/20/99 | (H3N2)-like virus | virus | |
| | (H1N1)-like virus | (A/Panama/2007/99 is | (B/Guangdong/120/2000, | |
| | | an A/Moscow/10/99 | B/Johannesburg/5/99 | |
| | | (H3N2)-like virus) | and B/Victoria/504/ | |
| | | | 2000 are | |
| | | | B/Sichuan/379/99-like | |
| | | | viruses) | |
| 2003 | A/New | A/Moscow/10/99 | B/Hong | / |
| | Caledonia/20/99 | (H3N2)-like virus | Kong/330/2001-like | |
| | (H1N1)-like virus | (A/Panama/2007/99 is | virus (Used vaccine | |
| | | an A/Moscow/10/99 | strains include | |
| | | (H3N2)-like virus) | B/Shandong/7/97, | |
| | | | B/Hong | |
| | | | Kong/330/2001, | |
| | | | B/Hong | |
| | | | Kong/1434/2002. | |
| | | | B/Brisbane/32/2002 is | |
| | | | also available as a | |
| | | | vaccine virus) | |
| 2004 | A/New | A/Fujian/411/2002(H3N2)- | B/Hong | / |
| | Caledonia/20/99 | like virus (Used | Kong/330/2001-like | |
| | (H1N1)-like virus | vaccine virus is | virus (Used vaccine | |
| | | A/Wyoming/3/2003. A/ | strains include | |
| | | Kumamoto/102/2002 | B/Shandong/7/97, | |
| | | is also available as a | B/Hong | |
| | | vaccine virus) | Kong/330/2001, | |
| | | | B/Hong | |
| | | | Kong/1434/2002. | |
| | | | B/Brisbane/32/2002 is | |
| | | | also available as a | |
| | | | vaccine virus) | |
| 2005 | A/New | A/Wellington/1/ | B/Shanghai/361/2002- | / |
| | Caledonia/20/99 | 2004(H3N2)-like virus | like virus (Candidate | |
| | (H1N1)-like virus | | vaccine viruses | |
| | | | include | |
| | | | B/Shanghai/361/2002, | |
| | | | B/Jiangsu/10/2003 and | |
| | | | B/Jilin/20/2003) | |
| 2006 | A/New | A/California/7/ | B/Malaysia/2506/ | / |
| | Caledonia/20/99 | 2004(H3N2)-like virus | 2004-like virus | |
| | (H1N1)-like virus | (A/New York/55/2004 | | |
| | | is available as a | | |
| | | vaccine virus) | | |
| 2007 | A/New | A/Wisconsin/67/2005 | B/Malaysia/2506/ | / |
| | Caledonia/20/99 | (H3N2)-like virus | 2004-like virus | |
| | (H1N1)-like virus | (A/Wisconsin/67/2005 | | |
| | | (H3N2) and | | |
| | | A/Hiroshima/52/2005) | | |
| 2008 | A/Solomon | A/Brisbane/10/2007 | B/Florida/4/2006-like | / |
| | Islands/3/2006 | (H3N2)-like virus | virus | |
| | (H1N1)-like virus | | | |
| 2009 | A/Brisbane/59/2007 | A/Brisbane/10/2007 | B/Florida/4/2006-like | / |
| | (H1N1)-like virus | (H3N2)-like virus | virus | |
| | (A/South | (A/Brisbane/10/2007 | | |
| | Dakota/6/2007 (an | and A/Uruguay/716/ | | |
| | A/Brisbane/59/2007- | 2007) | | |
| | like virus) is a vaccine | | | |
| | virus used in live | | | |
| | attenuated vaccines) | | | |
| 2010 | A/California/7/2009 | A/Perth/16/2009 | B/Brisbane/60/2008- | / |
| | (H1N1)-like virus | (H3N2)-like virus | like virus | |
| 2011 | A/California/7/2009 | A/Perth/16/2009 | B/Brisbane/60/2008- | / |
| | (H1N1)-like virus | (H3N2)-like virus | like virus | |
| | | (A/Wisconsin/15/2009 | | |
| | | and A/Victoria/210/ | | |
| | | 2009 are | | |
| | | A/Perth/16/2009-like | | |
| | | viruses) | | |
| 2012 | A/California/7/2009 | A/Perth/16/2009 | B/Brisbane/60/2008- | / |
| | (H1N1)pdm09-like | (H3N2)-like virus | like virus | |
| | virus | | | |
| 2013 | A/California/7/2009 | A/Victoria/361/2011 | B/Wisconsin/1/2010- | B/Brisbane/60/2008- |
| | (H1N1)pdm09-like | (H3N2)-like virus | like virus (B/Hubei- | like virus |
| | virus | (A/Ohio/2/2012, | Wujiagang/158/2009 | (B/Brisbane/33/2008 |
| | (A/Christchurch/16/2010 | A/Maryland/2/2012, | and B/Texas/6/2011 | is a B/Brisbane/60/ |
| | is an A/California/7/ | A/South | are B/Wisconsin/1/ | 2008-like virus) |
| | 2009-like virus) | Australia/30/2012, | 2010-like viruses) | |
| | | A/Brisbane/1/2012 and | | |
| | | A/Brisbane/6/2012 are | | |
| | | A/Victoria/361/2011- | | |
| | | like viruses) | | |
| 2014 | A/California/7/2009 | A/Texas/50/2012 | B/Massachusetts/2/2012- | B/Brisbane/60/2008- |
| | (H1N1)pdm09-like | (H3N2)-like virus | like virus | like virus |
| | virus | (A/Texas/50/2012 is | | |
| | (A/Christchurch/16/2010 | an A(H3N2) virus that | | |
| | is an A/California/7/ | following adaptation to | | |
| | 2009-like virus) | growth in eggs has | | |
| | | maintained antigenic | | |
| | | properties similar to | | |
| | | the majority of recently | | |
| | | circulating cell- | | |
| | | propagated A(H3N2) | | |
| | | viruses including | | |
| | | A/Victoria/361/2011) | | |
| 2015 | A/California/7/2009 | A/Switzerland/9715293/ | B/Phuket/3073/2013- | B/Brisbane/60/2008- |
| | (H1N1)pdm09-like | 2013 (H3N2)-like | like virus | like virus |
| | virus | virus (A/South | | |
| | | Australia/55/2014, | | |
| | | A/Norway/466/2014 | | |
| | | and A/Stockholm/6/ | | |
| | | 2014 are | | |
| | | A/Switzerland/9715293/ | | |
| | | 2013-like viruses) | | |
| 2016 | A/California/7/2009 | A/Hong | B/Brisbane/60/2008- | B/Phuket/3073/2013- |
| | (H1N1)pdm09-like | Kong/4801/2014 | like virus | like virus |
| | virus | (H3N2)-like virus | | |
| 2017 | A/Michigan/45/2015 | A/Hong | B/Brisbane/60/2008- | B/Phuket/3073/2013- |
| | (H1N1)pdm09-like | Kong/4801/2014 | like virus | like virus |
| | virus | (H3N2)-like virus | | |
| 2018 | A/Michigan/45/2015 | A/Singapore/INFIMH- | B/Phuket/3073/2013- | B/Brisbane/60/2008- |
| | (H1N1)pdm09-like | 16-0019/2016 (H3N2)- | like virus | like virus |
| | virus | like virus | | |
| 2019 | A/Michigan/45/2015 | A/Switzerland/8060/2017 | B/Colorado/06/2017- | B/Phuket/3073/2013- |
| | (H1N1)pdm09-like | (H3N2)-like virus or | like virus | like virus |
| | virus | A/Singapore/INFIMH- | (B/Victoria/2/87 | (B/Yamagata/16/88 |
| | | 16-0019/2016-like | lineage) | lineage) |
| | | virus | | |
| 2020 | A/Brisbane/02/2018 | A/South | B/Washington/02/2019- | B/Phuket/3073/2013- |
| | (H1N1)pdm09-like | Australia/34/2019 | like (B/Victoria | like (B/Yamagata |
| | virus | (H3N2)-like virus | lineage) virus | lineage) virus |
| 2021 | A/Victoria/2570/2019 | A/Hong | B/Washington/02/2019 | B/Phuket/3073/2013 |
| | (H1N1)pdm09-like | Kong/2671/2019 | (B/Victoria lineage)- | (B/Yamagata lineage)- |
| | virus or | (H3N2)-like virus or | like virus | like virus |
| | A/Wisconsin/588/2019 | A/Hong Kong/45/2019 | | |
| | (H1N1)pdm09-like | (H3N2)-like virus | | |
| | virus | | | |
| 2022 | A/Victoria/2570/2019 | A/Darwin/9/2021 | B/Austria/1359417/2021 | B/Phuket/3073/2013 |
| | (H1N1)pdm09-like | (H3N2)-like virus or | (B/Victoria lineage)- | (B/Yamagata lineage)- |
| | virus or | A/Darwin/6/2021 | like virus | like virus |
| | A/Wisconsin/588/2019 | (H3N2)-like virus | | |
| | (H1N1)pdm09-like | | | |
| | virus | | | |
| 2023 | A/Sydney/5/2021 | A/Darwin/9/2021 | B/Austria/1359417/2021 | B/Phuket/3073/2013 |
| | (H1N1)pdm09-like | (H3N2)-like virus or | (B/Victoria lineage)- | (B/Yamagata lineage)- |
| | virus | A/Darwin/6/2021 | like virus | like virus |
| | | (H3N2)-like virus | | |
| 2024 | A/Victoria/4897/2022 | A/Thailand/8/2022 | B/Austria/1359417/2021 | B/Phuket/3073/2013 |
| | (H1N1)pdm09-like | (H3N2)-like virus or | (B/Victoria lineage)- | (B/Yamagata lineage)- |
| | virus or | A/Massachusetts/18/2022 | like virus | like virus |
| | AWisconsin/67/2022 | (H3N2)-like virus | | |
| | (H1N1)pdm09-like | | | |
| | virus | | | |

Exemplary HA antigens are known in the art and are publicly available, for example, NCBI's Influenza Virus Resource (https://www.ncbi.nlm.nih.gov/genomes/FLU/Database/nph-select.cgi?go=database) and GISRS (https://gisaid.org/resources/human-Influenza-vaccine-composition/).

In some embodiments, said second HA antigen comprises or consists of an amino acid sequence having at least 90%, 95%, 98% or 99% identity to the amino acid sequence set forth in any one of SEQ ID NO: 1, 11, 19, 23, 27, 29, 39, 41 or 43, or fragment or variant thereof.

In some embodiments, said second HA antigen comprises or consists of an amino acid sequence set forth in any one of SEQ ID NO: 1, 11, 19, 23, 27, 29, 39, 41 or 43, or fragment or variant thereof.

In some embodiments, said first HA antigen comprises or consists of an amino acid sequence having at least 90%, 95%, 98% or 99% identity to the amino acid sequence set forth in any one of SEQ ID NO: 3, 9, 13, 15, 21, 25, 31, 33, 37, 45 or 47 or fragment or variant thereof.

In some embodiments, said first HA antigen comprises or consists of an amino acid sequence set forth in any one of SEQ ID NO: 3, 9, 13, 15, 21, 25, 31, 33, 37, 45 or 47 or fragment or variant thereof.

In some embodiments, said first and/or second HA antigen is a polypeptide comprising a full-length Influenza HA protein. Suitably, said first and/or second HA antigen is a polypeptide consisting of a full-length Influenza HA protein.

In some embodiments, said first and/or second HA antigen is a fragment of a hemagglutinin protein, such as a truncated hemagglutinin protein. In some embodiments, the fragment is a headless hemagglutinin, meaning the fragment does not comprise the head domain. In some embodiments, the fragment comprises a portion of the head domain. In some embodiments, the fragment is a stalk domain. In some embodiments, the fragment does not comprise the cytoplasmic domain. In some embodiments, the fragment does not comprise the transmembrane domain. In such embodiments, the fragment may be referred to as a soluble or secreted hemagglutinin protein or fragment.

In some embodiments, said ratio of (a):(b) is comprised between 1.5:1 and 5:1, optionally between 2:1 and 5:1, optionally between 3:1 and 5:1, optionally between 4:1 and 5:1, optionally between 1.5:1 and 4:1, optionally between 1.5:1 and 3:1, optionally between 2:1 and 4:1, optionally between 2:1 and 3:1.

In some embodiments, said ratio of (a):(b) is selected from about 1.5:1, about 2:1, about 2.2:1, about 2.4:1, about 2.6:1, about 2.8:1, about 3:1, about 3.2:1, about 3.4:1, about 3.6:1, about 3.8:1, about 4:1, about 4.2:1, about 4.4:1, about 4.6:1, about 4.8:1 or about 5:1. In some embodiments, said ratio of (a):(b) is selected from about 1.5:1, 2:1, 2.2:1, 2.4:1, 2.6:1, 2.8:1, 3:1, 3.2:1, 3.4:1, 3.6:1, 3.8:1, 4:1, 4.2:1, 4.4:1, 4.6:1, 4.8:1 or 5:1.

In some embodiments, said ratio of (a):(b) is comprised between 2:1 and 4:1, suitably between 2:1 and 3:1, suitably is 2:1 or 3:1.

In some embodiments, said ratio of (a):(b) is about 2:1, suitably 2.1.

In some embodiments, said ratio of (a):(b) is about 3:1, suitably 3.1.

In some embodiments, said ratio of (a):(b) is greater than 3:1 and lower or equal to 20:1.

In some embodiments, said ratio of (a):(b) is greater than 3:1 and lower or equal to 5:1.

In some embodiments, the immunogenic composition comprises:
(a) as defined herein; suitably being a first hemagglutinin (HA) antigen or a first nucleic acid, suitably mRNA, encoding the first HA antigen wherein the first HA antigen is derived from a strain of subtype H3 of Influenza A virus; and
(b) as defined herein; suitably being a second HA antigen or a second nucleic acid, suitably mRNA, encoding the second HA antigen wherein the second HA antigen is derived from a strain of subtype H1 of Influenza A virus,
   wherein the ratio of (a):(b) is comprised between 2:1 and 4:1, suitably between 2:1 and 3:1, suitably is 2:1 or 3:1.

In some embodiments, the immunogenic composition comprises:
(a) as defined herein; suitably being a first hemagglutinin (HA) antigen or a first nucleic acid, suitably mRNA, encoding the first HA antigen wherein the first HA antigen is derived from a strain of subtype H3 of Influenza A virus; and
(b) as defined herein; suitably being a second HA antigen or a second nucleic acid, suitably mRNA, encoding the second HA antigen wherein the second HA antigen is derived from a strain of subtype H1 of Influenza A virus,
   wherein the ratio of (a):(b) is greater than 3:1 and lower or equal to 20:1, suitably is greater than 3:1 and lower or equal to 5:1.

In some embodiments, the immunogenic composition further comprises:
(c) at least one further antigen or at least one further nucleic acid, suitably mRNA, encoding the at least one further antigen, wherein the at least one further antigen is derived from a strain of Influenza virus.

In some embodiments, (a), (b) and (c) are different.

In some embodiments, said strain of Influenza virus of (c) is selected from the group consisting of Influenza A virus and Influenza B virus.

In some embodiments, said strain of Influenza virus of (c) is a strain of Influenza A virus.

In some embodiments, said strain of Influenza A virus is a strain of subtype H1 of Influenza A virus, optionally selected from Influenza A viruses characterized by a neuraminidase (NA) selected from the group consisting of N1 and N2, more suitably N1, optionally selected from the group consisting of H1N1 and H1N2, suitably H1N1.

In some embodiments, said strain of Influenza A virus is a strain of subtype H3 of Influenza A virus, optionally selected from Influenza A viruses characterized by a neuraminidase (NA) selected from the group consisting of N1, N2, and N8, suitably N2, optionally selected from the group consisting of H3N1, H3N2 and H3N8, suitably H3N2.

In some embodiments, said at least one further antigen or at least one further nucleic acid, suitably mRNA, encoding the at least one further antigen is at least one further HA antigen or at least one further nucleic acid, suitably mRNAs, encoding the at least one further HA antigen derived from a strain of subtype H3 of Influenza A virus, suitably from H3N2, and wherein the ratio of (a) and (c) to (b) is greater than 1:1 and lower or equal to 20:1, optionally greater than 1:1 and lower of equal to 5:1, optionally comprised between 1.5:1 and 5:1.

In some embodiments, said immunogenic composition comprises a plurality of HA antigens or a plurality of nucleic acids, suitably mRNAs, encoding the plurality of HA antigens derived from at least two different strains of subtype H3 of Influenza A virus, suitably from H3N2, wherein the ratio of the plurality of HA antigens or the plurality of nucleic acids, suitably mRNAs, encoding the plurality of HA antigens to (b) is greater than 1:1 and lower or equal to 20:1, optionally greater than 1:1 and lower of equal to 5:1, optionally comprised between 1.5:1 and 5:1.

In some embodiments, said strain of Influenza virus of (c) is a strain of Influenza B virus.

In some embodiments, said strain of Influenza B virus is selected from the group consisting of B/Victoria lineage and B/Yamagata lineage.

In some embodiments, said strain of Influenza B virus is a strain from B/Victoria lineage.

In some embodiments, said strain of Influenza B virus is selected from the group consisting of B/Beijing/184/93-like virus, B/Harbin/94-like virus, B/Shangdong/7/97-like virus,

B/Yamanashi/166/98-like virus, B/Sichuan/379/99-like virus, B/Guangdong/120/2000, B/Johannesburg/5/99, B/Victoria/504/2000, B/Hong Kong/330/2001-like virus, B/Hong Kong/1434/2002, B/Brisbane/32/2002, B/Shanghai/361/2002-like virus, B/Jiangsu/10/2003, B/Jilin/20/2003, B/Malaysia/2506/2004-like virus, B/Malaysia/2506/2004 virus, B/Ohio/1/2005, B/Florida/4/2006-like virus, B/Brisbane/3/2007, B/Brisbane/60/2008-like virus, B/Brisbane/33/2008, B/Wisconsin/1/2010-like virus, B/Hubei-Wujiagang/158/2009, B/Texas/6/2011, B/Massachusetts/2/2012-like virus, B/Phuket/3073/2013-like virus, B/Austria/1359417/2021-like virus, B/Washington/02/2019-like virus and B/Colorado/06/2017-like virus.

In some embodiments, said strain of Influenza B virus is selected from an Influenza B virus as listed in Table 1 and/or Table 2.

In some embodiments, said strain of Influenza B virus is selected from an Influenza B virus which is recommended for I vaccine composition by the WHO (https://www.who.int/teams/global-influenza-programme/vaccines/who-recommendations).

In some embodiments, said at least one further antigen comprises or consists of a peptide or protein selected or derived from an Influenza virus hemagglutinin (HA), neuraminidase (NA), nucleoprotein (NP), matrix protein 1 (M1), matrix protein 2 (M2), non-structural protein 1 (NS1), non-structural protein 2 (NS2), nuclear export protein (NEP), polymerase acidic protein (PA), polymerase basic protein PB1, PB1-F2, and/or polymerase basic protein 2 (PB2), or an immunogenic fragment or an immunogenic variant thereof.

In some embodiments, said at least one further antigen comprises or consists of a peptide or protein selected or derived from an Influenza virus hemagglutinin (HA) or neuraminidase (NA) or an immunogenic fragment or an immunogenic variant thereof.

In some embodiments, the immunogenic composition comprises a combination of HA antigens or nucleic acids, suitably mRNAs, encoding said HA antigens, said at least one further antigen comprising or consisting of a peptide or protein selected or derived from an Influenza virus HA or fragment or variant thereof.

In some embodiments, the immunogenic composition comprises a combination of HA and NA antigens or nucleic acids, suitably mRNAs, encoding said HA and NA antigens, said at least one further antigen comprising or consisting of a peptide or protein selected or derived from an Influenza virus NA or fragment or variant thereof.

Like HA, neuraminidase (NA) is a major surface glycoprotein of Influenza virus. Naturally acquired or vaccine-induced NA-inhibiting (NAI) antibodies have been shown to contribute to influenza disease protection in naturally occurring Influenza or in experimental human challenge studies. NAI antibodies appear to have an independent role in vaccine efficacy/effectiveness as compared to Hemagglutinin inhibition antibodies. Antigenic drifts of HA and NA have been reported to be independent suggesting that NA-specific immunity is likely to provide a level of protection when drift in HA occurs.

In some embodiments, said HA antigen is a polypeptide comprising a full-length Influenza HA protein. Suitably, said HA antigen is a polypeptide consisting of a full-length Influenza HA protein.

In some embodiments, said HA antigen is a fragment of a hemagglutinin protein, such as a truncated hemagglutinin protein. In some embodiments, the fragment is a headless hemagglutinin, meaning the fragment does not comprise the head domain. In some embodiments, the fragment comprises a portion of the head domain. In some embodiments, the fragment is a stalk domain. In some embodiments, the fragment does not comprise the cytoplasmic domain. In some embodiments, the fragment does not comprise the transmembrane domain. In such embodiments, the fragment may be referred to as a soluble or secreted hemagglutinin protein or fragment.

In some embodiments, said NA antigen is a polypeptide comprising a full-length Influenza NA protein. Suitably, said NA antigen is a polypeptide consisting of a full-length Influenza NA protein.

In some embodiments, said NA antigen is a fragment of a neuraminidase protein, such as a truncated neuraminidase protein.

In some embodiments, the HA and NA antigens or nucleic acids, suitably mRNAs, encoding said HA and NA antigens are present in equimolar proportions, suitably with respect to H1 HA antigen.

In some embodiments, the HA and NA antigens or nucleic acids, suitably mRNAs, encoding said HA and NA antigens are not present in equimolar proportions.

In some embodiments, the dose (e.g. weight dose or molar dose, suitably weight dose) of said at least one NA antigen or nucleic acid, suitably mRNA, encoding such is different compared to the dose (e.g. weight dose or molar dose, suitably weight dose) of the HA antigens or nucleic acids, suitably mRNAs, encoding said HA antigens.

In some embodiments, the dose (e.g. weight dose or molar dose, suitably weight dose) of said at least one NA antigen or nucleic acid, suitably mRNA, encoding such is different compared to the dose (e.g. weight dose or molar dose, suitably weight dose) of the H1 HA antigens or nucleic acids, suitably mRNAs, encoding said H1 HA antigens.

In some embodiments, the ratio of HA: NA antigens or nucleic acids, suitably mRNAs, encoding such, is comprised between 4:1 and 1:4, suitably, 3:1 and 1:3, suitably 2:1 and 2:1.

In some embodiments, the ratio of H1HA: NA antigens or nucleic acids, suitably mRNAs, encoding such, is comprised between 4:1 and 1:4, suitably, 3:1 and 1:3, suitably 2:1 and 2:1.

In some embodiment, the ratio of HA: NA antigens or nucleic acids, suitably mRNAs, encoding such is 4:1 or 1:4.

In some embodiment, the ratio of HA: NA antigens or nucleic acids, suitably mRNAs, encoding such is 3:1 or 1:3.

In some embodiment, the ratio of HA: NA antigens or nucleic acids, suitably mRNAs, encoding such is 2:1 or 1:2.

In some embodiment, the ratio of HA: NA antigens or nucleic acids, suitably mRNAs, encoding such is 3:2 or 2:3.

In some embodiment, the ratio of HA: NA antigens or nucleic acids, suitably mRNAs, encoding such is 4:3 or 3:4.

In some embodiment, the ratio of H1HA: NA antigens or nucleic acids, suitably mRNAs, encoding such is 4:1 or 1:4.

In some embodiment, the ratio of H1HA: NA antigens or nucleic acids, suitably mRNAs, encoding such is 3:1 or 1:3.

In some embodiment, the ratio of H1HA: NA antigens or nucleic acids, suitably mRNAs, encoding such is 2:1 or 1:2.

In some embodiment, the ratio of H1HA: NA antigens or nucleic acids, suitably mRNAs, encoding such is 3:2 or 2:3.

In some embodiment, the ratio of H1HA: NA antigens or nucleic acids, suitably mRNAs, encoding such is 4:3 or 3:4.

In some embodiment, the ratio of HA: NA antigens or nucleic acids, suitably mRNAs, encoding such is about 1:1. In some embodiment, the dose ratio of HA: NA antigens or nucleic acids, suitably mRNAs, encoding such is 1:1.

In some embodiment, the ratio of H1HA: NA antigens or nucleic acids, suitably mRNAs, encoding such is about 1:1. In some embodiment, the dose ratio of H1HA: NA antigens or nucleic acids, suitably mRNAs, encoding such is 1:1.

In some embodiments, the ratio is a weight/weight ratio or a molar ratio. Suitably, the ratio is a weight/weight ratio.

In some embodiments, said at least one further antigen comprises or consists of an amino acid sequence having at least 90%, 95%, 98% or 99% identity to the amino acid sequence set forth in any one of SEQ ID NO: 1 to 48, or fragment thereof.

In some embodiments, said at least one further antigen comprises or consists of an amino acid sequence set forth in any one of SEQ ID NO: 1 to 48, or fragment thereof.

In some embodiments, said at least one further antigen comprises or consists of an amino acid sequence having at least 90%, 95%, 98% or 99% identity to the amino acid sequence set forth in any one of SEQ ID NO: 1, 3, 5, 7, 9, 11, 13, 15, 17, 19, 21, 23, 25, 27, 29, 31, 33, 35, 37, 39, 41, 43, 45 or 47 or fragment or variant thereof.

In some embodiments, said at least one further antigen comprises or consists of the amino acid sequence set forth in any one of SEQ ID NO: 1, 3, 5, 7, 9, 11, 13, 15, 17, 19, 21, 23, 25, 27, 29, 31, 33, 35, 37, 39, 41, 43, 45 or 47 or fragment or variant thereof.

In some embodiments, said at least one further antigen comprises or consists of an amino acid sequence having at least 90%, 95%, 98% or 99% identity to the amino acid sequence set forth in any one of SEQ ID NO: 5, 7, 17 or 35, or fragment or variant thereof.

In some embodiments, said at least one further antigen comprises or consists of an amino acid sequence set forth in any one of SEQ ID NO: 5, 7, 17 or 35, or fragment or variant thereof.

In some embodiments, said at least one further antigen comprises or consists of an amino acid sequence having at least 90%, 95%, 98% or 99% identity to the amino acid sequence set forth in any one of SEQ ID NO: 1, 3, 9, 11, 13, 15, 19, 21, 23, 25, 27, 29, 31, 33, 37, 39, 41, 43, 45 or 47 or fragment or variant thereof.

In some embodiments, said at least one further antigen comprises or consists of an amino acid sequence set forth in any one of SEQ ID NO: 1, 3, 9, 11, 13, 15, 19, 21, 23, 25, 27, 29, 31, 33, 37, 39, 41, 43, 45 or 47 or fragment or variant thereof.

In some embodiments, said at least one further antigen comprises or consists of an amino acid sequence having at least 90%, 95%, 98% or 99% identity to the amino acid sequence set forth in any one of SEQ ID NO: 1, 11, 19, 23, 27, 29, 39, 41 or 43, or fragment or variant thereof.

In some embodiments, said at least one further antigen comprises or consists of an amino acid sequence set forth in any one of SEQ ID NO: 1, 11, 19, 23, 27, 29, 39, 41 or 43, or fragment or variant thereof.

In some embodiments, said at least one further antigen comprises or consists of an amino acid sequence having at least 90%, 95%, 98% or 99% identity to the amino acid sequence set forth in any one of SEQ ID NO: 3, 9, 13, 15, 21, 25, 31, 33, 37, 45 or 47 or fragment or variant thereof.

In some embodiments, said at least one further antigen comprises or consists of an amino acid sequence set forth in any one of SEQ ID NO: 3, 9, 13, 15, 21, 25, 31, 33, 37, 45 or 47 or fragment or variant thereof.

In some embodiments, said at least one further antigen comprises or consists of an amino acid sequence having at least 90%, 95%, 98% or 99% identity to the amino acid sequence set forth in any one of SEQ ID NO: 2, 4, 6, 8, 10, 12, 14, 16, 18, 20, 22, 24, 26, 28, 30, 32, 34, 36, 38, 40, 42, 44, 46 or 48 or fragment or variant thereof.

In some embodiments, said at least one further antigen comprises or consists of the amino acid sequence set forth in any one of SEQ ID NO: SEQ ID NO: 2, 4, 6, 8, 10, 12, 14, 16, 18, 20, 22, 24, 26, 28, 30, 32, 34, 36, 38, 40, 42, 44, 46 or 48 or fragment or variant thereof.

In some embodiments, said at least one further antigen comprises or consists of an amino acid sequence having at least 90%, 95%, 98% or 99% identity to the amino acid sequence set forth in any one of SEQ ID NO: 6, 8, 18, 36, or fragment or variant thereof.

In some embodiments, said at least one further antigen comprises or consists of the amino acid sequence set forth in any one of SEQ ID NO: SEQ ID NO: 6, 8, 18, 36, or fragment or variant thereof.

In some embodiments, said at least one further antigen comprises or consists of an amino acid sequence having at least 90%, 95%, 98% or 99% identity to the amino acid sequence set forth in any one of SEQ ID NO: 2, 4, 10, 12, 14, 16, 20, 22, 24, 26, 28, 30, 32, 34, 38, 40, 42, 44, 46 or 48 or fragment or variant thereof.

In some embodiments, said at least one further antigen comprises or consists of the amino acid sequence set forth in any one of SEQ ID NO: SEQ ID NO: 2, 4, 10, 12, 14, 16, 20, 22, 24, 26, 28, 30, 32, 34, 38, 40, 42, 44, 46 or 48 or fragment or variant thereof.

In some embodiments, said at least one further antigen comprises or consists of an amino acid sequence having at least 90%, 95%, 98% or 99% identity to the amino acid sequence set forth in any one of SEQ ID NO: 2, 12, 20, 24, 28, 30, 40, 42 or 44, or fragment or variant thereof.

In some embodiments, said at least one further antigen comprises or consists of the amino acid sequence set forth in any one of SEQ ID NO: SEQ ID NO: 2, 12, 20, 24, 28, 30, 40, 42 or 44, or fragment or variant thereof.

In some embodiments, said at least one further antigen comprises or consists of an amino acid sequence having at least 90%, 95%, 98% or 99% identity to the amino acid sequence set forth in any one of SEQ ID NO: 4, 10, 14, 16, 22, 26, 32, 34, 38, 46 or 48 or fragment or variant thereof.

In some embodiments, said at least one further antigen comprises or consists of the amino acid sequence set forth in any one of SEQ ID NO: SEQ ID NO: 4, 10, 14, 16, 22, 26, 32, 34, 38, 46 or 48 or fragment or variant thereof.

In some embodiments, the composition is a multivalent composition, said strain of Influenza virus of (a) and/or said strain of Influenza virus of (b) and/or said strain of Influenza virus of (c) being different.

In some embodiments, the composition is a trivalent composition, said strain of Influenza virus of (a), said strain of Influenza virus of (b) and said strain of Influenza virus of (c) being different.

In some embodiments, said strain of Influenza virus of (c) is a strain of Influenza B virus and the ratio of (a):(b): (c) is greater than 1:1:1, optionally greater than 1:1:1 and lower or equal to 20:1:1, optionally greater than 1:1:1 and lower or equal to 5:1:1.

In some embodiments, said strain of Influenza virus of (c) is a strain of Influenza B virus and the ratio of (a):(b): (c) is comprised between 1.5:1:1 and 5:1:1, optionally between 2:1:1 and 5:1:1, optionally between 3:1:1 and 5:1:1, optionally between 4:1:1 and 5:1:1, optionally between 1.5:1:1 and 4:1:1, optionally between 1.5:1:1 and 3:1:1, optionally between 2:1:1 and 4:1:1, optionally between 2:1:1 and 3:1:1.

In some embodiments, said strain of Influenza virus of (c) is a strain of Influenza B virus and the ratio of (a):(b): (c) is selected from about 1.5:1:1, about 2:1:1, about 2.2:1:1, about 2.4:1:1, about 2.6:1:1, about 2.8:1:1, about 3:1:1, about 3.2:1:1, about 3.4:1:1, about 3.6:1:1, about 3.8:1:1, about 4:1:1, about 4.2:1:1, about 4.4:1:1, about 4.6:1:1, about 4.8:1:1 or about 5:1:1.

In some embodiments, said strain of Influenza virus of (c) is a strain of Influenza B virus and the ratio of (a):(b): (c) is selected from 1.5:1:1, 2:1:1, 2.2:1:1, 2.4:1:1, 2.6:1:1, 2.8:1:1, 3:1:1, 3.2:1:1, 3.4:1:1, 3.6:1:1, 3.8:1:1, 4:1:1, 4.2:1:1, 4.4:1:1, 4.6:1:1, 4.8:1:1 or 5:1:1.

In some embodiments, said strain of Influenza virus of (c) is a strain of Influenza B virus and the ratio of (a):(b): (c) is comprised between 2:1:1 and 4:1:1, suitably between 2:1:1 and 3:1:1, suitably is 2:1:1 or 3:1:1.

In some embodiments, said strain of Influenza virus of (c) is a strain of Influenza B virus and the ratio of (a):(b): (c) is greater than 3:1:1 and lower or equal to 20:1:1.

In some embodiments, said strain of Influenza virus of (c) is a strain of Influenza B virus and the ratio of (a):(b): (c) is greater than 3:1:1 and lower or equal to 5:1:1.

In some embodiments, (c) is a third HA antigen or a third nucleic acid, suitably mRNA, encoding the third HA antigen wherein the third HA antigen is derived from a first strain of Influenza B virus, suitably from B/Victoria lineage.

In some embodiments, (c) is a third HA antigen or a third nucleic acid, suitably mRNA, encoding the third HA antigen wherein the third HA antigen is derived from a first strain of Influenza B virus, suitably from B/Victoria lineage, and the ratio of (a):(b): (c) is greater than 1:1:1, optionally greater than 1:1:1 and lower or equal to 20:1:1, optionally greater than 1:1:1 and lower or equal to 5:1:1.

In some embodiments, (c) is a third HA antigen or a third nucleic acid, suitably mRNA, encoding the third HA antigen wherein the third HA antigen is derived from a first strain of Influenza B virus, suitably from B/Victoria lineage, and the ratio of (a):(b): (c) is comprised between 1.5:1:1 and 5:1:1, optionally between 2:1:1 and 5:1:1, optionally between 3:1:1 and 5:1:1, optionally between 4:1:1 and 5:1:1, optionally between 1.5:1:1 and 4:1:1, optionally between 1.5:1:1 and 3:1:1, optionally between 2:1:1 and 4:1:1, suitably between 2:1:1 and 3:1:1, suitably is 2:1:1 or 3:1:1.

In some embodiments, (c) is a third HA antigen or a third nucleic acid, suitably mRNA, encoding the third HA antigen wherein the third HA antigen is derived from a first strain of Influenza B virus, suitably from B/Victoria lineage, and the ratio of (a):(b): (c) is comprised between 1.5:1:1.5 and 20:1:20.

In some embodiments, (c) is a third HA antigen or a third nucleic acid, suitably mRNA, encoding the third HA antigen wherein the third HA antigen is derived from a first strain of Influenza B virus, suitably from B/Victoria lineage, and the ratio of (a):(b): (c) is comprised between 1.5:1:5.5 and 20:1:20.

In some embodiments, (c) is a third HA antigen or a third nucleic acid, suitably mRNA, encoding the third HA antigen wherein the third HA antigen is derived from a first strain of Influenza B virus, suitably from B/Victoria lineage, and the ratio of (a):(b): (c) is comprised between 1.5:1:1.5 and 5:1:5, optionally between 2:1:2 and 5:1:5, optionally between 3:1:3 and 5:1:5, optionally between 4:1:4 and 5:1:5, optionally between 1.5:1:1.5 and 4:1:4, optionally between 1.5:1:1.5 and 3:1:3, optionally between 2:1:2 and 4:1:4, suitably between 2:1:2 and 3:1:3, suitably is 2:1:2, 3:1:2, 2:1:3 or 3:1:3.

In some embodiments, (c) is a third HA antigen or a third nucleic acid, suitably mRNA, encoding the third HA antigen wherein the third HA antigen is derived from a first strain of Influenza B virus, suitably from B/Victoria lineage, and the ratio of (a):(b): (c) is comprised between 4:1:1.5 and 20:1:20.

In some embodiments, the ratio is a weight/weight ratio or a molar ratio. Suitably, the ratio is a weight/weight ratio.

In some embodiments, the immunogenic composition comprises:
(a) as defined herein; suitably being a first hemagglutinin (HA) antigen or a first nucleic acid, suitably mRNA, encoding the first HA antigen wherein the first HA antigen is derived from a strain of subtype H3 of Influenza A virus, suitably H3N2;
(b) as defined herein; suitably being a second HA antigen or a second nucleic acid, suitably mRNA, encoding the second HA antigen wherein the second HA antigen is derived from a strain of subtype H1 of Influenza A virus, suitably H1N1, and
(c) as defined herein; suitably being at least one further antigen or at least one further nucleic acid, suitably mRNA, encoding the at least one further antigen, wherein the at least one further antigen is derived from a strain of Influenza virus, suitably from B/Victoria lineage,
   wherein (a), (b) and (c) are different, and wherein the ratio of (a):(b): (c) is comprised between 1.5:1:1 and 20:1:1, optionally between 1.5:1:1 and 5:1:1.

In some embodiments, said antigens of (a), (b) and/or (c) are derived from at least two, or three strains of Influenza virus, suitably three strains of Influenza virus.

In some embodiments, the composition comprises three antigens or nucleic acids, suitably mRNAs, encoding such.

In some embodiments, the immunogenic composition comprises a combination of three HA antigens or three nucleic acids, suitably mRNAs, encoding said three HA antigens.

In some embodiments, the immunogenic composition comprises:
(a) as defined herein; suitably being a first hemagglutinin (HA) antigen or a first nucleic acid, suitably mRNA, encoding the first HA antigen wherein the first HA antigen is derived from a strain of subtype H3 of Influenza A virus, suitably H3N2;
(b) as defined herein; suitably being a second HA antigen or a second nucleic acid, suitably mRNA, encoding the second HA antigen wherein the second HA antigen is derived from a strain of subtype H1 of Influenza A virus, suitably H1N1, and
(c) as defined herein; suitably being a third HA antigen or a third nucleic acid, suitably mRNA, encoding the third HA antigen, wherein the third HA antigen is derived from a strain of Influenza B virus, suitably from B/Victoria lineage,
wherein (a), (b) and (c) are different, and wherein the ratio of (a):(b): (c) is comprised between 1.5:1:1 and 20:1:1, optionally between 1.5:1:1 and 5:1:1.

In some embodiments, the ratio of (a):(b): (c) is greater than 3:1:1 and lower of equal to 20:1:1.

In some embodiments, the ratio of (a):(b): (c) is greater than 3:1:1 and lower of equal to 5:1:1.

In some embodiments, the immunogenic composition comprises:
(a) as defined herein; suitably being a first hemagglutinin (HA) antigen or a first nucleic acid, suitably mRNA, encoding the first HA antigen wherein the first HA antigen is derived from a strain of subtype H3 of Influenza A virus, suitably H3N2;
(b) as defined herein; suitably being a second HA antigen or a second nucleic acid, suitably mRNA, encoding the second HA antigen wherein the second HA antigen is derived from a strain of subtype H1 of Influenza A virus, suitably H1N1, and
(c) as defined herein; suitably being a third HA antigen or a third nucleic acid, suitably mRNA, encoding the third HA antigen, wherein the third HA antigen is derived from a strain of Influenza B virus, suitably from B/Victoria lineage, wherein (a), (b) and (c) are different.

In some embodiments, the immunogenic composition comprises a plurality of (c), such as (c¹), (c²), (c³), (c⁴), (c⁵) and/or (c⁶) as defined herein.

In some embodiments, the composition comprises at least four, five, six, seven or eight antigens or nucleic acids, suitably mRNAs, encoding such, optionally four to ten antigens or nucleic acids, suitably mRNAs, encoding such, optionally four, six, seven or eight antigens or nucleic acids, suitably mRNAs, encoding such.

In some embodiments, said antigens of (a), (b) and/or (c) are derived from at least two, three or four strains of Influenza virus.

In some embodiments, the composition comprises four antigens or nucleic acids, suitably mRNAs, encoding such.

In some embodiments, the immunogenic composition comprises a combination of four HA antigens or four nucleic acids, suitably mRNAs, encoding said four HA antigens.

In some embodiments, the immunogenic composition comprises:
(a) and (b) as defined herein; suitably (a) being a first hemagglutinin (HA) antigen or a first nucleic acid, suitably mRNA, encoding the first HA antigen wherein the first HA antigen is derived from a strain of subtype H3 of Influenza A virus, suitably H3N2, and/or (b) being a second HA antigen or a second nucleic acid, suitably mRNA, encoding the second HA antigen wherein the second HA antigen is derived from a strain of subtype H1 of Influenza A virus, suitably H1N1;
(c¹) a third HA antigen or a third nucleic acid, suitably mRNA, encoding the third HA antigen wherein the third HA antigen is derived from a first strain of Influenza B virus, suitably from a B/Victoria lineage; and
(c²) optionally, a fourth HA antigen or a fourth nucleic acid, suitably mRNA, encoding the fourth HA antigen wherein the fourth HA antigen is derived from a second strain of Influenza B virus, suitably from B/Yamagata lineage,
   wherein (a), (b), (c¹) and (c²) are different, and wherein the ratio of (a):(b) is comprised between 1.5:1 and 20:1, optionally between 1.5:1 and 5:1, optionally between 2:1 and 5:1, optionally between 3:1 and 5:1, optionally between 4:1 and 5:1, optionally between 1.5:1 and 4:1, optionally between 1.5:1 and 3:1, 2:1 and 4:1, suitably between 2:1 and 3:1, suitably is 2:1 or 3:1.

In some embodiments, the ratio of (a):(b) is greater than 1:1, optionally greater than 1:1 and lower or equal to 20:1, optionally greater than 1:1 and lower or equal to 5:1.

In some embodiments, the ratio of (a):(b) is greater than 3:1 and lower of equal to 20:1. In some embodiments, the ratio of (a):(b) is greater than 3:1 and lower of equal to 5:1. In some embodiments, the immunogenic composition comprises:
(a) and (b) as defined herein; suitably (a) being a first hemagglutinin (HA) antigen or a first nucleic acid, suitably mRNA, encoding the first HA antigen wherein the first HA antigen is derived from a strain of subtype H3 of Influenza A virus, suitably H3N2, and/or (b) being a second HA antigen or a second nucleic acid, suitably mRNA, encoding the second HA antigen wherein the second HA antigen is derived from a strain of subtype H1 of Influenza A virus, suitably H1N1;
(c¹) as defined herein; suitably being a third HA antigen or a third nucleic acid, suitably mRNA, encoding the third HA antigen wherein the third HA antigen is derived from a first strain of Influenza B virus, suitably from B/Victoria lineage; and optionally, (c²) as defined herein; suitably being a fourth HA antigen or a fourth nucleic acid, suitably mRNA, encoding the fourth HA antigen wherein the fourth HA antigen is derived from a second strain of Influenza B virus, suitably from B/Yamagata lineage, wherein (a), (b), (c¹) and (c²) are different, and wherein the ratio of (a):(b): (c¹) is comprised between 1.5:1:1 and 20:1:1, optionally between 1.5:1:1 and 5:1:1, optionally between 2:1:1 and 5:1:1, optionally between 3:1:1 and 5:1:1, optionally between 4:1:1 and 5:1:1, optionally between 1.5:1:1 and 4:1:1, optionally between 1.5:1:1 and 3:1:1, 2:1:1 and 4:1:1, suitably between 2:1:1 and 3:1:1, suitably is 2:1:1 or 3:1:1.

In some embodiments, the ratio of (a):(b): (c¹) is greater than 1:1:1, optionally greater than 1:1:1 and lower or equal to 20:1:1, optionally greater than 1:1:1 and lower or equal to 5:1:1.

In some embodiments, the ratio of (a):(b): (c¹) is greater than 3:1:1 and lower of equal to 20:1:1.

In some embodiments, the ratio of (a):(b): (c¹) is greater than 3:1:1 and lower of equal to 5:1:1. In some embodiments, the ratio is a weight/weight ratio or a molar ratio. Suitably, the ratio is a weight/weight ratio.

In some embodiments, the immunogenic composition further comprises (c²) said fourth mRNA encoding a HA of a second strain of Influenza B virus, suitably from B/Yamagata lineage.

In some embodiments, the immunogenic composition comprises:
(a) and (b) as defined herein; suitably (a) being a first hemagglutinin (HA) antigen or a first nucleic acid, suitably mRNA, encoding the first HA antigen wherein the first HA antigen is derived from a strain of subtype H3 of Influenza A virus, suitably H3N2, and/or (b) being a second HA antigen or a second nucleic acid, suitably mRNA, encoding the second HA antigen wherein the second HA antigen is derived from a strain of subtype H1 of Influenza A virus, suitably H1N1;
(c¹) as defined herein; suitably being a third HA antigen or a third nucleic acid, suitably mRNA, encoding the third HA antigen wherein the third HA antigen is derived from a first strain of Influenza B virus, suitably from B/Victoria lineage, and (c²) as defined herein; suitably being a fourth HA antigen or a fourth nucleic acid, suitably mRNA, encoding the fourth HA antigen wherein the fourth HA antigen is derived from a second strain of Influenza B virus, suitably from B/Yamagata lineage,
   wherein (a), (b), (c¹) and (c²) are different, and wherein the ratio of (a):(b): (c¹): (c²) is comprised between 1.5:1:1:1 and 20:1:1:1; optionally between 1.5:1:1:1 and 5:1:1:1.

In some embodiments, the ratio of (a):(b): (c¹): (c²) is greater than 3:1:1:1 and lower of equal to 20:1:1:1.

In some embodiments, the ratio of (a):(b): (c¹): (c²) is greater than 3:1:1:1 and lower of equal to 5:1:1:1.

In some embodiments, the ratio is a weight/weight ratio or a molar ratio. Suitably, the ratio is a weight/weight ratio.

In some embodiments, the immunogenic composition comprises:
(a) as defined herein; suitably being a first hemagglutinin (HA) antigen or a first nucleic acid, suitably mRNA, encoding the first HA antigen wherein the first HA antigen is derived from a strain of subtype H3 of Influenza A virus, suitably H3N2;
(b) as defined herein; suitably being a second HA antigen or a second nucleic acid, suitably mRNA, encoding the second HA antigen wherein the second HA antigen is derived from a strain of subtype H1 of Influenza A virus, suitably H1N1;
(c¹) as defined herein; suitably being a third HA antigen or a third nucleic acid, suitably mRNA, encoding the third HA antigen wherein the third HA antigen is derived from a first strain of Influenza B virus, suitably from B/Victoria lineage, and
(c²) as defined herein; suitably being a fourth HA antigen or a fourth nucleic acid, suitably mRNA, encoding the fourth HA antigen wherein the fourth HA antigen is derived from a second strain of Influenza B virus, suitably from B/Yamagata lineage,
   wherein (a), (b), (c¹) and (c²) are different.

In some embodiments, the ratio of (a):(b): (c¹): (c²) is comprised between 1.5:1:1:1 and 20:1:1:1, optionally between 1.5:1:1:1 and 5:1:1:1.

In some embodiments, the ratio of (a):(b): (c¹): (c²) is greater than 1:1:1:1, optionally greater than 1:1:1:1 and lower or equal to 20:1:1:1, optionally greater than 1:1:1:1 and lower or equal to 5:1:1:1.

In some embodiments, the ratio of (a):(b): (c¹): (c²) is comprised between 1.5:1:1:1 and 5:1:1:1, optionally between 2:1:1:1 and 5:1:1:1, optionally between 3:1:1:1 and 5:1:1:1, optionally between 4:1:1:1 and 5:1:1:1, optionally between 1.5:1:1:1 and 4:1:1:1, optionally between 1.5:1:1:1 and 3:1:1:1, optionally between 2:1:1:1 and 4:1:1:1, optionally between 2:1:1:1 and 3:1:1:1.

In some embodiments, the ratio of (a):(b): (c¹): (c²) is about 1.5:1:1:1, about 2:1:1:1, about 2.2:1:1:1, about 2.4:1:1:1, about 2.6:1:1:1, about 2.8:1:1:1, about 3:1:1:1, about 3.2:1:1:1, about 3.4:1:1:1, about 3.6:1:1:1, about 3.8:1:1:1, about 4:1:1:1, about 4.2:1:1:1, about 4.4:1:1:1, about 4.6:1:1:1, about 4.8:1:1:1 or about 5:1:1:1.

In some embodiments, the ratio of (a):(b): (c¹): (c²) is 1.5:1:1:1, 2:1:1:1, 2.2:1:1:1, 2.4:1:1:1, 2.6:1:1:1, 2.8:1:1:1, 3:1:1:1, 3.2:1:1:1, 3.4:1:1:1, 3.6:1:1:1, 3.8:1:1:1, 4:1:1:1, 4.2:1:1:1, 4.4:1:1:1, 4.6:1:1:1, 4.8:1:1:1 or 5:1:1:1.

In some embodiments, the ratio of (a):(b): (c¹): (c²) is comprised between 2:1:1:1 and 4:1:1:1, suitably between 2:1:1:1 and 3:1:1:1, suitably is 2:1:1:1 or 3:1:1:1.

In some embodiments, the ratio of (a):(b): (c¹): (c²) is greater than 3:1:1:1 and lower of equal to 20:1:1:1.

In some embodiments, the ratio of (a):(b): (c¹): (c²) is greater than 3:1:1:1 and lower of equal to 5:1:1:1.

In some embodiments, the ratio of (a):(b): (c¹): (c²) is comprised between 1.5:1:1.5 and 20:1:20.

In some embodiments, the ratio of (a):(b): (c¹): (c²) is comprised between 1.5:1:5.5 and 20:1:20.

In some embodiments, the ratio of (a):(b): (c¹): (c²) is comprised between 4:1:1.5 and 20:1:20.

In some embodiments, the ratio of (a):(b): (c¹): (c²) is comprised between 4:1:5.5 and 20:1:20.

In some embodiments, the ratio of (a):(b): (c¹): (c²) is comprised between 1.5:1:1.5:1.5 and 5:1:5:5, optionally between 2:1:2:2 and 5:1:5:5, optionally between 3:1:3:3 and 5:1:5:5, optionally between 4:1:4:4 and 5:1:5:5, optionally between 1.5:1:1.5:1.5 and 4:1:4:4, optionally between 1.5:1:1.5:1.5 and 3:1:3:3, optionally between 2:1:2:2 and 4:1:4:4, optionally between 2:1:2:2 and 3:1:3:3, suitably is 2:1:2:2, 2:1:3:3, 3:1:2:2 or 3:1:3:3.

In some embodiments, the composition comprises seven antigens or nucleic acids, suitably mRNAs, encoding such.

In some embodiments, the immunogenic composition comprises a combination of four HA antigens or four nucleic acids, suitably mRNAs, encoding said four HA antigens, and three NA antigens or three nucleic acids, suitably mRNAs, encoding said three NA antigens.

In some embodiments, the immunogenic composition comprises:
(a), (b), (c¹) and (c²) as defined herein; suitably (a) being a first HA antigen or a first nucleic acid, suitably mRNA, encoding the first HA antigen wherein the first HA antigen is derived from a strain of subtype H3 of Influenza A virus, suitably H3N2, and/or (b) being a second HA antigen or a second nucleic acid, suitably mRNA, encoding the second HA antigen wherein the second HA antigen is derived from a strain of subtype H1 of Influenza A virus, suitably H1N1, and/or (c¹) being a third HA antigen or a third nucleic acid, suitably mRNA, encoding the third HA antigen wherein the third HA antigen is derived from a first strain of Influenza B virus, suitably from B/Victoria lineage, and/or (c²) being a fourth HA antigen or a fourth nucleic acid, suitably mRNA, encoding the fourth HA antigen wherein the fourth HA antigen is derived from a second strain of Influenza B virus, suitably from B/Yamagata lineage;
wherein the immunogenic composition further comprises:
(c³) a first NA antigen or a first nucleic acid, suitably mRNA, encoding the first NA antigen wherein the first NA antigen is derived from the strain of subtype H1 of Influenza A virus;
(c⁴) a second NA antigen or a second nucleic acid, suitably mRNA, encoding the second NA antigen wherein the second NA antigen is derived from the strain of subtype H3 of Influenza A virus; and
(c⁵) a third NA antigen or a third nucleic acid, suitably mRNA, encoding the third NA antigen wherein the third NA antigen is derived from the first strain of Influenza B virus,
   wherein (a), (b), (c¹), (c²), (c³), (c⁴) and (c⁵) are different.

In some embodiments, wherein the ratio of (a):(b): (c¹): (c²) is comprised between 1.5:1:1:1 and 20:1:1:1, optionally between 1.5:1:1:1 and 5:1:1:1.

In some embodiments, the ratio of (a):(b): (c¹): (c²) is greater than 1:1:1:1, optionally greater than 1:1:1:1 and lower or equal to 20:1:1:1, optionally greater than 1:1:1:1 and lower or equal to 5:1:1:1.

In some embodiments, the ratio of (a):(b): (c¹): (c²) is comprised between 1.5:1:1:1 and 5:1:1:1, optionally between 2:1:1:1 and 5:1:1:1, optionally between 3:1:1:1 and 5:1:1:1, optionally between 4:1:1:1 and 5:1:1:1, optionally between 1.5:1:1:1 and 4:1:1:1, optionally between 1.5:1:1:1 and 3:1:1:1, optionally between 2:1:1:1 and 4:1:1:1, optionally between 2:1:1:1 and 3:1:1:1.

In some embodiments, the ratio of (a):(b): (c¹): (c²) is about 1.5:1:1:1, about 2:1:1:1, about 2.2:1:1:1, about 2.4:1:1:1, about 2.6:1:1:1, about 2.8:1:1:1, about 3:1:1:1, about 3.2:1:1:1, about 3.4:1:1:1, about 3.6:1:1:1, about 3.8:1:1:1, about 4:1:1:1, about 4.2:1:1:1, about 4.4:1:1:1, about 4.6:1:1:1, about 4.8:1:1:1 or about 5:1:1:1.

In some embodiments, the ratio of (a):(b): (c¹): (c²) is 1.5:1:1:1, 2:1:1:1, 2.2:1:1:1, 2.4:1:1:1, 2.6:1:1:1, 2.8:1:1:1, 3:1:1:1, 3.2:1:1:1, 3.4:1:1:1, 3.6:1:1:1, 3.8:1:1:1, 4:1:1:1, 4.2:1:1:1, 4.4:1:1:1, 4.6:1:1:1, 4.8:1:1:1 or 5:1:1:1.

In some embodiments, the ratio of (a):(b): (c¹): (c²) is comprised between 2:1:1:1 and 4:1:1:1, suitably between 2:1:1:1 and 3:1:1:1, suitably is 2:1:1:1 or 3:1:1:1.

In some embodiments, the ratio of (a):(b): (c¹): (c²) is greater than 3:1:1:1 and lower of equal to 20:1:1:1.

In some embodiments, the ratio of (a):(b): (c¹): (c²) is greater than 3:1:1:1 and lower of equal to 5:1:1:1.

In some embodiments, the ratio of (a):(b): (c¹): (c²) is comprised between 1.5:1:1.5:1.5 and 20:1:20:20.

In some embodiments, the ratio of (a):(b): (c¹): (c²) is comprised between 1.5:1:5.5:5.5 and 20:1:20:20.

In some embodiments, the ratio of (a):(b): (c¹): (c²) is comprised between 4:1:1.5:1.5 and 20:1:20:20.

In some embodiments, the ratio of (a):(b): (c¹): (c²) is comprised between 4:1:5.5:5.5 and 20:1:20:20.

In some embodiments, the ratio of (a):(b): (c¹): (c²) is comprised between 1.5:1:1.5:1.5 and 5:1:5:5, optionally between 2:1:2:2 and 5:1:5:5, optionally between 3:1:3:3 and 5:1:5:5, optionally between 4:1:4:4 and 5:1:5:5, optionally between 1.5:1:1.5:1.5 and 4:1:4:4, optionally between 1.5:1:1.5:1.5 and 3:1:3:3, optionally between 2:1:2:2 and 4:1:2:2, optionally between 2:1:2:2 and 3:1:3:3, suitably is 2:1:2:2, 2:1:3:3, 3:1:2:2 or 3:1:3:3.

In some embodiments, the ratio of (a):(b): (c¹): (c²): (c³): (c⁴): (c⁵) is comprised between 9:3:3:3:1:1:1 and 3:1:1:1:3:3:3, suitably between 6:2:2:2:1:1:1 and 3:1:1:1:2:2:2, suitably is 6:2:2:2:1:1:1 or 3:1:1:1:2:2:2.

In some embodiments, the ratio of (a):(b): (c¹): (c²): (c³): (c⁴): (c⁵) is comprised between 15:3:3:3:1:1:1 and 5:1:1:1:3:3:3, suitably is 10:2:2:2:1:1:1 or 15:3:3:3:1:1:1 or 8:2:2:2:1:1:1 or 12:3:3:3:1:1:1.

In some embodiments, the ratio of (a):(b): (c¹): (c²): (c³): (c⁴): (c⁵) is comprised between 9:3:9:9:1:1:1 and 3:1:3:3:3:3:3, suitably between 6:2:6:6:1:1:1 and 3:1:3:3:2:2:2, suitably is 6:2:6:6:1:1:1 or 3:1:3:3:2:2:2.

In some embodiments, the ratio of (a):(b): (c¹): (c²): (c³): (c⁴): (c⁵) is comprised between 15:3:15:15:1:1:1 and 5:1:5:5:3:3:3, suitably is 10:2:10:10:1:1:1 or 15:3:15:15:1:1:1 or 8:2:8:8:1:1:1 or 12:3:12:12:1:1:1.

In some embodiments, the ratio is a weight/weight ratio or a molar ratio. Suitably, the ratio is a weight/weight ratio.

In some embodiments, the composition comprises eight antigens or nucleic acids, suitably mRNAs, encoding such.

In some embodiments, the immunogenic composition comprises a combination of four HA antigens or four nucleic acids, suitably mRNAs, encoding said four HA antigens, and four NA antigens or four nucleic acids, suitably mRNAs, encoding said four NA antigens.

In some embodiments, the composition further comprises:
(c⁶) a fourth NA antigen or a fourth nucleic acid, suitably mRNA, encoding the fourth NA antigen wherein the fourth NA antigen is derived from the second strain of Influenza B virus.

In some embodiments, the composition comprises:
(c⁶) a fourth NA antigen or a fourth nucleic acid, suitably mRNA, encoding the fourth NA antigen wherein the fourth NA antigen is derived from the second strain of Influenza B virus
   wherein (a), (b), (c¹), (c²), (c³), (c⁴), (c⁵) and (c⁶) are different.

In some embodiments, the ratio of (a):(b): (c¹): (c²) is comprised between 1.5:1:1:1 and 20:1:1:1, optionally between 1.5:1:1:1 and 5:1:1:1.

In some embodiments, the ratio of (a):(b): (c¹): (c²) is greater than 1:1:1:1, optionally greater than 1:1:1:1 and lower or equal to 20:1:1:1, optionally greater than 1:1:1:1 and lower or equal to 5:1:1:1.

In some embodiments, the ratio of (a):(b): (c¹): (c²) is comprised between 1.5:1:1:1 and 5:1:1:1, optionally between 2:1:1:1 and 5:1:1:1, optionally between 3:1:1:1 and 5:1:1:1, optionally between 4:1:1:1 and 5:1:1:1, optionally between 1.5:1:1:1 and 4:1:1:1, optionally between 1.5:1:1:1 and 3:1:1:1, optionally between 2:1:1:1 and 4:1:1:1, optionally between 2:1:1:1 and 3:1:1:1.

In some embodiments, the ratio of (a):(b): (c¹): (c²) is about 1.5:1:1:1, about 2:1:1:1, about 2.2:1:1:1, about 2.4:1:1:1, about 2.6:1:1:1, about 2.8:1:1:1, about 3:1:1:1, about 3.2:1:1:1, about 3.4:1:1:1, about 3.6:1:1:1, about 3.8:1:1:1, about 4:1:1:1, about 4.2:1:1:1, about 4.4:1:1:1, about 4.6:1:1:1, about 4.8:1:1:1 or about 5:1:1:1.

In some embodiments, the ratio of (a):(b): (c¹): (c²) is 1.5:1:1:1, 2:1:1:1, 2.2:1:1:1, 2.4:1:1:1, 2.6:1:1:1, 2.8:1:1:1, 3:1:1:1, 3.2:1:1:1, 3.4:1:1:1, 3.6:1:1:1, 3.8:1:1:1, 4:1:1:1, 4.2:1:1:1, 4.4:1:1:1, 4.6:1:1:1, 4.8:1:1:1 or 5:1:1:1.

In some embodiments, the ratio of (a):(b): (c¹): (c²) is comprised between 2:1:1:1 and 4:1:1:1, suitably between 2:1:1:1 and 3:1:1:1, suitably is 2:1:1:1 or 3:1:1:1.

In some embodiments, the ratio of (a):(b): (c¹): (c²) is greater than 3:1:1:1 and lower of equal to 20:1:1:1.

In some embodiments, the ratio of (a):(b): (c¹): (c²) is greater than 3:1:1:1 and lower of equal to 5:1:1:1.

In some embodiments, the ratio of (a):(b): (c¹): (c²) is comprised between 1.5:1:1.5:1.5 and 20:1:20:20.

In some embodiments, the ratio of (a):(b): (c¹): (c²) is comprised between 1.5:1:5.5:5.5 and 20:1:20:20.

In some embodiments, the ratio of (a):(b): (c¹): (c²) is comprised between 4:1:1.5:1.5 and 20:1:20:20.

In some embodiments, the ratio of (a):(b): (c¹): (c²) is comprised between 4:1:5.5:5.5 and 20:1:20:20.

In some embodiments, the ratio of (a):(b): (c¹): (c²) is comprised between 1.5:1:1.5:1.5 and 5:1:5:5, optionally between 2:1:2:2 and 5:1:5:5, optionally between 3:1:3:3 and 5:1:5:5, optionally between 4:1:4:4 and 5:1:5:5, optionally between 1.5:1:1.5:1.5 and 4:1:4:4, optionally between 1.5:1:1.5:1.5 and 3:1:3:3, optionally between 2:1:2:2 and 4:1:2:2, optionally between 2:1:2:2 and 3:1:3:3, suitably is 2:1:2:2, 2:1:3:3, 3:1:2:2 or 3:1:3:3.

In some embodiments, the ratio of (a):(b): (c¹): (c²): (c³): (c⁴): (c⁵): (c⁶) is comprised between 9:3:3:3:1:1:1:1 and 3:1:1:1:3:3:3:3, suitably between 6:2:2:2:1:1:1:1 and 3:1:1:1:2:2:2:2, suitably is 6:2:2:2:1:1:1:1 or 3:1:1:1:2:2:2:2.

In some embodiments, the ratio of (a):(b): (c¹): (c²): (c³): (c⁴): (c⁵): (c⁶) is comprised between 15:3:3:3:1:1:1:1 and 5:1:1:1:3:3:3:3, suitably is 10:2:2:2:1:1:1:1 or 15:3:3:3:1:1:1:1 or 8:2:2:2:1:1:1:1 or 12:3:3:3:1:1:1:1.

In some embodiments, the ratio of (a):(b): (c¹): (c²): (c³): (c⁴): (c⁵): (c⁶) is comprised between 9:3:9:9:1:1:1:1 and 3:1:3:3:3:3:3:3, suitably between 6:2:6:6:1:1:1:1 and 3:1:3:3:2:2:2:2, suitably is 6:2:6:6:1:1:1:1, 3:1:3:3:2:2:2:2, 4:2:6:6:1:1:1:1 or 2:1:3:3:2:2:2:2.

In some embodiments, the ratio of (a):(b): (c¹): (c²): (c³): (c⁴): (c⁵): (c⁶) is comprised between 15:3:15:15:1:1:1:1 and 5:1:5:5:3:3:3:3, suitably is 10:2:10:10:1:1:1:1 or 15:3:15:15:1:1:1:1 or 8:2:8:8:1:1:1:1 or 12:3:12:12:1:1:1:1.

In some embodiments, the ratio is a weight/weight ratio or a molar ratio. Suitably, the ratio is a weight/weight ratio.

In some embodiments, the composition comprises six antigens or nucleic acids, suitably mRNAs, encoding such.

In some embodiments, the immunogenic composition comprises a combination of three HA antigens or three nucleic acids, suitably mRNAs, encoding said three HA antigens, and three NA antigens or three nucleic acids, suitably mRNAs, encoding said three NA antigens.

In some embodiments, the immunogenic composition comprises:
(a) as defined herein; suitably being a first HA antigen or a first nucleic acid, suitably mRNA, encoding the first HA antigen wherein the first HA antigen is derived from a strain of subtype H3 of Influenza A virus, suitably H3N2;
(b) as defined herein; suitably being a second HA antigen or a second nucleic acid, suitably mRNA, encoding the second HA antigen wherein the second HA antigen is derived from a strain of subtype H1 of Influenza A virus, suitably H1N1;
(c¹) as defined herein; suitably being a third HA antigen or a third nucleic acid, suitably mRNA, encoding the third HA antigen wherein the third HA antigen is derived from a first strain of Influenza B virus, suitably from B/Victoria lineage;
(c³) as defined herein; suitably being a first NA antigen or a first nucleic acid, suitably mRNA, encoding the first NA antigen wherein the first NA antigen is derived from the strain of subtype H1 of Influenza A virus;
(c⁴) as defined herein; suitably being a second NA antigen or a second nucleic acid, suitably mRNA, encoding the second NA antigen wherein the second NA antigen is derived from the strain of subtype H3 of Influenza A virus; and
(c⁵) as defined herein; suitably being a third NA antigen or a third nucleic acid, suitably mRNA, encoding the third NA antigen wherein the third NA antigen is derived from the first strain of Influenza B virus;
   wherein (a), (b), (c¹), (c³), (c⁴) and (c⁵) are different.

In some embodiments, the ratio of (a):(b): (c¹) is comprised between 1.5:1:1 and 20:1:1, optionally between 1.5:1:1 and 5:1:1.

In some embodiments, the ratio of (a):(b): (c¹) is greater than 1:1:1, optionally greater than 1:1:1 and lower or equal to 20:1:1, optionally greater than 1:1:1 and lower or equal to 5:1:1.

In some embodiments, the ratio of (a):(b): (c¹) is comprised between 1.5:1:1 and 5:1:1, optionally between 2:1:1 and 5:1:1, optionally between 3:1:1 and 5:1:1, optionally between 4:1:1 and 5:1:1, optionally between 1.5:1:1 and 4:1:1, optionally between 1.5:1:1 and 3:1:1, optionally between 2:1:1 and 4:1:1, optionally between 2:1:1 and 3:1:1.

In some embodiments, the ratio of (a):(b): (c¹) is about 1.5:1:1, about 2:1:1, about 2.2:1:1, about 2.4:1:1, about 2.6:1:1, about 2.8:1:1, about 3:1:1, about 3.2:1:1, about 3.4:1:1, about 3.6:1:1, about 3.8:1:1, about 4:1:1, about 4.2:1:1, about 4.4:1:1, about 4.6:1:1, about 4.8:1:1 or about 5:1:1.

In some embodiments, the ratio of (a):(b): (c¹) is 1.5:1:1, 2:1:1, 2.2:1:1, 2.4:1:1, 2.6:1:1, 2.8:1:1, 3:1:1, 3.2:1:1, 3.4:1:1, 3.6:1:1, 3.8:1:1, 4:1:1, 4.2:1:1, 4.4:1:1, 4.6:1:1, 4.8:1:1 or 5:1:1.

In some embodiments, the ratio of (a):(b): (c¹) is comprised between 2:1:1 and 4:1:1, suitably between 2:1:1 and 3:1:1, suitably is 2:1:1 or 3:1:1.

In some embodiments, the ratio of (a):(b): (c¹) is greater than 3:1:1 and lower of equal to 20:1:1.

In some embodiments, the ratio of (a):(b): (c¹) is greater than 3:1:1 and lower of equal to 5:1:1.

In some embodiments, the ratio of (a):(b): (c¹) is comprised between 1.5:1:1.5 and 20:1:20.

In some embodiments, the ratio of (a):(b): (c¹) is comprised between 1.5:1:5.5 and 20:1:20.

In some embodiments, the ratio of (a):(b): (c¹) is comprised between 4:1:1.5 and 20:1:20.

In some embodiments, the ratio of (a):(b): (c¹) is comprised between 4:1:5.5 and 20:1:20.

In some embodiments, the ratio of (a):(b): (c¹) is comprised between 1.5:1:1.5 and 5:1:5, optionally between 2:1:2 and 5:1:5, optionally between 3:1:3 and 5:1:5, optionally between 4:1:4 and 5:1:5, optionally between 1.5:1:1.5 and 4:1:4, optionally between 1.5:1:1.5 and 3:1:3, optionally between 2:1:2 and 4:1:2, optionally between 2:1:2 and 3:1:3, suitably is 2:1:2, 2:1:3, 3:1:2 or 3:1:3.

In some embodiments, the ratio of (a):(b): (c¹): (c³): (c⁴): (c⁵) is comprised between 9:3:3:1:1:1 and 3:1:1:3:3:3, suitably between 6:2:2:1:1:1 and 3:1:1:2:2:2, suitably is 6:2:2:1:1:1 or 3:1:1:2:2:2.

In some embodiments, the ratio of (a):(b): (c¹): (c³): (c⁴): (c⁵) is comprised between 15:3:3:1:1:1 and 5:1:1:3:3:3, suitably is 10:2:2:1:1:1 or 15:3:3:1:1:1 or 8:2:2:1:1:1 or 12:3:3:1:1:1.

In some embodiments, the ratio of (a):(b): (c¹): (c³): (c⁴): (c⁵) is comprised between 9:3:9:1:1:1 and 3:1:3:3:3:3, suitably between 6:2:6:1:1:1 and 3:1:3:2:2:2, suitably is 6:2:6:1:1:1 or 3:1:3:2:2:2.

In some embodiments, the ratio of (a):(b): (c¹): (c³): (c⁴): (c⁵) is comprised between 15:3:15:1:1:1 and 5:1:5:3:3:3, suitably is 10:2:10:1:1:1 or 15:3:15:1:1:1 or 8:2:8:1:1:1 or 12:3:12:1:1:1.

In some embodiments, the ratio is a weight/weight ratio or a molar ratio. Suitably, the ratio is a weight/weight ratio.

It has to be noted that specific features and embodiments that are described in the context of the first aspect of the invention, that is the immunogenic composition of the invention, are likewise applicable to the second aspect (vaccine of the invention), the third aspect (kit or kit of parts of the invention), or further aspects including e.g. medical uses (first and second medical uses) and e.g. method of treatments.

### Nucleic Acids

In some embodiments, at least one nucleic acid of the immunogenic composition, suitably of (a), (b), (c), (c¹), (c²), (c³), (c⁴), (c⁵) and/or (c⁶), is DNA or RNA, suitably mRNA. In some embodiments, as least one nucleic acid of the immunogenic composition, suitably of (a), (b), (c), (c¹), (c²), (c³), (c⁴), (c⁵) and/or (c⁶), is a DNA.

In some embodiments, at least one nucleic acid of the immunogenic composition, suitably of (a), (b), (c), (c¹), (c²), (c³), (c⁴), (c⁵) and/or (c⁶), is an artificial nucleic acid, e.g. an artificial DNA or an artificial RNA, suitably mRNA.

Nucleic acid-based vaccination including DNA or RNA, suitably mRNA, represents a promising technique for novel vaccines against emerging viruses and for the provision of combination vaccines. Nucleic acids can be genetically engineered and administered to a human subject. Transfected cells directly produce the encoded antigen (e.g. provided by a DNA or an RNA, in particular an mRNA), which results in protective immunological responses.

Nucleic acids according to the invention, e.g. DNAs or RNAs, suitably mRNAs, form the basis for a nucleic acid based immunogenic composition or a nucleic acid based vaccine.

Such nucleic acid based immunogenic compositions (first aspect) or nucleic acid-based vaccines (second aspect) as provided herein have advantages over classical vaccine approaches.

In general, protein-based vaccines, or live attenuated vaccines are suboptimal for use in developing countries due to their high production costs. In addition, protein-based vaccines, or live attenuated vaccines require long development times and are not suitable for rapid responses of epidemic virus outbreaks such as e.g. the Influenza virus outbreaks. Indeed, because traditional methods for producing standard inactivated flu vaccines take a long period of time, the GISRS recommendation is made six to seven months prior the start of the Influenza season, during which the Influenza viruses may continue to evolve.

In contrast, the nucleic acid-based immunogenic compositions and vaccines according to the invention allow very fast and cost-effective manufacturing. Therefore, in comparison with known vaccines, compositions/vaccines based on nucleic acids can be produced and manufactured significantly cheaper and faster, which is very advantageous particularly for use in developing countries or in the context of annual epidemics or a global pandemic. The nucleic acid-based compositions/vaccines offer the GISRS additional time to monitor circulating viruses and make its recommendation closer to the Influenza season. This extension of the GISRS monitoring timeline should allow the GISRS predictions to be more accurate, resulting in more effective vaccines designated to target circulating viruses closer to Influenza season. Furthermore, the different nucleic acid encoding different antigens (e.g. of different Influenza strains) can be combined in one immunogenic composition/vaccine to ensure or increase the effectiveness of the immune response against Influenza virus.

The use of RNA, suitably mRNA, in or as a vaccine overcomes the disadvantages of conventional genetic vaccination involving incorporating DNA into cells in terms of safeness, feasibility, applicability, and effectiveness to generate immune responses. RNA molecules, suitably mRNAs, are considered to be significantly safer than DNA vaccines, as RNAs, suitably mRNAs, are more easily degraded. They are cleared quickly out of the organism and cannot integrate into the genome and influence the cell's gene expression in an uncontrollable manner. It is also less likely for RNA, suitably mRNA, vaccines to cause severe side effects like the generation of autoimmune disease or anti-DNA antibodies. Transfection with RNA, suitably mRNA, requires only insertion into the cell's cytoplasm, which is easier to achieve than into the nucleus.

In some embodiments, at least one nucleic acid of the immunogenic composition, suitably of (a), (b), (c), (c¹), (c²), (c³), (c⁴), (c⁵) and/or (c⁶), is an RNA.

Therefore, in some embodiments, (a) is a first RNA encoding the first HA antigen and/or (b) is a second RNA encoding the second HA antigen.

In some embodiments, (c) is at least one further RNA encoding the at least one further antigen.

In some embodiments, the immunogenic composition comprises a plurality of (c) being RNAs.

In some embodiments, (a), (b), (c), (c¹), (c²), (c³), (c⁴), (c⁵) and/or (c⁶) is an RNA.

Messenger RNA (mRNA) is a single-stranded RNA molecule that corresponds to the genetic sequence of a gene and is read by ribosomes in the process of producing a protein. mRNA vaccines may utilise non-replicating mRNA or self-replicating RNA (also referred to as self-amplifying mRNA or SAM). Non-replicating mRNA-based vaccines typically encode an antigen of interest and contain 5′ and 3′ untranslated regions (UTRs), a 5′ cap and a poly(A) tail; whereas self-amplifying RNAs also encode viral replication machinery that enables intracellular RNA amplification.

mRNA-based Influenza vaccine candidates are currently under clinical trials. For instance, mRNA-1010 is an mRNA vaccine candidate that encodes for HA glycoproteins of the four influenza strains recommended by the WHO for the prevention of influenza. In phase I study, mRNA-1010 was evaluated at 50, 100 and 200 μg total dose levels in equimolar proportions in younger adults and older adult cohorts.

In some embodiments, at least one nucleic acid of the immunogenic composition, suitably of (a), (b), (c), (c¹), (c²), (c³), (c⁴), (c⁵) and/or (c⁶), is a mRNA.

In some embodiments, (a) is a first mRNA encoding the first HA antigen and/or (b) is a second mRNA encoding the second HA antigen.

In some embodiments, a dose of each said first mRNA and/or said second mRNA is 1 to 200 μg, suitably 1 to 60 μg, suitably 2 to 25 μg.

In some embodiments, a dose of each said first mRNA and/or said second mRNA is 2 to 25 μg, optionally 2 to 18 μg, optionally 2 to 9 μg, optionally 2 to 6 μg, optionally 3 to 25 μg, 3 to 18 μg, 3 to 9 μg, optionally 3 to 6 μg.

In some embodiments, a dose of each said first mRNA and/or said second mRNA is 1, 2, 3, 4, 5, 6, 7, 8, 9, 10, 11, 12, 13, 14, 15, 16, 17, 18, 19, 20, 21, 22, 23, 24 or 25 μg, optionally 2, 3, 6, 9 or 18 μg.

In some embodiments, a dose of each said first mRNA and/or said second mRNA is 3, 6, 9, 12 or 18 μg.

In some embodiments, a dose of each said first and/or second mRNA is 0.5 to 200 μg, optionally 2 to 25 μg, optionally 2 to 30 μg, optionally 2 to 40 μg, optionally 2 to 45 μg, optionally 2 to 50 μg, optionally 2 to 75 μg.

In some embodiments, a dose of each said first and/or second mRNA is 0.5, 1, 2, 3, 4, 5, 6, 7, 8, 9, 10, 11, 12, 13, 14, 15, 16, 17, 18, 19, 20, 21, 22, 23, 24, 25, 30, 35, 36, 37, 38, 39, 40, 45, 46, 47, 48, 49, 50, 55, 60, 65, 70, 71, 72, 73, 74 or 75 μg, optionally 3, 6, 9, 24, 36, 48 or 72 μg.

In some embodiments, (c) is at least one further mRNA encoding the at least one further antigen.

In some embodiments, a dose of each said at least one further mRNA is 1 to 200 μg, suitably 1 to 60 μg, suitably 2 to 25 μg.

In some embodiments, a dose of each said at least one further mRNA is 2 to 25 μg, optionally 2 to 18 μg, optionally 2 to 9 μg, optionally 2 to 6 μg, optionally 3 to 25 μg, 3 to 18 μg, 3 to 9 μg, optionally 3 to 6 μg.

In some embodiments, a dose of each said at least one further mRNA is 1, 2, 3, 4, 5, 6, 7, 8, 9, 10, 11, 12, 13, 14, 15, 16, 17, 18, 19, 20, 21, 22, 23, 24 or 25 μg, optionally 2, 3, 6, 9 or 18 μg.

In some embodiments, a dose of each said at least one further mRNA is 3, 6, 9, 12 or 18 μg.

In some embodiments, a dose of each said at least one further mRNA is 0.5 to 200 μg, optionally 2 to 10 μg, 2 to 25 μg, optionally 2 to 30 μg, optionally 2 to 40 μg, optionally 2 to 45 μg, optionally 2 to 50 μg, optionally 2 to 75 μg.

In some embodiments, a dose of each said at least one further mRNA is 1, 2, 3, 4, 5, 6, 7, 8, 9, 10, 11, 12, 13, 14, 15, 16, 17, 18, 19, 20, 21, 22, 23, 24, 25, 30, 35, 36, 37, 38, 39, 40, 45, 46, 47, 48, 49, 50, 55, 60, 65, 70, 71, 72, 73, 74 or 75 μg, optionally 3, 6, 9, 24, 36, 48 or 72 μg.

In some embodiments, the immunogenic composition comprises a plurality of (c) being mRNAs.

In some embodiments, the composition comprises at least three, four, five, six, seven or eight mRNAs, optionally three to ten mRNAs, optionally three or six mRNAs.

In some embodiments, the third, fourth, fifth, sixth, seventh and/or eighth nucleic acid is an mRNA.

In some embodiments, (a), (b), (c), (c¹), (c²), (c³), (c⁴), (c⁵) and/or (c⁶) is an mRNA.

In some embodiments, a dose of each (a), (b), (c), (c¹), (c²), (c³), (c⁴), (c⁵) and/or (c⁶) is 1 to 200 μg, suitably 1 to 60 μg, suitably 1 to 25 μg, suitably 2 to 25 μg.

In some embodiments, a dose of each (a), (b), (c), (c¹), (c²), (c³), (c⁴), (c⁵) and/or (c⁶) is 1 to 25 μg, optionally 2 to 25 μg, optionally 2 to 18 μg, optionally 2 to 9 μg, optionally 2 to 6 μg, optionally 3 to 25 μg, 3 to 18 μg, 3 to 9 μg, optionally 3 to 6 μg.

In some embodiments, a dose of each (a), (b), (c), (c¹), (c²), (c³), (c⁴), (c⁵) and/or (c⁶) is 1, 2, 3, 4, 5, 6, 7, 8, 9, 10, 11, 12, 13, 14, 15, 16, 17, 18, 19, 20, 21, 22, 23, 24 or 25 μg, optionally 1, 2, 3, 6, 9 or 18 μg.

In some embodiments, a dose of each (a), (b), (c), (c¹), (c²), (c³), (c⁴), (c⁵) and/or (c⁶) is 1, 2, 3, 6, 9, 12 or 18 μg.

In some embodiments, a dose of each (a), (b), (c), (c¹), (c²), (c³), (c⁴), (c⁵) and/or (c⁶) is 0.5 to 200 μg, optionally 2 to 10 μg, 2 to 25 μg, optionally 2 to 30 μg, optionally 2 to 40 μg, optionally 2 to 45 μg, optionally 2 to 50 μg, optionally 2 to 75 μg.

In some embodiments, a dose of each (a), (b), (c), (c¹), (c²), (c³), (c⁴), (c⁵) and/or (c⁶) is 0.5, 1, 2, 3, 4, 5, 6, 7, 8, 9, 10, 11, 12, 13, 14, 15, 16, 17, 18, 19, 20, 21, 22, 23, 24, 25, 30, 35, 36, 37, 38, 39, 40, 45, 46, 47, 48, 49, 50, 55, 60, 65, 70, 71, 72, 73, 74 or 75 μg, optionally 3, 6, 9, 24, 36, 48 or 72 μg.

Also provided herein is an immunogenic composition comprising:
(a) a first mRNA encoding a HA of a strain of subtype H3 of Influenza A virus, suitably H3N2;
(b) a second mRNA encoding a HA of a strain of subtype H1 of Influenza A virus, suitably H1N1;
(c¹) a third mRNA encoding a HA of a first strain of Influenza B virus, suitably from B/Victoria lineage; and
(c²) optionally, a fourth mRNA encoding a HA of a second strain of Influenza B virus, suitably from B/Yamagata lineage,
   wherein the ratio of (a):(b): (c¹) is comprised between 1.5:1:1 and 20:1:1, optionally between 1.5:1:1 and 5:1:1, suitably between 2:1:1 and 4:1:1, suitably between 2:1:1 and 3:1:1, suitably is 2:1:1 or 3:1:1.

In some embodiments, the immunogenic composition further comprises:
(c²) a fourth mRNA encoding a HA of a second strain of Influenza B virus, suitably from B/Yamagata lineage,
   wherein the ratio of (a):(b): (c¹): (c²) is comprised between 1.5:1:1:1 and 5:1:1:1, suitably between 2:1:1:1 and 4:1:1:1, suitably between 2:1:1:1 and 3:1:1:1, suitably is 2:1:1:1 or 3:1:1:1.

In some embodiments, the ratio is a weight/weight ratio or a molar ratio. Suitably, the ratio is a weight/weight ratio.

In some embodiments, a dose of (a) is 5 to 50 μg, optionally 10 to 40 μg, optionally 12 to 36 μg.

In some embodiments, a dose of (a) is 10, 11, 12, 13, 14, 15, 16, 17, 18, 19, 20, 21, 22, 23, 24, 25, 26, 27, 28, 29, 30, 31, 32, 33, 34, 35,36, 37, 38, 39 or 40 μg.

In some embodiments, a dose of (a) is 0.5 to 200 μg, optionally 2 to 10 μg, 2 to 25 μg, optionally 2 to 30 μg, optionally 2 to 40 μg, optionally 2 to 45 μg, optionally 2 to 50 μg, optionally 2 to 75 μg.

In some embodiments, a dose of (a) is 0.5, 1, 2, 3, 4, 5, 6, 7, 8, 9, 10, 11, 12, 13, 14, 15, 16, 17, 18, 19, 20, 21, 22, 23, 24, 25, 30, 35, 36, 37, 38, 39, 40, 45, 46, 47, 48, 49, 50, 55, 60, 65, 70, 71, 72, 73, 74 or 75 μg, optionally 3, 6, 9, 24, 36, 48 or 72 μg.

In some embodiments, a dose of each of (b), (c¹) and/or (c²) is 2 to 20 μg, optionally 5 to 15 μg, optionally 6 to 12 μg.

In some embodiments, a dose of each of (b), (c¹) and/or (c²) is 2, 3, 4, 5, 6, 7, 8, 9, 10, 11, 12, 13, 14, 15, 16, 17, 18, 19 or 20 μg.

In some embodiments, a dose of each of (b), (c¹) and/or (c³) is 5, 6, 7, 8, 9, 10, 11, 12, 13, 14 or 15 μg.

In some embodiments, a dose of each of (b), (c¹) and/or (c²) is 0.5 to 200 μg, optionally 2 to 10 μg, 2 to 25 μg, optionally 2 to 30 μg, optionally 2 to 40 μg, optionally 2 to 45 μg, optionally 2 to 50 μg, optionally 2 to 75 μg.

In some embodiments, a dose of each of (b), (c¹) and/or (c²) is 0.5, 1, 2, 3, 4, 5, 6, 7, 8, 9, 10, 11, 12, 13, 14, 15, 16, 17, 18, 19, 20, 21, 22, 23, 24, 25, 30, 35, 36, 37, 38, 39, 40, 45, 46, 47, 48, 49, 50, 55, 60, 65, 70, 71, 72, 73, 74 or 75 μg, optionally 3, 6, 9, 24, 36, 48 or 72 μg.

In some embodiments, a dose of (a), (b) and (c¹) is 25 to 150 μg, optionally 25 to 100 μg, optionally 30 to 90 μg.

In some embodiments, a dose of (a), (b) and (c¹) is 25 to 100 μg.

In some embodiments, a dose of (a), (b) and (c¹) is 25, 26, 27, 28, 29, 30, 31, 32, 33, 34, 35, 36, 37, 38, 39, 40, 45, 50, 51, 52, 53, 54, 55, 56, 57, 58, 59, 60, 65, 70, 75, 80, 85, 86, 87, 88, 89, 90, 91, 92, 93, 94, 95 or 100 μg.

In some embodiments, a dose of (a), (b), (c¹) and (c²) is 5 to 75 μg, optionally 10 to 60 μg, optionally 12 to 48 μg.

In some embodiments, a dose of (a), (b), (c¹) and (c²) is 35 to 75 μg.

In some embodiments, a dose of (a), (b), (c¹) and (c²) is 35, 36, 37, 38, 39, 40, 45, 46, 47, 48, 49, 50, 51, 52, 53, 54, 55, 70, 71, 72, 73, 74 or 75 μg.

In some embodiments, a dose of (a), (b), (c¹) and (c²) is 10, 11, 12, 13, 14, 15, 16, 17, 18, 19, 20, 21, 22, 24, 24, 25, 26, 27, 28, 29, 30, 45, 46, 47, 48, 49, 50, 55, 60 μg. In some embodiments, a dose of (a) is 5 to 50 μg, optionally 10 to 40 μg, optionally 12 to 36 μg, and a dose of each of (b), (c¹) and/or (c²) is 2 to 20 μg, optionally 5 to 15 μg, optionally 6 to 12 μg.

In some embodiments, a dose of (a) is 10, 11, 12, 13, 14, 15, 16, 17, 18, 19, 20, 21, 22, 23, 24, 25, 26, 27, 28, 29, 30, 31, 32, 33, 34, 35,36, 37, 38, 39 or 40 μg, and a dose of each of (b), (c¹) and/or (c²) is 5, 6, 7, 8, 9, 10, 11, 12, 13, 14 or 15 μg.

In some embodiments, the immunogenic composition further comprises:
(c³) a first mRNA encoding a NA of the strain of subtype H1 of Influenza A virus, suitably H1N1;
(c⁴) a second mRNA encoding a NA of the strain of subtype H2 of Influenza A virus, suitably H3N2; and
(c⁵) a third mRNA encoding a NA of the first strain of Influenza B virus, suitably from B/Victoria lineage,
   wherein the ratio of (a):(b): (c¹): (c²) is comprised between 1.5:1:1:1 and 20:1:1:1, optionally between 1.5:1:1:1 and 5:1:1:1, suitably between 2:1:1:1 and 4:1:1:1, suitably between 2:1:1:1 and 3:1:1:1, suitably is 2:1:1:1 or 3:1:1:1.

In some embodiments, the ratio of (a):(b): (c¹): (c²): (c³): (c⁴): (c⁵) is comprised between 9:3:3:3:1:1:1 and 3:1:1:1:3:3:3, suitably between 6:2:2:2:1:1:1 and 3:1:1:1:2:2:2, suitably is 6:2:2:2:1:1:1 or 3:1:1:1:2:2:2.

In some embodiments, the ratio of (a):(b): (c¹): (c²): (c³): (c⁴): (c⁵) is comprised between 15:3:3:3:1:1:1:1 and 5:1:1:1:3:3:3:3, suitably is 10:2:2:2:1:1:1:1 or 15:3:3:3:1:1:1:1 or 8:2:2:2:1:1:1:1 or 12:3:3:3:1:1:1:1.

In some embodiments, a dose of (a), (b), (c¹) and (c²) is 5 to 75 μg, optionally 10 to 60 μg, optionally 12 to 48 μg.

In some embodiments, a dose of (a), (b), (c¹) and (c²) is 35 to 75 μg.

In some embodiments, a dose of (a), (b), (c¹) and (c²) is 35, 36, 37, 38, 39, 40, 45, 46, 47, 48, 49, 50, 51, 52, 53, 54, 55, 70, 71, 72, 73, 74 or 75 μg. In some embodiments, a dose of (a), (b), (c¹) and (c²) is 10, 11, 12, 13, 14, 15, 16, 17, 18, 19, 20, 21, 22, 24, 24, 25, 26, 27, 28, 29, 30, 45, 46, 47, 48, 49, 50, 55, 60 μg.

In some embodiments, a dose of (c³), (c⁴) and (c⁵) is 2 to 50 μg, optionally 2 to 30 μg, optionally 5 to 20, optionally 9 to 18 μg. In some embodiments, a dose of (c³), (c⁴) and (c⁵) is 5, 6, 7, 8, 9, 10, 11, 12, 13, 14, 15, 16, 17, 18, 19, 20.

In some embodiments, a dose of (c³), (c⁴) and (c⁵) is 9 to 36 μg.

In some embodiments, a dose of (c³), (c⁴) and (c⁵) is 9, 18, 27 or 36 μg.

In some embodiments, a dose of (c³), (c⁴) and (c⁵) is 0.5 to 50 μg, optionally 2 to 20 μg, optionally 5 to 15. In some embodiments, a dose of (e1), (e2) and (e3) is 5, 6, 7, 8, 9, 10, 11, 12, 13, 14 or 15 μg.

In some embodiments, a dose of (c³), (c⁴) and (c⁵) is 5 to 15 μg.

In some embodiments, a dose of (c³), (c⁴) and (c⁵) is 9 μg.

In some embodiments, a dose of each of (c³), (c⁴) and (c⁵) is 0.5 to 20 μg, optionally 0.5 to 10 μg, optionally 0.5 to 5, optionally 1 to 5 μg, optionally 2 to 5 μg. In some embodiments, a dose of each of (e1), (e2) and (e3) is 0.5, 1, 2, 3, 4, 5, 6, 7, 8, 9 or 10 μg.

In some embodiments, a dose of each of (c³), (c⁴) and (c⁵) is 1 to 5 μg.

In some embodiments, a dose of each of (c³), (c⁴) and (c⁵) is 3 μg.

In some embodiments, the immunogenic composition further comprises:
(c⁶) a fourth mRNA encoding a NA of the second strain of Influenza B virus, suitably from B/Yamagata lineage,
   wherein the ratio of (a):(b): (c¹): (c²) is comprised between 1.5:1:1:1 and 20:1:1:1, optionally between 1.5:1:1:1 and 5:1:1:1, suitably between 2:1:1:1 and 4:1:1:1, suitably between 2:1:1:1 and 3:1:1:1, suitably is 2:1:1:1 or 3:1:1:1.

In some embodiments, the ratio of (a):(b): (c¹): (c²): (c³): (c⁴): (c⁵): (c⁶) is comprised between 9:3:3:3:1:1:1:1 and 3:1:1:1:3:3:3:3, suitably between 6:2:2:2:1:1:1:1 and 3:1:1:1:2:2:2:2, suitably is 6:2:2:2:1:1:1:1 or 3:1:1:1:2:2:2:2.

In some embodiments, the ratio of (a):(b): (c¹): (c²): (c³): (c⁴): (c⁵): (c⁶) is comprised between 15:3:3:3:1:1:1:1 and 5:1:1:1:3:3:3:3, suitably is 10:2:2:2:1:1:1:1 or 15:3:3:3:1:1:1:1 or 8:2:2:2:1:1:1:1 or 12:3:3:3:1:1:1:1.

In some embodiments, a dose of (c³), (c⁴) and (c⁵) is 5 to 50 μg, optionally 10 to 30 μg, optionally 12 to 24 μg.

In some embodiments, a dose of (c³), (c⁴) and (c⁵) is 10, 11, 12, 13, 14, 15, 16, 17, 18, 19, 20, 21, 22, 23, 24, 25, 26, 27, 28, 29, 30 μg.

In some embodiments, a dose of (c³), (c⁴), (c⁵) and (c⁶) is 5 to 50 μg, optionally 10 to 50 μg, optionally 12 to 48 μg.

In some embodiments, a dose of (c³), (c⁴), (c⁵) and (c⁶) is 10, 11, 12, 13, 14, 15, 20, 21, 22, 23, 24, 25, 26, 27, 28, 29, 30, 35, 36, 37, 38, 39, 40, 45, 46, 47, 48 μg.

In some embodiments, a dose of (c³), (c⁴), (c⁵) and (c⁶) is comprised between 0.5 to 50 μg, optionally between 2 to 25 μg, optionally between 5 to 20 μg.

In some embodiments, a dose of (c³), (c⁴), (c⁵) and (c⁶) is 5, 6, 7, 8, 9, 10, 11, 12, 13, 14, 15, 16, 17, 18, 19 or 20 μg.

In some embodiments, a dose of (c³), (c⁴), (c⁵) and (c⁶) is 12 μg.

In some embodiments, a dose of each of (c³), (c⁴), (c⁵) and (c⁶) is 0.5 to 20 μg, optionally 0.5 to 10 μg, optionally 0.5 to 5, optionally 1 to 5 μg, optionally 2 to 5 μg. In some embodiments, a dose of each of (e1), (e2) and (e3) is 0.5, 1, 2, 3, 4, 5, 6, 7, 8, 9 or 10 μg.

In some embodiments, a dose of each of (c³), (c⁴), (c⁵) and (c⁶) is 1 to 5 μg.

In some embodiments, a dose of each of (c³), (c⁴), (c⁵) and (c⁶) is 3 μg.

In some embodiments, a dose of each (a), (b), (c), (c¹), (c²), (c³), (c⁴), (c⁵) and/or (c⁶) is 1 to 200 μg, suitably 1 to 60 μg, suitably 1 to 25 μg, suitably 2 to 25 μg.

In some embodiments, a dose of each (a), (b), (c), (c¹), (c²), (c³), (c⁴), (c⁵) and/or (c⁶) is 1 to 25 μg, optionally 2 to 25 μg, optionally 2 to 18 μg, optionally 2 to 9 μg, optionally 2 to 6 μg, optionally 3 to 25 μg, 3 to 18 μg, optionally 3 to 12 μg, optionally 3 to 9 μg, optionally 3 to 6 μg.

In some embodiments, a dose of each (a), (b), (c), (c¹), (c²), (c³), (c⁴), (c⁵) and/or (c⁶) is 1, 2, 3, 4, 5, 6, 7, 8, 9, 10, 11, 12, 13, 14, 15, 16, 17, 18, 19, 20, 21, 22, 23, 24 or 25 μg, optionally 1, 2, 3, 6, 9 or 18 μg.

In some embodiments, a dose of each (a), (b), (c), (c¹), (c²), (c³), (c⁴), (c⁵) and/or (c⁶) is 1, 2, 3, 6, 9, 12 or 18 μg.

In some embodiments, a dose of each (a), (b), (c), (c¹), (c²), (c³), (c⁴), (c⁵) and/or (c⁶) is 0.5 to 200 μg, optionally 2 to 10 μg, 2 to 25 μg, optionally 2 to 30 μg, optionally 2 to 40 μg, optionally 2 to 45 μg, optionally 2 to 50 μg, optionally 2 to 75 μg.

In some embodiments, a dose of each (a), (b), (c), (c¹), (c²), (c³), (c⁴), (c⁵) and/or (c⁶) is 0.5, 1, 2, 3, 4, 5, 6, 7, 8, 9, 10, 11, 12, 13, 14, 15, 16, 17, 18, 19, 20, 21, 22, 23, 24, 25, 30, 35, 36, 37, 38, 39, 40, 45, 46, 47, 48, 49, 50, 55, 60, 65, 70, 71, 72, 73, 74 or 75 μg, optionally 3, 6, 9, 24, 36, 48 or 72 μg.

mRNAs used herein are suitably provided in purified or substantially purified form i.e. substantially free from proteins (e.g., enzymes), other nucleic acids (e.g. DNA and nucleoside phosphate monomers), and the like, generally being at least about 50% pure (by weight), and usually at least 90% pure, such as at least 95% or at least 98% pure.

mRNAs used herein may be prepared in many ways e.g. by chemical synthesis in whole or in part, by digesting longer nucleic acids using nucleases (e.g. restriction enzymes), by joining shorter nucleic acids or nucleotides (e.g. using ligases or polymerases), from genomic or cDNA libraries, etc. In particular, mRNA may be prepared enzymatically using a DNA template.

mRNAs used herein may be an artificial nucleic acid. The term “artificial nucleic acid” as used herein is intended to refer to a nucleic acid that does not occur naturally. In other words, an artificial nucleic acid may be understood as a non-natural nucleic acid molecule. Such nucleic acid molecules may be non-natural due to its individual sequence (e.g. G/C content modified coding sequence, UTRs) and/or due to other modifications, e.g. structural modifications of nucleotides. Typically, artificial nucleic acid may be designed and/or generated by genetic engineering to correspond to a desired artificial sequence of nucleotides. In this context, an artificial nucleic acid is a sequence that may not occur naturally, i.e. a sequence that differs from the wild type or reference sequence/the naturally occurring sequence by at least one nucleotide (via e.g. codon modification as further specified below). The term “artificial nucleic acid” is not restricted to mean “one single molecule” but is understood to comprise an ensemble of essentially identical nucleic acid molecules. Accordingly, it may relate to a plurality of essentially identical nucleic acid molecules.

In some embodiments, the mRNAs used herein may be a modified and/or stabilized nucleic acid, suitably a modified and/or stabilized artificial nucleic acid.

According to some embodiments, the mRNAs used herein may thus be provided as a “stabilized artificial nucleic acid” or “stabilized coding nucleic acid” that is to say a nucleic acid showing improved resistance to in vivo degradation and/or a nucleic acid showing improved stability in vivo, and/or a nucleic acid showing improved translatability in vivo. In the following, specific suitable modifications/adaptations in this context are described which are suitably to “stabilize” the nucleic acid.

mRNAs used herein may also be codon optimized. In some embodiments, the mRNAs used herein comprises at least one codon modified coding sequence. In some embodiments, the coding sequence of the mRNAs used herein is a codon modified coding sequence. Suitably, the amino acid sequence encoded by the codon modified coding sequence is not being modified compared to the amino acid sequence encoded by the corresponding wild type or reference coding sequence.

In some embodiments, the mRNAs of (a), (b), (c), (c¹), (c²), (c³), (c⁴), (c⁵) and/or (c⁶) comprises a coding sequence which is a codon modified coding sequence, wherein the amino acid sequence encoded by the codon modified coding sequence is optionally not being modified compared to the amino acid sequence encoded by the corresponding wild type or reference coding sequence.

In some embodiments, mRNAs used herein may be codon optimized for expression in human cells. By “codon optimized” is intended modification with respect to codon usage may increase translation efficacy and/or half-life of the nucleic acid. The term “codon modified coding sequence” relates to coding sequences that differ in at least one codon (triplets of nucleotides coding for one amino acid) compared to the corresponding wild type or reference coding sequence. Suitably, a codon modified coding sequence in the context of the invention may show improved resistance to in vivo degradation and/or improved stability in vivo, and/or improved translatability in vivo. Codon modifications in the broadest sense make use of the degeneracy of the genetic code wherein multiple codons may encode the same amino acid and may be used interchangeably (cf. Table 1 of WO2020002525) to optimize/modify the coding sequence for in vivo applications as outlined herein.

In embodiments, the mRNAs used herein may be modified, wherein the C content of the at least one coding sequence may be increased, suitably maximized, compared to the C content of the corresponding wild type or reference coding sequence (herein referred to as “C maximized coding sequence”). The amino acid sequence encoded by the C maximized coding sequence of the mRNA is suitably not modified compared to the amino acid sequence encoded by the respective wild type or reference coding sequence. The generation of a C maximized nucleic acid sequences may suitably be carried out using a modification method according to WO2015/062738. In this context, the disclosure of WO2015/062738 is included herewith by reference.

In some embodiments, the mRNAs used herein may be modified, wherein the codons in the at least one coding sequence may be adapted to human codon usage (herein referred to as “human codon usage adapted coding sequence”). Codons encoding the same amino acid occur at different frequencies in humans. Accordingly, the coding sequence of the mRNAs used herein is suitably modified such that the frequency of the codons encoding the same amino acid corresponds to the naturally occurring frequency of that codon according to the human codon usage. For example, in the case of the amino acid Ala, the wild type or reference coding sequence is suitably adapted in a way that the codon “GCC” is used with a frequency of 0.40, the codon “GCT” is used with a frequency of 0.28, the codon “GCA” is used with a frequency of 0.22 and the codon “GCG” is used with a frequency of 0.10 etc. (see e.g. Table 1 of WO2020002525).

Accordingly, such a procedure (as exemplified for Ala) is applied for each amino acid encoded by the coding sequence of the RNA to obtain sequences adapted to human codon usage.

In embodiments, the mRNAs used herein may be modified, wherein the codon adaptation index (CAI) may be increased or suitably maximised in the at least one coding sequence (herein referred to as “CAI maximized coding sequence”). In some embodiments, all codons of the wild type or reference nucleic acid sequence that are relatively rare in e.g. a human are exchanged for a respective codon that is frequent in the e.g. a human, wherein the frequent codon encodes the same amino acid as the relatively rare codon. Suitably, the most frequent codons are used for each amino acid of the encoded protein (see Table 1 of WO2020002525, most frequent human codons are marked with asterisks). Suitably, the mRNAs used herein comprise at least one coding sequence, wherein the codon adaptation index (CAI) of the at least one coding sequence is at least 0.5, at least 0.8, at least 0.9 or at least 0.95. In some embodiments, the codon adaptation index (CAI) of the at least one coding sequence is 1 (CAI=1). For example, in the case of the amino acid Ala, the wild type or reference coding sequence may be adapted in a way that the most frequent human codon “GCC” is always used for the amino acid. Accordingly, such a procedure (as exemplified for Ala) may be applied for each amino acid encoded by the coding sequence of the mRNA to obtain CAI maximized coding sequences.

In embodiments, the mRNAs used herein may be modified, wherein the G/C content of the at least one coding sequence may be modified compared to the G/C content of the corresponding wild type or reference coding sequence (herein referred to as “G/C content modified coding sequence”). In this context, the terms “G/C optimization” or “G/C content modification” relate to a nucleic acid that comprises a modified, suitably an increased number of guanosine and/or cytosine nucleotides as compared to the corresponding wild type or reference coding sequence. Such an increased number may be generated by substitution of codons containing adenosine or thymidine nucleotides by codons containing guanosine or cytosine nucleotides. Suitably, nucleic acid sequences having an increased G/C content are more stable or show a better expression than sequences having an increased A/U. The amino acid sequence encoded by the G/C content modified coding sequence of the mRNA is suitably not modified as compared to the amino acid sequence encoded by the respective wild type or reference sequence. In some embodiments, the G/C content of the coding sequence of the nucleic acid is increased by at least 10%, 20%, 30%, suitably by at least 40% compared to the G/C content of the coding sequence of the corresponding wild type or reference nucleic acid sequence. The generation of a G/C content optimized mRNA sequence may be carried out using a method according to WO2002/098443. In this context, the disclosure of WO2002/098443 is included in its full scope in the present invention.

In embodiments, the mRNAs used herein may be modified by altering the number of A and/or U nucleotides in the nucleic acid sequence with respect to the number of A and/or U nucleotides in the original nucleic acid sequence (e.g. the wild type or reference sequence). In some embodiments, such an AU alteration is performed to modify the retention time of the individual nucleic acids in a composition, to (i) allow co-purification using a HPLC method, and/or to allow analysis of the obtained nucleic acid composition. Such a method is described in detail in published PCT application WO2019092153A1. Claims 1 to 70 of WO2019092153A1 herewith incorporated by reference.

In some embodiments, the modified RNA sequence is selected from C maximized coding sequence, CAI maximized coding sequence, human codon usage adapted coding sequence, G/C content modified (or optimized) sequence, A/U alteration, or any combination thereof.

In some embodiments, the RNA sequence has a G/C content of at least about 45%, 50%, 55%, or 60%. In particular embodiments, the RNA sequence has a G/C content of at least about 50%, 51%, 52%, 53%, 54%, 55%, 56%, 57%, 58%, 59%, 60%, 61%, 62%, 63%, 64%, 65%, 66%, 67%, 68%, 69%, or 70%.

Suitably, when transfected into mammalian host cells, the mRNA comprising a modified sequence has a stability of between 12-18 hours, or greater than 18 hours, e.g., 24, 36, 48, 60, 72, or greater than 72 hours and are capable of being expressed by the mammalian host cell (e.g. a muscle cell).

Suitably, when transfected into mammalian host cells, the mRNA comprising a modified RNA sequence is translated into protein, wherein the amount of protein is at least comparable to, or suitably at least 10% more than, or at least 20% more than, or at least 30% more than, or at least 40% more than, or at least 50% more than, or at least 100% more than, or at least 200% or more than the amount of protein obtained by a naturally occurring or wild type or reference coding sequence transfected into mammalian host cells.

In some embodiments, the mRNAs used herein comprise at least one poly (N) sequence, e.g. at least one poly (A) sequence, at least one poly (U) sequence, at least one poly (C) sequence, or combinations thereof.

In some embodiments, the mRNAs used herein comprise at least one poly (A) sequence. Suitably, a poly A tail (e.g., of about 30 adenosine residues or more) may be attached to the 3′ end of the RNA to increase its half-life.

The terms “poly (A) sequence”, “poly (A) tail” or “3′-poly(A) tail” as used herein will be recognized and understood by the person of ordinary skill in the art, and are e.g. intended to be a sequence of adenosine nucleotides, typically located at the 3′-end of a linear RNA (or in a circular RNA), of up to about 1000 adenosine nucleotides. In some embodiments, the poly(A) sisequence is essentially homopolymeric, e.g. a poly (A) sequence of e.g. 100 adenosine nucleotides has essentially the length of 100 nucleotides. In other embodiments, the poly(A) sisequence may be interrupted by at least one nucleotide different from an adenosine nucleotide, e.g. a poly (A) sequence of e.g. 100 adenosine nucleotides may have a length of more than 100 nucleotides (comprising 100 adenosine nucleotides and in addition the at least one nucleotide- or a stretch of nucleotides-different from an adenosine nucleotide).

The poly (A) sequence may comprise about 10 to about 500 adenosine nucleotides, about 10 to about 200 adenosine nucleotides, about 40 to about 200 adenosine nucleotides, or about 40 to about 150 adenosine nucleotides. In some embodiments, the length of the poly(A) sisequence may be at least about or even more than about 10, 50, 64, 75, 100, 200, 300, 400, or 500 adenosine nucleotides.

In some embodiments, the mRNAs used herein comprise at least one poly (A) sequence comprising about 30 to about 200 adenosine nucleotides. In some embodiments, the poly (A) sequence comprises about 64 adenosine nucleotides (A64). In other some embodiments, the poly (A) sequence comprises about 100 adenosine nucleotides (A100). In other embodiments, the poly (A) sequence comprises about 150 adenosine nucleotides.

In further embodiments, the mRNAs used herein comprise at least one poly (A) sequence comprising about 100 adenosine nucleotides, wherein the poly(A) sisequence is interrupted by non-adenosine nucleotides, suitably by 10 non-adenosine nucleotides (A30-N10-A70).

The poly (A) sequence as defined herein may be located directly at the 3′ terminus of the mRNA. In some embodiments, the 3′-terminal nucleotide (that is the last 3′-terminal nucleotide in the polynucleotide chain) is the 3′-terminal A nucleotide of the at least one poly (A) sequence. The term “directly located at the 3′ terminus” has to be understood as being located exactly at the 3′ terminus—in other words, the 3′ terminus of the nucleic acid consists of a poly (A) sequence terminating with an A nucleotide.

In an embodiment, the mRNAs used herein comprise a poly (A) sequence of at least 70 adenosine nucleotides, suitably consecutive at least 70 adenosine nucleotides, wherein the 3′-terminal nucleotide is an adenosine nucleotide.

In embodiments, the poly(A) sisequence of the nucleic acid is obtained from a DNA template during RNA in vitro transcription. In other embodiments, the poly(A) sisequence is obtained in vitro by common methods of chemical synthesis without being necessarily transcribed from a DNA template. In other embodiments, poly (A) sequences are generated by enzymatic polyadenylation of the RNA (after RNA in vitro transcription) using commercially available polyadenylation kits and corresponding protocols known in the art, or alternatively, by using immobilized poly (A) polymerases e.g. using a methods and means as described in WO2016174271.

In some embodiments, the mRNAs of (a), (b), (c), (c¹), (c²), (c³), (c⁴), (c⁵) and/or (c⁶) comprises at least one poly (A) tail sequence comprising 30 to 200 adenosine nucleotides, preferably 100 adenosine nucleotides, wherein the 3′ terminal nucleotide of said RNA is an adenosine.

In embodiments, the mRNAs used herein comprise at least one poly (C) sequence.

The term “poly (C) sequence” as used herein is intended to be a sequence of cytosine nucleotides of up to about 200 cytosine nucleotides. In embodiments, the poly (C) sequence comprises about 10 to about 200 cytosine nucleotides, about 10 to about 100 cytosine nucleotides, about 20 to about 70 cytosine nucleotides, about 20 to about 60 cytosine nucleotides, or about 10 to about 40 cytosine nucleotides. In an embodiment, the poly (C) sequence comprises about 30 cytosine nucleotides.

In some embodiments, the mRNAs of (a), (b), (c), (c¹), (c²), (c³), (c⁴), (c⁵) and/or (c⁶) comprises a poly(A) tail sequence, preferably comprising 30 to 200 adenosine nucleotides and/or at least one poly (C) sequence, preferably comprising 10 to 40 cytosine nucleotides.

In embodiments, the mRNAs used herein comprise at least one histone stem-loop (hSL) or histone stem loop structure. In some embodiments, the mRNAs of (a), (b), (c), (c¹), (c²), (c³), (c⁴), (c⁵) and/or (c⁶) comprises at least one histone stem-loop.

The term “histone stem-loop” (abbreviated as “hSL” in e.g. the sequence listing) is intended to refer to nucleic acid sequences that form a stem-loop secondary structure predominantly found in histone mRNAs.

Histone stem-loop sequences/structures may suitably be selected from histone stem-loop sequences as disclosed in WO2012019780, the disclosure relating to histone stem-loop sequences/histone stem-loop structures incorporated herewith by reference. A histone stem-loop sequence that may be used may be derived from formulae (I) or (II) of WO2012019780. According to a further embodiment, the mRNA comprises at least one histone stem-loop sequence derived from at least one of the specific formulae (Ia) or (IIa) of the patent application WO2012019780.

In some embodiments, said first mRNA and/or said second mRNA comprises at least one histone stem-loop.

In other embodiments, the mRNAs used herein does not comprise a hsL as defined herein.

The mRNAs used herein may be modified by the addition of a 5′-cap structure, which suitably stabilizes the RNA and/or enhances expression of the encoded antigen and/or reduces the stimulation of the innate immune system (after administration to a subject).

The term “5′-cap structure” as used herein will be recognized and understood by the person of ordinary skill in the art, and is e.g. intended to refer to a 5′ modified nucleotide, particularly a guanine nucleotide, positioned at the 5′-end of an RNA, e.g. an mRNA.

For example, the 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap 0 structure plays an important role in maintaining the stability and translational efficacy of the RNA molecule. The 5′ cap of the mRNA molecule may be further modified by a 2′-O-Methyltransferase which results in the generation of a cap 1 structure (m7Gppp[m2′-O] N), which may further increase translation efficacy.

In some embodiments, the 5′-cap structure is connected via a 5′-5′-triphosphate linkage to the RNA.

5′-cap structures which may be suitable are cap0 (methylation of the first nucleobase, e.g. m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In some embodiments, the mRNAs used herein, suitably the mRNAs of (a), (b), (c), (c¹), (c²), (c³), (c⁴), (c⁵) and/or (c⁶), comprises a 5′ cap, preferably m7G, cap0, cap1, cap2, a modified cap0 or a modified cap1 structure, suitably a 5′-cap1 structure.

A 5′-cap (cap0 or cap1) structure may be formed in chemical RNA synthesis or in RNA in vitro transcription (co-transcriptional capping) using cap analogues.

The term “cap analogue” as used herein will be recognized and understood by the person of ordinary skill in the art, and is e.g. intended to refer to a non-polymerizable di-nucleotide or tri-nucleotide that has cap functionality in that it facilitates translation or localization, and/or prevents degradation of a nucleic acid molecule, particularly of an RNA molecule, when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent polymerase, particularly, by template-dependent RNA polymerase. Examples of cap analogues include, but are not limited to, a chemical structure selected from the group consisting of m7GpppG, m7GpppA, m7GpppC; unmethylated cap analogues (e.g. GpppG); dimethylated cap analogue (e.g. m2,7GpppG), trimethylated cap analogue (e.g. m2,2,7GpppG), dimethylated symmetrical cap analogues (e.g. m7Gpppm7G), or anti reverse cap analogues (e.g. ARCA; m7,2′OmeGpppG, m7,2′dGpppG, m7,3′OmeGpppG, m7,3′dGpppG and their tetraphosphate derivatives). Further cap analogues have been described previously (WO2008016473, WO2008157688, WO2009149253, WO2011015347, and WO2013059475). Further suitable cap analogues in that context are described in WO2017066793, WO2017066781, WO2017066791, WO2017066789, WO2017/053297, WO2017066782, WO2018075827 and WO2017066797 wherein the disclosures referring to cap analogues are incorporated herewith by reference.

In embodiments, a modified cap1 structure is generated using tri-nucleotide cap analogue as disclosed in WO2017053297, WO2017066793, WO2017066781, WO2017066791, WO2017066789, WO2017066782, WO2018075827 and WO2017066797. In particular, any cap structures derivable from the structure disclosed in claim 1-5 of WO2017053297 may be suitably used to co-transcriptionally generate a modified cap1 structure. Further, any cap structures derivable from the structure defined in claim 1 or claim 21 of WO2018075827 may be suitably used to co-transcriptionally generate a modified cap1 structure.

In embodiments, the mRNAs used herein comprises a cap1 structure.

In embodiments, the 5′-cap structure may be added co-transcriptionally using tri-nucleotide cap analogue as defined herein, suitably in an RNA in vitro transcription reaction as defined herein.

In embodiments, the cap1 structure of the mRNA is formed using co-transcriptional capping using tri-nucleotide cap analogues m7G (5′) ppp (5′) (2′OMeA) pG or m7G (5′) ppp (5′) (2′OMeG) pG. A suitable cap1 analogues in that context is m7G (5′) ppp (5′) (2′OMeA) pG.

In other embodiments, the cap1 structure of the mRNA is formed using co-transcriptional capping using tri-nucleotide cap analogue 3′OMe-m7G (5′) ppp (5′) (2′OMeA) pG.

In other embodiments, a cap0 structure of the mRNAs used herein is formed using co-transcriptional capping using cap analogue 3′OMe-m7G (5′) ppp (5′) G.

In other embodiments, the 5′-cap structure is formed via enzymatic capping using capping enzymes (e.g. vaccinia virus capping enzymes and/or cap-dependent 2′-O methyltransferases) to generate cap0 or cap1 or cap2 structures. The 5′-cap structure (cap0 or cap1) may be added using immobilized capping enzymes and/or cap-dependent 2′-O methyltransferases using methods and means disclosed in WO2016193226.

For determining the presence/absence of a cap0 or a cap1 structure, a capping assays as described in published PCT application WO2015101416, in particular, as described in claims 27 to 46 of published PCT application WO2015101416 can be used. Other capping assays that may be used to determine the presence/absence of a cap0 or a cap1 structure of an RNA are described in PCT/EP2018/08667, or published PCT applications WO2014152673 and WO2014152659.

In embodiments, the mRNAs used herein comprise an m7G (5′) ppp (5′) (2′OMeA) cap structure. In such embodiments, the mRNAs comprise a 5′-terminal m7G cap, and an additional methylation of the ribose of the adjacent nucleotide of m7GpppN, in that case, a 2′O methylated Adenosine. In some embodiments, about 70%, 75%, 80%, 85%, 90%, 95% of the RNA (species) comprises such a cap1 structure as determined using a capping assay.

In other embodiments, the mRNAs used herein comprise an m7G (5′) ppp (5′) (2′OMeG) cap structure. In such embodiments, the mRNAs comprise a 5′-terminal m7G cap, and an additional methylation of the ribose of the adjacent nucleotide, in that case, a 2′O methylated guanosine. In some embodiments, about 70%, 75%, 80%, 85%, 90%, 95% of the coding RNA (species) comprises such a cap1 structure as determined using a capping assay.

Accordingly, the first nucleotide of the mRNA sequence, that is, the nucleotide downstream of the m7G (5′) ppp structure, may be a 2′O methylated guanosine or a 2′O methylated adenosine.

Suitably, the mRNAs used herein comprise a ribosome binding site, also referred to as “Kozak sequence”. In embodiments, the A/U (A/T) content in the environment of the ribosome binding site of the mRNAs used herein may be increased compared to the A/U (A/T) content in the environment of the ribosome binding site of its respective wild type or reference nucleic acid. This modification (an increased A/U (A/T) content around the ribosome binding site) increases the efficiency of ribosome binding to the mRNA. An effective binding of the ribosomes to the ribosome binding site in turn has the effect of an efficient translation the mRNA.

In some embodiments, the mRNAs used herein may comprise at least one heterologous untranslated region (UTR), e.g. a 5′ UTR and/or a 3′ UTR.

The term “untranslated region” or “UTR” or “UTR element” will be recognized and understood by the person of ordinary skill in the art, and are e.g. intended to refer to a part of a nucleic acid molecule typically located 5′ or 3′ of a coding sequence. An UTR is not translated into protein. An UTR may be part of a nucleic acid, e.g. a DNA or an RNA. An UTR may comprise elements for controlling gene expression, also called regulatory elements. Such regulatory elements may be, e.g., ribosomal binding sites, miRNA binding sites, promotor elements etc.

In embodiments, the mRNAs used herein comprise a protein-coding region (“coding sequence” or “cds”), and 5′-UTR and/or 3′-UTR. Notably, UTRs may harbor regulatory sequence elements that determine nucleic acid, e.g. RNA turnover, stability, and localization. Moreover, UTRs may harbor sequence elements that enhance translation. In medical application of nucleic acid sequences (including DNA and RNA), translation of the nucleic acid into at least one peptide or protein is of paramount importance to therapeutic efficacy. Certain combinations of 3′-UTRs and/or 5′-UTRs may enhance the expression of operably linked coding sequences encoding peptides or proteins of the invention. Nucleic acid molecules harboring the UTR combinations advantageously enable rapid and transient expression of antigenic peptides or proteins after administration to a subject, suitably after intramuscular administration. Accordingly, the mRNA comprising certain combinations of 3′-UTRs and/or 5′-UTRs as provided herein is particularly suitable for administration as a vaccine, in particular, suitable for administration into the muscle, the dermis, or the epidermis of a subject.

In some embodiments, the mRNAs used herein comprise at least one heterologous 5′-UTR and/or at least one heterologous 3′-UTR. The heterologous 5′-UTRs or 3′-UTRs may be derived from naturally occurring genes or may be synthetically engineered. In embodiments, the mRNA comprises at least one coding sequence as defined herein operably linked to at least one (heterologous) 3′-UTR and/or at least one (heterologous) 5′-UTR.

In embodiments, the mRNAs used herein comprise at least one heterologous 3′-UTR.

In some embodiments, the mRNAs of (a), (b), (c), (c¹), (c²), (c³), (c⁴), (c⁵) and/or (c⁶) comprises a 3′ UTR.

The term “3′-untranslated region” or “3′-UTR” or “3′-UTR element” will be recognized and understood by the person of ordinary skill in the art, and are e.g. intended to refer to a part of a nucleic acid molecule located 3′ (i.e. downstream) of a coding sequence and which is not translated into protein. A 3′-UTR may be part of a nucleic acid, e.g. a DNA or an RNA, located between a coding sequence and an (optional) terminal poly (A) sequence. A 3′-UTR may comprise elements for controlling gene expression, also called regulatory elements. Such regulatory elements may be, e.g., ribosomal binding sites, miRNA binding sites etc.

In some embodiments, the mRNAs used herein comprise a 3′-UTR, which may be derivable from a gene that relates to an RNA with enhanced half-life (i.e. that provides a stable RNA).

In some embodiments, a 3′-UTR comprises one or more of a polyadenylation signal, a binding site for proteins that affect a nucleic acid stability of location in a cell, or one or more miRNA or binding sites for miRNAs.

In embodiments, the mRNAs used herein comprise at least one heterologous 3′-UTR, wherein the at least one heterologous 3′-UTR comprises a nucleic acid sequence is derived or selected from a 3′-UTR of a gene selected from PSMB3, ALB7, alpha-globin (referred to as “muag”), CASP1, COX6B1, GNAS, NDUFA1 and RPS9, or from a homolog, a fragment or variant of any one of these genes.

In some embodiments, the mRNAs of (a), (b), (c), (c¹), (c²), (c³), (c⁴), (c⁵) and/or (c⁶) comprises a 3′ UTR comprising or consisting of a nucleic acid sequence derived from a 3′-UTR of a gene selected from PSMB3, ALB7, CASP1, COX6B1, GNAS, NDUFA1 and RPS9, or from a homolog, a fragment or a variant of any one of these genes.

Nucleic acid sequences in that context can be derived from published PCT application WO2019077001A1, in particular, claim 9 of WO2019077001A1. The corresponding 3′-UTR sequences of claim 9 of WO2019077001A1 are herewith incorporated by reference.

In some embodiments, the mRNAs used herein may comprise a 3′-UTR as described in WO2016107877, the disclosure of WO2016107877 relating to 3′-UTR sequences herewith incorporated by reference. Suitable 3′-UTRs are SEQ ID NOs: 1-24 and SEQ ID NOs: 49-318 of WO2016107877, or fragments or variants of these sequences. In other embodiments, the mRNAs used herein comprise a 3′-UTR as described in WO2017036580, the disclosure of WO2017036580 relating to 3′-UTR sequences herewith incorporated by reference. Suitable 3′-UTRs are SEQ ID NOs: 152-204 of WO2017036580, or fragments or variants of these sequences.

In other embodiments, the mRNAs used herein comprise a 3′-UTR as described in WO2016022914, the disclosure of WO2016022914 relating to 3′-UTR sequences herewith incorporated by reference. Particularly suitable 3′-UTRs are nucleic acid sequences according to SEQ ID NOs: 20-36 of WO2016022914, or fragments or variants of these sequences.

In embodiments, the mRNAs used herein comprise at least one heterologous 5′-UTR.

In some embodiments, the mRNAs used herein, suitably the mRNAs of (a), (b), (c), (c¹), (c²), (c³), (c⁴), (c⁵) and/or (c⁶), comprises a 5′ untranslated region (UTR).

The terms “5′-untranslated region” or “5′-UTR” or “5′-UTR element” will be recognized and understood by the person of ordinary skill in the art, and are e.g. intended to refer to a part of a nucleic acid molecule located 5′ (i.e. “upstream”) of a coding sequence and which is not translated into protein. A 5′-UTR may be part of a nucleic acid located 5′ of the coding sequence. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g. by enzymatic or post-transcriptional addition of a 5′-cap structure (e.g. for mRNA as defined herein). In some embodiments, the mRNAs used herein comprise a 5′-UTR, which may be derivable from a gene that relates to an RNA with enhanced half-life (i.e. that provides a stable RNA).

In some embodiments, a 5′-UTR comprises one or more of a binding site for proteins that affect an RNA stability or RNA location in a cell, or one or more miRNA or binding sites for miRNAs.

In embodiments, the mRNAs used herein, suitably the mRNAs of (a), (b), (c), (c¹), (c²), (c³), (c⁴), (c⁵) and/or (c⁶), comprise at least one heterologous 5′-UTR, wherein the at least one heterologous 5′-UTR comprises a nucleic acid sequence is derived or selected from a 5′-UTR of gene selected from HSD17B4, RPL32, ASAH1, ATP5A1, MP68, NDUFA4, NOSIP, RPL31, SLC7A3, TUBB4B, and UBQLN2, or from a homolog, a fragment or variant of any one of these genes.

Nucleic acid sequences in that context can be selected from published PCT application WO2019077001A1, in particular, claim 9 of WO2019077001A1. The corresponding 5′-UTR sequences of claim 9 of WO2019077001A1 are herewith incorporated by reference (e.g., SEQ ID NOs: 1-20 of WO2019077001A1, or fragments or variants thereof).

In some embodiments, the mRNAs used herein may comprise a 5′-UTR as described in WO2013143700, the disclosure of WO2013143700 relating to 5′-UTR sequences herewith incorporated by reference. Particularly suitable 5′-UTRs are nucleic acid sequences derived from SEQ ID NOS: 1-1363, SEQ ID NO: 1395, SEQ ID NO: 1421 and SEQ ID NO: 1422 of WO2013143700, or fragments or variants of these sequences. In other embodiments, the mRNAs used herein comprise a 5′-UTR as described in WO2016107877, the disclosure of WO2016107877 relating to 5′-UTR sequences herewith incorporated by reference. Particularly suitable 5′-UTRs are nucleic acid sequences according to SEQ ID NOs: 25-30 and SEQ ID NOS: 319-382 of WO2016107877, or fragments or variants of these sequences. In other embodiments, the nucleic acid comprises a 5′-UTR as described in WO2017036580, the disclosure of WO2017036580 relating to 5′-UTR sequences herewith incorporated by reference. Particularly suitable 5′-UTRs are nucleic acid sequences according to SEQ ID NOs: 1-151 of WO2017036580, or fragments or variants of these sequences. In other embodiments, the nucleic acid comprises a 5′-UTR as described in WO2016022914, the disclosure of WO2016022914 relating to 5′-UTR sequences herewith incorporated by reference. Particularly suitable 5′-UTRs are nucleic acid sequences according to SEQ ID NOs: 3-19 of WO2016022914, or fragments or variants of these sequences.

In some embodiments, the mRNAs of (a), (b), (c), (c¹), (c²), (c³), (c⁴), (c⁵) and/or (c⁶) comprises an heterologous 5′-UTR that comprises or consists of a nucleic acid sequence derived from a 5′-UTR from HSD17B4 and at least one heterologous 3′-UTR comprises or consists of a nucleic acid sequence derived from a 3′-UTR of PSMB3.

In some embodiments, the mRNAs used herein, suitably the mRNAs of (a), (b), (c), (c¹), (c²), (c³), (c⁴), (c⁵) and/or (c⁶), comprises from 5′ to 3′:
i) 5′-cap1 structure;
ii) 5′-UTR derived from a 5′-UTR of a HSD17B4 gene;
iii) the coding sequence;
iv) 3′-UTR derived from a 3′-UTR of a PSMB3 gene;
v) optionally, a histone stem-loop sequence; and
vi) poly (A) sequence comprising about 100 A nucleotides, wherein the 3′ terminal nucleotide of said RNA is an adenosine.

In embodiments, RNA, suitably mRNA, of the composition has an RNA integrity ranging from about 40% to about 100%.

The term “RNA integrity” generally describes whether the complete RNA sequence is present in the composition. Low RNA integrity could be due to, amongst others, RNA degradation, RNA cleavage, incorrect or incomplete chemical synthesis of the RNA, incorrect base pairing, integration of modified nucleotides or the modification of already integrated nucleotides, lack of capping or incomplete capping, lack of polyadenylation or incomplete polyadenylation, or incomplete RNA in vitro transcription. RNA is a fragile molecule that can easily degrade, which may be caused e.g. by temperature, ribonucleases, pH or other factors (e.g. nucleophilic attacks, hydrolysis etc.), which may reduce the RNA integrity and, consequently, the functionality of the RNA.

The skilled person can choose from a variety of different chromatographic or electrophoretic methods for determining an RNA integrity. Chromatographic and electrophoretic methods are well-known in the art. In case chromatography is used (e.g. RP-HPLC), the analysis of the integrity of the RNA may be based on determining the peak area (or “area under the peak”) of the full length RNA in a corresponding chromatogram. The peak area may be determined by any suitable software which evaluates the signals of the detector system. The process of determining the peak area is also referred to as integration. The peak area representing the full-length RNA is typically set in relation to the peak area of the total RNA in a respective sample. The RNA integrity may be expressed in % RNA integrity.

In the context of aspects of the invention, RNA integrity may be determined using analytical (RP) HPLC. Typically, a test sample of the composition comprising lipid based carrier encapsulating RNA may be treated with a detergent (e.g. about 2% Triton X100) to dissociate the lipid based carrier and to release the encapsulated RNA. The released RNA may be captured using suitable binding compounds, e.g. Agencourt AMPure XP beads (Beckman Coulter, Brea, CA, USA) essentially according to the manufacturer's instructions. Following preparation of the RNA sample, analytical (RP) HPLC may be performed to determine the integrity of RNA. Typically, for determining RNA integrity, the RNA samples may be diluted to a concentration of 0.1 g/l using e.g. water for injection (WFI). About 10 μl of the diluted RNA sample may be injected into an HPLC column (e.g. a monolithic poly(styrene-divinylbenzene) matrix). Analytical (RP) HPLC may be performed using standard conditions, for example: Gradient 1: Buffer A (0.1M TEAA (pH 7.0)); Buffer B (0.1M TEAA (pH 7.0) containing 25% acetonitrile). Starting at 30% buffer B the gradient extended to 32% buffer B in 2 min, followed by an extension to 55% buffer B over 15 minutes at a flow rate of 1 ml/min. HPLC chromatograms are typically recorded at a wavelength of 260 nm. The obtained chromatograms may be evaluated using a software and the relative peak area may be determined in percent (%) as commonly known in the art. The relative peak area indicates the amount of RNA that has 100% RNA integrity. Since the amount of the RNA injected into the HPLC is typically known, the analysis of the relative peak area provides information on the integrity of the RNA. Thus, if e.g. 100 ng RNA have been injected in total, and 100 ng are determined as the relative peak area, the RNA integrity would be 100%. If, for example, the relative peak area would correspond to 80 ng, the RNA integrity would be 80%. Accordingly, RNA integrity in the context of the invention is determined using analytical HPLC, suitably analytical RP-HPLC.

In embodiments, RNA, suitably mRNA, of the composition has an RNA integrity ranging from about 40% to about 100%. In embodiments, the RNA, suitably mRNA, has an RNA integrity ranging from about 50% to about 100%. In embodiments, the RNA, suitably mRNA, has an RNA integrity ranging from about 60% to about 100%. In embodiments, the RNA, suitably mRNA, has an RNA integrity ranging from about 70% to about 100%. In embodiments, the RNA, suitably mRNA, integrity is for example about 50%, about 60%, about 70%, about 80%, or about 90%. RNA integrity is suitably determined using analytical HPLC, suitably analytical RP-HPLC.

In embodiments, the RNA, suitably mRNA, of the composition has an RNA integrity of at least about 50%, suitably of at least about 60%, more suitably of at least about 70%, most suitably of at least about 80% or about 90%. RNA integrity is suitably determined using analytical HPLC, more suitably analytical RP-HPLC.

In some embodiments, the RNAs, suitably mRNAs, used herein does not comprise a replicase element (e.g. a nucleic acid encoding a replicase).

In some embodiments, the RNAs used herein, suitably the mRNAs used herein, suitably the mRNAs of (a), (b), (c), (c¹), (c²), (c³), (c⁴), (c⁵) and/or (c⁶), optionally each, are not self-replicating.

In some embodiments, the RNAs used herein, suitably the mRNA used herein, suitably the mRNAs of (a), (b), (c), (c¹), (c²), (c³), (c⁴), (c⁵) and/or (c⁶), optionally each, are self-replicating.

### Chemical Modifications

In embodiments, the RNA, suitably mRNA used herein comprise a coding sequence that consists only of G, C, A and U nucleotides and therefore does not comprise modified nucleotides (except of the 5′ terminal cap structure (cap0, cap1, cap2)).

In some embodiments, the RNAs, suitably the mRNAs, used herein does not comprise chemically modified nucleotides.

In some embodiments, the mRNAs of (a), (b), (c), (c¹), (c²), (c³), (c⁴), (c⁵) and/or (c⁶) does not comprise chemically modified nucleotides.

In some embodiments, the RNAs, suitably the mRNAs, used herein are modified RNAs, suitably mRNAs, wherein the modification refers to chemical modifications comprising backbone modifications as well as sugar modifications or base modifications.

A modified RNA, suitably mRNA, may comprise one or more nucleotide analogs or modified nucleotides (nucleotide analogues/modifications, e.g. backbone modifications, sugar modifications or base modifications). As used herein, “nucleotide analog” or “modified nucleotide” refers to a nucleotide that contains one or more chemical modifications (e.g., substitutions) in or on the nitrogenous base of the nucleoside (e.g. cytosine (C), thymine (T) or uracil (U)), adenine (A) or guanine (G)) and/or one or more chemical modifications in or one the phosphates of the backbone. A nucleotide analog can contain further chemical modifications in or on the sugar moiety of the nucleoside (e.g. ribose, modified ribose, six-membered sugar analog, or open-chain sugar analog), or the phosphate. The preparation of nucleotides and modified nucleotides and nucleosides are well-known in the art, see the following references: U.S. Pat. Nos. 4,373,071, 4,458,066, 4,500,707, 4,668,777, 4,973,679, 5,047,524, 5,132,418, 5,153,319, 5,262,530, 5,700,642. Many modified nucleosides and modified nucleotides are commercially available.

A backbone modification as described herein is a modification, in which phosphates of the backbone of the nucleotides of the RNA, suitably the mRNA, are chemically modified. A sugar modification as described herein is a chemical modification of the sugar of the nucleotides of the RNA, suitably mRNA. Furthermore, a base modification as described herein is a chemical modification of the base moiety of the nucleotides of the RNA, suitably mRNA. In this context, nucleotide analogues or modifications are suitably selected from nucleotide analogues which are applicable for transcription and/or translation.

In some embodiments, the RNAs, suitably the mRNAs, used herein comprise at least one chemical modification.

In some embodiments, the mRNAs of (a), (b), (c), (c¹), (c²), (c³), (c⁴), (c⁵) and/or (c⁶) comprises at least one chemical modification.

Modified nucleobases (chemical modifications) which can be incorporated into modified nucleosides and nucleotides and be present in the RNA, suitably mRNA, molecules include: m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl) adenosine); ms2io6A (2-methylthio-N6-(cis-hydroxyisopentenyl) adenosine); g6A (N6-glycinylcarbamoyladenosine); t6A (N6-threonyl carbamoyladenosine); ms216A (2-methylthio-N6-threonyl carbamoyladenosine); m616A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar (p) (2′-O-ribosyladenosine (phosphate)); I (inosine); mil (1-methylinosine); m′lm (1,2′-O-dimethylinosine); m3C (3-methylcytidine); Cm (2′-O-methylcytidine); s2C (2-thiocytidine); ac4C (N4-acetylcytidine); f5C (5-fonnylcytidine); m5Cm (5,2-O-dimethylcytidine); ac4Cm (N4-acetyl-2-O-methylcytidine); (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr (p) (2′-O-ribosylguanosine (phosphate)); yW (wybutosine); 02yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl) uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl) uridine)); mchm5U (5-(carboxyhydroxymethyl) uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-O-methyl uridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,2′-O-dimethyladenosine); rn62Am (N6,N6,0-2-trimethyladenosine); m2′7G (N2,7-dimethylguanosine); m2′2′7G (N2,N2,7-trimethylguanosine); m3Um (3,2′-O-dimethyluridine); m5D (5-methyldihydrouridine); f5Cm (5-formyl-2′-O-methylcytidine); mlGm (,2′-O-dimethylguanosine); m′Am (1,2-O-dimethyl adenosine) irinomethyluridine); tm5s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl) uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C₂-C₆)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Many of these modified nucleobases and their corresponding ribonucleosides are available from commercial suppliers.

In some embodiments, the nucleotide analogues/modifications which may be incorporated into a modified RNA, suitably mRNA, are selected from 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6, N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2, N2-dimethylguanosine, 8-oxo-guanosine, 7-methyl-8-oxo-guanosine, 1-methyl-6-thio-guanosine, N2-methyl-6-thio-guanosine, and N2, N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphate)-uridine, 5′-O-(1-thiophosphate)-pseudouridine, 6-aza-cytidine, 2-thio-cytidine, alpha-thio-cytidine, Pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thio-guanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thio-adenosine, 8-azido-adenosine, 7-deaza-adenosine.

In some embodiments, the chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

Particularly suitable in that context are pseudouridine (ψ), N1-methylpseudouridine (m1), 5-methylcytosine, and 5-methoxyuridine, more suitably pseudouridine (ψ) and N1-methylpseudouridine (m1ψ), still more suitably N1-methylpseudouridine (m1ψ).

In some embodiments, essentially all, e.g. essentially 100% of the uracil in the coding sequence of the RNAs, suitably mRNAs, used herein have a chemical modification, suitably a chemical modification is in the 5-position of the uracil.

In some embodiments, the RNAs, suitably mRNAs, used herein comprise the chemical modification being a uridine modification, preferably wherein 100% of the uridine positions in the mRNA are modified.

Incorporating modified nucleotides such as e.g. pseudouridine (ψ), N1-methylpseudouridine (m1ψ), 5-methylcytosine, and/or 5-methoxyuridine into the coding sequence of the RNAs, suitably mRNAs, used herein may be advantageous as unwanted innate immune responses (upon administration of the coding mRNA or the vaccine) may be adjusted or reduced (if required).

In embodiments, the coding sequence of the RNAs, suitably mRNAs, used herein comprise at least one modified nucleotide selected from pseudouridine (ψ) and N1-methylpseudouridine (m1ψ), suitably wherein all uracil nucleotides are replaced by pseudouridine (ψ) nucleotides and/or N1-methylpseudouridine (m1ψ) nucleotides, optionally wherein all uracil nucleotides are replaced by pseudouridine (ψ) nucleotides and/or N1-methylpseudouridine (m1 ψ) nucleotides.

In some embodiments, the RNAs, suitably mRNAs, used herein do not comprise N1-methylpseudouridine (m1ψ) substituted positions. In further embodiments, the RNAs, suitably mRNAs, used herein do not comprise pseudouridine (ψ), N1-methylpseudouridine (m1ψ), 5-methylcytosine, and 5-methoxyuridine substituted position.

In some embodiments, the chemical modification is N1-methylpseudouridine and/or pseudouridine. In some embodiments, the chemical modification is N1-methylpseudouridine.

In the context of nucleic acid-based vaccine or therapeutic production, it may be required to provide GMP-grade nucleic acid, e.g. a GMP grade RNA or DNA. GMP-grade RNA or DNA may be produced using a manufacturing process approved by regulatory authorities. Accordingly, in some embodiments, RNA production is performed under current good manufacturing practice (GMP), implementing various quality control steps on DNA and RNA level, suitably according to WO2016180430. In embodiments, the RNA, suitably mRNA of the invention is a GMP-grade RNA.

### RNA Synthesis

In some embodiments, the RNA, suitably mRNA, may be prepared using any method known in the art, including chemical synthesis such as e.g. solid phase RNA synthesis, as well as in vitro methods, such as RNA in vitro transcription reactions.

Suitably, the RNA, suitably mRNA, used herein is in vitro transcribed RNA.

The terms “RNA in vitro transcription” or “in vitro transcription” relate to a process wherein RNA is synthesized in a cell-free system (in vitro). RNA may be obtained by DNA-dependent in vitro transcription of an appropriate DNA template, which may be a linearized plasmid DNA template or a PCR-amplified DNA template. The promoter for controlling RNA in vitro transcription can be any promoter for any DNA-dependent RNA polymerase. Particular examples of DNA-dependent RNA polymerases are the T7, T3, SP6, or Syn5 RNA polymerases. In an embodiment of the present invention the DNA template is linearized with a suitable restriction enzyme, before it is subjected to RNA in vitro transcription. Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined herein; optionally, further modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCl2, which supplies Mg2+ ions as a co-factor for the polymerase; a buffer (TRIS or HEPES) to maintain a suitable pH value, which can also contain antioxidants (e.g. DTT), and/or polyamines such as spermidine at optimal concentrations, e.g. a buffer system comprising TRIS-Citrate as disclosed in WO2017109161.

In embodiments, the nucleotide mixture used in RNA in vitro transcription may additionally comprise modified nucleotides as defined herein. In that context, suitable modified nucleotides may in particular be selected from pseudouridine (ψ), N1-methylpseudouridine (m1ψ), 5-methylcytosine, and 5-methoxyuridine. In embodiments, uracil nucleotides in the nucleotide mixture are replaced (either partially or completely) by pseudouridine (ψ) and/or N1-methylpseudouridine (m1ψ) to obtain a modified RNA.

In other embodiments, the nucleotide mixture used in RNA in vitro transcription does not comprise modified nucleotides as defined herein. In embodiments, the nucleotide mixture used in RNA in vitro transcription only comprises G, C, A and U nucleotides, and, optionally, a cap analog as defined herein.

In some embodiments, the nucleotide mixture (i.e. the fraction of each nucleotide in the mixture) used for RNA in vitro transcription reactions may be optimized for the given RNA sequence, suitably as described in WO2015188933.

In this context, the in vitro transcription has been performed in the presence of a sequence optimized nucleotide mixture and optionally a cap analog.

### RNA Purification

In embodiments, the RNA, suitably mRNA, used herein is a purified RNA, suitably mRNA.

The term “purified RNA (or mRNA)” as used herein has to be understood as RNA which has a higher purity after certain purification steps (e.g. HPLC, TFF, Oligo d (T) purification, precipitation steps) than the starting material (e.g. in vitro transcribed RNA). Typical impurities that are essentially not present in purified RNA comprise peptides or proteins (e.g. enzymes derived from DNA dependent RNA in vitro transcription, e.g. RNA polymerases, RNases, pyrophosphatase, restriction endonuclease, DNase), spermidine, BSA, abortive RNA sequences, RNA fragments (short double stranded RNA fragments, abortive sequences etc.), free nucleotides (modified nucleotides, conventional NTPs, cap analogue), template DNA fragments, buffer components (HEPES, TRIS, MgCl2) etc. Other potential impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. It is also desirable for the degree of RNA purity that the amount of full-length RNA transcripts is as close as possible to 100%. Accordingly, “purified RNA (or mRNA)” as used herein has a degree of purity of more than 75%, 80%, 85%, very particularly 90%, 91%, 92%, 93%, 94%, 95%, 96%, 97%, 98% and most favorably 99% or more. The degree of purity may for example be determined by an analytical HPLC, wherein the percentages provided above correspond to the ratio between the area of the peak for the target RNA and the total area of all peaks representing the by-products. Alternatively, the degree of purity may for example be determined by an analytical agarose gel electrophoresis or capillary gel electrophoresis.

In some embodiments, the RNA is purified using RP-HPLC, suitably using Reversed-Phase High pressure liquid chromatography (RP-HPLC) with a macroporous styrene/divinylbenzene column (e.g. particle size 30 μm, pore size 4000 Å) and additionally using a filter cassette with a cellulose based membrane with a molecular weight cutoff of about 100 kDa. The RNA may in particular be purified using PUREMESSENGER (CureVac, Tübingen, Germany; RP-HPLC according to WO2008077592) and/or tangential flow filtration (as described in WO2016193206) and/or oligo d (T) purification (see WO2016180430).

In some embodiments, the RNA, suitably mRNA, is purified by RP-HPLC and/or TFF to remove double-stranded RNA, non-capped RNA and/or RNA fragments.

The formation of double stranded RNA as side products during e.g. RNA in vitro transcription can lead to an induction of the innate immune response, particularly IFNalpha which is the main factor of inducing fever in vaccinated subjects, which is of course an unwanted side effect. Current techniques for immunoblotting of dsRNA (via dot Blot, serological specific electron microscopy (SSEM) or ELISA for example) are used for detecting and sizing dsRNA species from a mixture of nucleic acids.

In embodiments, the RNA, suitably mRNA, comprises about 5%, 10%, or 20% less double stranded RNA side products as an RNA, suitably mRNA, that has not been purified with RP-HPLC and/or TFF.

In some embodiments, the RP-HPLC and/or TFF purified RNA, suitably mRNA, comprises about 5%, 10%, or 20% less double stranded RNA side products as an RNA, suitably mRNA, that has been purified with Oligo dT purification, precipitation, filtration and/or AEX.

### Carriers

A range of carrier systems have been described which encapsulate or complex mRNA in order to facilitate mRNA delivery and consequent expression of encoded antigens as compared to mRNA which is not encapsulated or complexed. The present invention may utilise any suitable carrier system. Particular carrier systems of note are further described below.

In embodiments, the RNAs, suitably mRNAs, used herein are complexed, encapsulated, partially encapsulated, or associated with one or more lipids (e.g. cationic lipids and/or neutral lipids), thereby forming lipid-based carriers such as liposomes, lipid nanoparticles (LNPs), lipoplexes, and/or nanoliposomes, suitably lipid nanoparticles.

In some embodiments, the mRNAs of (a), (b), (c), (c¹), (c²), (c³), (c⁴), (c⁵) and/or (c⁶) are formulated in a lipid nanoparticle (LNP), either separately or together.

In some embodiments, the RNAs, suitably mRNAs, used herein are formulated separately (in any formulation or complexation agent defined herein), suitably wherein the RNAs, suitably mRNAs, used herein are formulated in separate liposomes, lipid nanoparticles (LNP), lipoplexes, and/or nanoliposomes.

In some embodiments, the RNAs used herein, suitably the mRNAs used herein, suitably the mRNAs of (a), (b), (c), (c¹), (c²), (c³), (c⁴), (c⁵) and/or (c⁶) are formulated separately.

In embodiments, the RNAs, suitably mRNAs, used herein are co-formulated (in any formulation or complexation agent defined herein), suitably wherein the RNAs, suitably mRNAs, used herein are formulated in separate liposomes, lipid nanoparticles (LNP), lipoplexes, and/or nanoliposomes.

In some embodiments, the RNAs used herein, suitably the mRNAs used herein, suitably the mRNAs of (a), (b), (c), (c¹), (c²), (c³), (c⁴), (c⁵) and/or (c⁶) are co-formulated, i.e. formulated together.

### LNPs

The term “lipid nanoparticle” (or “LNP”) refers to a non-virion particle in which nucleic acid molecules, such as RNA, can be encapsulated. LNPs are not restricted to any particular morphology, and include any morphology generated when an ionizable (or cationic) lipid and optionally one or more further lipids are combined, e.g. in an aqueous environment and/or in the presence of a nucleic acid, e.g. an RNA. For example, a liposome, a lipid complex, a lipoplex and the like are within the scope of a lipid nanoparticle (LNP). LNP delivery systems and methods for their preparation are known in the art.

The particles can include some external RNA, suitably mRNA, (e.g. on the surface of the particles), but desirably at least half of the RNA, suitably mRNA, (and suitably at least 85%, especially at least 95%, such as all of it) is encapsulated.

In some embodiments, LNPs are suitable for intramuscular and/or intradermal administration.

In embodiments, at least about 80%, 85%, 90%, 95% of lipid-based carriers, suitably the LNPs, have a spherical morphology.

LNPs typically comprise a cationic lipid and one or more excipients selected from neutral lipids, charged lipids, steroids and polymer conjugated lipids (e.g. PEGylated lipid). The RNAs, suitably mRNAs, may be encapsulated in the lipid portion of the LNP or an aqueous space enveloped by some or the entire lipid portion of the LNP. The RNAs, suitably mRNAs, or a portion thereof may also be associated and complexed with the LNP. An LNP may comprise any lipid capable of forming a particle to which the nucleic acids are attached, or in which the one or more nucleic acids are encapsulated. In some embodiments, the LNP comprising nucleic acids, suitably RNAs, more suitably mRNAs, comprises one or more cationic lipids, and one or more stabilizing lipids. Stabilizing lipids include neutral lipids and PEGylated lipids.

In some embodiments, the LNP comprises a PEG-modified lipid, a non-cationic lipid, a sterol, and a cationic lipid.

LNP can, for example, be formed of a mixture of (i) a PEG-modified lipid (ii) a non-cationic lipid (iii) a sterol (iv) an ionisable cationic lipid. Alternatively, LNP can for example be formed of a mixture of (i) a PEG-modified lipid (ii) a non-cationic lipid (iii) a sterol (iv) a non-ionisable cationic lipid.

In some embodiments, the non-cationic lipid is a neutral lipid.

In some embodiments, the cationic lipid is ionizable.

In vivo characteristics and behavior of LNPs can be modified by addition of a hydrophilic polymer coating, e.g. polyethylene glycol (PEG), to the LNP surface to confer steric stabilization. Furthermore, LNPs (or liposomes, nanoliposomes, lipoplexes) can be used for specific targeting by attaching ligands (e.g. antibodies, peptides, and carbohydrates) to its surface or to the terminal end of the attached PEG chains (e.g. via PEGylated lipids or PEGylated cholesterol).

In an embodiment, the RNA, suitably mRNA, is complexed with one or more lipids thereby forming lipid nanoparticles, wherein the LNP (or liposomes, nanoliposomes, lipoplexes) comprises a polymer conjugated lipid, suitably a PEGylated lipid/PEG lipid.

In some embodiments, the LNPs comprise a polymer conjugated lipid. The term “polymer conjugated lipid” refers to a molecule comprising both a lipid portion and a polymer portion. An example of a polymer conjugated lipid is a PEGylated lipid. The term “PEGylated lipid” or “PEG-modified lipid” refers to a molecule comprising both a lipid portion and a polyethylene glycol portion. PEGylated lipids are known in the art and include 1-(monomethoxy-polyethyleneglycol)-2,3-dimyristoylglycerol (PEG-s-DMG) and the like. The terms “PEGylated lipid” and “PEG-modified lipid” are used interchangeably herein.

A polymer conjugated lipid as defined herein, e.g. a PEG-lipid, may serve as an aggregation reducing lipid.

In certain embodiments, the LNP comprises a stabilizing-lipid which is a polyethylene glycol-lipid (PEGylated lipid). Suitable polyethylene glycol-lipids include PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly (ethylene glycol) 2000) carbamyl]-1,2-dimyristyloxlpropyl-3-amine (PEG-c-DMA). In some embodiments, the polyethylene glycol-lipid is PEG-2000-DMG. In one embodiment, the polyethylene glycol-lipid is PEG-c-DOMG). In other embodiments, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di (tetradecanoyloxy) propyl-1-O-(w-methoxy (polyethoxy)ethyl) butanedioate (PEG-S-DMG), a PEGylated ceramide (PEG-cer), or a PEG dialkoxypropylcarbamate such as w-methoxy (polyethoxy)ethyl-N-(2,3di (tetradecanoxy) propyl) carbamate or 2,3-di (tetradecanoxy) propyl-N-(w-methoxy (polyethoxy)ethyl) carbamate.

In some embodiments, the PEG-modified lipid comprises PEG-DMG or PEG-CDMA.

In embodiments, the PEGylated lipid is suitably derived from formula (IV) of published PCT patent application WO2018078053A1. Accordingly, PEGylated lipids derived from formula (IV) of published PCT patent application WO2018078053A1, and the respective disclosure relating thereto, are herewith incorporated by reference.

In some embodiments, the PEG-modified lipid has the formula IV:

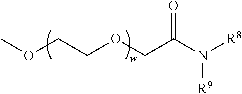

wherein R⁸ and R⁹ are each independently a straight or branched, saturated or unsaturated alkyl chain containing from 10 to 30 carbon atoms, wherein the alkyl chain is optionally interrupted by one or more ester bonds;
and ψ has a mean value ranging from 30 to 60.

In some embodiments, the PEG-modified lipid R⁸ and R⁹ are saturated alkyl chains.

In some embodiments, the RNA, suitably mRNA, is complexed with one or more lipids thereby forming LNPs, wherein the LNP comprises a polymer conjugated lipid, suitably a PEGylated lipid, wherein the PEG lipid is suitably derived from formula (IVa) of published PCT patent application WO2018078053A1. Accordingly, PEGylated lipid derived from formula (IVa) of published PCT patent application WO2018078053A1, and the respective disclosure relating thereto, is herewith incorporated by reference.

In some embodiments, the PEG lipid or PEGylated lipid is of formula (IVa):

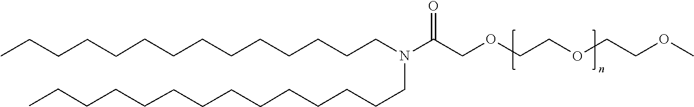

wherein n has a mean value ranging from 30 to 60, such as about 30±2, 32±2, 34±2, 36±2, 38±2, 40±2, 42±2, 44±2, 46±2, 48±2, 50±2, 52±2, 54±2, 56±2, 58±2, or 60±2. In an embodiment n is about 49. In another embodiment n is about 45. In further embodiments, the PEG lipid is of formula (IVa) wherein n is an integer selected such that the average molecular weight of the PEG lipid is about 2000 g/mol to about 3000 g/mol or about 2300 g/mol to about 2700 g/mol, suitably about 2500 g/mol.

In some embodiments, the PEG-modified lipid has the formula IVa:

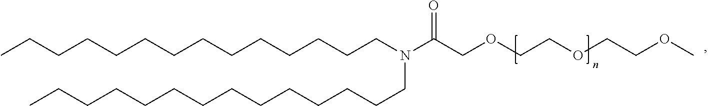

wherein n has a mean value ranging from 30 to 60, suitably wherein n has a mean value of about 45, 46, 47, 48, 49, 50, 51, 52, 53, 54, most suitably wherein n has a mean value of 49 or 45; or
wherein n is an integer selected such that the average molecular weight of the PEG lipid is about 2500 g/mol.

The lipid of formula IVa as suitably used herein has the chemical term 2 [(polyethylene glycol)-2000]-N, N-ditetradecylacetamide, also referred to as ALC-0159.

Further examples of PEG-lipids suitable in that context are provided in US20150376115A1 and WO2015199952, each of which is incorporated by reference in its entirety.

In some embodiments, LNPs include less than about 3, 2, or 1 mole percent of PEG or PEG-modified lipid, based on the total moles of lipid in the LNP.

In further embodiments, LNPs comprise from about 0.1% to about 20% of the PEG-modified lipid on a molar basis, e.g., about 0.5 to about 15%, about 0.5 to about 10%, about 0.5 to about 5%, about 10%, about 5%, about 3.5%, about 3%, about 2,5%, about 2%, about 1.5%, about 1%, about 0.5%, or about 0.3% on a molar basis (based on 100% total moles of lipids in the LNP). In embodiments, LNPs comprise from about 1.0% to about 2.0% of the PEG-modified lipid on a molar basis, e.g., about 1.2 to about 1.9%, about 1.2 to about 1.8%, about 1.3 to about 1.8%, about 1.4 to about 1.8%, about 1.5 to about 1.8%, about 1.6 to about 1.8%, in particular about 1.4%, about 1.5%, about 1.6%, about 1.7%, about 1.8%, about 1.9%, most suitably 1.7% (based on 100% total moles of lipids in the LNP). In various embodiments, the molar ratio of the cationic lipid to the PEGylated lipid ranges from about 100:1 to about 25:1.

In some embodiments, the LNP comprises a PEG-modified lipid at around 0.5 to 10 molar %, optionally 0.5 to 5 molar % or 0.5 to 3 molar %.

In embodiments, the LNP comprises one or more additional lipids, which stabilize the formation of particles during their formulation or during the manufacturing process (e.g. neutral lipid and/or one or more steroid or steroid analogue).

In embodiments, the RNA, suitably mRNA, is complexed with one or more lipids thereby forming lipid nanoparticles, wherein the LNP comprises one or more neutral lipid and/or one or more steroid or steroid analogue.

Suitable stabilizing lipids include neutral lipids and anionic lipids. The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

In some embodiments, the non-cationic lipid is a neutral lipid, such as 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE) or sphingomyelin (SM), preferably the neutral lipid is DSPC.

In embodiments, the LNP (or liposome, nanoliposome, lipoplex) comprises one or more neutral lipids, wherein the neutral lipid is selected from the group comprising distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE), or mixtures thereof.

In some embodiments, the LNPs comprise a neutral lipid selected from DSPC, DPPC, DMPC, DOPC, POPC, DOPE and SM. In various embodiments, the molar ratio of the cationic lipid to the neutral lipid ranges from about 2:1 to about 8:1.

In embodiments, the neutral lipid is 1,2-distearoyl-sn-glycero-3-phosphocholine (DSPC). Suitably, the molar ratio of the cationic lipid to DSPC may be in the range from about 2:1 to about 8:1.

In some embodiments, the steroid is sterol, suitably cholesterol.

In embodiments, the steroid is cholesterol. Suitably, the molar ratio of the cationic lipid to cholesterol may be in the range from about 2:1 to about 1:1. In some embodiments, the cholesterol may be PEGylated.

The sterol can be about 10 mol % to about 60 mol % or about 25 mol % to about 55 mol % or about 25 mol % to about 40 mol % of the lipid particle. In one embodiment, the sterol is about 10, 15, 20, 25, 30, 35, 40, 45, 50, 55, or about 60 mol % of the total lipid present in the lipid particle. In another embodiment, the LNPs include from about 5% to about 50% on a molar basis of the sterol, e.g., about 15% to about 45%, about 20% to about 40%, about 48%, about 40%, about 38.5%, about 35%, about 34.4%, about 31.5% or about 31% on a molar basis (based upon 100% total moles of lipid in the lipid nanoparticle).

The cationic lipid of an LNP may be ionizable, i.e. it becomes protonated as the pH is lowered below the pK of the ionizable group of the lipid but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

Such cationic lipids (for liposomes, lipid nanoparticles (LNP), lipoplexes, and/or nanoliposomes) include, but are not limited to, DSDMA, N, N-dioleyl-N, N-dimethylammonium chloride (DODAC), N, N-distearyl-N, N-dimethylammonium bromide (DDAB), 1,2-dioleoyltrimethyl ammonium propane chloride (DOTAP) (also known as N-(2,3-dioleoyloxy) propyl)-N,N,N-trimethylammonium chloride and 1,2-Dioleyloxy-3-trimethylaminopropane chloride salt), N-(1-(2,3-dioleyloxy) propyl)-N,N,N-trimethylammonium chloride (DOTMA), N,N-dimethyl-2,3-dioleyloxy) propylamine (DODMA), ckk-E12, ckk, 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (Y-DLenDMA), 98N12-5, 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), ICE (Imidazol-based), HGT5000, HGT5001, DMDMA, CLinDMA, CpLinDMA, DMOBA, DOcarbDAP, DLincarbDAP, DLinCDAP, KLin-K-DMA, DLin-K-XTC2-DMA, XTC (2,2-Dilinoleyl-4-dimethylaminoethyl-[1,3]-dioxolane) HGT4003, 1,2-Dilinoleoyl-3-trimethylaminopropane chloride salt (DLin-TAP.CI), 1,2-Dilinoleyloxy-3-(N-methylpiperazino) propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino) ethoxypropane (DLin-EG-DM A), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z, 12Z)-octadeca-9, 12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate (MC3), ALNY-100 ((3aR,5s,6aS)-N, N-dimethyl-2,2-di((9Z, 12Z)-octadeca-9, 12-dienyl)tetrahydro-3aH-cyclopenta[d][1, 3]dioxol-5-amine)), 1,1′-(2-(4-(2-((2-(bis(2-hydroxydodecyl)amino)ethyl) (2-hydroxydodecyl)amino)ethyl) piperazin-1-yl)ethylazanediyl) didodecan-2-ol (C12-200), 2,2-dilinoleyl-4-(2-dimethylaminoethyl)-[1,3]-dioxolane (DLin-K-C₂-DMA), 2,2-dilinoleyl-4-dimethylaminomethyl-[1,3]-dioxolane (DLin-K-DMA), NC98-5 (4,7, 13-tris(3-oxo-3-(undecylamino) propyl)-N,N 16-diundecyl-4,7, 10, 13-tetraazahexadecane-1, 16-diamide), (6Z,9Z,28Z,31Z)-heptatriaconta-6,9,28,31-tetraen-19-yl 4-(dimethylamino)butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31Z)-heptatriaconta-6,9,28,31-tetraen-19-yloxy)-N,N-dimethylbutan-1-amine (MC4 Ether), LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy) propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.) or any combination of any of the foregoing. Further suitable cationic lipids for use in the compositions and methods of the invention include those described in international patent publications WO2010053572 (and particularly, CI 2-200 described at paragraph [00225]) and WO2012170930, both of which are incorporated herein by reference, HGT4003, HGT5000, HGTS001, HGT5001, HGT5002 (see US20150140070A1).

In embodiments, the cationic lipid of the liposomes, lipid nanoparticles (LNP), lipoplexes, and/or nanoliposomes may be an amino lipid.

Representative amino lipids include, but are not limited to, 1,2-dilinoleyoxy-3-(dimethylamino) acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane salt chloride (DLin-TMA.CI), 1,2-dilinoleoyl-3-trimethylaminopropane chloride salt (DLin-TAP.CI), 1,2-dilinoleyloxy-3-(N-methylpiperazino) propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N, N-dimethylamino) ethoxypropane (DLin-EG-DMA), and 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA); dilinoleyl-methyl-4-dimethylaminobutyrate (DLin-MC3-DMA); MC3 (US20100324120).

In embodiments, the cationic lipid of the liposomes, lipid nanoparticles (LNP), lipoplexes, and/or nanoliposomes may an aminoalcohol lipidoid.

Aminoalcohol lipidoids may be prepared by the methods described in U.S. Pat. No. 8,450,298, herein incorporated by reference in its entirety. Suitable (ionizable) lipids can also be the compounds as disclosed in Tables 1, 2 and 3 and as defined in claims 1-24 of WO2017075531A1, hereby incorporated by reference.

In another embodiment, suitable lipids can also be the compounds as disclosed in WO2015074085A1 (i.e. ATX-001 to ATX-032 or the compounds as specified in claims 1-26), U.S. Appl. Nos. 61/905,724 and 15/614,499 or U.S. Pat. Nos. 9,593,077 and 9,567,296 hereby incorporated by reference in their entirety.

In other embodiments, suitable cationic lipids can also be the compounds as disclosed in WO2017117530A1 (i.e. lipids 13, 14, 15, 16, 17, 18, 19, 20, or the compounds as specified in the claims), hereby incorporated by reference in its entirety.

In some embodiments, ionizable or cationic lipids may also be selected from the lipids disclosed in WO2018078053A1 (i.e. lipids derived from formula I, II, and Ill of WO2018078053A1, or lipids as specified in claims 1 to 12 of WO2018078053A1), the disclosure of WO2018078053A1 hereby incorporated by reference in its entirety. In that context, lipids disclosed in Table 7 of WO2018078053A1 (e.g. lipids derived from formula 1-1 to I-41) and lipids disclosed in Table 8 of WO2018078053A1 (e.g. lipids derived from formula II-1 to 11-36) may be suitably used in the context of the invention. Accordingly, formula 1-1 to formula I-41 and formula II-1 to formula II-36 of WO2018078053A1, and the specific disclosure relating thereto, are herewith incorporated by reference.

In some embodiments, cationic lipids may be derived from formula III of published PCT patent application WO2018078053A1. Accordingly, formula III of WO2018078053A1, and the specific disclosure relating thereto, are herewith incorporated by reference.

In some embodiments, the RNA, suitably mRNA, is complexed with one or more lipids thereby forming LNPs (or liposomes, nanoliposomes, lipoplexes), wherein the cationic lipid of the LNP is selected from structures III-1 to III-36 of Table 9 of published PCT patent application WO2018078053A1. Accordingly, formula III-1 to III-36 of WO2018078053A1, and the specific disclosure relating thereto, are herewith incorporated by reference.

In some embodiments, the ionizable cationic lipid has the formula III:

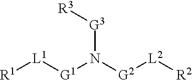

or a pharmaceutically acceptable salt, tautomer or stereoisomer thereof, wherein:
L¹ or L² is each independently —O(C═O)— or —(C═O)O—;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, or C₃-C₈ cycloalkenylene;
R¹ and R² are each independently, branched or linear, C₆-C₂₄ alkyl or C₆-C₂₄ alkenyl;
R³ is H, OR⁵, CN, —C(═O) OR⁴, —OC(═O)R⁴ or —NR⁵C(═O) R⁴;
R⁴ is C₁-C₁₂ alkyl;
R⁵ is H or C₁-C₆ alkyl.

In some embodiments, the ionizable cationic lipid has the formula III:

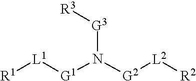

or a pharmaceutically acceptable salt, tautomer or stereoisomer thereof, wherein:
L¹ or L² is each independently —O(C═O)— or —(C═O)O—;
G¹ and G² are each independently unsubstituted C₁-C₁₂ alkylene;
G³ is C₁-C₂₄ alkylene;
R¹ and R² are each independently, branched or linear, C₆-C₂₄ alkyl;
R³ is OR⁵; and
R⁵ is H.

In some embodiments, the ionizable cationic lipid has the formula III and wherein R¹, R² or both R¹ and R² have one of the following structures:

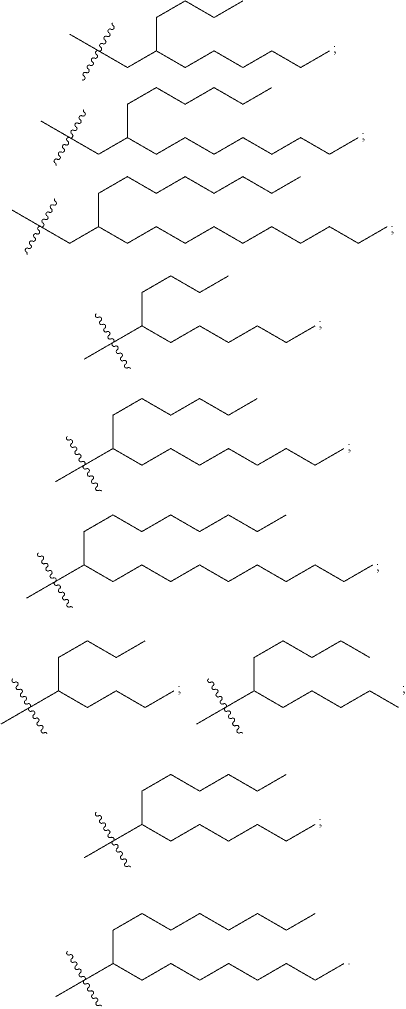

In some embodiments, R² has the structure:

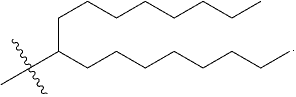 [SMILES: *C(CCCCCCCC)CCCCCCCC]

In some embodiments, the cationic lipid has the formula:

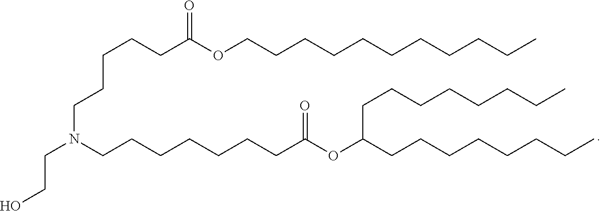 [SMILES: CCCCCCCCCCCOC(=O)CCCCCN(CCO)CCCCCCCC(=O)OC(CCCCCCCC)CCCCCCCC]

In some embodiments, the ionizable cationic lipid has the formula:

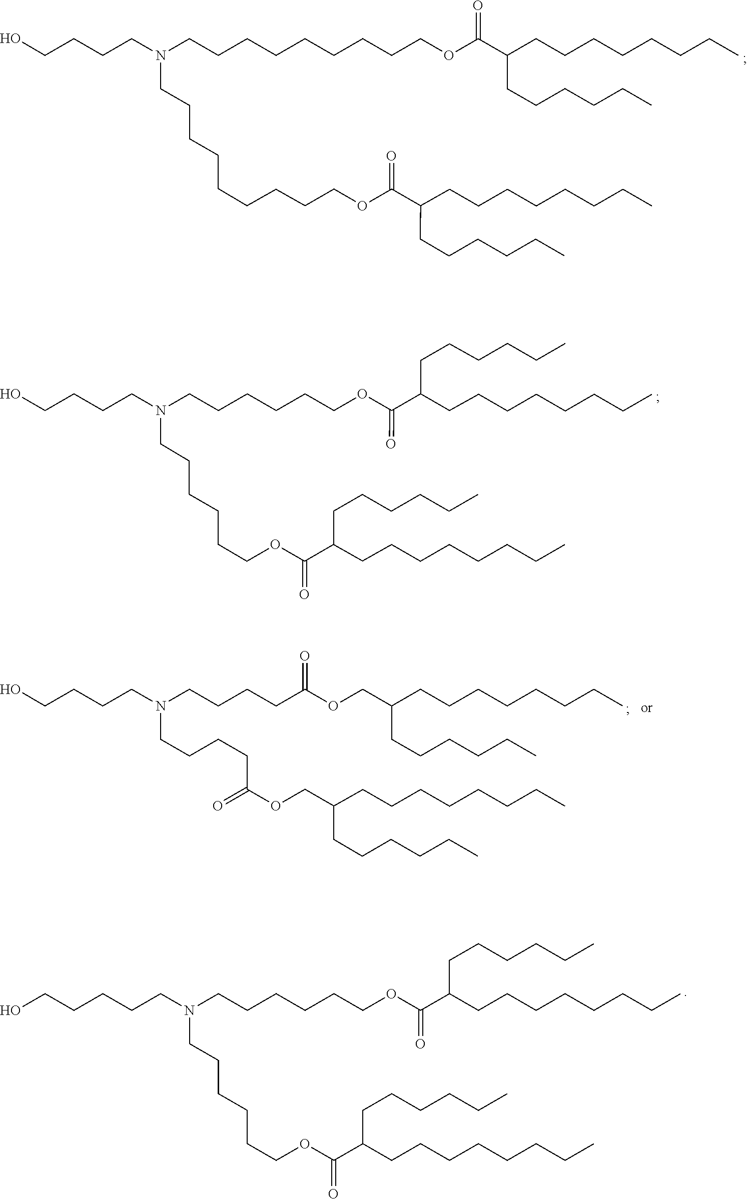 [SMILES: CCCCCCCCC(CCCCCC)C(=O)OCCCCCCCCCN(CCCCO)CCCCCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; CCCCCCCCC(CCCCCC)COC(=O)CCCCN(CCCCO)CCCCC(=O)OCC(CCCCCC)CCCCCCCC ; CCCCCCCCC(CCCCCC)C(=O)OCCCCCCN(CCCCCO)CCCCCCOC(=O)C(CCCCCC)CCCCCCCC]

In some embodiments, the ionizable cationic lipid has the formula III-3:

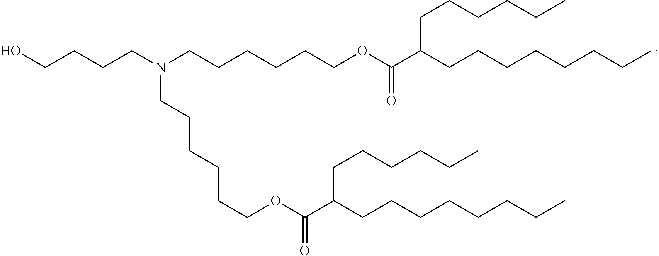 [SMILES: CCCCCCCCC(CCCCCC)C(=O)OCCCCCCN(CCCCO)CCCCCCOC(=O)C(CCCCCC)CCCCCCCC]

The lipid of formula III-3 as suitably used herein has the chemical term ((4-hydroxybutyl) azanediyl)bis(hexane-6,1-diyl)bis(2-hexyldecanoate), also referred to as ALC-0315 i.e. CAS Number 2036272-55-4.

In certain embodiments, the cationic lipid as defined herein, more suitably cationic lipid compound III-3 ((4-hydroxybutyl) azanediyl)bis(hexane-6,1-diyl)bis(2-hexyldecanoate)), is present in the LNP in an amount from about 30 mol % to about 80 mol %, suitably about 30 mol % to about 60 mol %, more suitably about 40 mol % to about 55 mol %, more suitably about 47.4 mol %, relative to the total lipid content of the LNP. If more than one cationic lipid is incorporated within the LNP, such percentages apply to the combined cationic lipids.

In some embodiments, the cationic lipid as defined herein is present in the LNP in an amount from about 20 mol % to about 60 mol %.

In some embodiments, the LNP comprises a cationic lipid having the following structure:

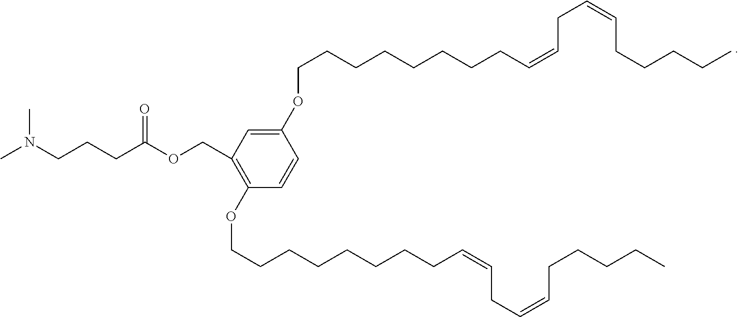 [SMILES: CCCCC/C=C\C/C=C\CCCCCCCCOc1ccc(OCCCCCCCC/C=C\C/C=C\CCCCC)c(COC(=O)CCCN(C)C)c1]

In embodiments, the cationic lipid is present in the LNP in an amount from about 30 mol % to about 70 mol %. In one embodiment, the cationic lipid is present in the LNP in an amount from about 40 mol % to about 60 mol %, such as about 40, 41, 42, 43, 44, 45, 46, 47, 48, 49, 50, 51, 52, 53, 54, 55, 56, 57, 58, 59 or 60 mol %, respectively. In embodiments, the cationic lipid is present in the LNP in an amount from about 47 mol % to about 48 mol %, such as about 47.0, 47.1, 47.2, 47.3, 47.4, 47.5, 47.6, 47.7, 47.8, 47.9, 50.0 mol %, respectively, wherein 47.4 mol % are particularly suitable.

In some embodiments, the cationic lipid is present in a ratio of from about 20 mol % to about 70 mol % or 75 mol % or from about 45 mol % to about 65 mol % or about 20, 25, 30, 35, 40, 45, 50, 55, 60, 65, or about 70 mol % of the total lipid present in the LNP. In further embodiments, the LNPs comprise from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In some embodiments, the ratio of cationic lipid to nucleic acid, suitably RNA, more suitably mRNA, is from about 3 to about 15, such as from about 5 to about 13 or from about 7 to about 11.

Other suitable (cationic or ionizable) lipids are disclosed in WO2009086558, WO2009127060, WO2010048536, WO2010054406, WO2010088537, WO2010129709, WO2011153493, WO 2013063468, US20110256175, US20120128760, US20120027803, U.S. Pat. No. 8,158,601, WO2016118724, WO2016118725, WO2017070613, WO2017070620, WO2017099823, WO2012040184, WO2011153120, WO2011149733, WO2011090965, WO2011043913, WO2011022460, WO2012061259, WO2012054365, WO2012044638, WO2010080724, WO201021865, WO2008103276, WO2013086373, WO2013086354, U.S. Pat. Nos. 7,893,302, 7,404,969, 8,283,333, 8,466,122 and 8,569,256 and US Patent Publication No. US20100036115, US20120202871, US20130064894, US20130129785, US20130150625, US20130178541, US20130225836, US20140039032 and WO2017112865. In that context, the disclosures of WO2009086558, WO2009127060, WO2010048536, WO2010054406, WO2010088537, WO2010129709, WO2011153493, WO 2013063468, US20110256175, US20120128760, US20120027803, U.S. Pat. No. 8,158,601, WO2016118724, WO2016118725, WO2017070613, WO2017070620, WO2017099823, WO2012040184, WO2011153120, WO2011149733, WO2011090965, WO2011043913, WO2011022460, WO2012061259, WO2012054365, WO2012044638, WO2010080724, WO201021865, WO2008103276, WO2013086373, WO2013086354, U.S. Pat. Nos. 7,893,302, 7,404,969, 8,283,333, 8,466,122 and 8,569,256 and US Patent Publication No. US20100036115, US20120202871, US20130064894, US20130129785, US20130150625, US20130178541, US20130225836 and US20140039032 and WO2017112865 specifically relating to (cationic) lipids suitable for LNPs (or liposomes, nanoliposomes, lipoplexes) are incorporated herewith by reference.

In other embodiments, the cationic or ionizable lipid is

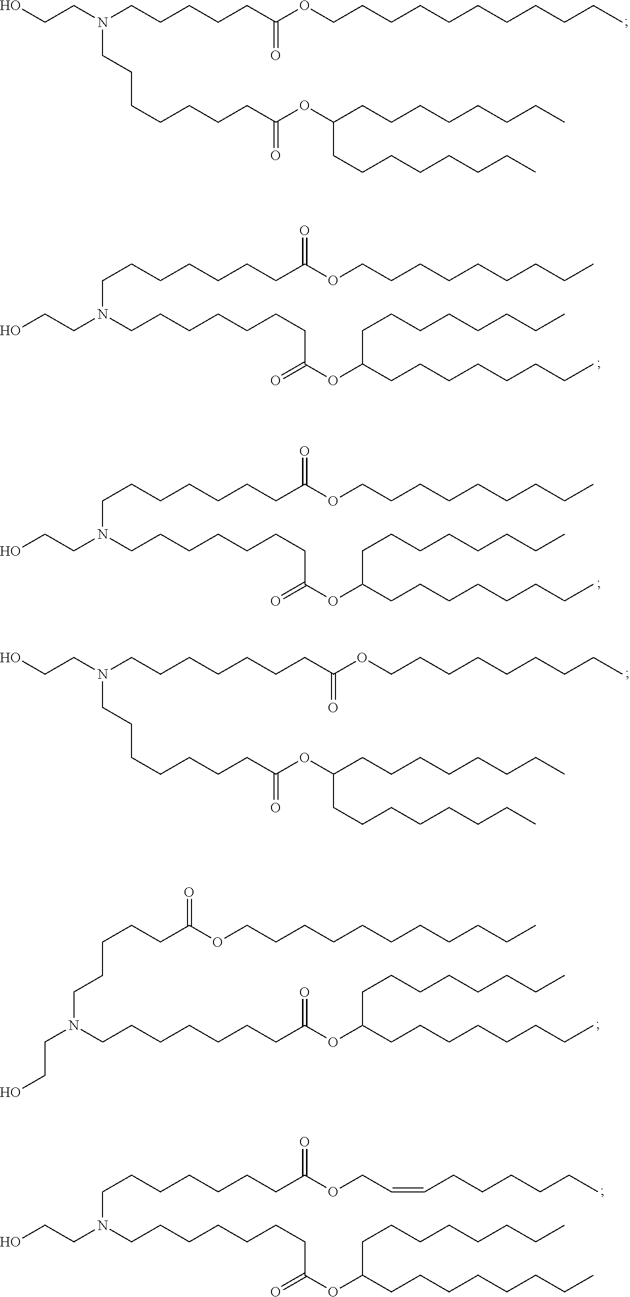

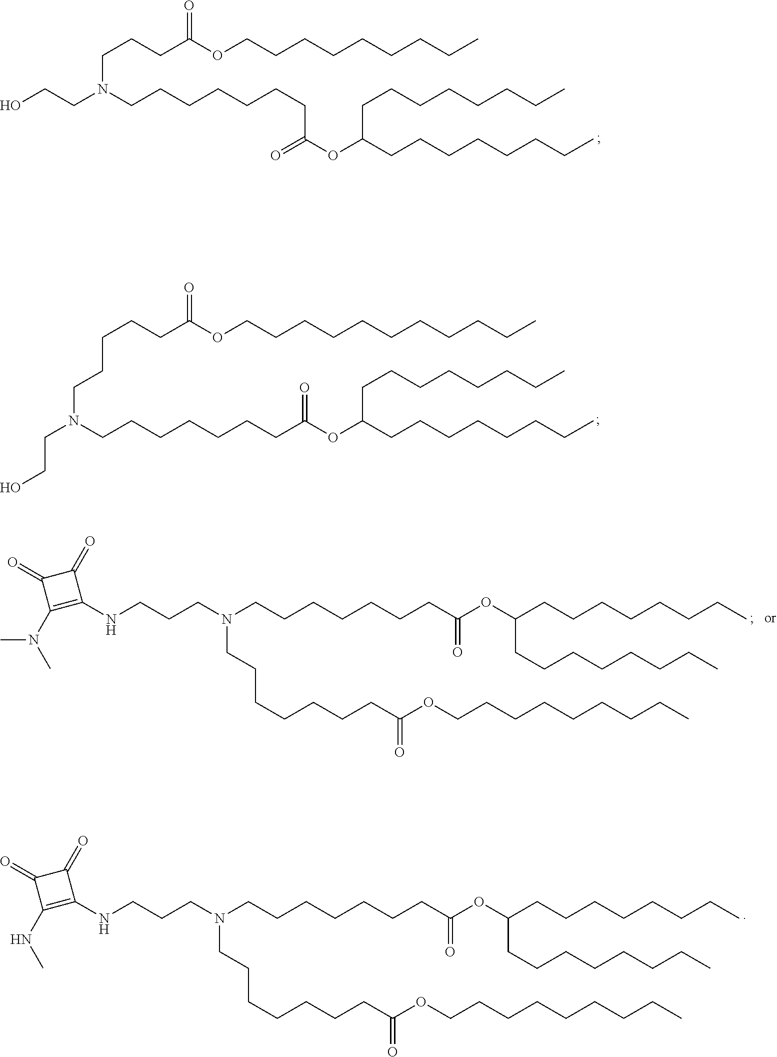

In embodiments, amino or cationic lipids as defined herein have at least one protonatable or deprotonatable group, such that the lipid is positively charged at a pH at or below physiological pH (e.g. pH 7.4), and neutral at a second pH, suitably at or above physiological pH. It will, of course, be understood that the addition or removal of protons as a function of pH is an equilibrium process, and that the reference to a charged or a neutral lipid refers to the nature of the predominant species and does not require that all of lipids have to be present in the charged or neutral form. Lipids having more than one protonatable or deprotonatable group, or which are zwitterionic, are not excluded and may likewise suitable in the context of the present invention. In some embodiments, the protonatable lipids have a pKa of the protonatable group in the range of about 4 to about 11, e.g., a pKa of about 5 to about 7.

LNPs (or liposomes, nanoliposomes, lipoplexes) can comprise two or more (different) cationic lipids as defined herein. Cationic lipids may be selected to contribute to different advantageous properties. For example, cationic lipids that differ in properties such as amine pKa, chemical stability, half-life in circulation, half-life in tissue, net accumulation in tissue, or toxicity can be used in the LNP (or liposomes, nanoliposomes, lipoplexes). In particular, the cationic lipids can be chosen so that the properties of the mixed-LNP are more desirable than the properties of a single-LNP of individual lipids.

The amount of the permanently cationic lipid or lipidoid may be selected taking the amount of the nucleic acid cargo into account. In one embodiment, these amounts are selected such as to result in an N/P ratio of the nanoparticle(s) or of the composition in the range from about 0.1 to about 20, or
(i) at an amount such as to achieve an N/P ratio in the range of about 1 to about 20, suitably about 2 to about 15, more suitably about 3 to about 10, even more suitably about 4 to about 9, most suitably about 6;
(ii) at an amount such as to achieve an N/P ratio in the range of about 5 to about 20, more suitably about 10 to about 18, even more suitably about 12 to about 16, most suitably about 14;
(iii) at an amount such as to achieve a lipid: mRNA weight ratio in the range of 20 to 60, suitably from about 3 to about 15, 5 to about 13, about 4 to about 8 or from about 7 to about 11; or
(iv) at an amount such as to achieve an N/P ratio in the range of about 6 for a lipid nanoparticle according to the invention, especially a lipid nanoparticle comprising the cationic lipid III-3.

In this context, the N/P ratio is defined as the mole ratio of the nitrogen atoms (“N”) of the basic nitrogen-containing groups of the lipid or lipidoid to the phosphate groups (“P”) of the nucleic acid which is used as cargo. The N/P ratio may be calculated on the basis that, for example, 1 μg RNA typically contains about 3 nmol phosphate residues, provided that the RNA exhibits a statistical distribution of bases. The “N”-value of the cationic lipid or lipidoid may be calculated on the basis of its molecular weight and the relative content of permanently cationic and—if present—cationisable groups. If more than one cationic lipid is present, the N-value should be calculated on the basis of all cationic lipids comprised in the lipid nanoparticles.

In some embodiments, the composition has a lipid to RNA molar ratio (N/P ratio) of about 2 to about 12, optionally a N/P ratio of 3 to about 8.

In one embodiment the lipid nanoparticles comprise about 40% cationic lipid LKY750, about 10% zwitterionic lipid DSPC, about 48% cholesterol, and about 2% PEGylated lipid DMG (w/w).

In some embodiments, LNPs comprise: (a) the RNAs, suitably mRNAs, used herein, (b) a cationic lipid, (c) an aggregation reducing agent (such as polyethylene glycol (PEG) lipid or PEG-modified lipid), (d) optionally a non-cationic lipid (such as a neutral lipid), and (e) optionally, a sterol.

In some embodiments, the cationic lipids (as defined above), non-cationic lipids (as defined above), cholesterol (as defined above), and/or PEG-modified lipids (as defined above) may be combined at various relative molar ratios. For example, the ratio of cationic lipid to non-cationic lipid to cholesterol-based lipid to PEGylated lipid may be between about 30-60:20-35:20-30:1-15, or at a ratio of about 40:30:25:5, 50:25:20:5, 50:27:20:3, 40:30:20:10, 40:32:20:8, 40:32:25:3 or 40:33:25:2, or at a ratio of about 50:25:20:5, 50:20:25:5, 50:27:20:3 40:30:20:10,40:30:25:5 or 40:32:20:8, 40:32:25:3 or 40:33:25:2, respectively.

In some embodiments, the LNPs (or liposomes, nanoliposomes, lipoplexes) comprise ALC-0315, the RNAs, suitably mRNAs, used herein, a neutral lipid which is DSPC, a steroid which is cholesterol and a PEGylated lipid which is ALC-0159.

In some embodiments, the LNP comprises a PEG-modified lipid at around 0.5 to 15 molar %, a non-cationic lipid at around 5 to 25 molar %, a sterol at around 25 to 55 molar % and an ionisable cationic lipid at around 20 to 60 molar %.

In an embodiment, the LNP consists essentially of (i) at least one cationic lipid; (ii) a neutral lipid; (iii) a sterol, e.g., cholesterol; and (iv) a PEG-lipid, e.g. PEG-DMG or PEG-CDMA, in a molar ratio of about 20-60% cationic lipid: 5-25% neutral lipid: 25-55% sterol; 0.5-15% PEG-lipid.

In some embodiments, the RNA, suitably mRNA, is complexed with one or more lipids thereby forming lipid nanoparticles, wherein the LNP comprises
I. at least one cationic lipid as defined herein, suitably lipid of formula III-3 (ALC-0315);
II. at least one neutral lipid as defined herein, suitably 1,2-distearoyl-sn-glycero-3-phosphocholine (DSPC);
III. at least one steroid or steroid analogue as defined herein, suitably cholesterol; and
IV. at least one polymer conjugated lipid, suitably a PEG-lipid as defined herein, e.g. PEG-DMG or PEG-cDMA, suitably a PEGylated lipid that is or is derived from formula (IVa-ALC-0159).

In some embodiments, the mRNA is complexed with one or more lipids thereby forming lipid nanoparticles (LNP), wherein the LNP comprises (i) to (iv) in a molar ratio of about 20-60% cationic lipid: 5-25% neutral lipid: 25-55% sterol; 0.5-15% polymer conjugated lipid, suitably PEG-lipid.

In some embodiments, the lipid nanoparticle (or liposome, nanoliposome, lipoplexe) comprises: a cationic lipid with formula (III-3) and/or PEG lipid with formula (IVa), optionally a neutral lipid, suitably 1,2-distearoyl-sn-glycero-3-phosphocholine (DSPC) and optionally a steroid, suitably cholesterol, wherein the molar ratio of the cationic lipid to DSPC is optionally in the range from about 2:1 to 8:1, wherein the molar ratio of the cationic lipid to cholesterol is optionally in the range from about 2:1 to 1:1.

In an embodiment, the composition comprises the RNA, suitably mRNA, lipid nanoparticles (LNPs), which have a molar ratio of approximately 50:10:38.5:1.5, suitably 47.5:10:40.8:1.7 or more suitably 47.4:10:40.9:1.7 (i.e. proportion (mol %) of cationic lipid (suitably lipid of formula III-3 (ALC-0315)), DSPC, cholesterol and polymer conjugated lipid, suitably PEG-lipid (suitably PEG-lipid of formula (IVa) with n=49, even more suitably PEG-lipid of formula (IVa) with n=45; ALC-0159); solubilized in ethanol).

WO2017/070620 provides general information on LNP compositions and is incorporated herein by reference. Other useful LNPs are described in the following references: WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053, which are also incorporated herein by reference.

In various embodiments, LNPs that suitably encapsulates the mRNA of the invention have a mean diameter of from about 50 nm to about 200 nm, from about 60 nm to about 200 nm, from about 70 nm to about 200 nm, from about 80 nm to about 200 nm, from about 90 nm to about 200 nm, from about 90 nm to about 190 nm, from about 90 nm to about 180 nm, from about 90 nm to about 170 nm, from about 90 nm to about 160 nm, from about 90 nm to about 150 nm, from about 90 nm to about 140 nm, from about 90 nm to about 130 nm, from about 90 nm to about 120 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 160 nm, 170 nm, 180 nm, 190 nm, or 200 nm and are substantially non-toxic. As used herein, the mean diameter may be represented by the z-average size as determined by dynamic light scattering as commonly known in the art.

Suitably the LNPs have a polydispersity of 0.4 or less, such as 0.3 or less. Typically, the PDI is determined by dynamic light scattering.

In some embodiments, the composition has a polydispersity index (PDI) value of less than about 0.4, suitably of less than about 0.3, more suitably of less than about 0.2, most suitably of less than about 0.1.

Suitably, at least 50%, more suitably at least 60%, 70% 80%, 85%, 90% or 95% of the RNA is encapsulated in the LNP. In this context, “encapsulated RNA” is understood as RNA (suitably mRNA) that is complexed with the lipids forming the LNP and/or that is contained within the interior space of the LNP. The proportion of encapsulated RNA can typically be determined using a RiboGreen assay.

Suitably, the composition contains less than about 30%, suitably less than 20%, 15%, 10% or 5% non-encapsulated RNA (or free RNA). In this context, the term “free RNA” or “non-encapsulated RNA” is understood as RNA (suitably mRNA) that is not encapsulated in the LNPs as defined herein. In a therapeutic composition, free RNA, may represent a contamination or an impurity.

### Vaccines and Combination Vaccines

The immunogenic composition as described herein is suitable for use as a vaccine.

In a second aspect, the invention relates to a vaccine comprising the immunogenic composition as described herein.

The vaccine may be a live attenuated vaccine, an inactivated vaccine, a recombinant vaccine or a nucleic acid-based vaccine.

The vaccine is suitable for active immunization against disease caused by Influenza virus, suitably Influenza subtype A viruses and Influenza type B viruses, contained in the vaccine.

In some embodiments, the vaccine is a multivalent vaccine.

In some embodiments, the vaccine is a trivalent (i.e. comprising immunogenic components derived from 3 strains of Influenza virus) or quadrivalent Influenza virus vaccine (i.e. comprising immunogenic components derived from 4 strains of Influenza virus) or pentavalent Influenza virus vaccine (i.e. comprising immunogenic components derived from 5 strains of Influenza virus).

In some embodiments, the vaccine is a trivalent Influenza virus vaccine.

In some embodiments, the trivalent Influenza virus vaccine comprises 3 HA antigens or nucleic acid, suitably mRNAs, encoding such.

In some embodiments, the trivalent Influenza virus vaccine comprises 2 HA antigens or nucleic acid, suitably mRNAs, encoding such derived from a strain of Influenza A virus, suitably from H1N1 and/or H3N2, and 1 HA antigens or nucleic acid, suitably mRNAs, encoding such derived from a strain of Influenza B virus, suitably from B/Victoria lineage.

In some embodiments, the trivalent Influenza virus vaccine comprises 3 mRNAs encoding 3 HA antigens.

In some embodiments, the trivalent Influenza virus vaccine comprises 2 mRNAs encoding 2 HA antigens derived from a strain of Influenza A virus, suitably from H1N1 and/or H3N2, and 1 mRNA encoding 1 HA antigen derived from a strain of Influenza B virus, suitably from B/Victoria lineage.

In some embodiments, the trivalent Influenza virus vaccine comprises 2 HA and 2 NA antigens or nucleic acid, suitably mRNAs, encoding such derived from a strain of Influenza A virus, suitably from H1N1 and/or H3N2, and 1 HA and 1 NA antigens or nucleic acid, suitably mRNAs, encoding such derived from a strain of Influenza B virus, suitably from B/Victoria lineage.

In some embodiments, the trivalent Influenza virus vaccine comprises 6 mRNAs encoding 3 HA and 3 NA antigens.

In some embodiments, the trivalent Influenza virus vaccine comprises 4 mRNAs encoding 2 HA and 2 NA antigens derived from a strain of Influenza A virus, suitably from H1N1 and/or H3N2, and 2 mRNA encoding 1 HA and 1 NA antigen derived from a strain of Influenza B virus, suitably from B/Victoria lineage.

In some embodiments, the trivalent Influenza virus vaccine comprises (a), (b) and (c) as defined herein.

In some embodiments, the trivalent Influenza virus vaccine comprises (a), (b), (c¹), (c³), (c⁴) and (c⁵) as defined herein.

In some embodiments, the trivalent Influenza virus vaccine comprises (a), (b) and (c) as defined herein or (a), (b), (c¹), (c³), (c⁴) and (c⁵) as defined herein, wherein the ratio of (a):(b): (c) or (a): (b): (c¹) is comprised between 1.5:1:1 and 20:1:1, optionally between 1.5:1:1 and 5:1:1, suitably between 2:1:1 and 4:1:1, suitably between 2:1:1 and 3:1:1, suitably is 2:1:1 or 3:1:1.

In some embodiments, the trivalent Influenza virus vaccine comprises (a), (b) and (c) as defined herein or (a), (b), (c¹), (c³), (c⁴) and (c⁵) as defined herein, wherein the ratio of (a):(b): (c) or (a): (b): (c¹) is greater than 3:1:1 and lower or equal to 20:1:1.

In some embodiments, the trivalent Influenza virus vaccine comprises (a), (b) and (c) as defined herein or (a), (b), (c¹), (c³), (c⁴) and (c⁵) as defined herein, wherein the ratio of (a):(b): (c) or (a): (b): (c¹) is greater than 3:1:1 and lower or equal to 5:1:1.

In some embodiments, the vaccine is a quadrivalent Influenza virus vaccine.

In some embodiments, the quadrivalent Influenza virus vaccine comprises 4 HA antigens or nucleic acid, suitably mRNAs, encoding such.

In some embodiments, the quadrivalent Influenza virus vaccine comprises 2 HA antigens or nucleic acids, suitably mRNAs, encoding such derived from a strain of Influenza A virus, suitably from H1N1 and/or H3N2, and 2 HA antigens or nucleic acids, suitably mRNAs, encoding such derived from a strain of Influenza B virus, suitably from B/Victoria lineage and/or B/Yamagata lineage.

In some embodiments, the quadrivalent Influenza virus vaccine comprises 2 HA antigens or nucleic acids, suitably mRNAs, encoding such derived from two strains of H3N2 Influenza A virus, 1 HA antigen or nucleic acid, suitably mRNA, encoding such derived from a strain of H1N1 Influenza A virus and 1 HA antigen or nucleic acid, suitably mRNA, encoding such derived from a strain of Influenza B virus, suitably from B/Victoria lineage.

In some embodiments, the quadrivalent Influenza virus vaccine comprises 4 mRNAs encoding 4 HA antigens.

In some embodiments, the quadrivalent Influenza virus vaccine comprises 2 mRNAs encoding 2 HA antigens derived from a strain of Influenza A virus, suitably from H1N1 and/or H3N2, and 2 mRNA encoding 2 HA antigen derived from a strain of Influenza B virus, suitably from B/Victoria lineage and/or B/Yamagata lineage.

In some embodiments, the quadrivalent Influenza virus vaccine comprises 2 mRNAs encoding 2 HA antigens derived from two strains of H3N2 Influenza A virus, 1 mRNA encoding 1 HA antigen derived from a strain of H1N1 Influenza A virus and 1 mRNA encoding 1 HA antigen derived from a strain of Influenza B virus, suitably from B/Victoria lineage.

In some embodiments, the quadrivalent Influenza virus vaccine comprises (a), (b), (c¹) and (c²) as defined herein, wherein the ratio of (a):(b): (c¹): (c²) is comprised between 1.5:1:1:1 and 20:1:1:1, optionally between 1.5:1:1:1 and 5:1:1:1, suitably between 2:1:1:1 and 4:1:1:1, suitably between 2:1:1:1 and 3:1:1:1, suitably is 2:1:1:1 or 3:1:1:1.

In some embodiments, the quadrivalent Influenza virus vaccine comprises (a), (b), (c¹) and (c²) as defined herein, wherein the ratio of (a):(b): (c¹): (c²) is greater than 3:1:1:1 and lower or equal to 20:1:1:1.

In some embodiments, the quadrivalent Influenza virus vaccine comprises (a), (b), (c¹) and (c²) as defined herein, wherein the ratio of (a):(b): (c¹): (c²) is greater than 3:1:1:1 and lower or equal to 5:1:1:1.

In some embodiments, the quadrivalent Influenza virus vaccine comprises 4 HA antigens or nucleic acid, suitably mRNAs, encoding such, and 3 NA antigens or nucleic acid, suitably mRNAs, encoding such, such as (i.e. seven components quadrivalent Influenza virus vaccine).

In some embodiments, the quadrivalent Influenza virus vaccine comprises 4 mRNAs encoding 4 HA antigens and 3 mRNAs encoding 3 NA antigens.

In some embodiments, the quadrivalent Influenza virus vaccine comprises (a), (b), (c¹), (c²), (c³), (c⁴) and (c⁵) as defined herein, wherein the ratio of (a):(b): (c¹): (c²) is comprised between 1.5:1:1:1 and 20:1:1:1, optionally between 1.5:1:1:1 and 5:1:1:1, suitably between 2:1:1:1 and 4:1:1:1, suitably between 2:1:1:1 and 3:1:1:1, suitably is 2:1:1:1 or 3:1:1:1.

In some embodiments, the quadrivalent Influenza virus vaccine comprises (a), (b), (c¹), (c²), (c³), (c⁴) and (c⁵) as defined herein, wherein the ratio of (a):(b): (c¹): (c²) is greater than 3:1:1:1 and lower or equal to 20:1:1:1.

In some embodiments, the quadrivalent Influenza virus vaccine comprises (a), (b), (c¹), (c²), (c³), (c⁴) and (c⁵) as defined herein, wherein the ratio of (a):(b): (c¹): (c²) is greater than 3:1:1:1 and lower or equal to 5:1:1:1.

In some embodiments, the quadrivalent Influenza virus vaccine comprises 4 HA antigens or nucleic acid, suitably mRNAs, encoding such and 4 NA antigens or nucleic acid, suitably mRNAs, encoding such (i.e. eight components quadrivalent Influenza virus vaccine).

In some embodiments, the quadrivalent Influenza virus vaccine comprises 4 mRNAs encoding 4 HA antigens and 4 mRNAs encoding 4 NA antigens.

In some embodiments, the quadrivalent Influenza virus vaccine comprises (a), (b), (c¹), (c²), (c³), (c⁴), (c⁵) and (c⁶) as defined herein, wherein the ratio of (a):(b): (c¹): (c²) is comprised between 1.5:1:1:1 and 20:1:1:1, optionally between 1.5:1:1:1 and 5:1:1:1, suitably between 2:1:1:1 and 4:1:1:1, suitably between 2:1:1:1 and 3:1:1:1, suitably is 2:1:1:1 or 3:1:1:1.

In some embodiments, the quadrivalent Influenza virus vaccine comprises (a), (b), (c¹), (c²), (c³), (c⁴), (c⁵) and (c⁶) as defined herein, wherein the ratio of (a):(b): (c¹): (c²) is greater than 3:1:1:1 and lower or equal to 20:1:1:1.

In some embodiments, the quadrivalent Influenza virus vaccine comprises (a), (b), (c¹), (c²), (c³), (c⁴), (c⁵) and (c⁶) as defined herein, wherein the ratio of (a):(b): (c¹): (c²) is greater than 3:1:1:1 and lower or equal to 5:1:1:1.

In some embodiments, the vaccine is a pentavalent Influenza virus vaccine.

In some embodiments, the pentavalent Influenza virus vaccine comprises 5 HA antigens or nucleic acid, suitably mRNAs, encoding such.

In some embodiments, the pentavalent Influenza virus vaccine comprises 2 HA antigens or nucleic acids, suitably mRNAs, encoding such derived from two strains of H3N2 Influenza A virus, 1 HA antigen or nucleic acid, suitably mRNA, encoding such derived from a strain of H1N1 Influenza A virus and 2 HA antigen or nucleic acid, suitably mRNA, encoding such derived from a strain of Influenza B virus, suitably from B/Victoria lineage and/or B/Yamagata lineage.

In some embodiments, the pentavalent Influenza virus vaccine comprises 5 mRNAs encoding 5 HA antigens.

In some embodiments, the pentavalent Influenza virus vaccine comprises 2 mRNAs encoding 2 HA antigens derived from two strains of H3N2 Influenza A virus, 1 mRNA encoding 1 HA antigen derived from a strain of H1N1 Influenza A virus and 2 mRNA encoding 2 HA antigen derived from a strain of Influenza B virus, suitably from B/Victoria lineage.

In some embodiments, the pentavalent Influenza virus vaccine comprises 5 HA antigens or nucleic acid, suitably mRNAs, encoding such and 5 NA antigens or nucleic acid, suitably mRNAs, encoding such (i.e. ten components pentavalent Influenza virus vaccine).

In some embodiments, the pentavalent Influenza virus vaccine comprises 5 mRNAs encoding 5 HA antigens and 5 mRNAs encoding 5 NA antigens.

In some embodiments, the vaccine further comprises at least one antigen or at least one nucleic acid encoding said at least one antigen, such as at least one mRNA encoding an antigen from a further pathogen, suitably the pathogen being a virus, suitably a respiratory virus.

In some embodiments, said antigen is from further virus is selected from the group consisting of Coronavirus (e.g. SARS-COV-1, SARS-COV-2, MERS-COV), Pneumoviridae virus (e.g. Respiratory syncytial virus, Metapneumovirus) and Paramyxovidirae virus (e.g. Parainfluenza virus, Henipavirus), suitably said antigen from a further virus is a spike protein, or an antigenic fragment thereof, from a SARS-COV-2 virus or a mRNA encoding a spike protein, or an antigenic fragment thereof, from a SARS-COV-2 virus. For instance, the antigen can be a SARS-COV-2 virus spike protein or an antigenic fragment thereof selected from those provided in Table 1 of published PCT application WO2021156267A1 or in Table 1 of published PCT application WO2022137133A1, each of which is incorporated herein by reference.

### Kit or Kit of Parts

In a third aspect, the invention relates to a kit or kit of parts comprising the antigens or the nucleic acids and/or the mRNAs, suitably the mRNAs of (a), (b), (c), (c¹), (c²), (c³), (c⁴), (c⁵) and/or (c⁶), as in defined herein, optionally comprising a liquid vehicle for solubilizing, and, optionally, technical instructions providing information on administration and dosage of the components.

The technical instructions of the kit may contain information about administration and dosage and patient groups. Such kits, suitably kits of parts, may be applied e.g. for any of the applications or uses mentioned herein, suitably for the use of the immunogenic composition or the vaccine for the treatment or prophylaxis of an infection or diseases caused by an Influenza virus, suitably Influenza A and/or B virus.

In some embodiments, the immunogenic composition or the vaccine is provided in a separate part of the kit, wherein the immunogenic composition or the vaccine is suitably lyophilised or spray-dried or spray-freeze dried.

The kit may further contain as a part, a vehicle (e.g. buffer solution) for solubilising the dried or lyophilized nucleic composition or the vaccine.

In some embodiments, the antigens or the nucleic acids and/or the mRNAs, suitably the mRNAs of (a), (b), (c), (c¹), (c²), (c³), (c⁴), (c⁵) and/or (c⁶), as defined herein are formulated separately.

In some embodiments, the antigens or the nucleic acids and/or the mRNAs, suitably the mRNAs of (a), (b), (c), (c¹), (c²), (c³), (c⁴), (c⁵) and/or (c⁶), as defined herein are provided as one part of the kit.

In some embodiments, the antigens or the nucleic acids and/or the mRNAs, suitably the mRNAs of (a), (b), (c), (c¹), (c²), (c³), (c⁴), (c⁵) and/or (c⁶), as defined herein are each provided as a separate part of the kit. Suitably, the kit or kit of parts comprises at least two, at least three, at least four, at least five, at least six, at least seven, at least eight parts, each containing at least one of the nucleic acids and/or the mRNAs, suitably the mRNAs of (a), (b), (c), (c¹), (c²), (c³), (c⁴), (c⁵) and/or (c⁶), as defined herein.

In some embodiments, the kit or kit of parts as defined herein comprises a multi-dose container for administration of the composition/the vaccine and/or an administration device (e.g. an injector for intramuscular and/or intradermal injection).

### Formulation and Administration

In some embodiments, the antigens or the nucleic acids, suitably mRNAs, as defined herein are co-formulated. Suitably, the mRNAs of (a), (b), (c), (c¹), (c²), (c³), (c⁴), (c⁵) and/or (c⁶), as defined herein are co-formulated, i.e. formulated together.

In some embodiments, the antigens or the nucleic acids, suitably mRNAs, as defined herein of the kit or kit of parts are formulated separately. In some embodiments, the mRNAs of (a), (b), (c), (c¹), (c²), (c³), (c⁴), (c⁵) and/or (c⁶), as defined herein are formulated separately.

In some embodiments, the antigens or the nucleic acids, suitably mRNAs, as defined herein are co-filled. Suitably, the mRNAs of (a), (b), (c), (c¹), (c²), (c³), (c⁴), (c⁵) and/or (c⁶), as defined herein are co-filled, i.e. filled together, optionally after being formulated separately.

In some embodiments, the antigens or the nucleic acids, suitably mRNAs, as defined herein are formulated as a bedside mixing formulation. Suitably the mRNAs, suitably the mRNAs of (a), (b), (c), (c¹), (c²), (c³), (c⁴), (c⁵) and/or (c⁶), as defined herein are formulated as a bedside mixing formulation.

As described herein, a “bedside mixing formulation” must be understood as a formulation wherein some (such as one or more) of the immunogenic components (e.g. mRNA), suitably each, have been formulated (e.g. in LNPs) independently before being mixed to form the bedside mixing formulation.

In some embodiments, the bedside mixing formulation is obtained by a process comprising (1) formulating (e.g. in LNPs) each antigen or nucleic acid, suitably mRNAs, independently and (2) mixing each (LNP-) formulated antigen or nucleic acid, suitably mRNAs.

In some embodiments, the bedside mixing formulation is obtained by a process comprising (1) co-formulating (e.g. in LNPs) said antigen or nucleic acid, suitably mRNAs, encoding such derived from a strain of Influenza A virus (2) co-formulating said antigen or nucleic acid, suitably mRNAs, encoding such derived from a strain of Influenza B virus, and (3) mixing (LNP-) co-formulated antigens or nucleic acids, suitably mRNAs encoding such derived from a strain of Influenza A virus with (LNP-) co-formulated antigens or nucleic acids, suitably mRNAs encoding such derived from a strain of Influenza B virus.

In some embodiments, the bedside mixing formulation is obtained by a process comprising (1) co-formulating (e.g. in LNPs) said antigen or nucleic acid, suitably mRNAs, encoding such derived from a strain of Influenza A virus (2) formulating each antigen or nucleic acid, suitably mRNAs, encoding such derived from a strain of Influenza B virus independently, and (3) mixing (LNP-) co-formulated antigens or nucleic acids, suitably mRNAs encoding such derived from a strain of Influenza A virus and each (LNP-) formulated antigens or nucleic acids, suitably mRNAs encoding such derived from a strain of Influenza B virus.

The immunogenic composition may be administered via various suitable routes, including parenteral, such as intramuscular, intradermal, intranasal, or subcutaneous administration. Suitably the immunogenic composition, the vaccine or the kit or kit of parts as described herein is administered intramuscularly and/or intradermally.

In some embodiments, intramuscular administration of the immunogenic composition as described herein results in expression of the encoded antigen construct in a subject. Administration of the immunogenic composition as described herein results in translation of the mRNA and to a production of the encoded antigen in a subject.

The immunogenic composition described herein may be provided in liquid or dry (e.g. lyophilised) form.

In some embodiments, the immunogenic composition is provided in liquid form.

In some embodiments, the immunogenic composition is a dry composition.

The term “dry composition” as used herein may be a composition that has been lyophilized (e.g. according to WO2016165831, WO2011069586, WO2022/232585, WO2022/101461, WO2022/076562, WO2012/170889 or WO2022/036170), or spray-dried, or freeze-dried (e.g. according to WO2016184575, WO2016184576 or WO2021/216541) to obtain a dry composition, suitably a temperature stable composition, for example in the form of a powder.

In one embodiment, the composition according to the invention is a lyophilized, freeze-dried or spray-dried dry composition comprising one or more further excipients selected from cryoprotectants, plasticizers and polymers. Suitably, the lyophilized, freeze-dried or spray-dried dry composition is mixed with a liquid, suitably an aqueous liquid such as sterile water or saline, to form a “reconstituted liquid formulation” prior to administration to a patient.

In embodiments, lyophilized or spray-dried composition has a water content of less than about 10%.

In some embodiments, lyophilized or spray-dried composition has a water content of between about 0.5% and 5%.

In some embodiments, the lyophilized or spray-dried composition is stable for at least 2 months after storage at about 5° C., suitably for at least 3 months, 4 months, 5 months, 6 months.

Liquids used for reconstitution will be substantially aqueous, such as water for injection, phosphate buffered saline and the like. The requirement for buffer and/or tonicity modifying agents will depend on the on both the contents of the container being reconstituted and the subsequent use of the reconstituted contents. Buffers may be selected from acetate, citrate, histidine, maleate, phosphate, succinate, tartrate and TRIS. The buffer may be a phosphate buffer such as Na/Na₂PO₄, Na/K₂PO₄ or K/K₂PO₄.

Suitably, the formulations used in the present invention have a dose volume of between 0.05 ml and 1 ml, such as between 0.1 and 0.6 ml, in particular a dose volume of 0.45 to 0.55 ml, such as 0.5 ml. The volumes of the compositions used may depend on the subject, delivery route and location, with smaller doses being given by the intradermal route. A typical human dose for administration through routes such as intramuscular, is in the region of 200 μl to 750 μl, such as 400 to 600 μl, in particular about 500 μl, such as 500 μl.

The immunogenic composition as described herein may be provided in various physical containers such as vials or pre-filled syringes.

In some embodiments, the immunogenic composition is provided in the form of a single dose. In other embodiments, the immunogenic composition, the vaccine or the kit or kit of parts is provided in multidose form such containing 2, 5 or 10 doses.

It is common where liquids are to be transferred between containers, such as from a vial to a syringe, to provide ‘an overage’ which ensures that the full volume required can be conveniently transferred. The level of overage required will depend on the circumstances, but excessive overage should be avoided to reduce wastage and insufficient overage may cause practical difficulties. Overages may be of the order of 20 to 100 μl per dose, such as 30 μl or 50 μl.

Stabilisers may be present. Stabilisers may be of particular relevance where multidose containers are provided as doses of the final formulation(s) may be administered to subjects over a period of time.

### Formulations are Suitably Sterile.

Approaches for establishing strong and lasting immunity often include repeated immunisation, i.e. boosting an immune response by administration of one or more further doses. Such further administrations may be performed with the same immunogenic compositions (homologous boosting) or with different immunogenic compositions (heterologous boosting). The present invention may be applied as part of a homologous or heterologous prime/boost regimen, as either the priming or a/the boosting immunisation.

Administration of the immunogenic composition as described herein may therefore be part of a multi-dose administration regime. For example, the immunogenic composition as described herein may be provided as a priming dose in a multidose regime, especially a two- or three-dose regime, in particular a two-dose regime. The immunogenic composition as described herein may be provided as a boosting dose in a multidose regime, especially a two- or three-dose regime, such as a two-dose regime.

Priming and boosting doses may be homologous or heterologous. Consequently, the immunogenic composition as described herein may be provided as a priming dose and boosting dose(s) in a homologous multidose regime, especially a two- or three-dose regime, in particular a two-dose regime. Alternatively, the immunogenic composition as described herein may be provided as a priming dose or boosting dose in a heterologous multidose regime, especially a two- or three-dose regime, in particular a two-dose regime, and the boosting dose(s) may be different (e.g. an immunogenic composition as described herein; or an alternative antigen presentation—with or without adjuvant, such as squalene emulsion adjuvant).

The time between doses may be two weeks to six months, such as three weeks to three months. Periodic longer-term booster doses may also be provided, such as every 2 to 10 years.

In some embodiments, the immunogenic composition further comprises at least one pharmaceutically acceptable carrier.

The term “pharmaceutically acceptable carrier” or “pharmaceutically acceptable excipient” as used herein suitably includes the liquid or non-liquid basis of the composition for administration. If the composition is provided in liquid form, the carrier may be water, e.g. pyrogen-free water; isotonic saline or buffered (aqueous) solutions, e.g. phosphate, citrate etc. buffered solutions. Water or suitably a buffer, more suitably an aqueous buffer, may be used, containing a sodium salt, suitably at least 50 mM of a sodium salt, a calcium salt, suitably at least 0.01 mM of a calcium salt, and optionally a potassium salt, suitably at least 3 mM of a potassium salt. According to some embodiments, the sodium, calcium and, optionally, potassium salts may occur in the form of their halogenides, e.g. chlorides, iodides, or bromides, in the form of their hydroxides, carbonates, hydrogen carbonates, or sulfates, etc. Examples of sodium salts include NaCl, NaI, NaBr, Na₂CO₃, NaHCO₃, Na₂SO₄, examples of the optional potassium salts include KCl, KI, KBr, K₂CO₃, KHCO₃, K₂SO₄, and examples of calcium salts include CaCl₂), Cal₂, CaBr₂, CaCO₃, CaSO₄, Ca(OH)₂.

Furthermore, organic anions of the aforementioned cations may be in the buffer. Accordingly, in embodiments, the immunogenic composition may comprise pharmaceutically acceptable carriers or excipients using one or more pharmaceutically acceptable carriers or excipients to e.g. increase stability, increase cell transfection, permit the sustained or delayed, increase the translation of encoded antigenic peptides or proteins in vivo, and/or alter the release profile of encoded antigenic peptides or proteins protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with polynucleotides, hyaluronidase, nanoparticle mimics and combinations thereof. In embodiments, one or more compatible solid or liquid fillers or diluents or encapsulating compounds may be used as well, which are suitable for administration to a subject. The term “compatible” as used herein means that the constituents of the composition are capable of being mixed with the at least one nucleic acid of component A and/or component B and, optionally, a plurality of nucleic acids of the composition, in such a manner that no interaction occurs, which would substantially reduce the biological activity or the pharmaceutical effectiveness of the composition under typical use conditions (e.g., intramuscular or intradermal administration). Pharmaceutically acceptable carriers or excipients must have sufficiently high purity and sufficiently low toxicity to make them suitable for administration to a subject to be treated. Compounds which may be used as pharmaceutically acceptable carriers or excipients may be sugars, such as, for example, lactose, glucose, trehalose, mannose, and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate;

vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid.

The at least one pharmaceutically acceptable carrier or excipient of the immunogenic composition may be selected to be suitable for intramuscular or intradermal delivery/administration of the immunogenic composition. The immunogenic composition is suitably a composition suitable for intramuscular administration to a subject.

Subjects to which administration of the immunogenic compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In various embodiments, the immunogenic composition does not exceed a certain proportion of free RNA, suitably mRNA.

In this context, the term “free RNA, suitably mRNA” or “non-complexed RNA, suitably mRNA” or “non-encapsulated RNA, suitably mRNA” comprise the RNA, suitably mRNA molecules that are not encapsulated in the lipid-based carriers as defined herein. During formulation of the composition (e.g. during encapsulation of the RNA, suitably mRNA, into the lipid-based carriers), free RNA, suitably mRNA may represent a contamination or an impurity.

In embodiments, the immunogenic composition comprises free RNA, suitably mRNA, ranging from about 30% to about 0%. In embodiments, the composition comprises about 20% free RNA, suitably mRNA (and about 80% encapsulated RNA, suitably mRNA), about 15% free RNA, suitably mRNA (and about 85% encapsulated RNA, suitably mRNA), about 10% free RNA, suitably mRNA (and about 90% encapsulated RNA, suitably mRNA), or about 5% free RNA, suitably mRNA (and about 95% encapsulated RNA, suitably mRNA). In some embodiments, the composition comprises less than about 20% free RNA, suitably mRNA, suitably less than about 15% free RNA, suitably mRNA, more suitably less than about 10% free RNA, suitably mRNA, most suitably less than about 5% free RNA, suitably mRNA.

The term “encapsulated RNA, suitably mRNA” comprises the RNA, suitably mRNA, molecules that are encapsulated in the lipid-based carriers as defined herein. The proportion of encapsulated RNA, suitably mRNA, in the context of the invention is typically determined using a RiboGreen assay.

### Medical Uses (First and Second/Further Medical Uses) and Methods of Treatment

In a fourth aspect, the invention relates to the immunogenic composition, the vaccine, or the kit or kit of parts as described herein, for use as a medicament.

Also described herein is a use of the immunogenic composition, the vaccine, or the kit or kit of parts as described herein, as a medicament.

In a fifth aspect, the invention relates to the immunogenic composition, the vaccine, or the kit or kit of parts as described herein, for use in the treatment or prophylaxis of an infection with an Influenza virus, suitably an Influenza A and/or Influenza B.

Also described herein is a use of the immunogenic composition, the vaccine, or the kit or kit of parts as described herein, in the treatment or prophylaxis of an infection with an Influenza virus, suitably an Influenza A and/or Influenza B.

In some embodiments, a single dose of the immunogenic composition is 0.1 to 1000 μg, especially 1 to 500 μg, especially 2 to 500 μg, in particular 10 to 250 μg, suitably 25 to 150 μg of total mRNA.

In some embodiments, a single dose of the immunogenic composition is 10 to 150 μg, optionally 20 to 100 μg, optionally 35 to 100 μg of total mRNA.

In some embodiments, a single dose of the immunogenic composition is 35, 36, 37, 38, 39, 40, 41, 42, 43, 44, 45, 46, 47, 48, 49, 50, 55, 60, 61, 62, 63, 64, 65, 66, 67, 68, 69, 70, 75, 80, 85, 90, 95, 96, 97, 98, 99 or 100 μg of total mRNA.

In further embodiments, a single dose of the immunogenic composition comprises a mixture of 2, 3, 4, 5, 6, 7, 8, 9 or 10 different mRNA and is 1 to 200 μg, suitably 1 to 60 μg, suitably 1 to 25 μg, suitably 2 to 25 μg, suitably 3 to 18 μg of each mRNA.

In some embodiments, a single dose of the immunogenic composition comprises a mixture of 2, 3, 4, 5, 6, 7, 8, 9 or 10, suitably of 6, different mRNA and is 0.5 to 100 μg, suitably 0.5 to 75 μg, suitably 2 to 75 μg of each mRNA.

In some embodiment, a single dose of the composition is 2 to 500 μg, especially 10 to 250 μg of total mRNA, such as 10 to 75 μg of total mRNA.

In some embodiments, a single dose of the immunogenic composition is 10 to 100 μg of total mRNA.

In some embodiment, a single dose of the composition is 6, 12, 15, 16, 18, 24, 32, 36, 48, 54, 60, 72, 84, 96 or 120 μg of total mRNA.

In some embodiments, a single dose of the composition is 1 to 10 μg of each mRNA for younger adult e.g. 18 to 64 years old.

In some embodiments, a single dose of the composition is 1, 2, 3, 6 or 9 μg of each mRNA for younger adult e.g. 18 to 64 years old.

In some embodiments, a single dose of the composition is 15 to 50 μg of total mRNA for younger adult e.g. 18 to 64 years old.

In some embodiment, a single dose of the composition is 16, 32 or 48 μg of total mRNA for younger adult e.g. 18 to 64 years old.

In some embodiments, a single dose of the composition is 0.5 to 50 μg of each mRNA for younger adult e.g. 18 to 64 years old.

In some embodiments, a single dose of the composition is 0.5, 1, 2, 3, 4, 5, 6, 7, 8, 9, 10, 11, 12, 13, 14, 15, 20, 21, 22, 23, 24, 25, 30, 35, 36, 37, 38, 39, 40, 45, 46, 47, 48, 49 or 50 μg of each mRNA for younger adult e.g. 18 to 64 years old.

In some embodiments, a single dose of the composition is 25 to 75 μg of total mRNA for younger adult e.g. 18 to 64 years old.

In some embodiment, a single dose of the composition is 25, 30, 35, 36, 37, 38, 39, 40, 41, 42, 43, 44, 45, 46, 47, 48, 49, 50, 55, 60, 65, 66, 67, 68, 69, 70 or 75 μg of total mRNA for younger adult e.g. 18 to 64 years old.

In some embodiments, a single dose of the composition is 2 to 20 μg of each mRNA for older adult e.g. 65 years old and above.

In some embodiments, a single dose of the composition is 2, 3, 6, 9 or 18 μg of each mRNA for older adult e.g. 65 years old and above.

In some embodiments, a single dose of the composition is 30 to 100 μg of total mRNA for older adult e.g. 65 years old and above.

In some embodiments, a single dose of the composition is 32, 48 or 96 μg of total mRNA for older adult e.g. 65 years old and above.

In some embodiments, a single dose of the composition is 0.5 to 100 μg of each mRNA for older adult e.g. 65 years old and above.

In some embodiments, a single dose of the composition is 0.5, 1, 2, 3, 4, 5, 6, 7, 8, 9, 10, 11, 12, 13, 14, 15, 20, 21, 22, 23, 24, 25, 30, 35, 36, 37, 38, 39, 40, 45, 46, 47, 48, 49, 50, 55, 60, 65, 70, 71, 72, 73, 74, 75, 80, 85, 90, 95 or 100 μg of each mRNA for older adult e.g. 65 years old and above.

In some embodiments, a single dose of the composition is 35 to 150 μg of total mRNA for older adult e.g. 65 years old and above.

In some embodiments, a single dose of the composition is 35, 40, 41, 42, 43, 44, 45, 46, 47, 48, 49, 50, 55, 60, 61, 62, 63, 64, 65, 66, 67, 68, 69, 70, 75, 80, 85, 90, 95, 96, 97, 98, 99, 100, 110, 120, 130, 140 or 150 μg of total mRNA for older adult e.g. 65 years old and above.

In some embodiment, the use is for intramuscular administration and/or intradermal administration suitably intramuscular administration.

In some embodiments, the antigens or the nucleic acids and/or the mRNAs, suitably the mRNAs of (a), (b), (c), (c¹), (c²), (c³), (c⁴), (c⁵) and/or (c⁶) as described herein are administered at different sites of injection.

In some embodiments, the antigens or the nucleic acids and/or the mRNAs derived from a strain of Influenza A virus are administered at a site of injection which is different to the site of injection where the antigens or the nucleic acids and/or the mRNAs derived from a strain of Influenza B virus are administered.

In some embodiments, the antigens or the nucleic acids and/or the mRNAs derived from a strain of Influenza B virus are administered separately, suitably at different sites of injection.

In some embodiments, an immune response is elicited, suitably an adaptative immune response, more suitably a protective adaptative immune response against an Influenza virus, suitably an Influenza A and/or Influenza B.

In some embodiments, an immune response is elicited.

In some embodiments, an adaptative immune response is elicited.

In some embodiments, a protective adaptative immune response against an Influenza virus is elicited.

In some embodiments, a protective adaptative immune response against an Influenza A and/or B virus is elicited.

In some embodiments, a protective adaptative immune response against one or more Influenza A virus subtype and/or Influenza B virus lineage is elicited, suitably against Influenza A H1N1, Influenza A H3N2, Influenza B/Yamagata lineage and Influenza B/Victoria lineage.

In some embodiments, the elicited immune response comprises neutralizing antibody titers against an Influenza virus, suitably an Influenza A and/or B virus, more suitably one or more Influenza A virus subtype and/or Influenza B virus lineage, more suitably against Influenza A H1N1, Influenza A H3N2, Influenza B/Yamagata lineage and Influenza B/Victoria lineage.

In some embodiments, the elicited immune response comprises functional antibodies that can effectively neutralize the respective viruses.

In some embodiments, the elicited immune response is a cross-reactive immune response, wherein the functional antibodies that can effectively neutralize the respective viruses further neutralize viruses belonging to same and/or other Influenza A subtypes and/or Influenza B lineages.

In some embodiments, the cross-reactive immune response is homologous, heterologous or intrasubtypic and/or heterosubtypic.

The term “homologous” in the context of an elicited immune response will be recognized and understood by the person of ordinary skill in the art, and is e.g. an immune response which is elicited against the same strain, such as the same Influenza A strain or the same Influenza B strain. E.g. the immunogenic composition may comprise a HA antigen (or nucleic acid, suitably RNA, suitably mRNA, encoding such) derived from A/Michigan/45/2015 (H1N1pdm9) which may elicit an immune response against A/Michigan/45/2015 (H1N1pdm9) strain.

The term “heterologous” or “intrasubtypic” in the context of an elicited immune response will be recognized and understood by the person of ordinary skill in the art, and is e.g. an immune response which is elicited against different strains within a subtype (for Influenza A virus) or lineage (for Influenza B virus), such as different Influenza A strains within a subtype such as H1 or H3 subtypes. E.g. the immunogenic composition may comprise a HA antigen (or nucleic acid, suitably RNA, suitably mRNA, encoding such) derived from A/Michigan/45/2015 (H1N1pdm9) which may elicit an immune response against A/New Caledonia/20/1999 (H1N1) strain.

The term “heterosubtypic” in the context of an elicited immune response will be recognized and understood by the person of ordinary skill in the art, and is e.g. an immune response which is elicited against different strains within one or more different subtypes (for Influenza A virus) or lineages (for Influenza B virus). E.g. the immunogenic composition may comprise a HA antigen (or nucleic acid, suitably RNA, suitably mRNA, encoding such) derived from A/Michigan/45/2015 (H1N1pdm9) which may elicit an immune response against HongKong/4801/2014 (H3N2). In further embodiments, the elicited immune response comprises broad, functional cellular T-cell responses against the respective viruses. In particular, the elicited immune response comprises a CD4+ T cell immune response and/or a CD8+ T cell immune response.

In further embodiments, the elicited immune response comprises a well-balanced B cell and T cell response against the respective viruses.

In some embodiments, the elicited immune response comprises antigen-specific immune responses.

In some embodiments, the elicited immune response reduces partially or completely the severity of one or more symptoms and/or time over which one or more symptoms of Influenza virus infection are experienced by the subject.

In some embodiments, the elicited immune response reduces the likelihood of developing an established Influenza virus infection after challenge.

In some particular embodiments, the elicited immune response slows progression of

Influenza, suitably Influenza A and/or B.

In a sixth aspect, the invention relates to a method of treating or preventing a disorder caused by an Influenza virus, suitably an Influenza A and/or Influenza B, wherein the method comprises applying or administering to a subject in need thereof the immunogenic composition, the vaccine or the kit or kit of parts as described herein.

Preventing (Inhibiting) or treating a disease, in particular a virus infection relates to inhibiting the full development of a disease or condition, for example, in a subject who is at risk for a disease such as a virus infection. “Treatment” refers to a therapeutic intervention that ameliorates a sign or symptom of a disease or pathological condition after it has begun to develop. The term “ameliorating”, with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

In some embodiments, the composition, the vaccine or the kit or kit of parts is administered at a therapeutically effective amount.

In some embodiments, the disorder is an infection with an Influenza virus, suitably an Influenza A and/or B virus.

In some embodiments, the subject in need is a mammalian subject, suitably a human subject.

In a seventh aspect, the invention relates to a method of eliciting an immune response, wherein the method comprises applying or administering to a subject in need thereof the immunogenic composition, the vaccine or the kit or kit of parts as described herein.

In some embodiments, the immune response is an adaptative immune response, suitably a protective adaptative immune response against an Influenza virus, suitably against an Influenza A virus and/or an Influenza B virus.

In some embodiments, an immune response is elicited.

In some embodiments, an adaptative immune response is elicited.

In some embodiments, a protective adaptative immune response against an Influenza virus is elicited.

In some embodiments, a protective adaptative immune response against an Influenza A and/or B virus is elicited.

In some embodiments, a protective adaptative immune response against one or more Influenza A virus subtype and/or Influenza B virus lineage is elicited, suitably against Influenza A H1N1, Influenza A H3N2, Influenza B/Yamagata lineage and Influenza B/Victoria lineage.

In some embodiments, the elicited immune response comprises neutralizing antibody titers against an Influenza virus, suitably an Influenza A and/or B virus, more suitably one or more

Influenza A virus subtype and/or Influenza B virus lineage, more suitably against Influenza A H1N1, Influenza A H3N2, Influenza B/Yamagata lineage and Influenza B/Victoria lineage.

In some embodiments, the elicited immune response comprises functional antibodies that can effectively neutralize the respective viruses.

In some embodiments, the elicited immune response is a cross-reactive immune response, wherein the functional antibodies that can effectively neutralize the respective viruses further neutralize viruses belonging to same and/or other Influenza A subtypes and/or Influenza B lineages.

In some embodiments, the cross-reactive immune response is homologous, heterologous or intrasubtypic and/or heterosubtypic.

In further embodiments, the elicited immune response comprises broad, functional cellular T-cell responses against the respective viruses. In particular, the elicited immune response comprises a CD4+ T cell immune response and/or a CD8+ T cell immune response.

In further embodiments, the elicited immune response comprises a well-balanced B cell and T cell response against the respective viruses.

In some embodiments, the elicited immune response comprises antigen-specific immune responses.

In some embodiments, the elicited immune response reduces partially or completely the severity of one or more symptoms and/or time over which one or more symptoms of Influenza virus infection are experienced by the subject.

In some embodiments, the elicited immune response reduces the likelihood of developing an established Influenza virus infection after challenge.

In some particular embodiments, the elicited immune response slows progression of Influenza, suitably Influenza A and/or B.

In some embodiments, the subject in need is a mammalian subject, suitably a human subject.

In some embodiments, the composition, the vaccine or the kit or kit of parts as described herein is administered in an amount effective to induce a T cell response against Influenza A H1N1, Influenza A H3N2, Influenza B/Yamagata lineage and Influenza B/Victoria lineage.

In some embodiments, the composition, the vaccine or the kit or kit of parts as described is administered in an amount effective to induce a neutralizing antibody response against Influenza A H1N1, Influenza A H3N2, Influenza B/Yamagata lineage and Influenza B/Victoria lineage.

In embodiments, administration of the immunogenic composition, the vaccine or the kit or kit to a subject elicits neutralizing antibodies and does not elicit disease enhancing antibodies. In particular, administration of the immunogenic composition, the vaccine or the kit or kit to a subject does not elicit immunopathological effects, like e.g. enhanced disease and/or antibody dependent enhancement (ADE).

### Further Definitions

For the sake of clarity and readability the following definitions are provided. Any technical feature mentioned for these definitions may be read on each and every embodiment of the invention. Additional definitions and explanations may be specifically provided in the context of these embodiments.

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus, a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

As used herein, the singular forms “a,” “an” and “the” include plural references unless the content clearly dictates otherwise.

Unless specifically stated, a process comprising a step of mixing two or more components does not require any specific order of mixing. Thus, components can be mixed in any order. Where there are three components then two components can be combined with each other, and then the combination may be combined with the third component, etc.

The term “immunogenic fragment” or “immunogenic variant” has to be understood as any fragment/variant of the corresponding Influenza antigen that is capable of raising an immune response in a subject.

Percentages in the context of numbers should be understood as relative to the total number of the respective items. In other cases, and unless the context dictates otherwise, percentages should be understood as percentages by weight (wt.-%).

About: The term “about” is used when determinants or values do not need to be identical, i.e. 100% the same. Accordingly, “about” means, that a determinant or values may diverge by 1% to 20%, for example by 1% to 10%; in particular, by 1%, 2%, 3%, 4%, 5%, 6%, 7%, 8%, 9%, 10%, 11%, 12%, 13%, 14%, 15%, 16%, 17%, 18%, 19%, 20%. The skilled person knows that e.g. certain parameters or determinants can slightly vary based on the method how the parameter has been determined. For example, if a certain determinants or value is defined herein to have e.g. a length of “about 100 nucleotides”, the length may diverge by 1% to 20%. Accordingly, the skilled person knows that in that specific example, the length may diverge by 1 to 20 nucleotides. Accordingly, a length of “about 100 nucleotides” may encompass sequences ranging from 80 to 120 nucleotides.

Adaptive immune response: The term “adaptive immune response” as used herein will be recognized and understood by the person of ordinary skill in the art, and is e.g. intended to refer to an antigen-specific response of the immune system (the adaptive immune system). Antigen specificity allows for the generation of responses that are tailored to specific pathogens or pathogen-infected cells. The ability to mount these tailored responses is usually maintained in the body by “memory cells” (B-cells).

Antigen: The term “antigen” as used herein will be recognized and understood by the person of ordinary skill in the art, and is e.g. intended to refer to a substance which may be recognized by the immune system, for example by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells. Also fragments, variants and derivatives of peptides or proteins comprising at least one epitope are understood as antigens.

Antigenic peptide, polypeptide or protein: The term “antigenic peptide or protein” or “immunogenic peptide or protein” will be recognized and understood by the person of ordinary skill in the art, and is e.g. intended to refer to a peptide, protein derived from a (antigenic or immunogenic) protein which stimulates the body's adaptive immune system to provide an adaptive immune response. Therefore an antigenic/immunogenic peptide or protein comprises at least one epitope (as defined herein) or antigen (as defined herein) of the protein it is derived from.

Cationic: Unless a different meaning is clear from the specific context, the term “cationic” means that the respective structure bears a positive charge, either permanently or not permanently, but in response to certain conditions such as pH. Thus, the term “cationic” covers both “permanently cationic” and “cationisable”. The term “permanently cationic” means, e.g., that the respective compound, or group, or atom, is positively charged at any pH value or hydrogen ion activity of its environment. Typically, the positive charge results from the presence of a quaternary nitrogen atom.

Cationisable: The term “cationisable” as used herein means that a compound, or group or atom, is positively charged at a lower pH and uncharged at a higher pH of its environment. Also in non-aqueous environments where no pH value can be determined, a cationisable compound, group or atom is positively charged at a high hydrogen ion concentration and uncharged at a low concentration or activity of hydrogen ions. It depends on the individual properties of the cationisable or polycationisable compound, in particular the pKa of the respective cationisable group or atom, at which pH or hydrogen ion concentration it is charged or uncharged. In diluted aqueous environments, the fraction of cationisable compounds, groups or atoms bearing a positive charge may be estimated using the so-called Henderson-Hasselbalch equation which is well-known to a person skilled in the art. E.g., in some embodiments, if a compound or moiety is cationisable, it is suitable that it is positively charged at a pH value of about 1 to 9, preferably 4 to 9, 5 to 8 or even 6 to 8, for example of a pH value of or below 9, of or below 8, of or below 7, for example at physiological pH values, e.g. about 7.3 to 7.4, i.e. under physiological conditions, particularly under physiological salt conditions of the cell in vivo. In other embodiments, it is suitable that the cationisable compound or moiety is predominantly neutral at physiological pH values, e.g. about 7.0-7.4, but becomes positively charged at lower pH values. In some embodiments, the range of pKa for the cationisable compound or moiety is about 5 to about 7.

Coding sequence/coding region: The terms “coding sequence” or “coding region” and the corresponding abbreviation “cds” as used herein will be recognized and understood by the person of ordinary skill in the art, and are e.g. intended to refer to a sequence of several nucleotide triplets, which may be translated into a peptide or protein. A coding sequence in the context of the present invention may be an RNA sequence consisting of a number of nucleotides that may be divided by three, which starts with a start codon and which for example terminates with a stop codon.

Derived from: The term “derived from” as used throughout the present specification in the context of a nucleic acid, i.e. for a nucleic acid “derived from” (another) nucleic acid, means that the nucleic acid, which is derived from (another) nucleic acid, shares e.g. at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% sequence identity with the nucleic acid from which it is derived. The skilled person is aware that sequence identity is typically calculated for the same types of nucleic acids, i.e. for DNA sequences or for RNA sequences. Thus, it is understood, if a DNA is “derived from” an RNA or if an RNA is “derived from” a DNA, in a first step the RNA sequence is converted into the corresponding DNA sequence (in particular by replacing the uracils (U) by thymines (T) throughout the sequence) or, vice versa, the DNA sequence is converted into the corresponding RNA sequence (in particular by replacing the T by U throughout the sequence). Thereafter, the sequence identity of the DNA sequences or the sequence identity of the RNA sequences is determined. For example, a nucleic acid “derived from” a nucleic acid also refers to nucleic acid, which is modified in comparison to the nucleic acid from which it is derived, e.g. in order to increase

RNA stability even further and/or to prolong and/or increase protein production. In the context of amino acid sequences (e.g. antigenic peptides or proteins) the term “derived from” means that the amino acid sequence, which is derived from (another) amino acid sequence, shares e.g. at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence from which it is derived.

Epitope: The term “epitope” (also called “antigen determinant” in the art) as used herein will be recognized and understood by the person of ordinary skill in the art, and is e.g. intended to refer to T cell epitopes and B cell epitopes. T cell epitopes or parts of the antigenic peptides or proteins and may comprise fragments preferably having a length of about 6 to about 20 or even more amino acids, e.g. fragments as processed and presented by MHC class| molecules, preferably having a length of about 8 to about 10 amino acids, e.g. 8, 9, or 10, (or even 11, or 12 amino acids), or fragments as processed and presented by MHC class II molecules, preferably having a length of about 13 to about 20 or even more amino acids. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens, preferably having 5 to 15 amino acids, more preferably having 5 to 12 amino acids, even more preferably having 6 to 9 amino acids, which may be recognized by antibodies, i.e. in their native form. Such epitopes of proteins or peptides may furthermore be selected from any of the herein mentioned variants of such proteins or peptides. In this context epitopes can be conformational or discontinuous epitopes which are composed of segments of the proteins or peptides as defined herein that are discontinuous in the amino acid sequence of the proteins or peptides as defined herein but are brought together in the three-dimensional structure or continuous or linear epitopes which are composed of a single polypeptide chain.

Fragment: The term “fragment” as used throughout the present specification in the context of a nucleic acid sequence (e.g. RNA or a DNA) or an amino acid sequence may typically be a shorter portion of a full-length sequence of e.g. a nucleic acid sequence or an amino acid sequence. Accordingly, a fragment typically consists of a sequence that is identical to the corresponding stretch within the full-length sequence. A particular fragment of a sequence in the context of the present invention, consists of a continuous stretch of entities, such as nucleotides or amino acids corresponding to a continuous stretch of entities in the molecule the fragment is derived from, which represents at least 40%, 50%, 60%, 70%, 80%, 90%, 95% of the total (i.e. full-length) molecule from which the fragment is derived (e.g. a virus protein). The term “fragment” as used throughout the present specification in the context of proteins or peptides may, typically, comprise a sequence of a protein or peptide as defined herein, which is, with regard to its amino acid sequence, N-terminally and/or C-terminally truncated compared to the amino acid sequence of the original protein. The term “fragment” as used throughout the present specification in the context of RNA sequences may, typically, comprise an RNA sequence that is 5′-terminally and/or 3′-terminally truncated compared to the reference RNA sequence. Such truncation may thus occur either on the amino acid level or correspondingly on the nucleic acid level. A sequence identity with respect to such a fragment as defined herein may therefore for example refer to the entire protein or peptide as defined herein or to the entire (coding) nucleic acid molecule of such a protein or peptide. Fragments of proteins or peptides may comprise at least one epitope of those proteins or peptides.

Heterologous: The terms “heterologous” or “heterologous sequence” as used throughout the present specification in the context of a nucleic acid sequence or an amino acid sequence refers to a sequence (e.g. RNA, DNA, amino acid) has to be understood as a sequence that is derived from another gene, another allele, or e.g. another species or virus. Two sequences are typically understood to be “heterologous” if they are not derivable from the same gene or from the same allele. I.e., although heterologous sequences may be derivable from the same organism or virus, in nature, they do not occur in the same nucleic acid or protein.

Humoral immune response: The terms “humoral immunity” or “humoral immune response” will be recognized and understood by the person of ordinary skill in the art, and are e.g. intended to refer to B-cell mediated antibody production and optionally to accessory processes accompanying antibody production. A humoral immune response may be typically characterized, e.g. by Th2 activation and cytokine production, germinal center formation and isotype switching, affinity maturation and memory cell generation. Humoral immunity may also refer to the effector functions of antibodies, which include pathogen and toxin neutralization, classical complement activation, and opsonin promotion of phagocytosis and pathogen elimination.

Identity (of a sequence): The term “identity” as used throughout the present specification in the context of a nucleic acid sequence or an amino acid sequence will be recognized and understood by the person of ordinary skill in the art, and is e.g. intended to refer to the percentage to which two sequences are identical. To determine the percentage to which two sequences are identical, e.g. nucleic acid sequences or amino acid (aa) sequences as defined herein, for example the aa sequences encoded by the nucleic acid sequence as defined herein or the aa sequences themselves, the sequences can be aligned in order to be subsequently compared to one another. Therefore, e.g. a position of a first sequence may be compared with the corresponding position of the second sequence. If a position in the first sequence is occupied by the same residue as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using an algorithm, e.g. an algorithm integrated in the BLAST program. Sequence identity can be determined by using the EMBOSS Water sequence tool alignment at the EMBL-EBI website https://www.ebi.ac.uk/Tools/psa/emboss_water/with the parameters gap open=12, gap extend=1 and matrix=BLOSUM62 for protein sequences or matrix=fullDNA for DNA/RNA sequences, or by using the EMBOSS Needle sequence alignment tool at the EMBL-EBI website https://www.ebi.ac.uk/Tools/psa/emboss_needle/with default parameters (e.g. gap open=10, gap extend=0.5, end gap penalty=false, end gap open=10 and end gap extend=0.5 and matrix=BLOSUM62 for protein sequences or matrix=fullDNA for DNA/RNA sequences). Unless specified otherwise, where the application refers to sequence identity to a particular reference sequence, the identity is intended to be calculated over the entire length of that reference sequence.

Immunogen, Immunogen: The terms “immunogen” or “immunogenic” will be recognized and understood by the person of ordinary skill in the art, and are e.g. intended to refer to a compound that is able to stimulate/induce an (adaptive) immune response. An immunogen may be a peptide, polypeptide, or protein.

Immune response: The term “immune response” will be recognized and understood by the person of ordinary skill in the art, and is e.g. intended to refer to a specific reaction of the adaptive immune system to a particular antigen (so called specific or adaptive immune response) or an unspecific reaction of the innate immune system (so called unspecific or innate immune response), or a combination thereof.

Innate immune system: The term “innate immune system” (also known as non-specific or unspecific immune system) will be recognized and understood by the person of ordinary skill in the art, and is e.g. intended to refer to a system typically comprising the cells and mechanisms that defend the host from infection by other organisms in a non-specific manner. This means that the cells of the innate system may recognize and respond to pathogens in a generic way, but unlike the adaptive immune system, it does not confer long-lasting or protective immunity to the host. The innate immune system may be activated by ligands of pattern recognition receptor e.g. Toll-like receptors, NOD-like receptors, or RIG-I like receptors etc.,

Lipidoid compound: A lipidoid compound, also simply referred to as lipidoid, is a lipid-like compound, i.e. an amphiphilic compound with lipid-like physical properties. In the context of the present invention, the term lipid is considered to encompass lipidoid compounds.

Nucleic acid, nucleic acid molecule: The terms “nucleic acid” or “nucleic acid molecule” as used herein, will be recognized and understood by the person of ordinary skill in the art. The terms “nucleic acid” or “nucleic acid molecule” particularly refers to DNA (molecules) or RNA molecules). The term is used synonymously with the term polynucleotide. For example, a nucleic acid or a nucleic acid molecule is a polymer comprising or consisting of nucleotide monomers that are covalently linked to each other by phosphodiester-bonds of a sugar/phosphate-backbone. The terms “nucleic acid” or “nucleic acid molecule” also encompasses modified nucleic acid (molecules), such as base-modified, sugar-modified or backbone-modified DNA or RNA (molecules) as defined herein.

Nucleic acid sequence, DNA sequence, RNA sequence: The terms “nucleic acid sequence”, “DNA sequence”, “RNA sequence” will be recognized and understood by the person of ordinary skill in the art, and e.g. refer to a particular and individual order of the succession of its nucleotides.

Permanently cationic: The term “permanently cationic” as used herein will be recognized and understood by the person of ordinary skill in the art, and means, e.g., that the respective compound, or group, or atom, is positively charged at any pH value or hydrogen ion activity of its environment. Typically, the positive charge results from the presence of a quaternary nitrogen atom. Where a compound carries a plurality of such positive charges, it may be referred to as permanently polycationic.

Stabilized RNA: The term “stabilized RNA” refer to an RNA that is modified such, that it is more stable to disintegration or degradation, e.g., by environmental factors or enzymatic digest, such as by exo- or endonuclease degradation, compared to an RNA without such modification. Preferably, a stabilized RNA in the context of the present invention is stabilized in a cell, such as a prokaryotic or eukaryotic cell, preferably in a mammalian cell, such as a human cell. The stabilization effect may also be exerted outside of cells, e.g. in a buffer solution etc., e.g., for storage of a composition comprising the stabilized RNA.

T-cell responses: The terms “cellular immunity” or “cellular immune response” or “cellular T-cell responses” as used herein will be recognized and understood by the person of ordinary skill in the art, and are for example intended to refer to the activation of macrophages, natural killer cells (NK), antigen-specific cytotoxic T-lymphocytes, and the release of various cytokines in response to an antigen. In more general terms, cellular immunity is not based on antibodies, but on the activation of cells of the immune system. Typically, a cellular immune response may be characterized e.g. by activating antigen-specific cytotoxic T-lymphocytes that are able to induce apoptosis in cells, e.g. specific immune cells like dendritic cells or other cells, displaying epitopes of foreign antigens on their surface.

RNA: The term “RNA” is the usual abbreviation for ribonucleic acid. It is a nucleic acid molecule, i.e. a polymer consisting of nucleotide monomers. These nucleotides are usually adenosine-monophosphate (AMP), uridine-monophosphate (UMP), guanosine-monophosphate (GMP) and cytidine-monophosphate (CMP) monomers or analogs thereof, which are connected to each other along a so-called backbone. The backbone is typically formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. In general, RNA can be obtained by transcription of a DNA sequence, e.g. inside a cell or in vitro. In the context of the invention, the RNA may be obtained by RNA in vitro transcription. Alternatively, RNA may be obtained by chemical synthesis.

RNA in vitro transcription: The terms “RNA in vitro transcription” or “in vitro transcription” relate to a process wherein RNA is synthesized in a cell-free system in vitro. RNA may be obtained by DNA-dependent in vitro transcription of an appropriate DNA template, which is typically a linear DNA template (e.g. linearized plasmid DNA or PCR product). The promoter for controlling RNA in vitro transcription can be any promoter for any DNA-dependent RNA polymerase. Particular examples of DNA-dependent RNA polymerases are the T7, T3, SP6, or Syn5 RNA polymerases. In one embodiment of the present invention the DNA template is linearized with a suitable restriction enzyme before it is subjected to RNA in vitro transcription. Reagents typically used in RNA in vitro transcription include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); optionally, a cap analogue as defined herein; optionally, modified nucleotides as defined herein; a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, pyrophosphatase; MgCl2; a buffer (TRIS or HEPES) to maintain a suitable pH value, which can also contain antioxidants (e.g. DTT), and/or polyamines such as spermidine.

Variant (of a sequence): The term “variant” as used throughout the present specification in the context of a nucleic acid sequence will be recognized and understood by the person of ordinary skill in the art, and is e.g. intended to refer to a variant of a nucleic acid sequence derived from another nucleic acid sequence. E.g., a variant of a nucleic acid sequence may exhibit one or more nucleotide deletions, insertions, additions and/or substitutions compared to the nucleic acid sequence from which the variant is derived. A variant of a nucleic acid sequence may at least 50%, 60%, 70%, 80%, 90%, or 95% identical to the nucleic acid sequence the variant is derived from. The variant is a functional variant in the sense that the variant has retained at least 50%, 60%, 70%, 80%, 90%, or 95% or more of the function of the sequence where it is derived from. A “variant” of a nucleic acid sequence may have at least 70%, 75%, 80%, 85%, 90%, 95%, 98% or 99% nucleotide identity over a stretch of at least 10, 20, 30, 50, 75 or 100 nucleotides of such nucleic acid sequence.

The term “variant” as used throughout the present specification in the context of proteins or peptides is e.g. intended to refer to a proteins or peptide variant having an amino acid sequence which differs from the original sequence in one or more mutation(s)/substitution(s), such as one or more substituted, inserted and/or deleted amino acid(s). Suitably, these fragments and/or variants have the same, or a comparable specific antigenic property (immunogenic variants, antigenic variants). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra). A “variant” of a protein or peptide may have at least 70%, 75%, 80%, 85%, 90%, 95%, 98% or 99% amino acid identity over a stretch of at least 10, 20, 30, 50, 75 or 100 amino acids of such protein or peptide. Alternatively, a “variant” of a protein or polypeptide may have from 1 to 20, for example from 1 to 10 single amino acid mutations compared to such protein or peptide, for example, 1, 2, 3, 4, 5, 6, 7, 8, 9, 10, 11, 12, 13, 15, 16, 17, 18, 19 or 20 single amino acid mutations. For mutations we mean or include substitution, insertion or deletion. In one embodiment, a variant of a protein comprises a functional variant of the protein, which means, in the context of the invention, that the variant exerts essentially the same, or at least 40%, 50%, 60%, 70%, 80%, 90% of the immunogenicity as the protein it is derived from.

Multivalent vaccine/composition: the multivalent vaccine or combination of the invention provides more than one valence (e.g. an antigen) derived from more than one virus (e.g. at least one Influenza virus as defined herein and at least one further Influenza virus as defined herein).

### Numbered Embodiments

In the following, embodiments of the present invention are provided as a numbered embodiment list (Embodiment 1 to Embodiment 92).
Embodiment 1. An Immunogenic composition comprising:
   (a) a first hemagglutinin (HA) antigen or a first nucleic acid encoding the first HA antigen wherein the first HA antigen is derived from a strain of subtype H3 of Influenza A virus; and
   (b) a second HA antigen or a second nucleic acid encoding the second HA antigen wherein the second HA antigen is derived from a strain of subtype H1 of Influenza A virus, wherein the ratio of (a):(b) is comprised between 1.5:1 and 20:1, optionally between 1.5:1 and 5:1.
Embodiment 2. The immunogenic composition according to embodiment 1, wherein (a) is a first RNA encoding the first HA antigen and/or (b) is a second RNA encoding the second HA antigen.
Embodiment 3. The immunogenic composition according to embodiment 1 or 2, wherein (a) is a first mRNA encoding the first HA antigen and/or (b) is a second mRNA encoding the second HA antigen.
Embodiment 4. The immunogenic composition according to any of embodiments 1 to 3, wherein said ratio of (a):(b) is 1.5:1, 2:1, 2.2:1, 2.4:1, 2.6:1, 2.8:1, 3:1, 3.2:1, 3.4:1, 3.6:1, 3.8:1, 4:1, 4.2:1, 4.4:1, 4.6:1, 4.8:1 or 5:1.
Embodiment 5. The immunogenic composition according to any of embodiments 1 to 4, wherein said ratio of (a):(b) is comprised between 2:1 and 4:1, suitably between 2:1 and 3:1, suitably is 2:1 or 3:1.
Embodiment 6. The immunogenic composition according to any of embodiments 1 to 3, wherein the ratio of (a):(b) is greater than 3:1 and lower or equal to 20:1, optionally greater than 3:1 and lower or equal to 5:1.
Embodiment 7. The immunogenic composition according to any of embodiments 1 to 6, wherein a dose of each said first mRNA and/or said second mRNA is 1 to 200 μg, suitably 1 to 60 μg, suitably 2 to 25 μg.
Embodiment 8. The immunogenic composition according to any of embodiments 1 to 7, further comprising:
   (c) at least one further antigen or at least one further nucleic acid encoding the at least one further antigen, wherein the at least one further antigen is derived from a strain of Influenza virus.
Embodiment 9. The immunogenic composition according to embodiment 8, wherein (c) is at least one further RNA encoding the at least one further antigen.
Embodiment 10. The immunogenic composition according to embodiment 11 or 12, wherein (c) is at least one further mRNA encoding the at least one further antigen.
Embodiment 11. The immunogenic composition according to any of embodiments 8 to 10, wherein said strain of Influenza virus of (c) is selected from the group consisting of Influenza A virus and Influenza B virus.
Embodiment 12. The immunogenic composition according to any of embodiments 8 to 11, wherein said at least one further antigen comprises or consists of a peptide or protein selected or derived from an Influenza virus hemagglutinin (HA), neuraminidase (NA), nucleoprotein (NP), matrix protein 1 (M1), matrix protein 2 (M2), non-structural protein 1 (NS1), non-structural protein 2 (NS2), nuclear export protein (NEP), polymerase acidic protein (PA), polymerase basic protein PB1, PB1-F2, and/or polymerase basic protein 2 (PB2), or an immunogenic fragment or an immunogenic variant thereof.
Embodiment 13. The immunogenic composition according to any of embodiments 8 to 12, wherein said at least one further antigen comprises or consists of a peptide or protein selected or derived from an Influenza virus hemagglutinin (HA) or neuraminidase (NA) or an immunogenic fragment or an immunogenic variant thereof.
Embodiment 14. The immunogenic composition according to any of embodiments 8 to 13, wherein said at least one further antigen or at least one further nucleic acid, suitably mRNA, encoding the at least one further antigen is at least one further HA antigen or at least one further nucleic acid, suitably mRNAs, encoding the at least one further HA antigen derived from a strain of subtype H3 of Influenza A virus, suitably from H3N2, and wherein the ratio of (a) and (c) to (b) is greater than 1:1 and lower or equal to 20:1, optionally greater than 1:1 and lower of equal to 5:1, optionally comprised between 1.5:1 and 5:1.
Embodiment 15. The immunogenic composition according to any of embodiments 8 to 13, wherein said strain of Influenza virus is a strain of Influenza B virus.
Embodiment 16. The immunogenic composition according to any of embodiments 8 to 15, wherein the composition comprises a plurality of (c).
Embodiment 17. The immunogenic composition according to embodiment 16, wherein the composition comprises at least three, four, five, six, seven or eight antigens or nucleic acids encoding such, optionally three to ten antigens or nucleic acids encoding such, optionally three, four, six, seven or eight antigens or nucleic acids encoding such.
Embodiment 18. The immunogenic composition according to embodiment 16 or 17, wherein the composition comprises at least three, four, five, six, seven or eight mRNAs, optionally three to ten mRNAs, optionally three, four, six, seven or eight mRNAs.
Embodiment 19. The immunogenic composition according to any of embodiments 16 to 18, wherein the composition is a multivalent composition, said strain of subtype H3 of Influenza virus of (a) and/or said strain of subtype H1 of Influenza virus of (b) and/or said strain of Influenza virus of (c) being different.
Embodiment 20. The immunogenic composition according to any of embodiments 16 to 19, wherein said antigens of (a), (b) and/or (c) are derived from at least two, three or four strains of

Influenza virus.
Embodiment 21. The immunogenic composition according to any of embodiments 1 to 20, wherein said strain of subtype H3 of Influenza A virus is selected from influenza A viruses characterized by a neuraminidase (NA) selected from the group consisting of N1, N2, and N8, suitably N2.
Embodiment 22. The immunogenic composition according to embodiment 21, wherein said strain of subtype H3 of Influenza A virus is selected from the group consisting of H3N1, H3N2 and H3N8, suitably H3N2.
Embodiment 23. The immunogenic composition according to any of embodiments 1 to 22, wherein said strain of subtype H1 of Influenza A virus is selected from Influenza A viruses characterized by a neuraminidase (NA) selected from the group consisting of N1 and N2, more suitably N1.
Embodiment 24. The immunogenic composition according to embodiment 23, wherein said strain of subtype H1 of Influenza A virus is selected from the group consisting of H1N1 and H1N2, suitably H1N1.
Embodiment 25. The immunogenic composition according to any of embodiments 8 to 24, wherein said strain of Influenza virus of (c) is a strain of Influenza B virus.
Embodiment 26. The immunogenic composition according to embodiment 25, wherein said strain of Influenza B virus is selected from the group consisting of B/Victoria lineage and B/Yamagata lineage.
Embodiment 27. The immunogenic composition according to embodiment 26, wherein said strain of Influenza B virus is selected from the group consisting of B/Beijing/184/93-like virus, B/Harbin/94-like virus, B/Shangdong/7/97-like virus, B/Yamanashi/166/98-like virus, B/Sichuan/379/99-like virus, B/Guangdong/120/2000, B/Johannesburg/5/99, B/Victoria/504/2000, B/Hong Kong/330/2001-like virus, B/Hong Kong/1434/2002, B/Brisbane/32/2002, B/Shanghai/361/2002-like virus, B/Jiangsu/10/2003, B/Jilin/20/2003, B/Malaysia/2506/2004-like virus, B/Malaysia/2506/2004 virus, B/Ohio/1/2005, B/Florida/4/2006-like virus, B/Brisbane/3/2007, B/Brisbane/60/2008-like virus, B/Brisbane/33/2008, B/Wisconsin/1/2010-like virus, B/Hubei-Wujiagang/158/2009, B/Texas/6/2011, B/Massachusetts/2/2012-like virus, B/Phuket/3073/2013-like virus, B/Austria/1359417/2021-like virus, B/Washington/02/2019-like virus and B/Colorado/06/2017-like virus.
Embodiment 28. Immunogenic composition comprising:
   (a) a first mRNA encoding a HA of a strain of subtype H3 of Influenza A virus;
   (b) a second mRNA encoding a HA of a strain of subtype H1 of Influenza A virus;
   (c¹) a third mRNA encoding a HA of a first strain of Influenza B virus; and
   (c²) optionally, a fourth mRNA encoding a HA of a second strain of Influenza B virus,

wherein the ratio of (a):(b): (c¹): (c²) is comprised between 1.5:1:1:1 and 20:1:1:1, optionally between 1.5:1:1:1 and 5:1:1:1.
Embodiment 29. The immunogenic composition of embodiment 28 further comprising:
   (c³) a first mRNA encoding a NA of the strain of subtype H1 of Influenza A virus;
   (c⁴) a second mRNA encoding a NA of the strain of subtype H3 of Influenza A virus; and
   (c⁵) a third mRNA encoding a NA of the first strain of Influenza B virus,

wherein the ratio of (a):(b): (c¹): (c²) is comprised between 1.5:1:1:1 and 20:1:1:1, optionally between 1.5:1:1:1 and 5:1:1:1.
Embodiment 30. The immunogenic composition of embodiment 29 further comprising:
   (c⁶) an eighth mRNA encoding a NA of the second strain of Influenza B virus,

wherein (a), (b), (c¹), (c²), (c³), (c⁴), (c⁵) and (c⁶) are different.
Embodiment 31. The immunogenic composition of embodiment 30, wherein the ratio of (a): (b): (c¹): (c²) is comprised between 1.5:1:1:1 and 20:1:1:1, optionally between 1.5:1:1:1 and 5:1:1:1.

Embodiments 32. The immunogenic composition according to any of embodiments 28 to 31, wherein the ratio of (a):(b): (c¹): (c²) is greater than 3:1:1:1 and lower or equal to 20:1:1:1, optionally greater than 3:1:1:1 and lower or equal to 5:1:1:1.
Embodiment 33. The immunogenic composition according to embodiment 30, wherein the ratio of (a): (b): (c¹): (c²): (c³): (c⁴): (c⁵): (c⁶) is comprised between 9:3:3:3:1:1:1:1 and 3:1:1:1:3:3:3:3, suitably between 6:2:2:2:1:1:1:1 and 3:1:1:1:2:2:2:2, suitably is 6:2:2:2:1:1:1:1 or 3:1:1:1:2:2:2:2.
Embodiment 34. The immunogenic composition according to any of embodiments 28 to 33, wherein the mRNAs of (a), (b), (c¹), (c²), (c³), (c⁴), (c⁵) and/or (c⁶) are formulated in a lipid nanoparticle (LNP), each separately or not.
Embodiment 35. The immunogenic composition according to embodiment 34, wherein the LNP comprises a PEG-modified lipid, a non-cationic lipid, a sterol, and a cationic lipid.
Embodiment 36. The immunogenic composition according to embodiment 35, wherein the cationic lipid is ionizable.
Embodiment 37. The immunogenic composition according to embodiment 36, wherein the ionizable cationic lipid has the formula III:

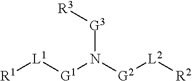

or a pharmaceutically acceptable salt, tautomer or stereoisomer thereof, wherein:
   L¹ or L² is each independently —O(C═O)— or —(C═O)O—;
   G¹ and G² are each independently unsubstituted C₁-C₁₂ alkylene or C₁-C₁₂ alkenylene;
   G³ is C₁-C₂₄ alkylene, C₁-C₂₄ alkenylene, C₃-C₈ cycloalkylene, or C₃-C₈ cycloalkenylene;
   R¹ and R² are each independently, branched or linear, C₆-C₂₄ alkyl or C₆-C₂₄ alkenyl;
   R³ is H, OR⁵, CN, —C(═O) OR⁴, —OC(═O) R⁴ or —NR⁵C(═O) R⁴;
   R⁴ is C₁-C₁₂ alkyl;
   R⁵ is H or C₁-C₆ alkyl.
Embodiment 38. The immunogenic composition according to embodiment 37, wherein the ionizable cationic lipid has the formula III:

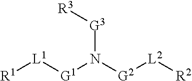

or a pharmaceutically acceptable salt, tautomer or stereoisomer thereof, wherein:
   L¹ or L² is each independently —O(C═O)— or —(C═O)O—;
   G¹ and G² are each independently unsubstituted C₁-C₁₂ alkylene;
   G³ is C₁-C₂₄ alkylene;
   R¹ and R² are each independently, branched or linear, C₆-C₂₄ alkyl;
   R³ is OR⁵; and
   R⁵ is H.
Embodiment 39. The immunogenic composition according to embodiment 36, wherein the ionizable cationic lipid has the formula III and wherein R¹, R² or both R¹ and R² have one of the following structures:

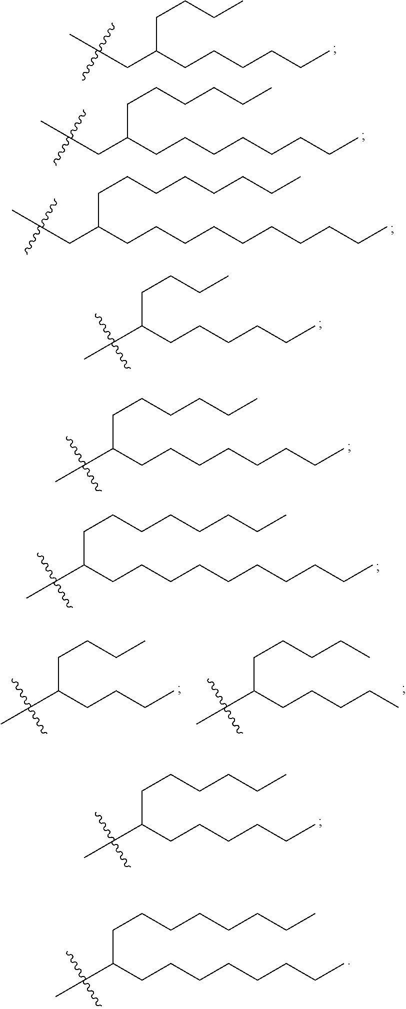

Embodiment 40. The immunogenic composition according to embodiment 39, wherein R² has the structure:

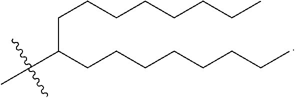 [SMILES: *C(CCCCCCCC)CCCCCCCC]

Embodiment 41. The immunogenic composition according to embodiment 35, wherein the cationic lipid has the formula:

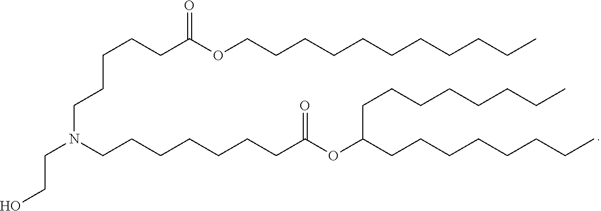 [SMILES: CCCCCCCCCCCOC(=O)CCCCCN(CCO)CCCCCCCC(=O)OC(CCCCCCCC)CCCCCCCC]

Embodiment 42. The immunogenic composition according to embodiment 35, wherein the cationic lipid has the formula:

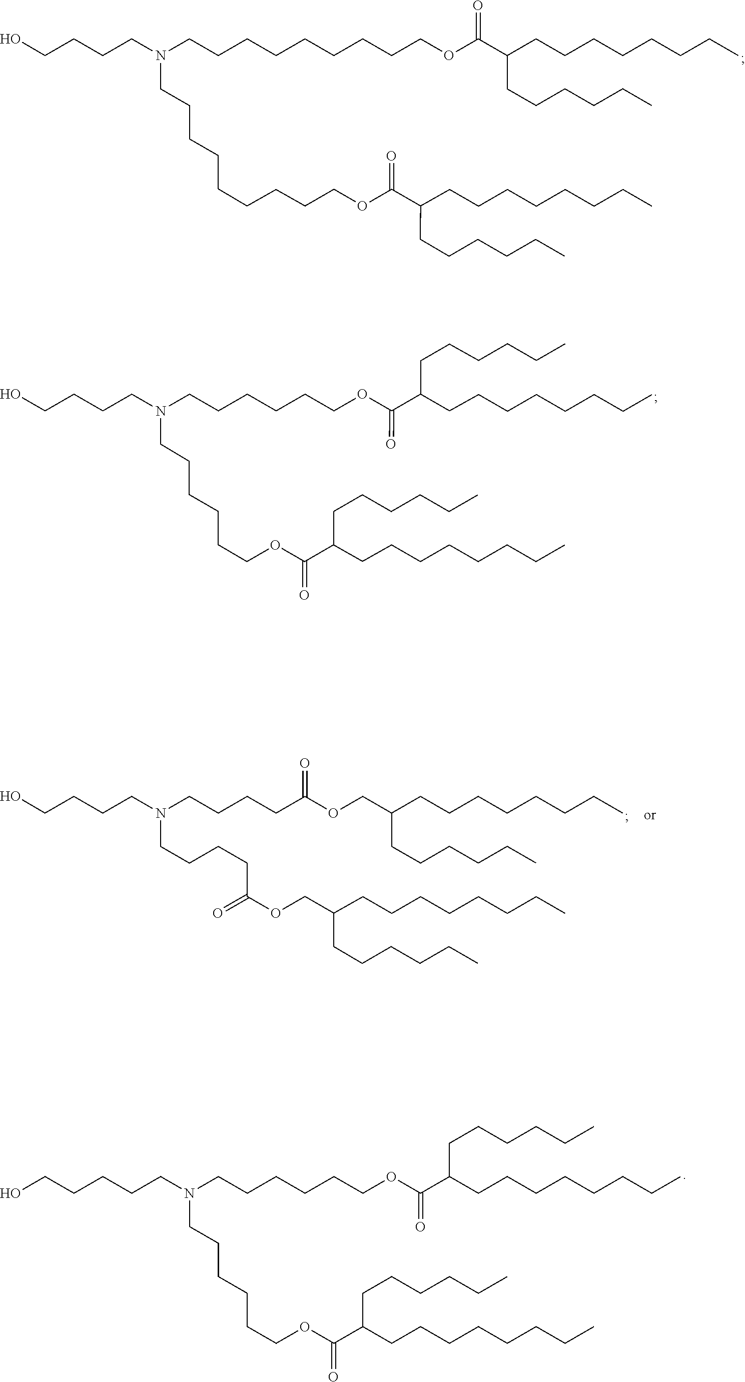 [SMILES: CCCCCCCCC(CCCCCC)C(=O)OCCCCCCCCCN(CCCCO)CCCCCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; CCCCCCCCC(CCCCCC)COC(=O)CCCCN(CCCCO)CCCCC(=O)OCC(CCCCCC)CCCCCCCC ; CCCCCCCCC(CCCCCC)C(=O)OCCCCCCN(CCCCCO)CCCCCCOC(=O)C(CCCCCC)CCCCCCCC]

Embodiment 43. The immunogenic composition according to claim 36, wherein the ionizable cationic lipid has the formula:

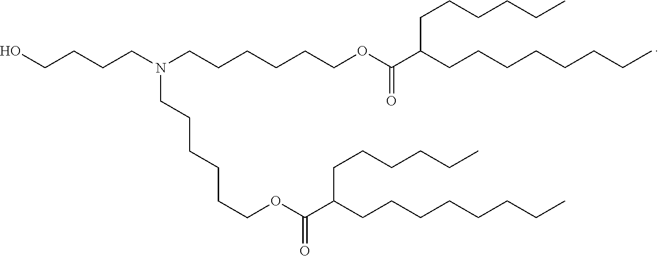 [SMILES: CCCCCCCCC(CCCCCC)C(=O)OCCCCCCN(CCCCO)CCCCCCOC(=O)C(CCCCCC)CCCCCCCC]

Embodiment 44. The immunogenic composition according to any of embodiments 35 to 43, wherein the PEG-modified lipid comprises PEG-DMG or PEG-CDMA.
Embodiment 45. The immunogenic composition according to any of embodiments 34 to 43, wherein the PEG-modified lipid has the formula IV:

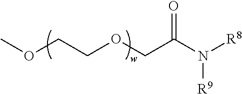

wherein R⁸ and R⁹ are each independently a straight or branched, saturated or unsaturated alkyl chain containing from 10 to 30 carbon atoms, wherein the alkyl chain is optionally interrupted by one or more ester bonds;

and ψ has a mean value ranging from 30 to 60.
Embodiment 46. The immunogenic composition according to embodiment 45, wherein in the PEG-modified lipid R⁸ and R⁹ are saturated alkyl chains.
Embodiment 47. The immunogenic composition according to claim 35, wherein the PEG-modified lipid has the formula IVa:

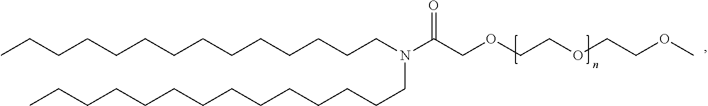

wherein n has a mean value ranging from 30 to 60, suitably wherein n has a mean value of about 45, 46, 47, 48, 49, 50, 51, 52, 53, 54, most suitably wherein n has a mean value of 49 or 45; or wherein n is an integer selected such that the average molecular weight of the PEG lipid is about 2500 g/mol.
Embodiment 48. The immunogenic composition according to any of embodiments 35 to 47, wherein the non-cationic lipid is a neutral lipid, such as 1,2-distearoyl-sn-glycero-3-phosphocholine (DSPC), 1,2-dipalmitoyl-sn-glycero-3-phosphocholine (DPPC), 1-palmitoyl-2-oleoyl-sn-glycero-3-phosphocholine (POPC), 1,2-dioleoyl-sn-glycero-3-phosphoethanolamine (DOPE) or sphingomyelin (SM), preferably the neutral lipid is DSPC.
Embodiment 49. The immunogenic composition according to any of embodiments 35 to 48, wherein the sterol is cholesterol.
Embodiment 50. The immunogenic composition according to any of embodiments 35 to 49, wherein the LNP comprises a PEG-modified lipid at around 0.5 to 15 molar %, a non-cationic lipid at around 5 to 25 molar %, a sterol at around 25 to 55 molar % and an ionisable cationic lipid at around 20 to 60 molar %.
Embodiment 51. The immunogenic composition according to embodiment 35, wherein the LNP comprises a PEG-modified lipid at around 0.5 to 10 molar %, optionally 0.5 to 5 molar % or 0.5 to 3 molar %.
Embodiment 52. The immunogenic composition according to any of embodiments 35 to 51, wherein the composition has a lipid to RNA molar ratio (N/P ratio) of about 2 to about 12, optionally a N/P ratio of 3 to about 8.
Embodiment 53. The immunogenic composition according to any of embodiments 28 to 52, wherein the mRNAs of (a), (b), (c¹), (c²), (c³), (c⁴), (c⁵) and/or (c⁶), optionally each, are not self-replicating.
Embodiment 54. The immunogenic composition according to any of embodiments 28 to 53, wherein the mRNAs of (a), (b), (c¹), (c²), (c³), (c⁴), (c⁵) and/or (c⁶) comprises a coding sequence which is a codon modified coding sequence, wherein the amino acid sequence encoded by the codon modified coding sequence is optionally not being modified compared to the amino acid sequence encoded by the corresponding wild type or reference coding sequence.
Embodiment 55. The immunogenic composition according to embodiment 54, wherein the codon modified coding sequence is selected from C maximized coding sequence, CAI maximized coding sequence, human codon usage adapted coding sequence, G/C content modified coding sequence, and G/C optimized coding sequence, or any combination thereof.
Embodiment 56. The immunogenic composition according to embodiment 55, wherein the codon modified coding sequence has a G/C content of at least about 45%, 50%, 55%, or 60%.
Embodiment 57. The immunogenic composition according to any of embodiments 28 to 56, wherein the mRNAs of (a), (b), (c¹), (c²), (c³), (c⁴), (c⁵) and/or (c⁶) comprises a 5′ cap, preferably m7G, cap0, cap1, cap2, a modified cap0 or a modified cap1 structure, preferably a 5′-cap1 structure.
Embodiment 58. The immunogenic composition according to any of embodiments 28 to 57, wherein the mRNAs of (a), (b), (c¹), (c²), (c³), (c⁴), (c⁵) and/or (c⁶) comprises a poly(A) tail sequence, suitably comprising 30 to 200 adenosine nucleotides and/or at least one poly (C) sequence, suitably comprising 10 to 40 cytosine nucleotides.
Embodiment 59. The immunogenic composition according to any of embodiments 28 to 58, wherein the mRNAs of (a), (b), (c¹), (c²), (c³), (c⁴), (c⁵) and/or (c⁶) comprises at least one histone stem-loop.
Embodiment 60. The immunogenic composition according to any of embodiments 28 to 59, wherein the mRNAs of (a), (b), (c¹), (c²), (c³), (c⁴), (c⁵) and/or (c⁶) comprises at least one poly (A) tail sequence comprising 30 to 200 adenosine nucleotides, preferably 100 adenosine nucleotides, wherein the 3′ terminal nucleotide of said RNA is an adenosine.
Embodiment 61. The immunogenic composition according to any of embodiments 28 to 60, wherein the mRNAs of (a), (b), (c¹), (c²), (c³), (c⁴), (c⁵) and/or (c⁶) comprises a 5′ untranslated region (UTR).
Embodiment 62. The immunogenic composition according to embodiment 61, wherein the 5′ UTR comprises or consists of a nucleic acid sequence derived from a 5′-UTR of a gene selected from HSD17B4, RPL32, ASAH1, ATP5A1, MP68, NDUFA4, NOSIP, RPL31, SLC7A3, TUBB4B and UBQLN2, or from a homolog, a fragment or variant of any one of these genes.
Embodiment 63. The immunogenic composition according to any of embodiments 28 to 62, wherein the mRNAs of (a), (b), (c¹), (c²), (c³), (c⁴), (c⁵) and/or (c⁶) comprises a 3′ UTR.
Embodiment 64. The immunogenic composition according to embodiment 63, wherein the 3′ UTR comprises or consists of a nucleic acid sequence derived from a 3′-UTR of a gene selected from PSMB3, ALB7, CASP1, COX6B1, GNAS, NDUFA1 and RPS9, or from a homolog, a fragment or a variant of any one of these genes.
Embodiment 65. The immunogenic composition according to any of embodiments 28 to 64, wherein the mRNAs of (a), (b), (c¹), (c²), (c³), (c⁴), (c⁵) and/or (c⁶) comprises an heterologous 5′-UTR that comprises or consists of a nucleic acid sequence derived from a 5′-UTR from HSD17B4 and at least one heterologous 3′-UTR comprises or consists of a nucleic acid sequence derived from a 3′-UTR of PSMB3.
Embodiment 66. The immunogenic composition according to any of embodiments 28 to 65, wherein the mRNAs of (a), (b), (c¹), (c²), (c³), (c⁴), (c⁵) and/or (c⁶) comprises from 5′ to 3′:
   i) 5′-cap1 structure;
   ii) 5′-UTR derived from a 5′-UTR of a HSD17B4 gene;
   iii) the coding sequence;
   iv) 3′-UTR derived from a 3′-UTR of a PSMB3 gene;
   v) optionally, a histone stem-loop sequence; and
   vi) poly (A) sequence comprising about 100 A nucleotides, wherein the 3′ terminal nucleotide of said RNA is an adenosine.
Embodiment 67. The immunogenic composition according to any of embodiments 28 to 66, wherein the mRNAs of (a), (b), (c¹), (c²), (c³), (c⁴), (c⁵) and/or (c⁶) does not comprise chemically modified nucleotides.
Embodiment 68. The immunogenic composition according to any of embodiments 28 to 66, wherein the mRNAs of (a), (b), (c¹), (c²), (c³), (c⁴), (c⁵) and/or (c⁶) comprises at least one chemical modification.
Embodiment 69. The immunogenic composition according to embodiment 68, wherein the chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.
Embodiment 70. The immunogenic composition according to embodiment 68 or 69, wherein the chemical modification is N1-methylpseudouridine and/or pseudouridine, suitably N1-methylpseudouridine.
Embodiment 71. The immunogenic composition according to any of embodiments 68 to 70, wherein the mRNAs of (a), (b), (c¹), (c²), (c³), (c⁴), (c⁵) and/or (c⁶) comprising the chemical modification is a uridine modification, preferably wherein 100% of the uridine positions in the mRNA are modified.
Embodiment 72. The immunogenic composition according to any of embodiments 1 to 71, wherein the ratio is a mass ratio (wt/wt ratio).
Embodiment 73. The immunogenic composition according to any of embodiments 1 to 72, further comprising at least one pharmaceutically acceptable carrier.
Embodiment 74. Vaccine comprising the immunogenic composition according to any of embodiments 1 to 73.
Embodiment 75. The vaccine according to embodiment 74, further comprising at least one antigen or at least one nucleic acid encoding said at least one antigen, such as at least one mRNA encoding an antigen from a further pathogen, suitably the pathogen being a virus.
Embodiment 76. The vaccine according to embodiment 75, said antigen further virus being selected from the group consisting of Coronavirus (e.g. SARS-COV-1, SARS-COV-2, MERS-COV), Pneumoviridae virus (e.g. Respiratory syncytial virus, Metapneumovirus) and Paramyxovidirae virus (e.g. Parainfluenza virus, Henipavirus), suitably said antigen from a further virus is a spike protein, or an antigenic fragment thereof, from a SARS-COV-2 virus or a mRNA encoding a spike protein, or an antigenic fragment thereof, from a SARS-COV-2 virus.
Embodiment 77. Kit or kit of parts comprising the antigens or the nucleic acids as defined in any of embodiments 1 to 27 or the mRNAs as defined in any of embodiments 3 to 73, suitably the mRNAs of (a), (b), (c¹), (c²), (c³), (c⁴), (c⁵) and/or (c⁶) as in defined in any of embodiments 28 to 73, optionally comprising a liquid vehicle for solubilizing, and, optionally, technical instructions providing information on administration and dosage of the components.
Embodiment 876. The kit or kit of parts according to embodiment 77, wherein the antigens or the nucleic acids or the mRNAs, suitably the mRNAs of (a), (b), (c¹), (c²), (c³), (c⁴), (c⁵) and/or (c⁶) are formulated separately.
Embodiment 79. The immunogenic composition according to any of embodiments 1 to 73, the vaccine according to any of embodiments 74 to 76 or the kit or kit of parts according to embodiment 77 or 78, wherein the antigens or the nucleic acids or the mRNAs, suitably the mRNAs of (a), (b), (c¹), (c²), (c³), (c⁴), (c⁵) and/or (c⁶) are formulated as a bedside mixing formulation.
Embodiment 80. The immunogenic composition according to any of embodiments 1 to 73, the vaccine according to any of embodiments 74 to 76 or the kit or kit of parts according to embodiment 77 or 78, wherein the antigens or the nucleic acids and/or the mRNAs of (a), (b), (c¹), (c²), (c³), (c⁴), (c⁵) and/or (c⁶) are co-formulated.
Embodiment 81. The immunogenic composition according to any of embodiments 1 to 73, the vaccine according to any of embodiments 74 to 76 or the kit or kit of parts according to embodiment 77 or 78, for use as a medicament.
Embodiment 82. The immunogenic composition according to any of embodiments 1 to 73, the vaccine according to any of embodiments 74 to 76 or the kit or kit of parts according to embodiment 77 or 78, for use in the treatment or prophylaxis of an infection with an influenza virus, suitably an influenza A and/or influenza B.
Embodiment 83. The immunogenic composition, the vaccine, the kit or kit of parts for use according to embodiment 82, wherein a single dose of the composition is 2 to 500 μg, especially 10 to 250 μg of total mRNA, such as 10 to 75 μg of total mRNA.
Embodiment 84. The immunogenic composition, the vaccine, the kit or kit of parts for use according to embodiment 82 or 83, for intramuscular administration.
Embodiment 85. The kit or kit of parts for use according to any of embodiments 82 to 84, wherein the antigens or the nucleic acids or the mRNAs, suitably the mRNAs of (a), (b), (c¹), (c²), (c³), (c⁴), (c⁵) and/or (c⁶) are administered at different sites of injection.
Embodiment 86. The immunogenic composition, the vaccine, the kit for use according to any of embodiments 82 to 85, wherein an immune response is elicited, suitably an adaptative immune response, more suitably a protective adaptative immune response against an influenza virus, suitably an influenza A and/or influenza B.
Embodiment 87. Method of treating or preventing a disorder caused by an influenza virus, suitably an influenza A and/or influenza B, wherein the method comprises applying or administering to a subject in need thereof the immunogenic composition according to any of embodiments 1 to 73, the vaccine according to any of embodiments 74 to 76 or the kit or kit of parts according to embodiment 77 or 78.
Embodiment 88. Method of eliciting an immune response, wherein the method comprises applying or administering to a subject in need thereof the immunogenic composition according to any of embodiments 1 to 73, the vaccine according to any of embodiments 74 to 76 or the kit or kit of parts according to embodiment 77 or 78.
Embodiment 89. The method according to embodiment 88, wherein the immune response is an adaptative immune response, suitably a protective adaptative immune response against an influenza virus, suitably against an influenza A virus and/or an influenza B virus.
Embodiment 90. The method according to embodiment 87 or the method according to embodiment 88 or 89, wherein the subject in need is a mammalian subject, suitably a human subject.
Embodiment 91. The method according to embodiment 87 or the method according to embodiment 88 or 89, wherein the immunogenic composition according to any of embodiments 1 to 73, the vaccine according to any of embodiments 74 to 76 or the kit or kit of parts according to embodiment 77 or 78 is administered in an amount effective to induce a T cell response against influenza A H1N1, influenza A H3N2, influenza B/Yamagata lineage and Influenza B/Victoria lineage.
Embodiment 92. The method according to embodiment 87 or the method according to embodiment 88 or 89, wherein the immunogenic composition according to any of embodiments 1 to 73, the vaccine according to any of embodiments 74 to 76 or the kit or kit of parts according to embodiment 77 or 78 is administered in an amount effective to induce a neutralizing antibody response against influenza A H1N1, influenza A H3N2, influenza B/Yamagata lineage and Influenza B/Victoria lineage.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods, which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Phase 1 Quadrivalent Influenza Vaccination Trail-Unmodified mRNA

A Phase 1 trial was designed to evaluate the safety, reactogenicity and immunogenicity of CVSQIV when administered as a single dose at different dose levels using an adaptive dose-finding design.

### 1. Trial Objectives and Endpoints

### Primary Objectives

To evaluate the safety and reactogenicity profile of CVSQIV at different dose levels.

### Secondary Objectives

To evaluate the humoral immune response to CVSQIV at different dose levels in terms of hemagglutination inhibition (HAI) antibody titers.

### Exploratory Objectives

To evaluate the humoral immune response to CVSQIV at different dose levels in terms of micro neutralization (MN) and NA inhibition (NI) antibody titers.
To evaluate the innate immune response to CVSQIV at different dose levels in all sentinel subjects.
To evaluate cross-reactivity to influenza antigens not included in the vaccine.

### Endpoints

### Primary

The frequencies of Grade 3 adverse reactions (ARs) and any serious adverse reaction (SAR) within at least 20 hours after the trial vaccine administration by dose level, for decisions on subsequent vaccination of additional sentinel subjects with the same dose level.
The frequencies of Grade 3 ARs and any SAR within at least 60 hours after the trial vaccine administration by dose level, for decisions on dose escalation as well as continuation of enrolment at the same dose level.
The frequencies, intensities and duration of solicited local ARs on the day of vaccination and the following 7 days by dose level, for the characterization of the safety and reactogenicity profile.
The frequencies, intensities, duration and relationship to trial vaccination of solicited systemic adverse events (AEs) on the day of vaccination and the following 7 days by dose level, for the characterization of the safety and reactogenicity profile.
The occurrence, intensities and relationship to trial vaccination of unsolicited AEs on the day of vaccination and the following 28 days by dose level, for the characterization of the safety and reactogenicity profile.
The occurrence and relationship to trial vaccination of serious adverse events (SAEs) and adverse events of special interest (AESIs) throughout the trial, for the characterization of the safety and reactogenicity profile.

### Secondary

Anti-HA antibody titers measured by HAI assay on Day 22 and Day 183.
Geometric mean titers (GMTs) of antigen-specific anti-HA antibody titers.
The proportion of subjects with antigen-specific seroconversion*.
Seroconversion for HA antigens measured by HAI assay is defined as a post-vaccination titer≥1:40 for subjects with a baseline titer≤1:10 and at least a 4-fold increase in post-vaccination titer relative to baseline for subjects with a baseline titer≥1:10.
The proportion of subjects with a 2-fold increase in antigen-specific post-vaccination anti-HA antibody titers relative to baseline.
The proportion of subjects with a 4-fold increase in antigen-specific post-vaccination anti-HA antibody titers relative to baseline.
The proportion of subjects with antigen-specific post-vaccination anti-HA antibody titer≥1:40 and ≥1:80.

### Exploratory

Anti-HA antibody titers measured by micro neutralization assay on Day 22 and Day 183.
GMTs of antigen-specific anti-HA antibody titers.
The proportion of subjects with a 2-fold increase in antigen-specific post-vaccination anti-HA antibody titers relative to baseline.
The proportion of subjects with a 4-fold increase in antigen-specific post-vaccination anti-HA antibody titers relative to baseline.

Anti-neuraminidase (NA) antibody titers measured by enzyme-linked lectin assay (ELLA) on Day 22 and Day 183.
GMTs of antigen-specific anti-NA titers.
The proportion of subjects with a 2-fold increase in antigen-specific post-vaccination anti-NA titers relative to baseline.
The proportion of subjects with a 4-fold increase in antigen-specific post-vaccination anti-NA titers relative to baseline.
The proportion of subjects with a post-vaccination anti-NA titer≥1:40 and ≥1:80.

### Cross-Reactivity to Antigens not Contained in the Vaccine.

Anti-NA antibody titers to B-Phuket.

### Innate Immune Response (Only in Sentinel Subjects)

Serum cytokine concentrations, including but not limited to IFN-α, IFN-γ, IL-6, chemokine ligand (CCL) 2 and IFN-γ-induced protein 10 (IP-10) on Day 2 and Day 22.

### 2. Trial Design

For the Phase 1 trial subjects were enrolled in a staggered manner in 5 dose levels (dose levels of 3, 6, 12, 20 and 28 μg). All subjects received a single dose of CVSQIV on Day 1. Subjects were enrolled in 2 age groups, including a Younger Adults group aged 18-55 years and an Older Adults group aged≥65 years. Each dose level was tested in 48 subjects (24 per age group) as follows:
12 sentinel subjects (6 per age group), and
36 additional subjects (18 per age group) after no safety concerns were identified in the sentinel subjects.

There were 3 protocol-scheduled visits on Day 1 (vaccination day), Day 22 (21 days post-vaccination) and Day 183 (6 months post-vaccination). Sentinel subjects had an additional visit on Day 2 (1 day post-vaccination) for collection of safety, reactogenicity and immunogenicity data. At each protocol-scheduled visit, blood samples were taken for safety and/or immunogenicity testing. In addition, there were 4 protocol-scheduled telephone contacts for collection of safety data (on Day 3, Day 8, Day 29 and Day 92) for all subjects with an additional telephone contact on Day 2 for non-sentinel subjects.

To ensure the safety of the subjects participating in the trial, enrollment in each dose level was initiated with sentinel subjects (i.e., a limited number of subjects from whom post-vaccination safety data were collected and evaluated before exposing a larger number of subjects to the same dose level). Within each dose level, safety data up to a minimum of 20 hours post-vaccination from the first 4 sentinel subjects (2 per age group) were collected and evaluated before proceeding with vaccination of an additional 8 sentinel subjects (4 per age group). Data from all 12 sentinel subjects in each dose level were subsequently collected for a minimum of 60 hours and evaluated, before exposing a larger number of subjects to the same dose level.

### 3. Trial Population

### Inclusion Criteria

Subjects were enrolled in this trial only if they met all of the following criteria:
1. Healthy male or female subjects between the ages of 18 and 55 years, inclusive, at enrollment (Younger Adults groups) or aged≥65 years at enrollment (Older Adults groups).
   A healthy subject is defined as an individual who is in good general health, according to the Investigator's assessment. Chronic health conditions are acceptable if the condition is considered stable and well controlled with treatment according to the discretion of the Investigator.
2. Signed informed consent obtained before any trial procedures.
3. Expected to be compliant with protocol procedures and available for clinical follow-up through the last planned contact.
4. Physical examination without clinically significant findings according to the Investigator's assessment.
5. Body mass index (BMI)≥18.0 and ≤32.0 kg/m2.
6. Females: At the time of enrollment, negative human chorionic gonadotropin (hCG) pregnancy test (serum) for women presumed to be of childbearing potential on the day of enrollment. On Day 1 (pre vaccination): negative urine pregnancy test (hCG), (only required if serum pregnancy test was performed more than 3 days before).
Note: Women that are postmenopausal (defined as amenorrhea for ≥12 consecutive months prior to enrollment without an alternative medical cause) or permanently sterilized will be considered as not having reproductive potential.
7. Females of childbearing potential must use highly effective methods of birth control from 1 month before until 3 months after the trial vaccine administration.

### Exclusion Criteria

Subjects were not enrolled in the trial if they meet any of the exclusion criteria.
1. Use of any investigational or non-registered product (vaccine or drug) other than the trial vaccine within 28 days preceding the trial vaccine administration, or planned use during the trial.
2. Receipt of any influenza vaccine within 90 days of enrollment.
3. Receipt of any mRNA vaccine within 2 months of enrollment.
4. Receipt of any other vaccines within 28 days prior to enrollment or planned receipt of any vaccine within 28 days of trial vaccine administration.
5. Any treatment with immunosuppressants or other immune-modifying drugs (including, but not limited to, corticosteroids, biologicals and methotrexate) for >14 days total within 6 months prior to the trial vaccine administration or planned use during the trial, with the exception of inhaled or topically-applied steroids. For corticosteroid use, this means prednisone or equivalent, 0.5 mg/kg/day for 14 days or more.
6. Any medically diagnosed or suspected immunosuppressive or immunodeficient condition based on medical history and physical examination, including known human immunodeficiency virus infection.
7. Chronic hepatitis B virus infection and chronic hepatitis C virus infection.
8. History of AEs with a suspected immune-mediated etiology (pIMD).
9. History of angioedema.
10. History of any neurological disorders or seizures including Guillain-Barre syndrome, with the exception of febrile seizures during childhood.
11. History of allergy to any component of CVSQIV, or to aminoglycoside or beta-lactam antibiotics.
12. History of any severe allergic reaction or anaphylactic reaction.
13. History of or current alcohol and/or drug abuse.
14. Administration of immunoglobulins and/or any blood products within the 3 months preceding the trial vaccine administration.
15. Presence or evidence of significant acute or chronic medical or psychiatric illness.
16. Current or past malignancy, unless completely resolved without sequelae for >5 years.
17. For females: pregnancy or lactation.
18. Subjects with impaired coagulation or any bleeding disorder in whom an intramuscular injection or a blood draw is contraindicated.
19. Subjects employed by the Sponsor, Investigator or trial site, or relatives of research staff working on this trial.

### 4. Trial Vaccine

CVSQIV is an investigational LNP-formulated RNACTIVE quadrivalent seasonal influenza vaccine containing 4 HA antigens and 3 NA antigens according to the WHO recommendation on the composition of cell- or recombinant based influenza virus vaccines for use in the 2020-2021 Northern hemisphere influenza season.

The IMP is composed of the following active pharmaceutical ingredients:
4 mRNAs encoding HA of influenza virus strains A/Hawaii/70/2019 (H1N1), A/Hong Kong/45/2019 (H3N2), B/Washington/02/2019 and B/Phuket/3073/2013;
3 mRNAs encoding NA of influenza virus strains A/Hawaii/70/2019 (H1N1), A/Hong Kong/45/2019 (H3N2) and B/Washington/02/2019;
4 lipid components: cholesterol, 1,2 distearoyl-sn-glycero-3-phosphocholine (DSPC), PEG-ylated lipid and a cationic lipid.

All subjects received a single dose of CVSQIV on Day 1. Injections were performed intramuscularly (IM) by needle in the deltoid area.

### 5. Trial Assessments and Procedures

The following trial visits/contacts were used:
For the sentinel subjects: 4 protocol-scheduled visits on Day 1, Day 2, Day 22 and Day 183; and 4 protocol-scheduled telephone contacts on Day 3, Day 8, Day 29 and Day 92.
For all additional subjects: 3 protocol-scheduled visits on Day 1, Day 22 and Day 183; and 5 protocol-scheduled telephone contacts on Day 2, Day 3, Day 8, Day 29 and Day 92.

At each protocol-scheduled visit, blood samples were taken for safety and/or immunogenicity testing.

The purpose of the telephone calls was to inquire on the subject's general well-being and to assess safety. An electronic diary was used for efficient real-time collection of post-vaccination solicited and unsolicited adverse events (AEs).

### 5.1 Safety Assessments

### Solicited Adverse Events

Reactogenicity was assessed daily on the day of vaccination and the following 7 days via collection of solicited local adverse reactions (ARs) (injection-site pain, redness, swelling and itching) and solicited systemic AEs (fever, headache, fatigue, chills, myalgia, arthralgia, nausea/vomiting and diarrhea) using electronic diaries.

Body temperature was measured orally and by using the thermometer provided to the subject at Visit 1.

Solicited AEs were assessed on an intensity scale of absent, mild, moderate and severe (Table 3 and Table 4).

By definition, all solicited local ARs occurring from the time of vaccination are considered related to trial vaccination. For solicited systemic AEs, the Investigator assessed the relationship between the trial vaccine and each occurrence of each AE.

TABLE 3
Intensity Grading for Solicited Local ARs

| AR | Grade | Definition | |
|---|---|---|---|
| Pain at | 0 | Absent | |
| injection | 1 (mild) | No interference with activity | |
| site | 2 (moderate) | Repeated use of non-narcotic pain reliever >24 hours or interferes | |
| | | with activity | |
| | 3 (severe) | Any use of narcotic pain reliever or prevents daily activity | |
| Redness | 0 | <2.5 | cm |
| | 1 (mild) | 2.5-5 | cm |
| | 2 (moderate) | 5.1-10 | cm |
| | 3 (severe) | >10 | cm |
| Swelling | 0 | <2.5 | cm |

| | | |
|---|---|---|
| | 1 (mild) | 2.5-5 cm and does not interfere with activity |
| | 2 (moderate) | 5.1-10 cm or interferes with activity |
| | 3 (severe) | >10 cm or prevents daily activity |
| Itching | 0 | Absent |
| | 1 (mild) | No interference with activity |
| | 2 (moderate) | Some interference with activity |
| | 3 (severe) | Significant, prevents daily activity |

Based on the United States Food and Drug Administration toxicity grading scale Coates et al. 2020.

TABLE 4
Intensity Grading for Solicited Systemic Adverse Events

| Adverse Event | Grade | Definition | |
|---|---|---|---|
| Fever | 0 | <38° | C. |
| | 1 (mild) | ≥38-38.4° | C. |
| | 2 (moderate) | ≥38.5-38.9° | C. |
| | 3 (severe) | ≥39° | C. |

| | | |
|---|---|---|
| Headache | 0 | Absent |
| | 1 (mild) | No interference with activity |
| | 2 (moderate) | Repeated use of non-narcotic pain reliever >24 hours or some |
| | | interference with activity |
| | 3 (severe) | Significant; any use of narcotic pain reliever and/or prevents daily activity |
| Fatigue | 0 | Absent |
| | 1 (mild) | No interference with activity |
| | 2 (moderate) | Some interference with activity |
| | 3 (severe) | Significant, prevents daily activity |
| Chills | 0 | Absent |
| | 1 (mild) | No interference with activity |
| | 2 (moderate) | Some interference with activity |
| | 3 (severe) | Significant, prevents daily activity |
| Myalgia | 0 | Absent |
| | 1 (mild) | No interference with activity |
| | 2 (moderate) | Some interference with activity |
| | 3 (severe) | Significant, prevents daily activity |
| Arthralgia | 0 | Absent |
| | 1 (mild) | No interference with activity |
| | 2 (moderate) | Some interference with activity |
| | 3 (severe) | Significant, prevents daily activity |
| Nausea/Vomiting | 0 | Absent |
| | 1 (mild) | No interference with activity and/or 1-2 episodes/24 hours |
| | 2 (moderate) | Some interference with activity and/or >2 episodes/24 hours |
| | 3 (severe) | Prevents daily activity, requires outpatient i.v. hydration |
| Diarrhea | 0 | Absent |
| | 1 (mild) | 2-3 loose stools or <400 g/24 hours |
| | 2 (moderate) | 4-5 stools or 400-800 g/24 hours |
| | 3 (severe) | 6 or more watery stools or >800 g/24 hours or |
| | | requires outpatient i.v. hydration |

i.v. = intravenous
Based on the United States Food and Drug Administration toxicity grading scale Coates et al. 2020.

### Unsolicited Adverse Events and Serious Adverse Events

Electronic diaries were used for collection of unsolicited AEs on the day of vaccination and the following 28 days.

The occurrence of AEs (serious and non-serious) was assessed by non-directive questioning of the subject at each visit/contact. AEs volunteered by the subject during or between visits/contacts in the diary or detected through observation, physical examination, laboratory tests, or other assessments during the entire trial, were recorded in the electronic case report forms (eCRF), if they fell within the reporting period. Subjects were instructed to report immediately any AEs with serious symptoms, subjective complaints or objective changes in their well-being to the Investigator or the site personnel, regardless of the perceived relationship between the event and the trial vaccine for assessment of the occurrence of SAEs, AESIs and non-serious intercurrent medical conditions that may affect the immune response, including influenza-like illness. Non-serious AEs that occurred after Day 29 were not to be collected unless they categorized as a pIMD, non-serious intercurrent medical condition, or if they lead to trial discontinuation.

For all AEs, the Investigator assessed the relationship between the trial vaccine and each occurrence of AE/SAE. Additionally, for unsolicited AEs reported on the day of vaccination and the following 28 days, the Investigator or site personnel also recorded if the subject received medical attention for the AE.

SAEs, non-serious intercurrent medical conditions that may affect the immune response, including influenza-like illness, and AEs leading to trial discontinuation were to be collected throughout the trial.

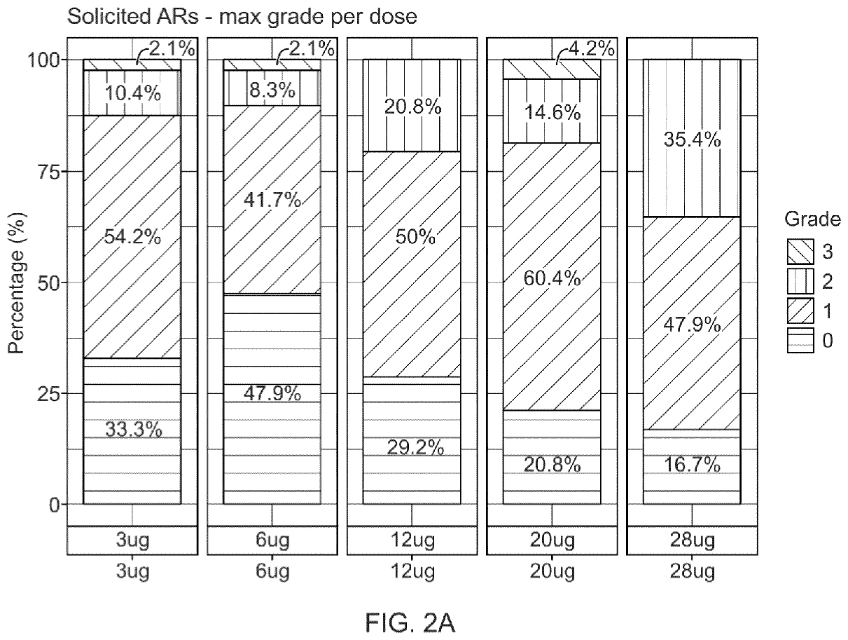

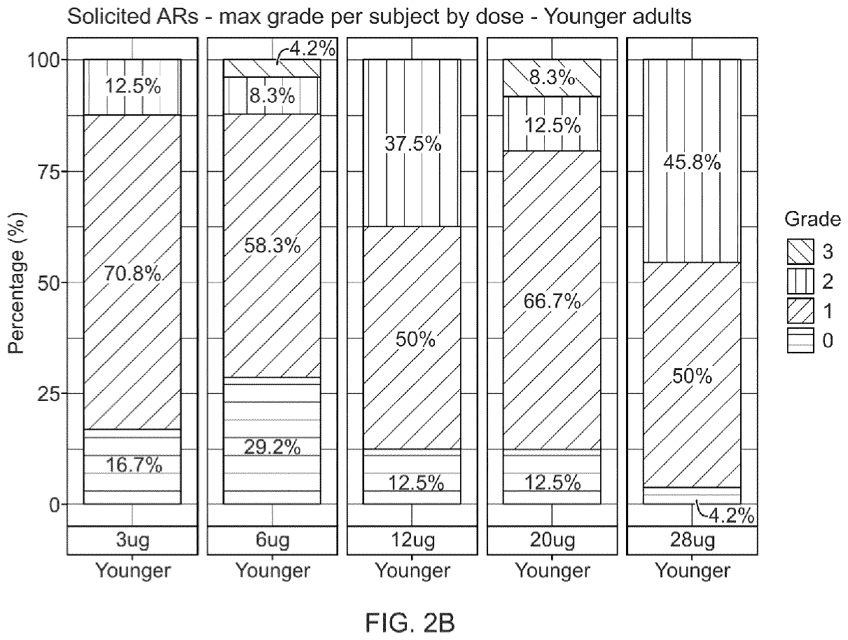

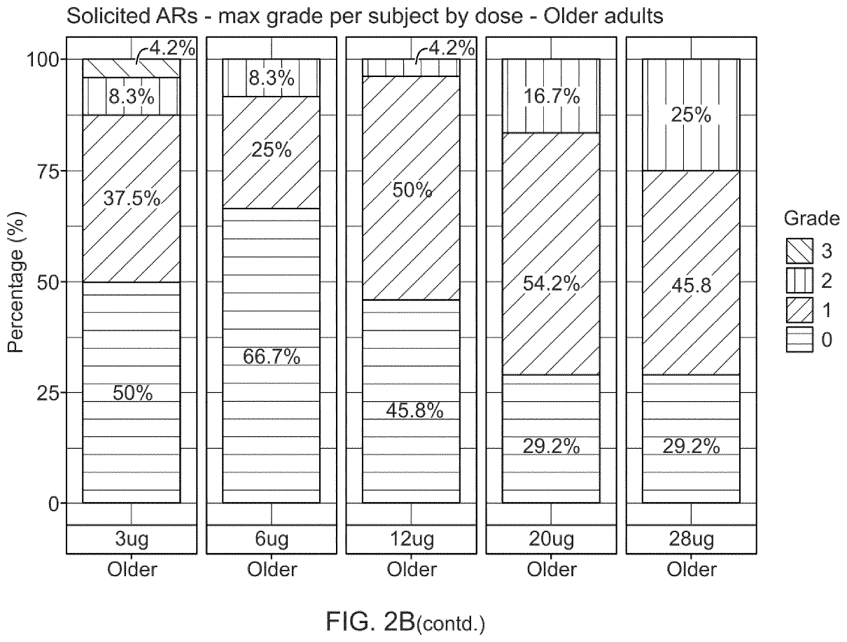

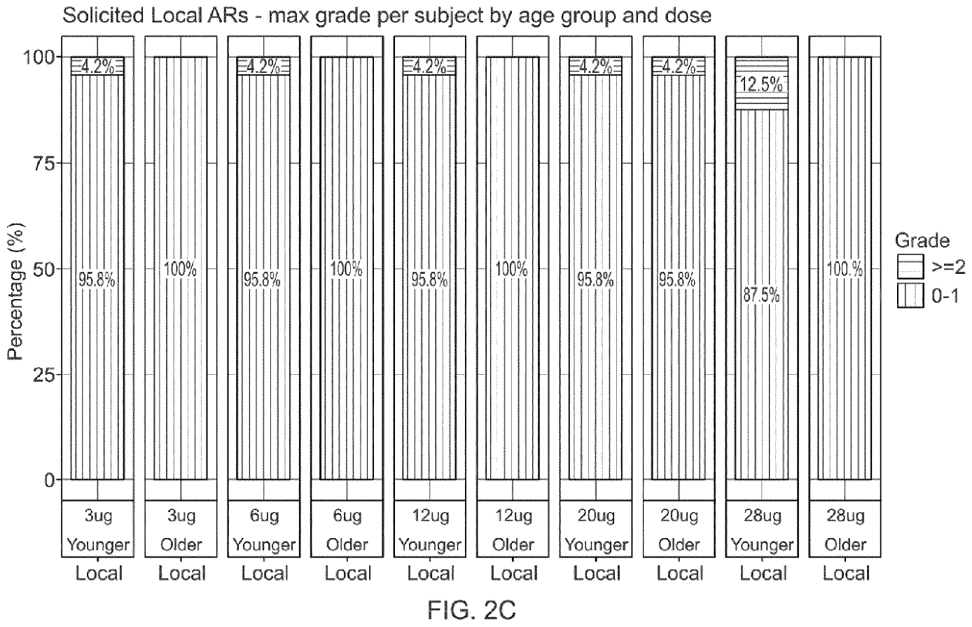

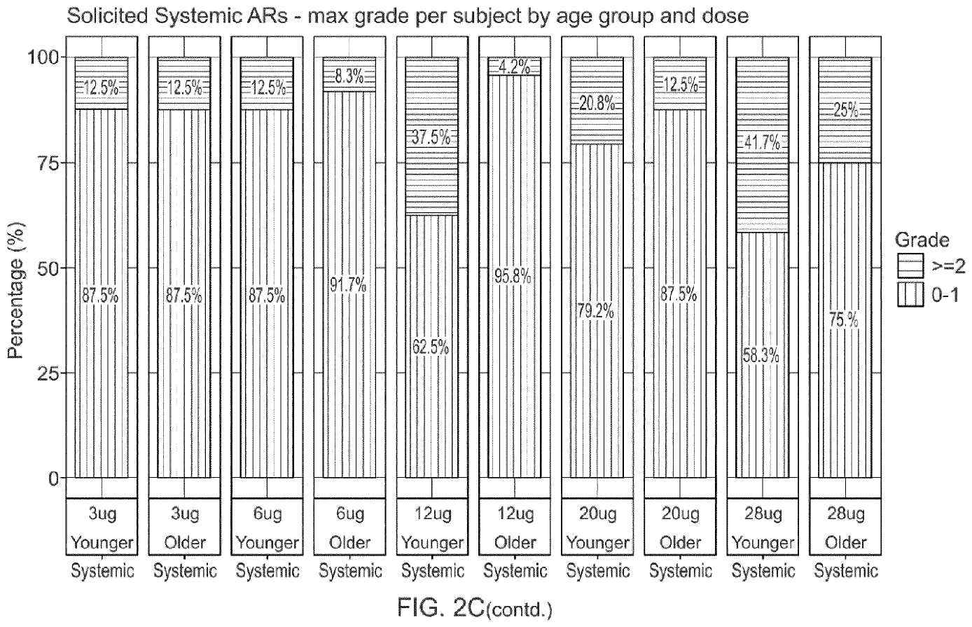

The results of the reactogenicity assessment of subjects in the CVSQIV trial are shown in FIG. 2. Solicited Adverse Events in subjects at the studied mRNA dose levels were assessed and are shown in the chart in FIG. 2A. Overall, a dose-dependent increase of reactogenicity was observed with a low rate of grade 3 reactogenicity. The results were also separately analyzed between younger and older adults. As shown in FIG. 2B, a higher rate of reactogenicity was seen in younger adults when compared to older adults. Grade 3 reactogenicity was exclusively observed in younger adults (note that a case of grade 3 diarrhea/vomiting in an older adult at 3 μg was found to be due to Amebiasis, and deemed not-related to the trial vaccine). The results were also separated between local and systemic events as shown in FIG. 2C. These results show that local reactogenicity (almost exclusively pain at the injection site) was typically seen at low severity. The overall reactogenicity profile was rather driven by systemic reactogenicity.

### Physical Examination, Vital Signs and Electrocardiogram

Physical examination and vital signs were performed/measured by a qualified healthcare professional.

Vital signs (body temperature, systolic/diastolic blood pressure, and heart rate) were recorded at each visit in a standardized manner after the subject had rested in the sitting position for 5 minutes. At the vaccination visit on Day 1, vital signs were measured pre- and post-vaccination prior to discharge. Subjects were observed for 4 hours following vaccination. Vital signs must have been within normal or clinically non-relevant abnormal ranges or have returned to pre-vaccination values for the subject to be discharged.

A complete physical examination was performed on Day 1, except if results of a complete physical examination performed within 21 days prior to Day 1 were available and sufficient in view of the protocol requirements, in which case a symptom-directed physical examination was performed on Day 1 prior to vaccination. The complete physical examination included: general appearance, eyes/ears/nose/throat, head/neck/thyroid, lymph node areas, cardiovascular system, lung/chest, abdomen, extremities and neurological examination, skin examination, and measurement of weight and height. At the other trial visits, a symptom-directed physical examination was performed at the discretion of the Investigator.

An ECG with conventional 12-lead traces was recorded prior to enrollment for all subjects.

Additionally, ECGs were performed as clinically indicated.

### Medical/Surgical and Medication/Vaccination History

All significant findings and pre-existing conditions present in a subject prior to enrollment were reported on the relevant medical history/current medical conditions screen of the eCRF.

Medication/vaccination taken within 6 months prior to enrollment was also recorded in the eCRF to establish eligibility.

### 5.2 Immunogenicity Assessments

Blood samples on Day 1 were collected prior to vaccination.

### Humoral Immune Response

The humoral immune response induced by vaccination with CVSQIV was evaluated by 3 assays on serum samples collected from all subjects on Day 22 and Day 183 and compared to the Day 1 pre-vaccination baseline sample:
Antibody titers to each HA antigen will be measured by HAI assay and MN assay (Trombetta et al., 2014 and Carnell et al., 2021, each of which is incorporated herein by reference).
Antibody titers for each NA antigen will be measured by ELLA (Gao et al., 2016 and Couzens et al., 2014, each of which is incorporated herein by reference).

The following measures were used for evaluation of the immune response to each HA and NA antigen on Day 1, Day 22 and Day 183: proportion of subjects with detectable antibody titers (≥1:10) at baseline, GMTs, fold increase in antigen-specific post-vaccination titer relative to baseline, and proportion of subjects with antigen-specific post-vaccination antibody titer≥1:40 and≥1:80. In addition, seroconversion rates will be evaluated for each HA antigen measured by HAI assay.

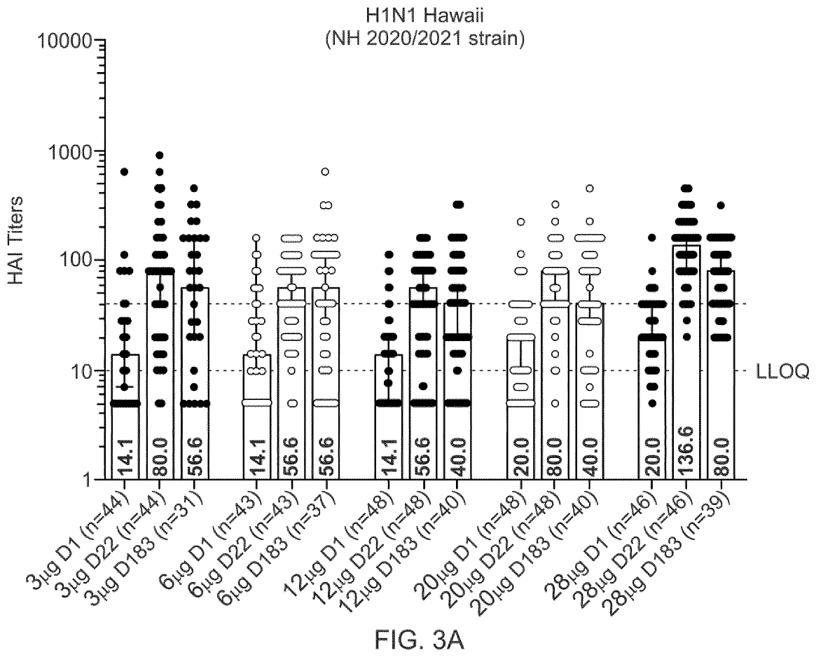

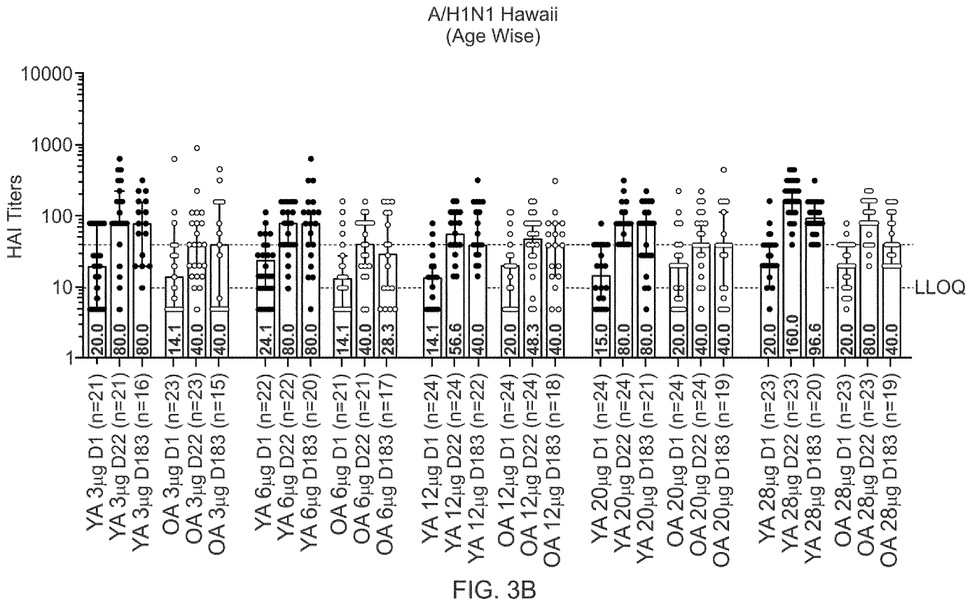

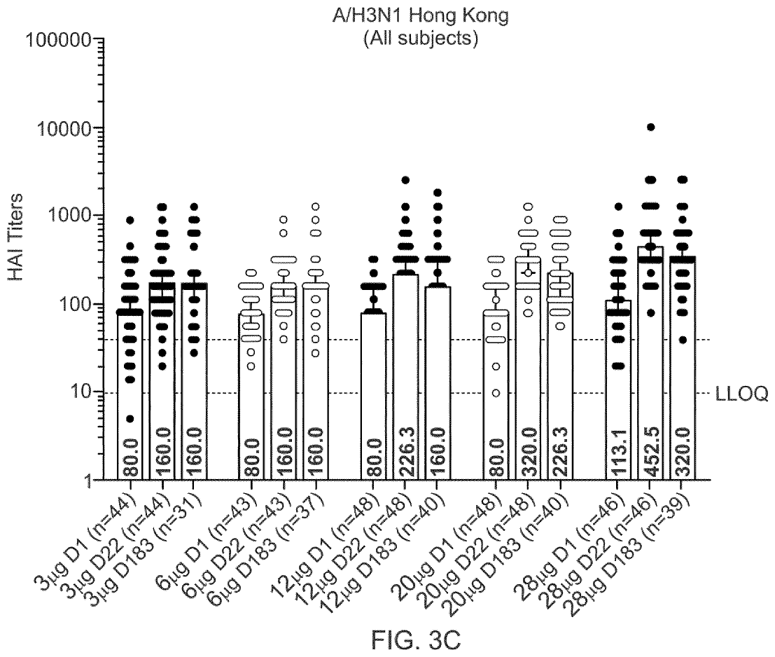

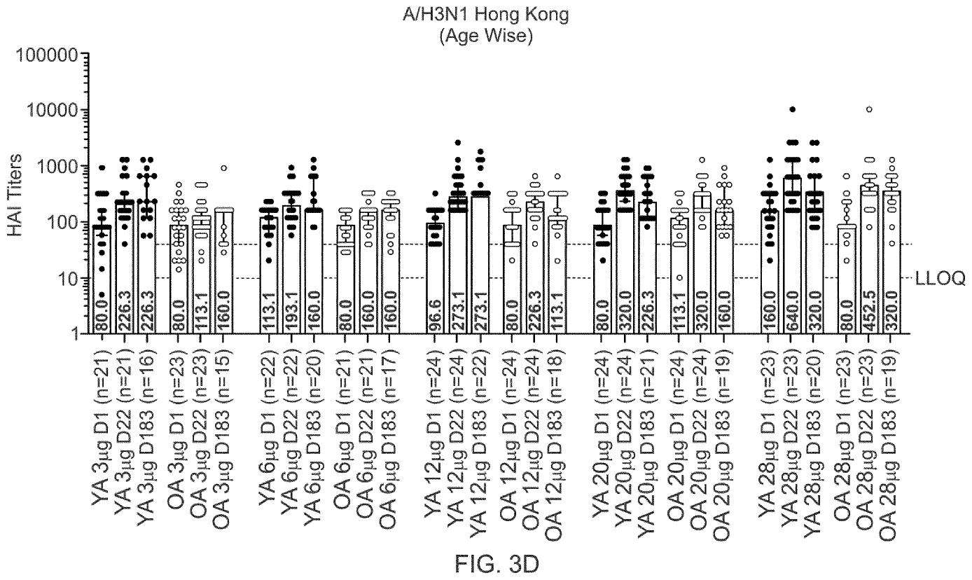

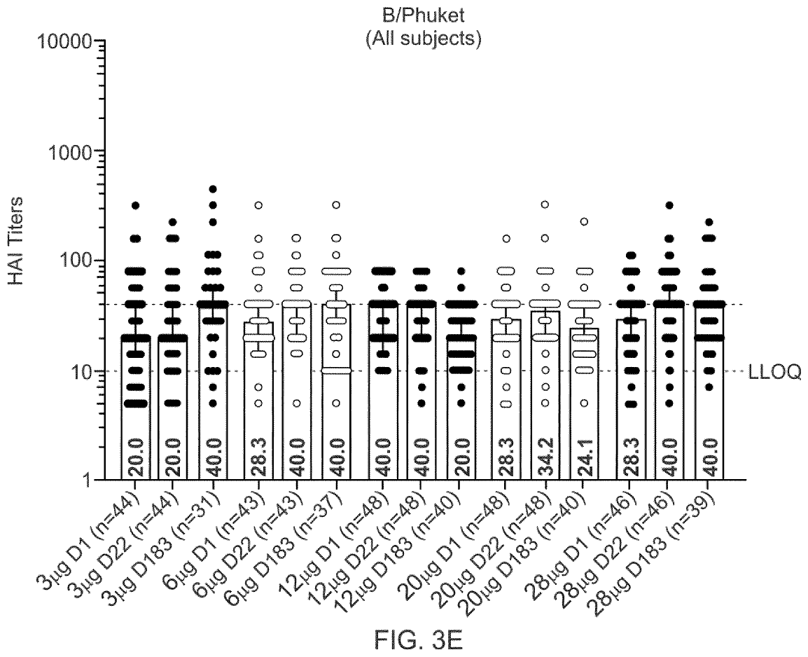

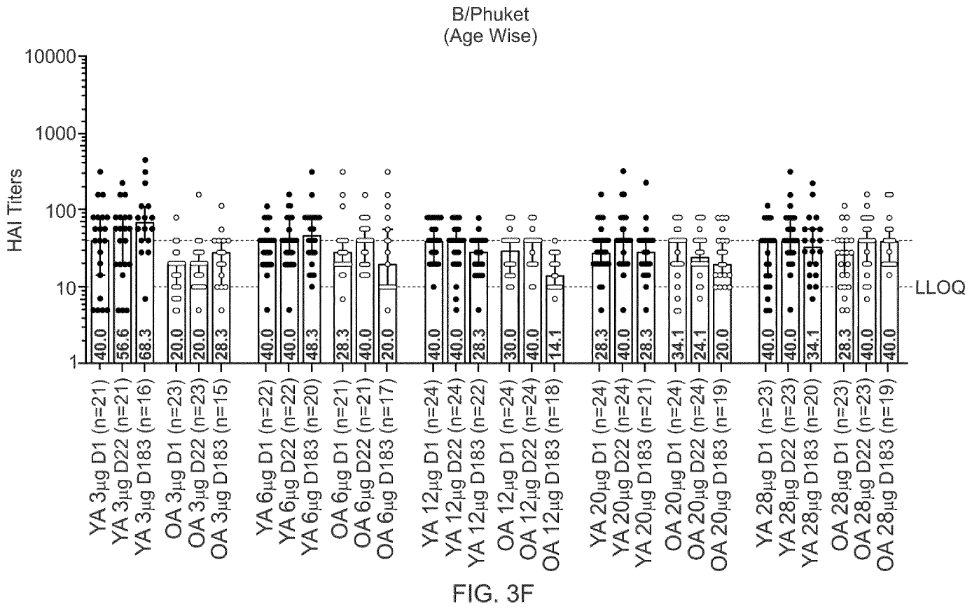

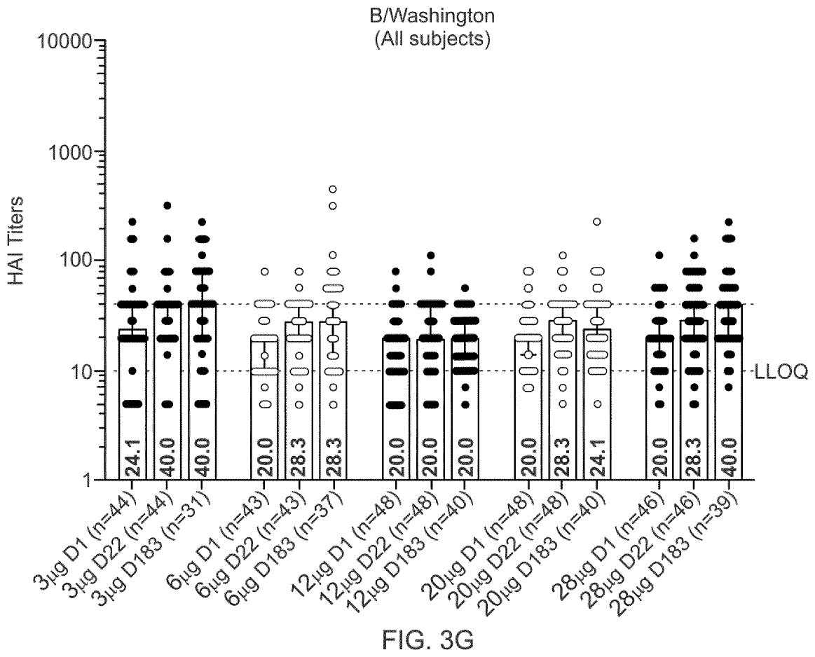

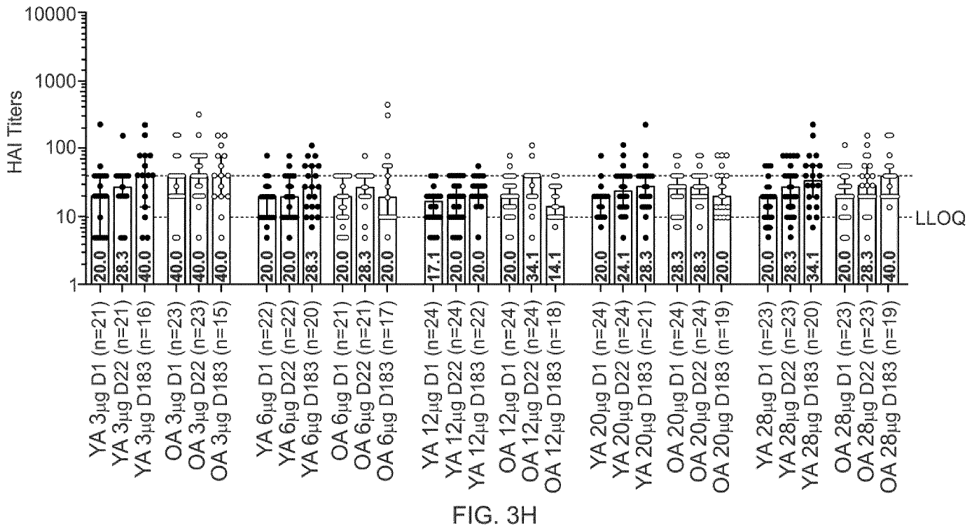

The results for the Antibody (HAI) Assay at days 0 and 22 are shown in FIG. 3. In these figures the left panels show the HAI titers for all subjects at Day 1 and Day 22 at the indicated vaccine mRNA dose levels. Data in the right panels are separated between younger adults and older adults at the indicated mRNA dose levels. Data are shown relative to each of the HA components encoded by the vaccine mRNA: H1N1 (FIG. 3A); H3N2 (FIG. 3B); B/Phuket (FIG. 3C); and B/Washington (FIG. 3D). For the influenza A HA antigens significant increase in GMTs were observed in both YA and OA at D22. However, for both of the influenza B HA antigens overall increase in GMTs at D22 was low.

These data are also summarized in the seroconversion rates (SCR) from HAI assay shown in FIG. 4. Again, SCR were robust for influenza A HA antigens, but low for influenza B HA antigens.

The HA data were also confirmed by microneutralization (MN) assay as shown in FIG. 5. These data show the percentage of study subjects that exhibited a≥four-fold increase in anti-HA titer by microneutralization assay. MN data confirmed the results obtained by HAI assay.

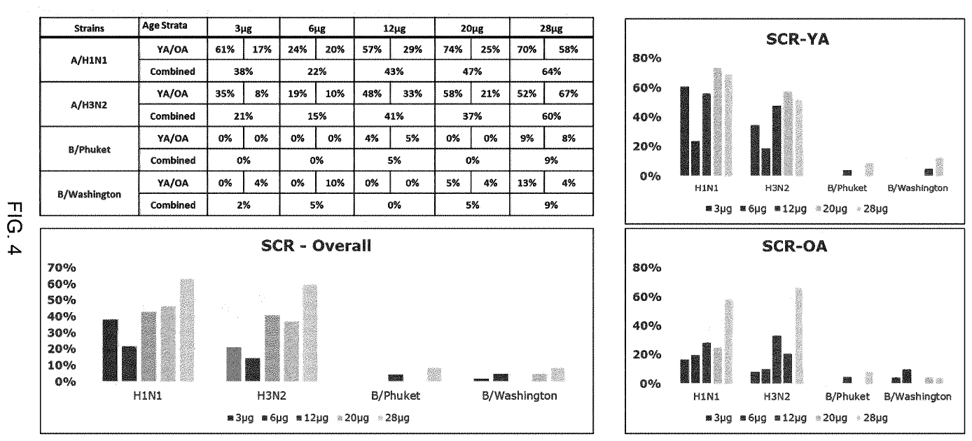

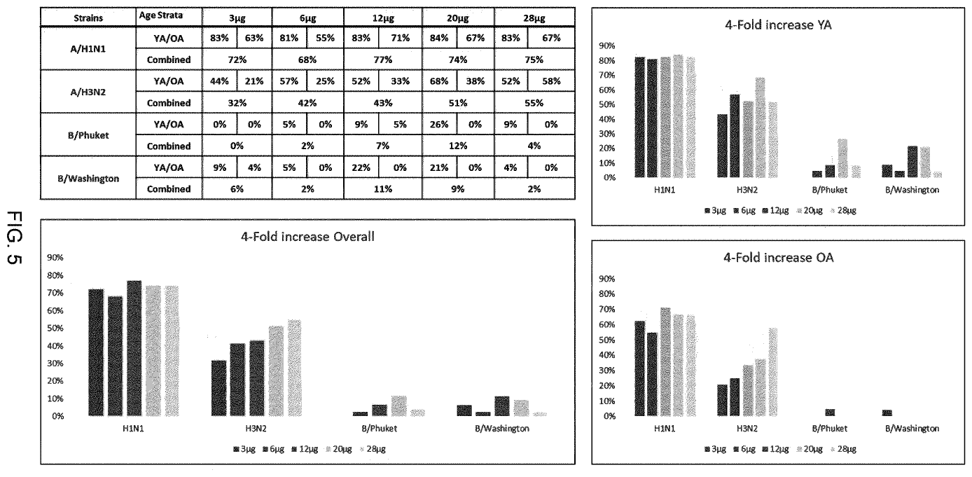

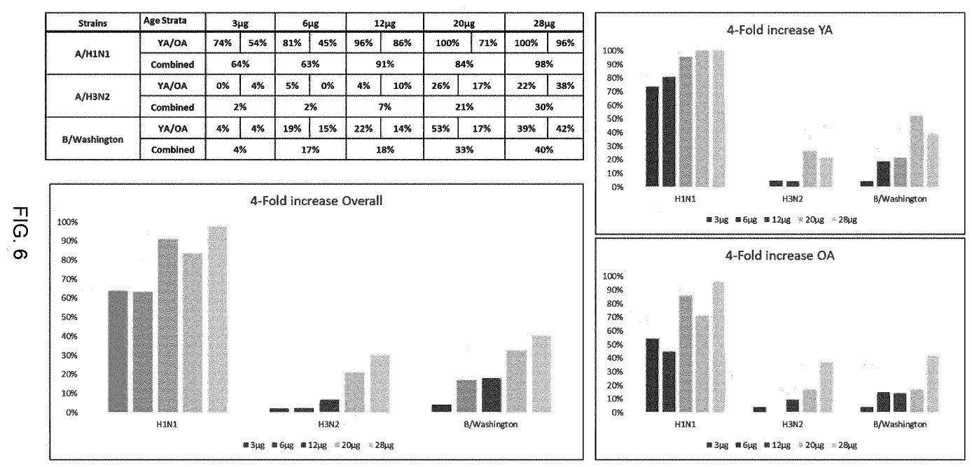

Finally, the immune response to NA antigens was assessed by enzyme linked lectin assay (ELLA). FIG. 6 shows the percentage of study subjects that exhibited a≥four-fold increase in anti-NA titer by ELLA. The most robust immune response in ELLA was seen to the N1 antigen.

### Innate Immune Response

The innate immune response induced by vaccination with CVSQIV was evaluated in sentinel subjects on Day 2 and Day 22 by measurement of serum cytokines, including but not limited to IFN-α, IFN-γ, IL 6, CCL2 and IP 10, and compared to the Day 1 pre-vaccination baseline sample.

### Example 2-Immunogenicity Study in Mice of 4-Component and 8-Component Flu Seasonal mRNA Formulations with Different Proportions Between the mRNA Sequences (Prophetic)

The objective of this study is the evaluation of immunogenicity and early innate stimulation upon immunization of mice with seasonal influenza virus 4-component and 8-component modified (N1-methylpseudouridine) mRNA vaccines containing combinations of the 4 HA- or 4 NA-encoding sequences that differ by the proportions between the mRNA sequences. Monovalent HA formulations will be included in this study to enable the assessment of potential immunological interference caused by the addition of the other mRNA sequences when combining 4 or 8 mRNAs. Different ratios between the HA will be evaluated. The proposed study design is presented in Table 5.

Mice will be vaccinated twice IM on Day 0 and Day 21 with seasonal influenza virus mRNA vaccines. Animals in the negative control group will be injected with 0.9% NaCl buffer. Animals in the positive control group will be immunized twice IM on Day 0 and Day 21 with one tenth of the human dose of FLUARIX Quadrivalent.

TABLE 5
Study design immunogenicity in mice with 4- and 8-component mRNA vaccines

| | | | Total | HA encoding mRNA-LNP | | | | NA encoding mRNA-LNP | | | |
|---|---|---|---|---|---|---|---|---|---|---|---|
| | | Vaccine | Dose | (DP) | | | | (DP) | | | |
| Gr | Animals | formulation | (μg) | H1 | H3 | B/Vic | B/Yam | N1 | N2 | B/Vic | B/Yam |
| 1 | Balb/c | 1HA H1 | 0.2 | 0.2 | — | — | — | — | — | — | — |
| 2 | mice | 1HA B/Vic | 0.6 | — | — | 0.6 | — | — | — | — | — |
| 3 | (n = 8F) | 1HA B/Yam | 0.6 | — | — | — | 0.6 | — | — | — | — |
| 4 | | 2HA | 1.2 | — | — | 0.6 | 0.6 | — | — | — | — |
| | | B/Vic + | | | | | | | | | |
| | | B/Yam | | | | | | | | | |
| 5 | | 4HA | 0.8 | 0.2 | 0.2 | 0.2 | 0.2 | — | — | — | — |
| | | equal dose | | | | | | | | | |
| 6 | | 4HA | 1.2 | 0.2 | 0.2 | 0.4 | 0.4 | — | — | — | — |
| | | MD A/HA + | | | | | | | | | |
| | | MD B/HA | | | | | | | | | |
| 7 | | 4HA | 1.6 | 0.2 | 0.2 | 0.6 | 0.6 | — | — | — | — |
| | | MD A/HA + | | | | | | | | | |
| | | HD B/HA | | | | | | | | | |
| 8 | | 4HA | 1.0 | 0.2 | 0.4 | 0.2 | 0.2 | — | — | — | — |
| | | MD H3/HA + | | | | | | | | | |
| | | MD B/HA | | | | | | | | | |
| 9 | | 4HA | 1.2 | 0.2 | 0.6 | 0.2 | 0.2 | — | — | — | — |
| | | HD H3/HA + | | | | | | | | | |
| | | MD B/HA | | | | | | | | | |
| 10 | | 4HA + 4NA | 1.2 | 0.2 | 0.2 | 0.2 | 0.2 | 0.1 | 0.1 | 0.1 | 0.1 |
| | | equal dose | | | | | | | | | |
| | | HA + | | | | | | | | | |
| | | LD NA | | | | | | | | | |
| 11 | | 4HA + 4NA | 1.6 | 0.2 | 0.2 | 0.4 | 0.4 | 0.1 | 0.1 | 0.1 | 0.1 |
| | | MD A/HA + | | | | | | | | | |
| | | MD B/HA + | | | | | | | | | |
| | | LD NA | | | | | | | | | |
| 12 | | 4HA + 4NA | 2.0 | 0.2 | 0.2 | 0.6 | 0.6 | 0.1 | 0.1 | 0.1 | 0.1 |
| | | MD A/HA + | | | | | | | | | |
| | | HD B/HA + | | | | | | | | | |
| | | LD NA | | | | | | | | | |
| 13 | | 4HA + 4NA | 2.4 | 0.2 | 0.2 | 0.6 | 0.6 | 0.2 | 0.2 | 0.2 | 0.2 |
| | | MD A/HA + | | | | | | | | | |
| | | HD B/HA + | | | | | | | | | |
| | | HD NA | | | | | | | | | |
| 14 | | *Fluarix QIV | 6 | 1.5 | 1.5 | 1.5 | 1.5 | n.d. | n.d. | n.d. | n.d. |
| | | NH22-23 | | | | | | | | | |
| 15 | n = 5 | Vehicle | N/A | N/A | N/A | N/A | N/A | N/A | N/A | N/A | N/A |
| | | (NaCl) | | | | | | | | | |

Haemagglutinin; IM = Intramuscular; N/A = Not applicable; NA = Neuraminidase; NH = Northern hemisphere, LD = low dose, MD = middle dose, HD = high dose;
*Commercially available influenza vaccine

The induction of functional antibodies against all antigenic components of the mRNA vaccines will be analyzed using the HI assay for the anti-HA responses and the ELLA for the anti-NA responses in serum collected two weeks post second immunization.

### Example 3-Phase 1/2 Quadrivalent Influenza Vaccination Trail-Modified mRNA (Scenario 1)

This Phase 1/2 study is an exploratory dose-finding (Phase 1) and dose-confirmation (Phase 2) study investigating quadrivalent seasonal influenza modified (N1-methyl-pseudouridine) mRNA vaccines with enrolment of younger adult (YA) participants aged 18-64 years in Phase 1 (Segment 1 and 2) and YAs and older adult (OA) participants aged 65-85 years in Segment 2 of Phase 1 and Phase 2.

In the exploratory Phase 1-Segment 1 and Phase 1-Segment 2, the safety, reactogenicity and immunogenicity of the Flu Seasonal modified mRNA investigational vaccine will be compared to the active control to select appropriate combination(s) for further investigation in Phase 2. In Phase 2 of this study, the selected mRNA investigational vaccine candidate(s) will be compared to active control.

### Study Design

Healthy or medically stable participants.
All study interventions are administered as single dose intramuscularly (IM).
Active control: GSK's Flu Dresden-Quadrivalent Influenza Vaccine, hereafter referred to as Flu D-QIV and commercially available as A-RIX-TETRA in Belgium will be chosen as active control in different phases of this study. It is indicated for active immunization for the prevention of disease caused by influenza A subtype viruses and type B viruses contained in the vaccine. The vaccine is approved for use in children above 6 months of age and adults.
Aspects of data collection: blood samples, safety events, vaccination experience questionnaire (VEQ).

### Phase 1:

FTiH
Single-blind (unblinded sponsor)

### Segment 1:

Participants aged 18-64 years.
11 groups enrolled in parallel (10 mRNA groups and 1 control group), detailed in Table 6.
Approximate number of participants: 264 (24 participants per group).
Strains included in study interventions will be based on WHO recommendation for influenza virus vaccine composition for Northern Hemisphere (NH) 2022-2023.

Each mRNA-coding sequence used in this phase will be manufactured as a separate lot and will be provided to the site staff/pharmacist to be combined at “bedside” before administration to each participant.

TABLE 6
List of study interventions for Phase 1 - Segment 1

| | | Age |
|---|---|---|
| | | Min-Max |
| Phase 1 - Segment 1 | Study interventions | Years |
| mRNA groups | HA-H1-mono | 18-64 |
| | HA-B-Vic-mono | 18-64 |
| | 4HA-1 (HA-H1, HA-H3, HA-B-Vic and HA-B-Yam [combination 1]) | 18-64 |
| | 4HA-2 (HA-H1, HA-H3, HA-B-Vic, HA-B-Yam [combination 2]) | 18-64 |
| | 4HA-3 (HA-H1, HA-H3, HA-B-Vic, HA-B-Yam [combination 3]) | 18-64 |
| | 4HA-4 (HA-H1, HA-H3, HA-B-Vic, HA-B-Yam [combination 4]) | 18-64 |
| | 4HA-5 (HA-H1, HA-H3, HA-B-Vic, HA-B-Yam [combination 5]) | 18-64 |
| | 4NA-1 (NA-N1, NA-N2, NA-B-Vic and NA-B-Yam [combination 1]) | 18-64 |
| | 4NA-2 (NA-N1, NA-N2, NA-B-Vic and NA-B-Yam [combination 2]) | 18-64 |
| | 4NA-3 (NA-N1, NA-N2, NA-B-Vic and NA-B-Yam [combination 3]) | 18-64 |
| Control group | Flu D-QIV* | 18-64 |

HA: Hemagglutinin; NA: Neuraminidase; H1: Influenza A virus subtype H1; H3: Influenza A virus subtype H3; B-Vic: B Victoria lineage; B-Yam: B-Yamagata lineage; mono: monovalent.
*Recommended composition of Flu D-QIV for use in the 2022-2023 NH season: an A/Victoria/2570/2019 (H1N1)pdm09-like virus; an A/Darwin/9/2021 (H3N2)-like virus; a B/Austria/1359417/2021 (B/Victoria lineage)-like virus; and a B/Phuket/3073/2013 (B/Yamagata lineage)-like virus.

### Segment 2:

Participants aged 18-64 and 65-85 years.
10 groups enrolled in parallel (4 mRNA groups and 1 control group for each age range), detailed in Table 7.
Approximate number of participants: 240 (24 participants per group).
Strains included in study interventions will be based on WHO recommendation for influenza virus vaccine composition for NH 2022-2023.

Each mRNA-coding sequence used in this phase will be manufactured as a separate lot and will be provided to the site staff/pharmacist to be combined at “bedside” before administration to each participant.

TABLE 7
List of study interventions for Phase 1, segment 2

| | | Age |
|---|---|---|
| | | Min-Max |
| Phase 1 - Segment 2 | Study interventions | Years |
| mRNA groups | 4HA/4NA-1* (HA-H1, HA-H3, HA-B-Vic, HA-B-Yam, NA-N1, NA- | 18-64 |
| (Flu mRNA-S2-1-YA to | N2, NA-B-Vic and NA-B-Yam [Combination 1]) | |
| Flu mRNA-S2-4-YA) | 4HA/4NA-2 (HA-H1, HA-H3, HA-B-Vic, HA-B-Yam, NA-N1, NA-N2, | 18-64 |
| | NA-B-Vic and NA-B-Yam [Combination 2]) | |
| | 4HA/4NA-3 (HA-H1, HA-H3, HA-B-Vic, HA-B-Yam, NA-N1, NA-N2, | 18-64 |
| | NA-B-Vic and NA-B-Yam [Combination 3]) | |
| | 4HA/4NA-4 (HA-H1, HA-H3, HA-B-Vic, HA-B-Yam, NA-N1, NA-N2, | 18-64 |
| | NA-B-Vic and NA-B-Yam [Combination 4]) | |
| Control group | Flu D-QIV** | 18-64 |
| mRNA groups | 4HA/4NA-1* (HA-H1, HA-H3, HA-B-Vic, HA-B-Yam, NA-N1, NA- | 65-85 |
| (Flu mRNA-S2-1-A to | N2, NA-B-Vic and NA-B-Yam [Combination 1]) | |
| Flu mRNA-S2-4-YA) | 4HA/4NA-2 (HA-H1, HA-H3, HA-B-Vic, HA-B-Yam, NA-N1, NA-N2, | 65-85 |
| | NA-B-Vic and NA-B-Yam [Combination 2]) | |
| | 4HA/4NA-3 (HA-H1, HA-H3, HA-B-Vic, HA-B-Yam, NA-N1, NA-N2, | 65-85 |
| | NA-B-Vic and NA-B-Yam [Combination 4]) | |
| | 4HA/4NA-4 (HA-H1, HA-H3, HA-B-Vic, HA-B-Yam, NA-N1, NA-N2, | 65-85 |
| | NA-B-Vic and NA-B-Yam [Combination 4]) | |
| Control group | Flu D-QIV** | 65-85 |

YA: Younger Adult; HA: Hemagglutinin; NA: Neuraminidase; H1: Influenza A virus subtype H1; H3: Influenza A virus subtype H3; B-Vic: B Victoria lineage; B-Yam: B-Yamagata lineage; mono: monovalent.
*Called “base-case” which is an eight-component combination, selected based on the immunogenicity/reactogenicity data obtained from Phase 1 - Segment 1 of the study, refer to Section Error! Reference source not found. for additional information.
**Recommended composition of Flu D-QIV for use in the 2022-2023 NH season: an A/Victoria/2570/2019 (H1N1)pdm09-like virus; an A/Darwin/9/2021 (H3N2)-like virus; a B/Austria/1359417/2021 (B/Victoria lineage)-like virus; and a B/Phuket/3073/2013 (B/Yamagata lineage)-like virus.

### Phase 2:

Participants aged 18-64 and 65-85 years
Observer-blind
4 groups enrolled in parallel (1 mRNA group and 1 control group for each age range), Table 8.
Approximate number of participants: 800 (200 participants per group)
Individuals who received any of study investigational vaccines 180 days prior to enrollment (e.g. from Phase 1-Segment 1), may be enrolled again for Phase 2. In this case, they will have to re-consent for the participation in the study and will be re-randomized.
Strains included in study interventions will be based on WHO recommendation for influenza virus vaccine composition for NH 2023-2024.

TABLE 8
List of study interventions for Phase 2

| | | Age |
|---|---|---|
| | | Min-Max |
| Phase 2 | Study interventions | Years |
| Flu mRNA- Selected | Selected formulation from | 18-64 |
| formulation from Phase | Phase 1 - Segment 2 | |
| 1 - Segment 2-YA | | |
| Control group | Flu D-QIV* | 18-64 |
| Flu mRNA- Selected | Selected formulation from | 65-85 |
| formulation from Phase | Phase 1 - Segment 2 | |
| 1 - Segment 2-OA | | |
| Control group | Flu D-QIV* | 65-85 |

YA: Younger Adult; HA: Hemagglutinin; NA: Neuraminidase; H1: Influenza A virus subtype H1; H3: Influenza A virus subtype H3; B-Vic: B Victoria lineage; B-Yam: B-Yamagata lineage; mono: monovalent.
*Composition of Flu D-QIV will be based on the WHO recommendation for influenza virus vaccine for use in the 2023-2024 NH season.

The vaccine candidate is based on modified nucleotides (i.e. N1-methyl-pseudouridine [1mψU]).

### Dose Selection for Phase 1-Segment 1:

Two monovalent HA-H1 and HA-B-Victoria (HA-B-Vic)-coding mRNA sequences, 5 combinations of 4 HA-coding mRNA sequences and 3 combinations of 4 NA-coding mRNA sequences are planned to be evaluated against an active comparator, in parallel groups in this segment of the study.

The combinations of the 4 HA-coding mRNA sequences and the combinations of the 4 NA-coding mRNA sequences will differ by the proportions between the mRNA sequences and the total mRNA dose.

TABLE 9
Example table of dose levels to be assessed in Phase
1 - Segment 1 and 2 and in Phase 2 for illustration.

| | | | | | | | | | | |
|---|---|---|---|---|---|---|---|---|---|---|
| | Age | | | | | HA- | | | | NA- |
| | Min- | Total | HA- | HA- | HA- | B- | NA- | NA- | NA- | B- |
| | Max | dose | H1 | H3 | B-Vic | Yam | N1 | N2 | B-Vic | Yam |
| Study interventions | years | μg | μg | μg | μg | μg | μg | μg | μg | μg |

Example for Phase 1 - Segment 1

| | | | | | | | | | | |
|---|---|---|---|---|---|---|---|---|---|---|
| HA-H1-mono | 18-64 | 6 | 6 | | | | | | | |
| HA-B-Vic-mono | 18-64 | 6 | | | 6 | | | | | |
| HA-H1, HA-H3, HA-B- | 18-64 | 12 | 3 | 3 | 3 | 3 | | | | |
| Vic and HA-B-Yam | | | | | | | | | | |
| [combination 1] | | | | | | | | | | |
| HA-H1, HA-H3, HA-B- | 18-64 | 18 | 3 | 3 | 6 | 6 | | | | |
| Vic, HA-B-Yam | | | | | | | | | | |
| [combination 2] | | | | | | | | | | |
| HA-H1, HA-H3, HA-B- | 18-64 | 24 | 3 | 3 | 9 | 9 | | | | |
| Vic, HA-B-Yam | | | | | | | | | | |
| [combination 3] | | | | | | | | | | |
| HA-H1, HA-H3, HA-B- | 18-64 | 24 | 6 | 6 | 6 | 6 | | | | |
| Vic, HA-B-Yam | | | | | | | | | | |
| [combination 4] | | | | | | | | | | |
| HA-H1, HA-H3, HA-B- | 18-64 | 48 | 6 | 6 | 18 | 18 | | | | |
| Vic, HA-B-Yam | | | | | | | | | | |
| [combination 5] | | | | | | | | | | |
| NA-N1, NA-N2, NA-B- | 18-64 | 12 | | | | | 3 | 3 | 3 | 3 |
| Vic and NA-B-Yam | | | | | | | | | | |
| [combination 1] | | | | | | | | | | |
| NA-N1, NA-N2, NA-B- | 18-64 | 24 | | | | | 6 | 6 | 6 | 6 |
| Vic and NA-B-Yam | | | | | | | | | | |
| [combination 3] | | | | | | | | | | |
| Flu D-QIV | 18-64 | 60 | 15 | 15 | 15 | 15 | | | | |

Example 1 for Phase 1 - Segment 2

| | | | | | | | | | | |
|---|---|---|---|---|---|---|---|---|---|---|
| HA-H1, HA-H3, HA-B- | 18-64 | 24 | 3 | 3 | 3 | 3 | 3 | 3 | 3 | 3 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 1) | | | | | | | | | | |
| HA-H1, HA-H3, HA-B- | 18-64 | 36 | 6 | 6 | 6 | 6 | 3 | 3 | 3 | 3 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 2) | | | | | | | | | | |
| HA-H1, HA-H3, HA-B- | 18-64 | 48 | 6 | 6 | 6 | 6 | 6 | 6 | 6 | 6 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 3) | | | | | | | | | | |
| HA-H1, HA-H3, HA-B- | 18-64 | 16 | 2 | 2 | 2 | 2 | 2 | 2 | 2 | 2 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 4) | | | | | | | | | | |
| HA-H1, HA-H3, HA-B- | 65-85 | 24 | 3 | 3 | 3 | 3 | 3 | 3 | 3 | 3 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 1) | | | | | | | | | | |
| HA-H1, HA-H3, HA-B- | 65-85 | 36 | 6 | 6 | 6 | 6 | 3 | 3 | 3 | 3 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 2) | | | | | | | | | | |
| HA-H1, HA-H3, HA-B- | 65-85 | 48 | 6 | 6 | 6 | 6 | 6 | 6 | 6 | 6 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 3) | | | | | | | | | | |
| HA-H1, HA-H3, HA-B- | 65-85 | 16 | 2 | 2 | 2 | 2 | 2 | 2 | 2 | 2 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 4) | | | | | | | | | | |
| Flu D-QIV | 18-64 | 60 | 15 | 15 | 15 | 15 | | | | |
| | 65-85 | | | | | | | | | |

Example 2 for Phase 1 - Segment 2

| | | | | | | | | | | |
|---|---|---|---|---|---|---|---|---|---|---|
| HA-H1, HA-H3, HA-B- | 18-64 | 72 | 6 | 6 | 18 | 18 | 6 | 6 | 6 | 6 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 1) | | | | | | | | | | |
| HA-H1, HA-H3, HA-B- | 18-64 | 60 | 6 | 6 | 18 | 18 | 3 | 3 | 3 | 3 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 2) | | | | | | | | | | |
| HA-H1, HA-H3, HA-B- | 18-64 | 48 | 3 | 3 | 9 | 9 | 6 | 6 | 6 | 6 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 3) | | | | | | | | | | |
| HA-H1, HA-H3, HA-B- | 18-64 | 36 | 3 | 3 | 9 | 9 | 3 | 3 | 3 | 3 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 4) | | | | | | | | | | |
| HA-H1, HA-H3, HA-B- | 65-85 | 72 | 6 | 6 | 18 | 18 | 6 | 6 | 6 | 6 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 1) | | | | | | | | | | |
| HA-H1, HA-H3, HA-B- | 65-85 | 60 | 6 | 6 | 18 | 18 | 3 | 3 | 3 | 3 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 2) | | | | | | | | | | |
| HA-H1, HA-H3, HA-B- | 65-85 | 48 | 3 | 3 | 9 | 9 | 6 | 6 | 6 | 6 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 3) | | | | | | | | | | |
| HA-H1, HA-H3, HA-B- | 65-85 | 36 | 3 | 3 | 9 | 9 | 3 | 3 | 3 | 3 |
| Vic, HA-B-Yam, NA-N1, | | | | | | | | | | |
| NA-N2, NA-B-Vic and | | | | | | | | | | |
| NA-B-Yam | | | | | | | | | | |
| (Combination 4) | | | | | | | | | | |
| Flu D-QIV | 18-64 | 60 | 15 | 15 | 15 | 15 | | | | |
| | 65-85 | | | | | | | | | |

HA: Hemagglutinin; NA: Neuraminidase; H1: Influenza A virus subtype H1; H3: Influenza A virus subtype H3; B-Vic: B Victoria lineage; B-Yam: B-Yamagata lineage.

Two examples for Phase 1-Segment 2 dose levels representing a scenario for the lowest dose levels and a scenario for the highest dose levels to be assessed are also included for illustrative purposes (Table 9). The 2 scenarios will allow for final dose levels within the dose ranges below:
For total dose of 8 components: ranging from 16 μg (lowest total dose) to 72 μg (highest total dose).
For each HA mRNA-coding sequence: ranging from 2 μg (lowest individual dose) to 18 μg (highest individual dose).
For each NA mRNA-coding sequence: ranging from 2 μg (lowest individual dose) to 6 μg (highest individual dose).

### Dose Selection for Phase 1-Segment 2:

Up to 4 provisional combinations of 4HA/4NA-coding mRNA sequences (8 components in total) will be evaluated in both YAs and OAs against an active comparator, in parallel groups in this segment of the study.

### Dose Selection for Phase 2:

One combination of 4HA/4NA-coding mRNA sequences (8 components in total) selected from Phase 1-Segment 2 will be evaluated in both YAs and OAs against an active comparator, in parallel groups in this phase of the study.

### Example 4-Phase 1/2 Quadrivalent Influenza Vaccination Trail-Modified mRNA (Scenario 2)

This study is a Phase 1/2, randomized, dose-finding/dose-confirmation study to evaluate the reactogenicity, safety and immunogenicity of mRNA-based multivalent seasonal influenza vaccine candidates administered in healthy younger (YA) and older (OA) adults.

The study consists of an exploratory dose-finding part (Phase 1) and dose-confirmation part (Phase 2). All study interventions will be based on the RNACTIVE technology platform, which uses sequence-optimized, capped, polyadenylated synthetic mRNA formulated with LNPs and modified nucleotides. The data generated in Phase 1 will be used to determine the study interventions in Phase 2. The investigational study interventions in Phase 1 and Phase 2 will be compared to split-inactivated licensed influenza vaccines.

1. Study design

### Phase 1

Participants aged 18-50 years.
Up to 13 groups enrolled in parallel (up to 11 mRNA groups and 1 control group), detailed in Table 10. Specific mRNA groups may not be started or lower dose levels may be used in the specified mRNA groups.
Approximate number of participants: Up to 312 (24 participants per group).
Blinding: single-blind.
Study interventions administered in Phase 1 will be manufactured as separate components and mixed at site by the site staff/pharmacist, before dosing each participant. This approach will only be applicable in the Phase 1 part to enable the option of adjusting the dose levels of the study interventions.
The comparator in Phase 1 is a licensed influenza vaccine approved for use in persons 18 years of age or older.

TABLE 10
Potential study interventions composition (Phase 1)

| | Total | | | HA-B- | HA-B- | | | NA-B- | NA-B- |
|---|---|---|---|---|---|---|---|---|---|
| Study | dose | HA-H1 | HA-H3 | Vic | Yam | NA-N1 | N1-N2 | Vic | Yam |
| groups* | (μg) | (μg) | (μg) | (μg) | (μg) | (μg) | (μg) | (μg) | (μg) |
| 1 | 18 | 18 | | | | | | | |
| 2 | 36 | 9 | 9 | 9 | 9 | | | | |
| 3 | 48 | 12 | 12 | 12 | 12 | | | | |
| 4 | 72 | 18 | 18 | 18 | 18 | | | | |
| 5 | 45 | 9 | 18 | 9 | 9 | | | | |
| 6 | 54 | 9 | 9 | 18 | 18 | | | | |
| 7 | 48 | 9 | 9 | 9 | 9 | 3 | 3 | 3 | 3 |
| 8 | 60 | 9 | 9 | 9 | 9 | 6 | 6 | 6 | 6 |
| 9 | 60 | 12 | 12 | 12 | 12 | 3 | 3 | 3 | 3 |
| 10 | 84 | 12 | 12 | 12 | 12 | 9 | 9 | 9 | 9 |
| 11 | 96 | 18 | 18 | 18 | 18 | 6 | 6 | 6 | 6 |
| 12 | 120 | 18 | 18 | 18 | 18 | 12 | 12 | 12 | 12 |

| | |
|---|---|
| Control | Licensed split-inactivated quadrivalent influenza |
| | vaccine (15 μg of HA per strain) - NH 2022-2023 |

HA: hemagglutinin;
NA: neuraminidase.
H1: H1: H1 hemagglutinin from Influenza A virus subtype H1N1;
H3: H3 hemagglutinin from Influenza A virus subtype H3N2;
N1: N1 neuraminidase from Influenza A virus subtype H1N1;
N2: N2 neuraminidase from Influenza A virus subtype H3N2;
B-Vic: B Victoria lineage;
B-Yam: B-Yamagata lineage
*Strains used to design study interventions are based on WHO recommendation for influenza virus vaccine composition for the 2022-2023 NH season.

Phase 1 will assess the reactogenicity, safety and immunogenicity of up to 11 different formulations and 1 active control to enable dose optimization of the investigational study intervention components (mRNAs encoding a single HA or NA antigen). Data from the prior study (see Example 1) showed that an equimolar ratio of vaccine components may be suboptimal as this induced highly variable immune responses against subtype A and B lineage strains and in particular suboptimal responses against B lineages. Hence, the overall aim for the Phase 1 part is to optimize the dose for each mRNA encoding a single HA or NA antigen to achieve an appropriate immune response against both subtype A and B lineage strains.

### Phase 2

Participants aged 18-64 and 65-85 years.
Up to 8 groups enrolled in parallel (up to 3 mRNA groups and 1 control group for each age range), Table 11. Dependent upon immunogenicity data generated in the Phase 1 part of the study, not all mRNA groups may be started.
Approximate number of participants: Up to 1200 (Up to 150 participants per group). Dependent upon immunogenicity data generated in the Phase 1 part of the study, the number of participants per group may be reduced.
Blinding: observer-blind.
Study interventions in Phase 2 will be provided to the site staff/pharmacist in vials containing the 8-component formulations, that will be reconstituted with diluent according to the instructions detailed in Pharmacy Manual, prior to dosing the participants.
To allow for age-appropriate comparisons, in Phase 2, the comparator for YAs will be a licensed influenza vaccine approved for use in persons 18 and older, and for OAs a licensed influenza vaccine approved for use in persons 65 years of age and older.
The total dose and individual dose level of the mRNAs encoding a single HA antigen or a single NA antigen in the 8-component formulations will be determined at the time of the first scheduled interim analysis (on reactogenicity, safety and immunogenicity data from all participants in Phase 1, up to Day 29 post-dosing).

TABLE 11
List of study interventions for Phase 2

| Study groups | Study interventions | Age min-max (years) |
|---|---|---|
| Formulation 1 based on Phase 1 | Selected formulation 1 based on | 18-64 |
| data - YA | Phase 1 | |
| Formulation 2 based on Phase 1 | Selected formulation 2 based on | |
| data - YA | Phase 1 | |
| Formulation 3 based on Phase 1 | Selected formulation 3 based on | |
| data - YA | Phase 1 | |
| Control | Licensed split-inactivated | |
| | quadrivalent influenza vaccine | |
| | (15 μg of HA per strain) - NH | |
| | 2023-2024 | |
| Formulation 4 based on Phase 1 | Selected formulation 4 based on | 65-85 |
| data - OA | Phase 1 | |
| Formulation 5 based on Phase 1 | Selected formulation 5 based on | |
| data - OA | Phase 1 | |
| Formulation 6 based on Phase 1 | Selected formulation 6 based on | |
| data - OA | Phase 1 | |
| Control | Licensed split-inactivated | |
| | quadrivalent influenza vaccine | |
| | (60 μg of HA per strain) - NH | |
| | 2023-2024 | |

YA: younger adult; OA: older adult.

Phase 2 will assess the reactogenicity, safety and immunogenicity of up to 3 different 8-component formulations (and 1 active control) in both YA and OAs. The specific aim for Phase 2 is to identify the formulation(s) to be selected for the Phase 3 studies. Given the high unmet need for an improved vaccine in OAs, the Phase 2 part will be conducted in both YAs and OAs to confirm that the selected formulation(s) induces an appropriate immune response in both age groups.

### Example 5-Immunogenicity Study in Mice and in Ferrets of 4-Component, 7-Component, And 8-Component Flu Seasonal mRNA Formulations with Equimolar Proportions Between The mRNA Sequences-Modified and Unmodified mRNA

Mice Studies-4-Component and 7-Component Flu Seasonal mRNA Formulations

In this study, 4-component (4HA) and 7-component (4HA+3NA) seasonal influenza mRNA vaccine formulations containing either unmodified (uridine) or modified (ψ and N1-mψ) nucleosides were investigated in vivo in mice for the induction of innate and adaptive humoral anti-HA and anti-NA immune responses. The tested formulations are based on the RNACTIVE technology platform, which uses sequence-optimized, capped, polyadenylated synthetic mRNA formulated with LNPs. The antigenic composition of the vaccines was based on the WHO recommendation for cell- or recombinant based quadrivalent influenza vaccines (QIV) for the Northern hemisphere (NH) influenza season 2021-22 and contains constructs encoding for HA and NA of influenza viruses A/Wisconsin/588/2019 (H1N1pdm09), A/Cambodia/e0826360/2020 (H3N2), B/Washington/02/2019 and HA of influenza virus B/Phuket/3073/2013.

Female Balb/c mice were vaccinated IM with 0.56 μg or 2.84 μg of the 4-component (4HA; unmodified, ψ and N1-mψ) and 1 μg or 2.84 μg of 7-component (4HA+3NA; unmodified, ψ and N1-mψ) mRNA-LNP vaccines administered on day 0 and 21. Control animals received either physiological saline (NaCl) or one tenth of the human dose of the licensed QIVs FLUARIX Tetra NH21-22 or FLUZONE HD NH21-22 administered IM on day 0 and 21.

The HI assay was used as primary serology readout to assess functional anti-HA antibody responses induced by the tested mRNA vaccines. The HI assay was performed on individual serum samples obtained at two weeks post second vaccination. Before the assay, to eliminate non-specific inhibitors of hemagglutination, serum samples were treated with receptor destroying enzyme (RDE) at 37° C. for 16-20 h, followed by heat inactivation at 56° C. for 30 min and pre-adsorption to red blood cells (RBCs) at 4° C. for 30-45 min. Chicken RBCs were used with serum samples intended for influenza H1N1 and both influenza B HI assays and turkey RBCs were used with serum samples intended for influenza H3N2 HI assays. To determine the viral titers the following influenza virus antigens were used: Influenza antigen A/Victoria/2570/2019 (IVR-215) (H1N1pdm09) and A/Cambodia/e0826360/2020 (IVR-224) (H3N2) (both influenza A viruses were purchased from NIBSC as formalin-inactivated, partially purified viruses), B/Phuket/3073/2013 and B/Washington/02/2019 (both influenza B viruses were provided by GSK as detergent-split vaccine antigens). For each HI assay positive and negative controls, treated in the same way as the samples, were included on one assay plate. Serum samples from NaCl buffer control mice served as negative control. Sheep serum anti-influenza virus HA A/Victoria/2570/2019 (H1N1pdm09), A/Cambodia/e0826360/2020 (H3N2), B/Phuket/3073/2013 and B/Washington/02/2019 (all from NIBSC) were used as positive controls in the respective HI assay. For the determination of viral and HI titers 96-well non-treated polypropylene V-bottom plates were used. Influenza HA antigen diluted to 4 HA units/25 μl was added to pre-diluted RDE-treated serum samples and incubated at RT for 45-60 min, followed by an incubation for 45-60 min with 50 μl 0.5% RBCs. Each sample was run in duplicate. The samples in each well were then read visually as either agglutinated in which RBCs formed a pattern whereas nonagglutinated RBCs formed a teardrop in the center of the V-bottom. The HI titer was defined as the reciprocal of the last dilution that showed agglutination inhibition.

The primary serology readout to assess the immunogenicity of the NA-encoding mRNA vaccine components was the ELLA, which allows the measurement of antibodies inhibiting the enzymatic activity of the NA. In brief, 96-well plates were coated with the carbohydrate fetuin, which was then exposed to NA through NA bearing single cycle pseudoviruses (PV) used as a surrogate virus. The lentiviral PVs express the HA of avian influenza H11 (derived influenza A/duck/Memphis/546/1974 (H11N9) and NA of influenza A/Wisconsin/588/2019 (H1N1pdm09), A/Cambodia/e0826360/2020 (H3N2), and B/Washington/02/2019. The NA enzyme cleaves terminal sialic acid residues from the fetuin, exposing galactose that is then bound by the peanut agglutinin, conjugated to horseradish peroxidase (PNA-HRPO). This reagent then forms the basis for colorimetric reading of NA activity. This activity can be inhibited by antibodies present in the serum of vaccinated mice. To measure the NA inhibition (NI) titers, each serum sample was heat treated at 56° C. for 45 min and then serially diluted in PBS-BSA. 50 μl of each dilution was added to duplicate wells of a fetuin-coated plate. An equal volume (50 μl) of the virus dilution was added to all serumcontaining wells in addition to at least 4 wells containing diluent without serum that served as a positive control (virus only). At least 4 wells were retained as a background control (PBS only). The plates were incubated for 16-18 h at 37° C. As described for the virus titration, the plates were washed and PNA-HRPO was added to all wells. After 2 h of incubation, the plates were washed, and an o-phenylenediamine dihydrochloride (OPD) (Sigma, St. Louis, MO, USA) substrate was added. The reaction was stopped by addition of chloric acid, and the absorbance was read at 490 nm. Mean absorbance of the background (Abkg) was subtracted from the test wells and positive control (Apos) wells. The percent NA activity was calculated by dividing the mean absorbance of duplicate test wells (Atest) by the mean absorbance of virus only wells and multiplied by 100, i.e. (Atest−Abkg)/(Apos−Abkg)×100. To determine percent NA inhibition, the percent activity was subtracted from 100. The NI titers were defined as the reciprocal of the last dilution that resulted in at least 50% inhibition.

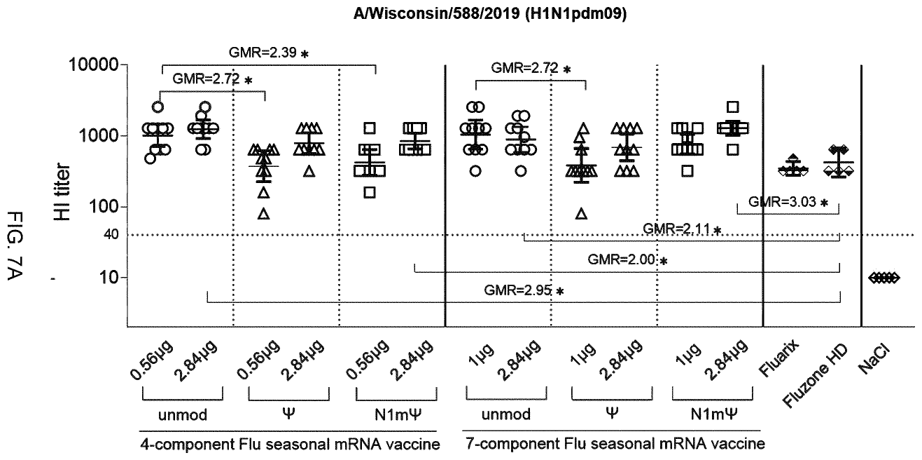

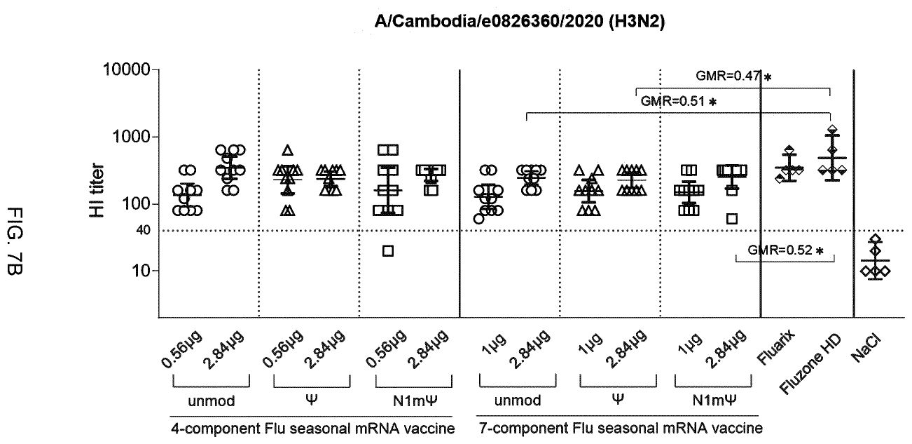

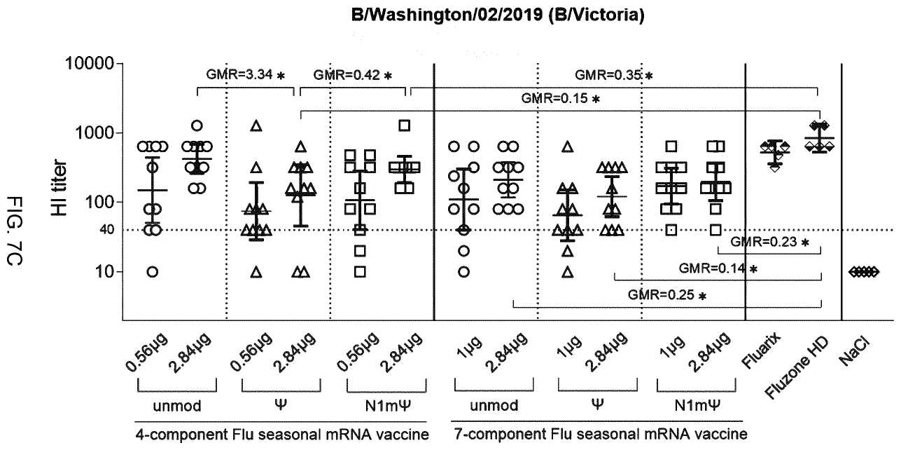

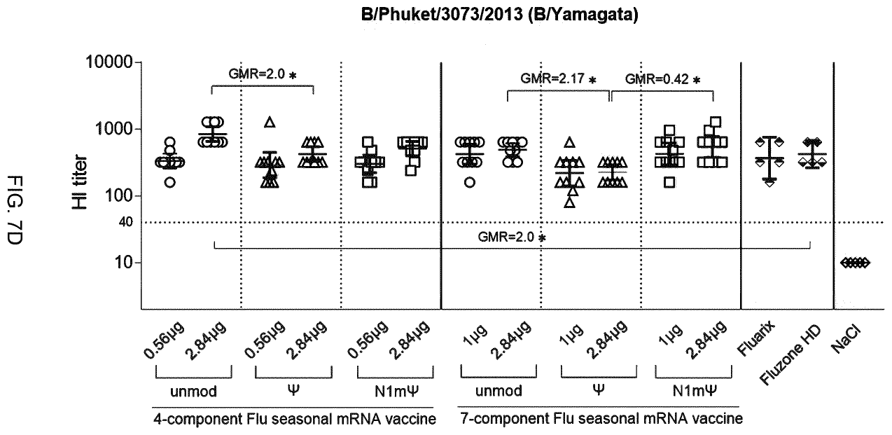

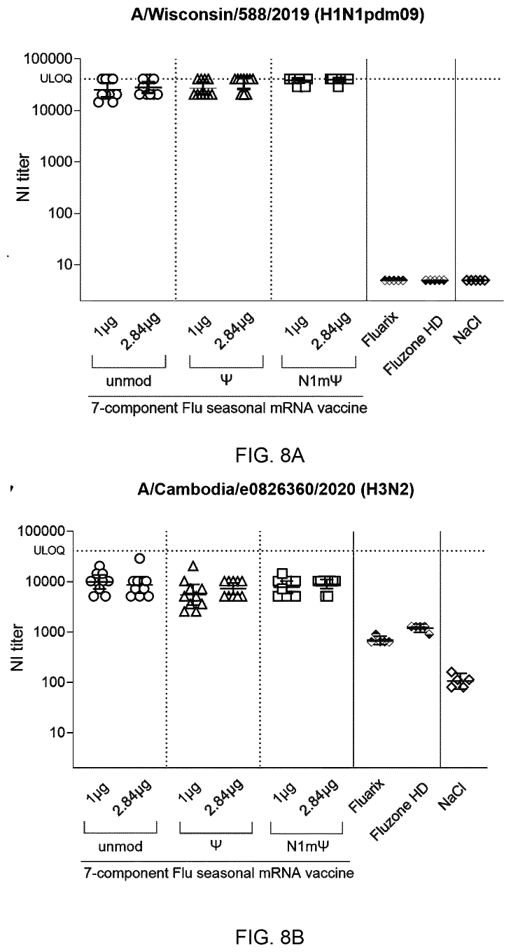

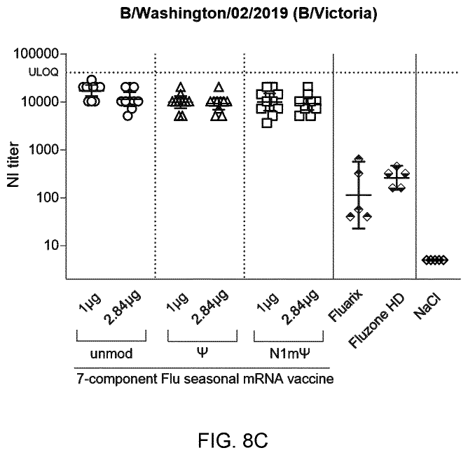

The induction of functional antibodies against all antigenic components of the 4- and 7-component mRNA vaccines was analyzed using the HI assay for the anti-HA responses and the ELLA for the anti-NA responses. HI titers against influenza A/Wisconsin/588/2019 (H1N1pdm09) (H1N1pdm09), B/Washington/02/2019 and B/Phuket/3073/2013 and neuraminidase inhibition (NI) titers against the NA of A/Wisconsin/588/2019 (H1N1pdm09) and B/Washington/02/2019 were determined in the serum collected two weeks post second immunization. Despite a reduced innate immune system activation by vaccine formulations containing modified nucleosides (ψ and N1-mw) (data not shown), the induction of HI responses (FIG. 7) against the four HA components and NI responses (FIG. 8) against the three NA components of the mRNA vaccines was retained.

Slightly lower HI titers against influenza A/Wisconsin/588/2019 (H1N1pdm09) were detected for the 4- or 7-component ψ mRNA vaccines compared to the respective mRNA vaccines containing unmodified nucleosides (FIG. 7A). Similar trend with lower HI titers was detected for influenza B/Phuket/3073/2013 for the 4- or 7-component mRNA vaccines containing ψ compared the respective mRNA vaccines containing unmodified nucleosides (FIG. 7D). Lower influenza B/Washington/02/2019-specific HI titers were detected at high dose for the 4-component ψ mRNA vaccine compared to the mRNA vaccine containing unmodified nucleosides (FIG. 7C). No difference between A/Cambodia/e0826360/2020-specific HI titers of modified and unmodified 4- and 7-component mRNA vaccines was observed (FIG. 7B).

FIG. 7 further shows lower HI titers detected for influenza A/Cambodia/e0826360/2020 (FIG. 7B) for the 4- or 7-component mRNA vaccines containing N1-mw compared the A/Wisconsin/588/2019 (H1N1pdm09)-specific HI titers (FIG. 7A).

Comparison to the QIV FLUZONE HD was performed for the groups that received the mRNA vaccines in the high dose (2.84 μg). Mice immunized with the mRNA vaccines showed higher (A/Wisconsin/588/2019) or mostly similar (B/Phuket/3073/2013) HI titers compared to FLUZONE HD (FIG. 7A and D, respectively) while the HA-specific responses induced by the mRNA vaccines indicated a pattern toward lower HI responses against influenza A/Cambodia/e0826360/2020 (FIG. 7B) and B/Washington/02/2019 compared FLUZONE HD immunized groups (FIG. 7C).

As shown in FIG. 8A-C, compared to the licensed QIVs, the 7-component unmodified, w and N1-my mRNA vaccines induced substantially superior NI responses against all three NA components of the mRNA vaccines. FIG. 8 further shows lower NI titers detected for influenza A/Cambodia/e0826360/2020 (FIG. 8B) compared the A/Wisconsin/588/2019 (H1N1pdm09)-specific NI titers (FIG. 8A).

Ferrets Studies—4-Component and 8-Component Flu Seasonal mRNA Formulations

In the present study 4- and 8-component Flu Seasonal N1mw mRNA vaccines were investigated for the induction of HI, microneutralization (MN) and NI titers upon two-dose i.m. immunization of naive ferrets. The antigenic composition of the vaccines was based on the WHO recommendation for cell- or recombinant based QIVs for the Northern hemisphere (NH) influenza season 2022-23 and contained constructs encoding for HA and NA of influenza viruses A/Wisconsin/588/2019 (H1N1pdm09), A/Darwin/6/2021 (H3N2), B/Austria/1359417/2021 and B/Phuket/3073/2013.

Female ferrets were vaccinated twice IM on Day 0 and Day 28 with 4-component and 8-component Flu seasonal N1mw mRNA vaccines as presented in Table 12. As controls, two groups were immunized twice IM on Day 0 and Day 28 with the QIVs FLUARIX Tetra NH22-23. Animals in the negative control group were injected with physiological saline (NaCl). Serum samples were collected four weeks post second immunization (on day 55) and functional antibody responses against all components of the Flu Seasonal mRNA vaccine formulations were analyzed using the HI assay for the HA components and the ELLA assay for the NA components. In addition, neutralizing antibody responses were measured using the microneutralization (MN) assay.

TABLE 12
4-component and 8-component Flu seasonal
N1mψ mRNA tested formulations

| | | | Total | Dose per |
|---|---|---|---|---|
| | Species/Gender/ | | dose | antigen |
| Group | Number | Vaccine | (μg) | (μg) |
| 1 | Ferrets | 8-component | 25 | 3.125 |
| 2 | Female | (4HA + 4NA) | 50 | 6.25 |
| 3 | N = 6/ | 4-component | 12.5 | 3.125 |
| 4 | group | (4HA) | 25 | 6.25 |
| 5 | | FLUARIX NH22-23 | 60 μg HA | 15 μg HA |
| 6 | | 0.9% NaCl buffer | N/A | |

The HI assay was used as serology readout to assess functional anti-HA antibody responses induced by the tested Flu Seasonal N1mw mRNA vaccines. The HI assay was performed on individual serum samples obtained four weeks post second vaccination. Before the assay, to eliminate non-specific inhibitors of hemagglutination, serum samples were treated with receptor destroying enzyme (RDE) at 37° C. for 16-20 h, followed by heat inactivation at 56° C. for 30 min and pre-adsorption to red blood cells (RBCs) (using chicken RBCs with serum samples intended for the influenza H1N1 and both influenza B HI assays or turkey RBCs with serum samples intended for the influenza H3N2 HI assays) at 4° C. for 30-45 min. The following influenza virus antigens were used: influenza A/Victoria/2570/2019 (IVR-215) (H1N1pdm09), A/Darwin/9/2021 (SAN-010) (H3N2), B/Austria/1359417/2021 (all influenza viruses were purchased from NIBSC as formalin-inactivated, partially purified viruses), and B/Phuket/3073/2013 (detergent-split virus from GSK). For each HI assay positive and negative controls, treated in the same way as the samples, were included on one assay plate. Serum samples from mice injected with NaCl buffer served as negative control. Sheep serum anti-HA of (H1N1pdm09), influenza viruses A/Victoria/2570/2019 A/Darwin/9/2021 (H3N2), B/Austria/1359417/2021, B/Phuket/3073/2013 (all from NIBSC) were used as positive controls in the respective HI assay. For the determination of viral and HI titers 96-well non-treated polypropylene V-bottom plates were used. Influenza HA antigen diluted to 4 HA units/25 μl was added to pre-diluted RDE-treated serum samples and incubated at RT for 50 min, followed by an incubation for 40-60 min with 50 μl 0.5% RBCs (chicken RBCs for influenza H1N1pdm09 and both influenza B virus antigens and turkey RBCs for the influenza H3N2 antigen). Each sample was run in duplicate. The samples in each well were then read visually as either agglutinated in which RBCs formed a pattern whereas non-agglutinated RBCs formed a teardrop in the center of the V-bottom. The HI titer was defined as the reciprocal of the last dilution that showed agglutination inhibition.

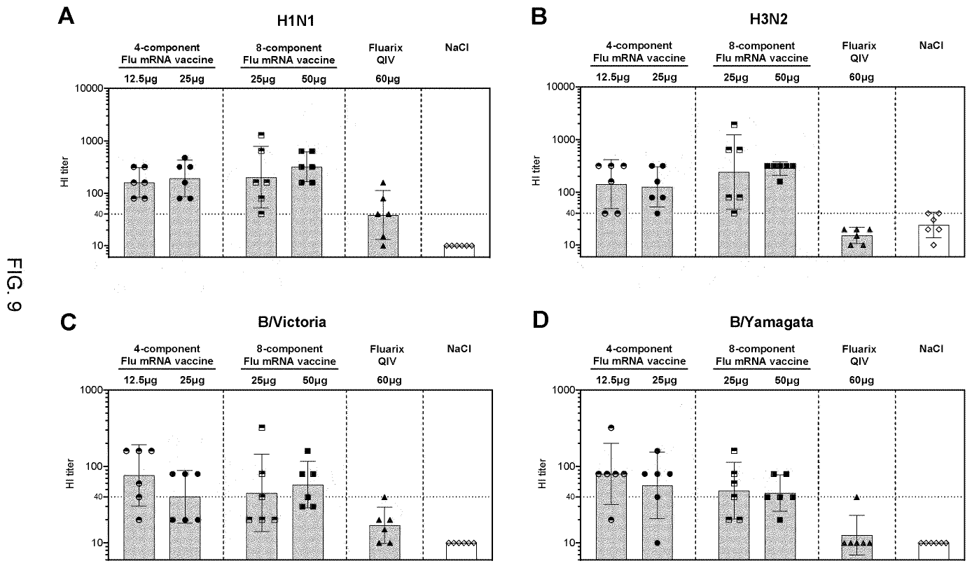

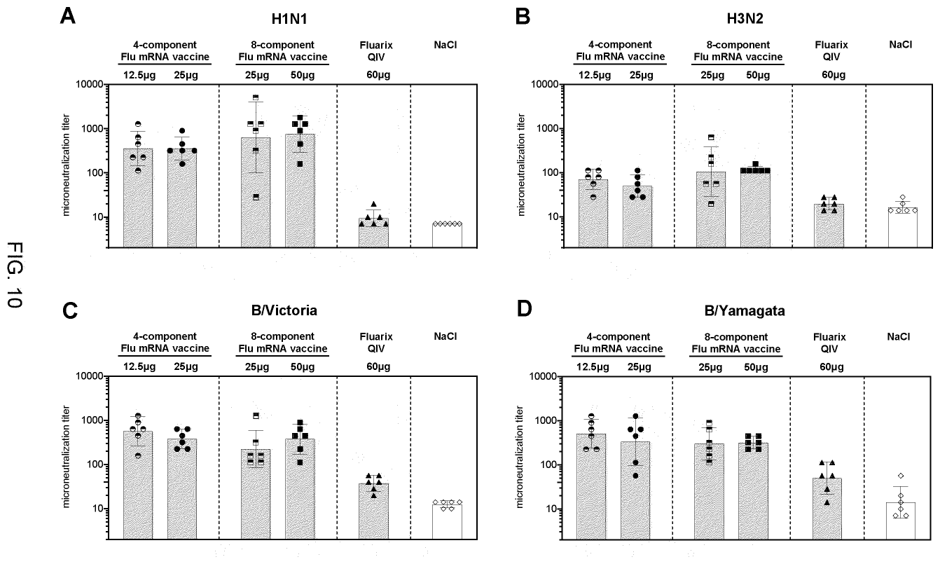

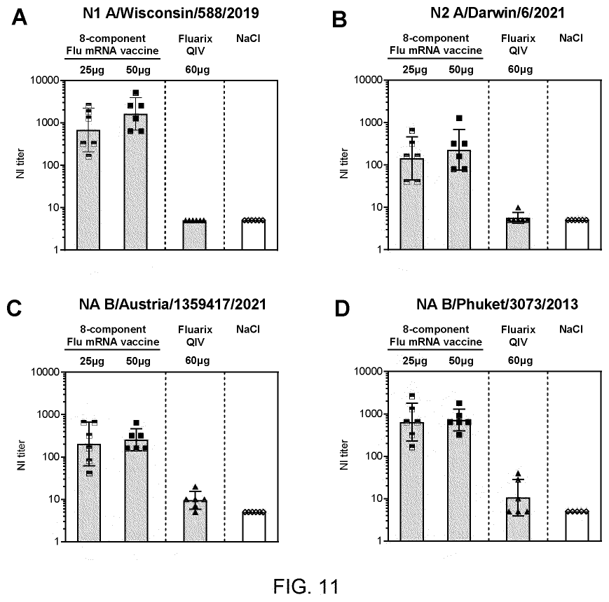

As shown in FIG. 9, the 4-component and 8-component Flu Seasonal N1my mRNA vaccines induced HI responses against all four encoded HA antigens with comparable levels of HI titers measured for both doses of both of the mRNA vaccines. In case of A/Wisconsin/588/2019 (FIG. 9A), A/Darwin/6/2021 (H3N2) (FIG. 9B) and B/Phuket/3073/2013 (FIG. 9D), the HI responses induced by the 4- and 8-component Flu Seasonal N1mw mRNA vaccines were significantly higher than the HI titers induced by FLUARIX Tetra NH22-23. For B/Austria/1359417/2021 (FIG. 9C), 50 μg of the 8-component and 12.5 μg of the 4-component Flu seasonal N1mw mRNA vaccine led to significantly higher HI titers compared to FLUARIX Tetra NH22-23.

The CPE-Based Microneutralization (CPE-Based MN) is a conventional serological method able to detect and titrate influenza virus-specific neutralizing antibodies in animal and human sera. To perform the MN test, where each well of a 96-well plate is examined by using a light-optical microscope to evaluate CPE in the cell monolayer, it is necessary to know the titer of the virus to be used (Tissue Culture Infective Dose 50% (TCID50)). “TCID50” represents the dose of the virus able to induce a cytopathic effect in 50% of the cell culture incubated with the live virus. For the virus titration, generally 8 repetitions of live virus titration are performed and transferred to a cell monolayer seeded the day before the titration. The viral stock is serially diluted (1 log 10 dilution or 0.5 log 10). This titer is used to calculate the dilution factor to have a working viral solution containing 2000 TCID 50/ml (10 3.3) or 200 TCID 50/100 μl (10^{2.3}). To obtain the dilution factor, the virus stock titer is divided by the titer that the working viral solution must have. The assay is performed in a 96-well plate format. It uses live influenza viruses. Serum samples are serially diluted in duplicate in two flat-bottomed 96-well microtiter plates. Virus is added to the serum samples and incubated for 1 hour to allow neutralization of the virus inoculum when neutralizing antibodies are present in the serum. The virus/serum mix is then added to MDCK cells, and the cells are grown for 48-72 hours to allow time for several rounds of full viral life cycle in the presence of antibodies. The incubation time and the temperature at which the plates are incubated before the reading is usually determined in the microneutralization set-up experiments. For example, for influenza B strains the incubation period is 8 days at 33±1° C., whereas for the A strains it is 5 days at 37±1° C. At the end of the incubation period, each well of the 96-well microtiter plate is checked under an optical microscope to assess the presence of local lesions (“CPE”) in the cell lawn. The neutralization titre (Nt) for each serum duplicate is calculated according to the following Spearman-Karber formula. The Nt in this test is defined as the serum dilution by means of which 50% of the wells are protected against a virus-induced cytopathogenic effect (CPE).

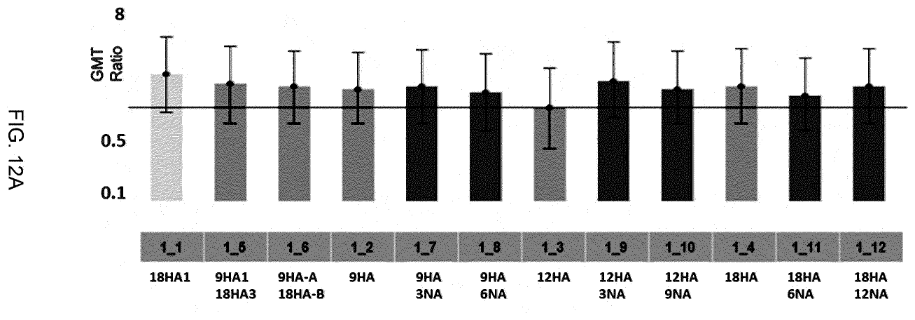

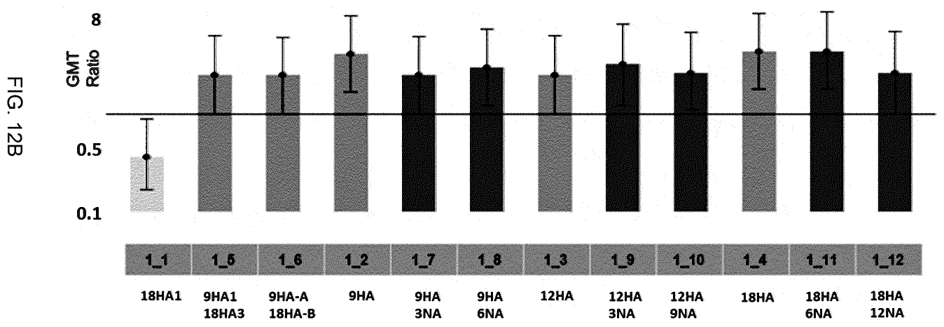

Comparable MN titers were induced between the 4-component and 8-component Flu Seasonal mRNA vaccines at the same dose of the HA antigen-encoding mRNA, suggesting that the addition of the NA antigens did not negatively impact the anti-HA functional antibody responses (FIG. 10A-D).

FIG. 10 further shows lower MN titers detected for influenza A/Darwin/6/2021 (H3N2) (FIG. 10B) for the 4- and 8-component mRNA vaccines compared the A/Wisconsin/588/2019 (H1N1pdm09)-specific MN titers (FIG. 10A). GM for A/Darwin/6/2021 (H3N2)-specific MN titers are only of 107 (25 μg) and 120 (50 μg) with 8-component Flu seasonal N1mw mRNA vaccines. Similarly, GM for A/Darwin/6/2021 (H3N2)-specific MN titers are of 71 (12.5 μg) and 50 (25 μg) with 4-component Flu seasonal N1mw mRNA vaccines. In comparison, GM for A/Wisconsin/588/2019 (H1N1pdm09)-specific MN titers are of 640 (25 μg) and 761 (50 μg) with 8-component Flu seasonal N1mw mRNA vaccines and 359 (12.5 and 25 μg) with 4-component Flu seasonal N1mw mRNA vaccines.

The ELLA (Enzyme-linked Lectin Assay) is a serological method able to detect the presence of antibodies directed against NA of influenza virus to assess the neuraminidase inhibition (NI) antibody titer in serum samples. NA cleaves terminal sialic acids from fetuin, exposing galactose. Peanut agglutinin (PNA) is a lectin with specificity for galactose and therefore the extent of desialylation can be quantified using a PNA-horseradish peroxidase conjugate, followed by addition of a chromogenic peroxidase substrate. The optical density that is measured is proportional to NA activity. This NA activity can be inhibited by antibodies present in the serum of vaccinated animals or humans.

The NA source was 2-fold serially diluted in sample diluent (phosphate buffered saline containing bovine serum albumin and Tween20) and was transferred to a fetuin coated plate, which was then placed in a humidified incubator overnight. The day after incubation, the plate was washed and incubated with a solution of Arachis hypogaea Lectin (PNA) conjugated with Horseradish Peroxidase (HRPO). Then, to the washed plate a chromogenic peroxidase substrate was added to develop a colorimetric reaction, which was stopped by chloric acid. The OD results versus the dilution were plotted in a graph. The optimal dilution is provided at the 90% of the maximum signal.

The serum samples were heat-inactivated for 30 minutes at 56° C., then 2-fold serially diluted in fetuin coated plates, mixed with an equal volume of NA-bearing pseudovirus at optimal dilution and placed in a humidified incubator overnight. For Day 0 and Day 28 samples (groups 1-2 and 5-7), the dilution series ranged from 1:10 up to 1:5120. For Day 56 samples, the dilution series ranged from 1:80 up to 1:40960 for the A/Wisconsin/588/2019 (N1) assay (groups 1-2), and from 1:20 up to 1:10240 for B/Phuket/3037/2013 and B/Austria/1359417 assays (groups 1-2). For Day 56 samples of groups 5-7 the dilution series ranged from 1:10 up to 1:5120. Duplicate runs for each sample were performed in the same plate. Each plate contained wells for background (negative signal) and wells dedicated to NA-bearing pseudovirus (which provided maximum signal, also called “virus control wells”). The day after incubation, the washed plates were incubated with PNA-HRPO solution. Then, to the washed plates a chromogenic peroxidase substrate was added to develop colorimetric reaction, which was stopped by addition of chloric acid. The OD results were used to determine the end-point titer, which is calculated by a cut-off for each plate expressed as 50% mean OD virus control wells. The Neuraminidase Inhibition titer (NIt) of each sample run corresponded to the reciprocal highest sample dilution that resulted in at least 50% inhibition of the maximum signal (OD under cut-off). If no inhibition was observed (Nlt<10) an arbitrary value of 5 was reported. For those samples tested using a different starting dilution higher than 1:10 (e.g. 1:20 or 1:80), the absence of inhibition was indicated taking into consideration this initial starting dilution.

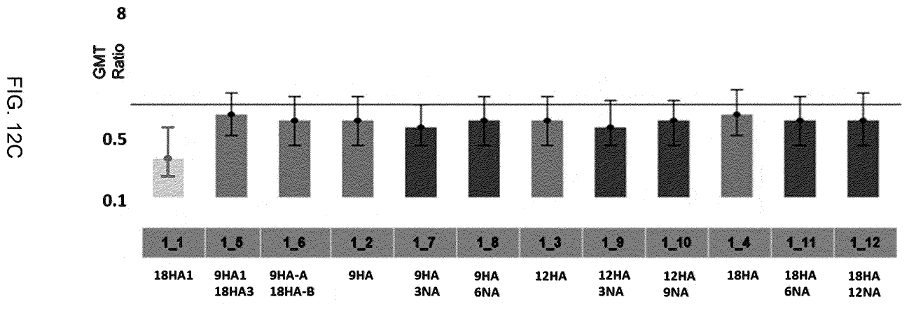

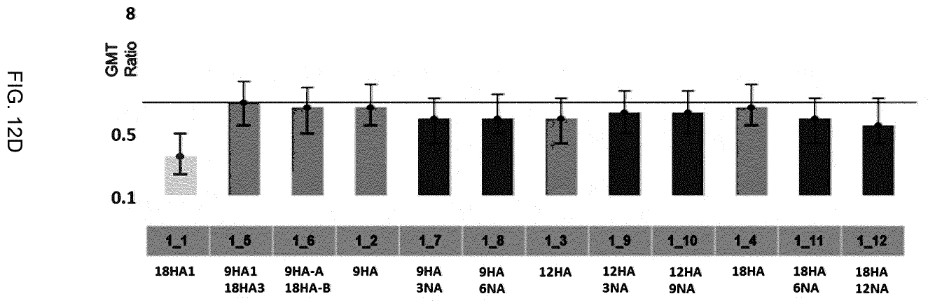

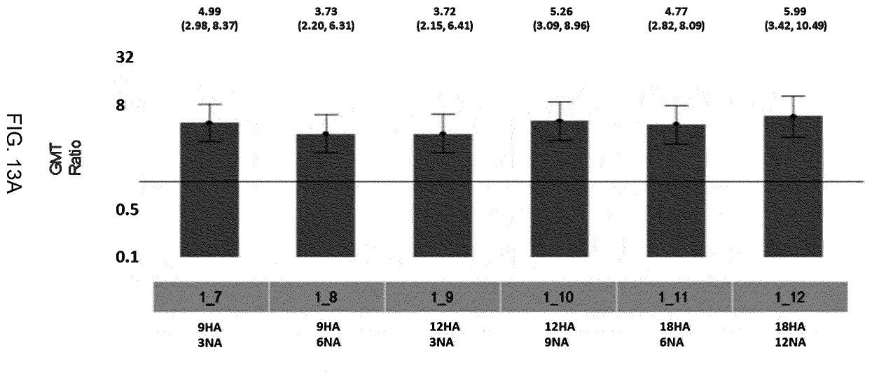

As compared to FLUARIX Tetra, the 8-component Flu Seasonal mRNA vaccines induced substantially superior NI responses against all four NA components of the mRNA vaccines, confirming the benefits of incorporating a NA antigen component to a quadrivalent Flu Seasonal mRNA vaccine (FIG. 11A-D).

FIG. 11 further shows lower NI titers detected for influenza A/Darwin/6/2021 (H3N2) (FIG. 11B) for the 8-component mRNA vaccines compared to the A/Wisconsin/588/2019 (H1N1pdm09)-specific NI titers (FIG. 11A). GM for A/Darwin/6/2021 (H3N2)-specific NI titers are only of 143 (25 μg) and 226 (50 μg) with 8-component Flu seasonal N1mw mRNA vaccines. In comparison, GM for A/Wisconsin/588/2019 (H1N1pdm09)-specific NI titers are of 1613 (50 μg) with 8-component Flu seasonal N1mw mRNA vaccines.

### Example 6-Results of a Phase 1 Study to Evaluate the Reactogenicity, Safety and Immunogenicity of mRNA-Based Multivalent Seasonal Influenza Vaccine Candidates Administered in Healthy Younger and Older Adults

A Phase 1 study has been conducted according to the study design of Example 4. The immunogenicity, reactogenicity, and safety of 12 different formulations of the mRNA-based multivalent seasonal influenza vaccine (composed of either 1 HA-, 4 HA-, or 4 HA-and 4 NA-encoding mRNAs) and a licensed influenza vaccine (FLU D-QIV) were investigated in healthy adults (Table 13). 270 participants 18 to 50 years of age have been exposed to the mRNA-based multivalent seasonal influenza vaccine. There have been no withdrawals from the study.

Data generated show no safety signal across all tested dose levels and formulations of the mRNA-based multivalent seasonal influenza vaccine candidate. The reactogenicity profile is similar to what has been observed with other mRNA-based vaccines, with most solicited events, including Grade 3 events, being short-lived and occurring within a few days after vaccination.

No vaccine-related SAEs, MAAEs, or AESIs, including plMDs were reported in any dose group. No AEs led to withdrawal from the study.

Immunogenicity of the mRNA-based multivalent seasonal influenza vaccine compositions were assessed in parallel with a licensed seasonal influenza vaccine (FLU D-QIV). Overall, mRNA-based multivalent seasonal influenza vaccine compositions were immunogenic in all study groups and elicited an immune response across the 4 strains for both antigens. Furthermore, the data suggest no immunological interference with the addition of additional components.

Compared to active control, the results suggest that the mRNA-based multivalent seasonal influenza vaccine elicit a higher HI response against influenza A strains. Importantly, the results also suggest that the mRNA-based multivalent seasonal influenza vaccine elicit a higher NI response compared to the licensed control, which may play an important role in the overall effectiveness of this vaccine candidate.

TABLE 13
mRNA doses in vaccine compositions administered in different groups of Phase 1

| | Total | | | | HA-B- | HA-B- | | | NA-B- | NA-B- |
|---|---|---|---|---|---|---|---|---|---|---|
| Study | dose | Abbreviated | HA-H1 | HA-H3 | Vic | Yam | NA-N1 | NA-N2 | Vic | Yam |
| groups | (μg) | composition | (μg) | (μg) | (μg) | (μg) | (μg) | (μg) | (μg) | (μg) |
| 1_1 | 18 | 18HA | 18 | | | | | | | |
| 1_2 | 36 | 9-9-9-9 HA | 9 | 9 | 9 | 9 | | | | |
| 1_3 | 48 | 12-12-12-12 | 12 | 12 | 12 | 12 | | | | |
| | | HA | | | | | | | | |
| 1_4 | 72 | 18-18-18-18 | 18 | 18 | 18 | 18 | | | | |
| | | HA | | | | | | | | |
| 1_5 | 45 | 9-18-9-9 HA | 9 | 18 | 9 | 9 | | | | |
| 1_6 | 54 | 9-9-18-18 HA | 9 | 9 | 18 | 18 | | | | |
| 1_7 | 48 | 9-9-9-9 HA/3- | 9 | 9 | 9 | 9 | 3 | 3 | 3 | 3 |
| | | 3-3-3 NA | | | | | | | | |
| 1_8 | 60 | 9-9-9-9 HA/6- | 9 | 9 | 9 | 9 | 6 | 6 | 6 | 6 |
| | | 6-6-6 NA | | | | | | | | |
| 1_9 | 60 | 12-12-12-12 | 12 | 12 | 12 | 12 | 3 | 3 | 3 | 3 |
| | | HA/3-3-3-3 | | | | | | | | |
| | | NA | | | | | | | | |
| 1_10 | 84 | 12-12-12-12 | 12 | 12 | 12 | 12 | 9 | 9 | 9 | 9 |
| | | HA/9-9-9-9 | | | | | | | | |
| | | NA | | | | | | | | |
| 1_11 | 96 | 18-18-18-18 | 18 | 18 | 18 | 18 | 6 | 6 | 6 | 6 |
| | | HA/6-6-6-6 | | | | | | | | |
| | | NA | | | | | | | | |
| 1_12 | 120 | 18-18-18-18 | 18 | 18 | 18 | 18 | 12 | 12 | 12 | 12 |
| | | HA/12-12-12- | | | | | | | | |
| | | 12 NA | | | | | | | | |

| | |
|---|---|
| Control | Flu D-QIV |

H1: H1 hemagglutinin from Influenza A virus subtype H1N1; H3: H3 hemagglutinin from Influenza A virus subtype H3N2; N1: N1 neuraminidase from Influenza A virus subtype H1N1; N2: N2 neuraminidase from Influenza A virus subtype H3N2; B-Vic: B Victoria lineage; B-Yam: B Yamagata lineage.
Control: Flu D-QIV (Flu Dresden- Quadrivalent Influenza Vaccine)
Strains used to design vaccine compositions are based on WHO recommendation for influenza virus vaccine composition for the 2022-2023 NH season.

### Methodology

### Solicited Events

Solicited administration site events were pain, redness, swelling, and lymphadenopathy. Solicited systemic events were fever, headache, myalgia, arthralgia, fatigue, and chills.

Laboratory data were graded according to the FDA Guidance for Industry “Toxicity Grading Scale for Healthy Adults and Adolescent Volunteers Enrolled in Preventive Vaccine Clinical Trials” dated September 2007 [FDA, 2007]. These laboratory values served as guidelines and were dependent upon institutional normal parameters. Institutional normal reference ranges were provided to demonstrate that they are appropriate.

### Unsolicited Adverse Events

AEs, occurring within 28 days (Day 1 to Day 28) post-vaccination, that were either i) not included in the list of solicited events, or ii) could be included in the list of solicited events but with an onset outside the specified period of follow-up for solicited symptoms, were recorded as unsolicited AEs.

### Adverse Events of Special Interest (AESI)

plMDs were considered as AESIs. plMDs include autoimmune diseases and other inflammatory and/or neurologic disorders of interest which may or may not have an autoimmune etiology. Out of the pIMD subset, Bell's Palsy and Guillain-Barre Syndrome are considered important potential risks in the Flu Seasonal mRNA program. The investigator(s) exercise their medical/scientific judgment to determine whether other diseases have an autoimmune origin (i.e., pathophysiology involving systemic or organ-specific pathogenic autoantibodies) and should also be recorded as a pIMD. The following events were also considered as AESIs:
Severe hypersensitivity reactions within 24 hours after study intervention administration (considered an important potential risk in the Flu Seasonal mRNA program)
Myocarditis/Pericarditis

### Serious Adverse Events (SAE)

All SAEs in enrolled participants were reported by the investigator within 24 hours after the investigator became aware of an SAE.

### Blood Sampling Schedules

Blood samples for humoral immunity are collected at Day 1 (pre-vaccination), Day 29, Day 92, and Day 183.

### HI Assay

Hemagglutination inhibition (HI) antibody titers are determined using the method derived from the WHO Manual on Animal Influenza Diagnosis and Surveillance, WHO/CDS/CSR/NCS/2002.5. Measurements are conducted on thawed frozen serum samples with a standardized and validated method. Serum samples are treated with receptor destroying enzyme overnight, diluted to 1:10, and serial diluted 2-fold in duplicate from 1:10 to 1:10240. After addition of an equal volume of standardized virus (4 HAU/25 μL), neutralization is performed for 1 hour at room temperature, followed by addition of the red blood cells. After 60-120 minutes, plates are tilted and the titer is the reciprocal of the last dilution that fully inhibited hemagglutination as compared to a red blood cell control well. Each serum sample is tested in duplicate within the same assay. The titer results are reported as the GMT for the duplicate.

### NI Assay

Neuraminidase enzyme inhibition (NI) by anti-NA titers are measured using the NA ELLA functional assay where NA is in the form of a virus with mismatched Has or a recombinant protein. The bottom of ELISA plates is coated with the fetuin substrate. The assay is based on the neuraminidase enzymatic activity which releases N-acetyl neuraminic acid from fetuin substrate.

After cleavage of the terminal neuraminic acid, R-D-galactose-N-acetyl-galactosamin is unmasked. Peroxidase-labelled peanut agglutinin binds specifically to the galactose residues and the enzymatic desialylation is detected and quantified by a colorimetric reaction using an enzyme substrate chromogen reagent. The neuraminidase inhibition titer of a serum is measured by mixing a fixed amount of neuraminidase with serial dilutions of serum and is set as the reciprocal of the serum dilution reducing the colorimetric signal resulting from desialylation by 50%.

### Immunogenicity Results

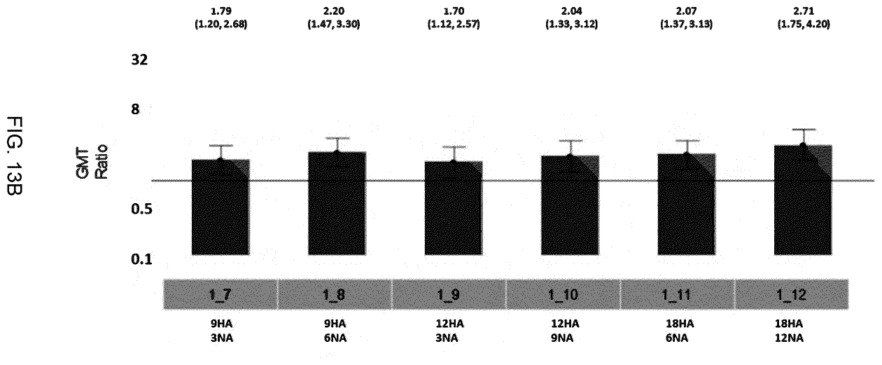

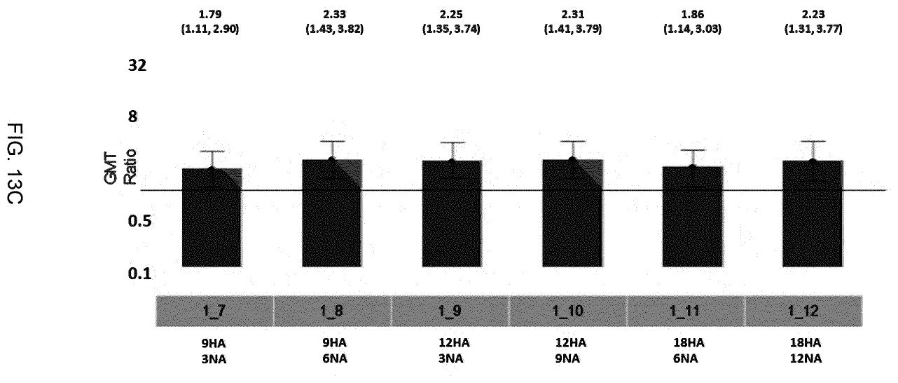

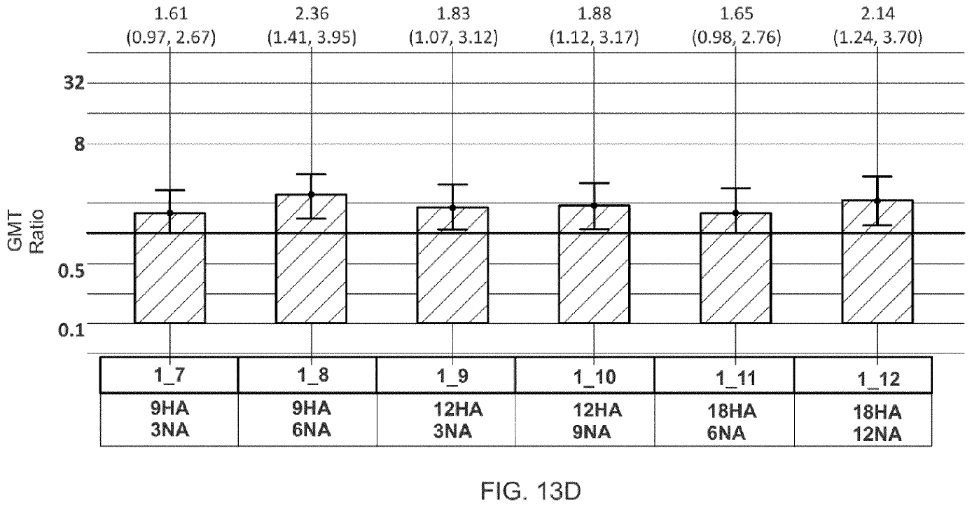

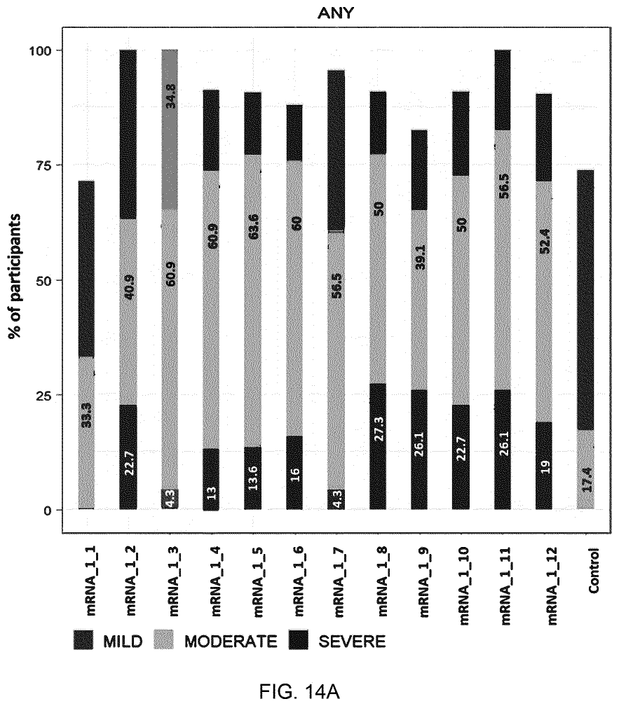

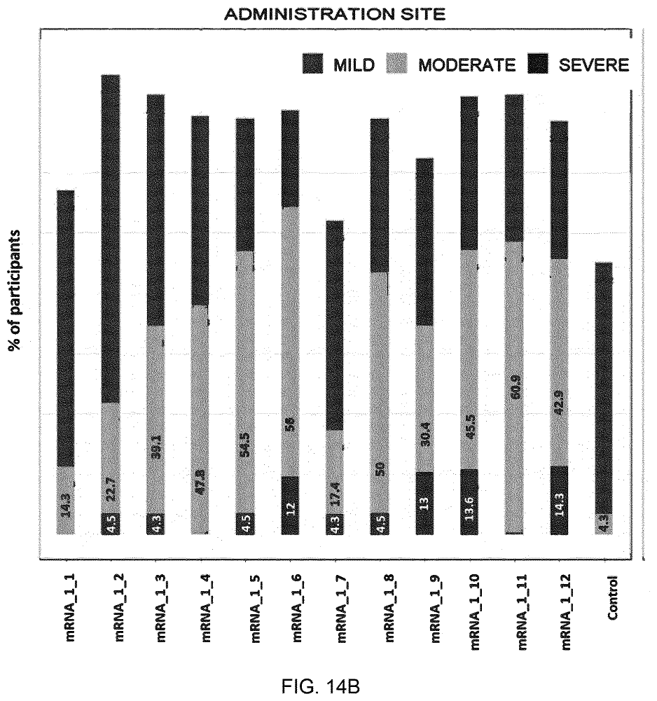

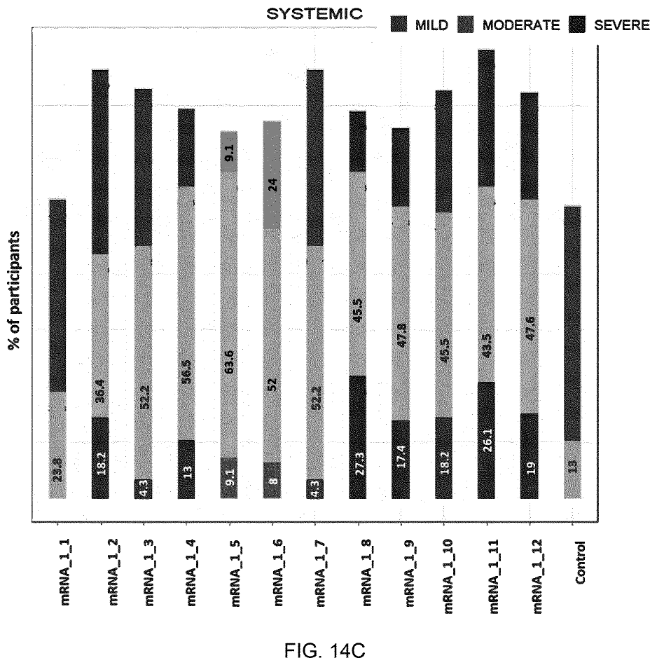

The mRNA-based multivalent seasonal influenza vaccine elicited immune response across the 4 strains for both antigens. An increase of GMT (geometric mean titer) level was observed in all dose groups and the control group across the 4 strains for both the antigens (FIG. 12A-D; FIG. 13A-D).

The addition of 3 mRNA encoding HA had no measured impact on the HI response elicited against the H1N1 strain by the mono-component vaccine, and the addition of mRNA encoding NA had no measured impact on the HI responses elicited by the 4-component HA vaccines (FIG. 12A-D).

HI responses against A strains elicited by the mRNA-based multivalent seasonal influenza vaccine were equal or higher than the control group (FIG. 12A-D).

NI responses against A and B strains elicited by the Flu Seasonal mRNA investigational vaccine were higher than the control group (FIG. 13A-D).

### Safety and Reactogenicity Data

No safety signal has been identified to date, across all tested dose levels and formulations. The reactogenicity profile is similar to what has been observed with other mRNA-based vaccines, with most solicited events, including Grade 3 events, being short-lived and occurring within a few days after vaccination. No apparent differences were observed for the Flu mRNA groups in terms of unsolicited AEs or laboratory abnormalities compared to the control group. No vaccine-related SAEs, fatal SAEs, pIMDs, or AESIs (including myocarditis and pericarditis) were observed in any dose group. No AEs led to withdrawal from the study.

### Solicited AEs

Solicited local (also referred to as administration site) and systemic events were recorded for 7 days following vaccination for all exposed participants. The percentage of participants in each group reporting solicited events is presented in FIG. 14A-D.

In the 1 component group, solicited events were reported in 71.4% of participants. In the 4 components groups, the percentage of participants reporting solicited events ranged from 90.9% to 100%. In the 8 components groups, the percentage of participants reporting solicited events ranged from 82.6% to 100%. In the control group, solicited events were reported in 73.9% of participants. The percentage of participants reporting at least 1 solicited event appeared higher in the 4 components and 8 components groups compared to the 1 component group and the control group.

Overall, in the investigational study intervention groups pain at the injection site and fatigue were the most frequently reported solicited administration site and systemic events, respectively.

In the 1 component group, no participant reported Grade 3 solicited events. In the 4 components groups, the percentage of participants reporting Grade 3 solicited events ranged from 4.3% to 22.7%. In the 8 components groups, the percentage of participants reporting Grade 3 solicited events ranged from 4.3% to 28.6%. In the control group, no participant reported Grade 3 solicited events. The duration of the majority of Grade 3 solicited events ranged between 1 and 2 days in the investigational study intervention groups (FIG. 14D).

### Unsolicited AEs

Unsolicited AEs were recorded on the vaccination day and the next 28 consecutive days. Unsolicited AEs were reported across all the study groups.

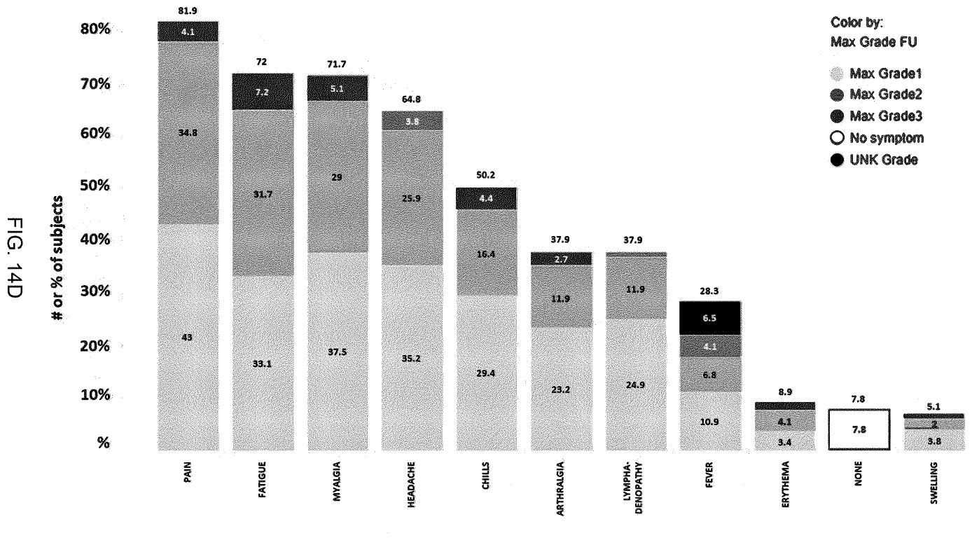

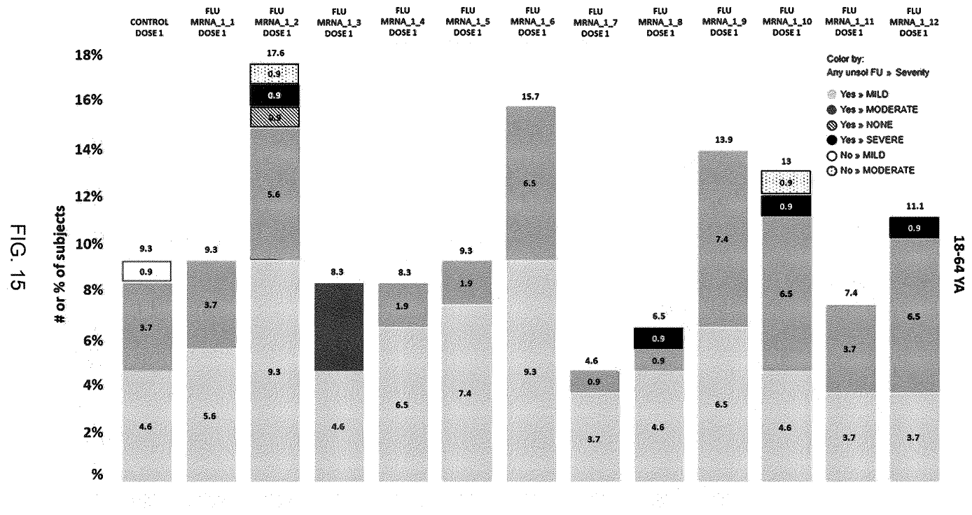

Overall, the percentage of participants with unsolicited AEs in the investigational study intervention groups ranged from 17.4% in Flu mRNA_1_7 group to 52.2% in Flu mRNA_1_9 group. In the 1 component group, unsolicited AEs were reported in 33.3% of participants. In the 4 components groups, the percentage of participants reporting unsolicited AEs ranged from 21.7% to 48.0%. In the 8 components groups, the percentage of participants reporting unsolicited AEs ranged from 17.4% to 52.2%. In the control group, 30.4% of participants reported unsolicited AEs (FIG. 15).

Grade 3 unsolicited AEs were reported only by participants in 8 components groups. One participant from each of Flu mRNA_1_8 group, Flu mRNA_1_10 group and Flu mRNA_1_12 group reported Grade 3 unsolicited AEs. No participant in the control group reported Grade 3 unsolicited AEs. Nausea, dizziness and neutrophil count decreased were the most common Grade 3 unsolicited AEs reported. All 3 events were considered by the investigator to be related to the study intervention (FIG. 15).

SAE, AESI, pIMDs

No vaccine-related SAEs, fatal SAEs, pIMDs, or AESIs (including myocarditis and pericarditis) were observed in any dose group. No AEs led to withdrawal from the study.

### REFERENCES

Coates S, Pohl O, Gotteland J P, et al. Efficient Design of Integrated and Adaptively Interlinked Protocols for Early-Phase Drug Development Programs. Ther Innov Regul Sci. 2020; 54 (1): 184-194.
Trombetta C M, Perini D, Mather S, et al. Overview of Serological Techniques for Influenza Vaccine Evaluation: Past, Present and Future. Vaccines (Basel). 2014; 2 (4): 707-734.
Carnell G W, Trombetta C M, Ferrara F, et al. Correlation of Influenza B Haemagglutination Inhibiton, Single-Radial Haemolysis and Pseudotype-Based Microneutralisation Assays for Immunogenicity Testing of Seasonal Vaccines. Vaccines (Basel). 2021 Jan. 28;9 (2): 100.
Gao J, Couzens L, Eichelberger M C. Measuring Influenza Neuraminidase Inhibition Antibody Titers by Enzyme-linked Lectin Assay. J Vis Exp. 2016 Sep. 6; (115): 54573.
Couzens L, Gao J, Westgeest K, et al. An optimized enzyme-linked lectin assay to measure influenza A virus neuraminidase inhibition antibody titers in human sera. J Virol Methods. 2014 Dec. 15; 210:7-14.
Russell et al. Viruses 2021, 13 (5), 746; https://doi.org/10.3390/v13050746

1-31. (canceled)

32. An immunogenic composition comprising:
(a) a first hemagglutinin (HA) antigen or a first nucleic acid, suitably mRNA, encoding the first HA antigen wherein the first HA antigen is derived from a strain of subtype H3 of Influenza A virus; and
(b) a second HA antigen or a second nucleic acid, suitably mRNA, encoding the second HA antigen wherein the second HA antigen is derived from a strain of subtype H1 of Influenza A virus,
wherein the ratio of (a):(b) is comprised between 1.5:1 and 20:1, optionally between 1.5:1 and 5:1.

33. The immunogenic composition according to claim 32, wherein said ratio of (a):(b) is comprised between 2:1 and 4:1, suitably between 2:1 and 3:1, suitably is 2:1 or 3:1.

34. The immunogenic composition according to claim 32, wherein said ratio of (a):(b) is greater than 3:1 and lower or equal to 20:1, optionally greater than 3:1 and lower or equal to 5:1.

35. The immunogenic composition according to claim 32, further comprising:
(c) at least one further antigen or at least one further nucleic acid, suitably mRNA, encoding the at least one further antigen, wherein the at least one further antigen is derived from a strain of Influenza virus.

36. The immunogenic composition according to claim 35, wherein said at least one further antigen comprises or consists of a peptide or protein selected or derived from an Influenza virus hemagglutinin (HA) or neuraminidase (NA) or an immunogenic fragment or an immunogenic variant thereof.

37. The immunogenic composition according to claim 35, wherein said at least one further antigen or at least one further nucleic acid, suitably mRNA, encoding the at least one further antigen is at least one further HA antigen or at least one further nucleic acid, suitably mRNAs, encoding the at least one further HA antigen derived from a strain of subtype H3 of Influenza A virus, suitably from H3N2, and wherein the ratio of (a) and (c) to (b) is greater than 1:1 and lower or equal to 20:1, optionally greater than 1:1 and lower or equal to 5:1, optionally comprised between 1.5:1 and 5:1.

38. The immunogenic composition according to claim 35, wherein said immunogenic composition comprises a plurality of HA antigens or a plurality of nucleic acids, suitably mRNAs, encoding the plurality of HA antigens derived from at least two different strains of subtype H3 of Influenza A virus, suitably from H3N2, wherein the ratio of the plurality of HA antigens or the plurality of nucleic acids, suitably mRNAs, encoding the plurality of HA antigens to (b) is greater than 1:1 and lower or equal to 20:1, optionally greater than 1:1 and lower or equal to 5:1, optionally comprised between 1.5:1 and 5:1.

39. An immunogenic composition comprising:
(a) a first mRNA encoding a HA of a strain of subtype H3 of Influenza A virus;
(b) a second mRNA encoding a HA of a strain of subtype H1 of Influenza A virus;
(c¹) a third mRNA encoding a HA of a first strain of Influenza B virus; and
(c²) optionally, a fourth mRNA encoding a HA of a second strain of Influenza B virus,
wherein the ratio of (a):(b): (c¹): (c²) is comprised between 1.5:1:1:1 and 20:1:1:1, optionally between 1.5:1:1:1 and 5:1:1:1.

40. The immunogenic composition of claim 39, further comprising:
(c³) a first mRNA encoding a NA of the strain of subtype H3 of Influenza A virus;
(c⁴) a second mRNA encoding a NA of the strain of subtype H1 of Influenza A virus; and
(c⁵) a third mRNA encoding a NA of the first strain of Influenza B virus,
wherein the ratio of (a):(b): (c¹): (c²) is comprised between 1.5:1:1:1 and 20:1:1:1, optionally between 1.5:1:1:1 and 5:1:1:1.

41. The immunogenic composition of claim 40, further comprising:
(c⁶) an eighth mRNA encoding a NA of the second strain of Influenza B virus,
wherein the ratio of (a):(b): (c¹): (c²) is comprised between 1.5:1:1:1 and 20:1:1:1, optionally between 1.5:1:1:1 and 5:1:1:1.

42. The immunogenic composition according to claim 41, wherein the mRNAs of (a), (b), (c¹), (c²), (c³), (c⁴), (c⁵) and/or (c⁶) are formulated in a lipid nanoparticle (LNP), each separately or not.

43. The immunogenic composition according to claim 41, wherein the mRNAs of (a), (b), (c¹), (c²), (c³), (c⁴), (c⁵) and/or (c⁶), optionally each, are not self-replicating.

44. The immunogenic composition according to claim 41, wherein the mRNAs of (a), (b), (c¹), (c²), (c³), (c⁴), (c⁵) and/or (c⁶) comprises at least one chemical modification, suitably the chemical modification is N1-methylpseudouridine and/or pseudouridine, suitably N1-methylpseudouridine.

45. A vaccine comprising the immunogenic composition according to claim 32.

46. The vaccine according to claim 45, further comprising at least one antigen or at least one nucleic acid encoding said at least one antigen, such as at least one mRNA encoding an antigen from a further pathogen, suitably the pathogen being a virus such as Coronavirus (e.g. SARS-COV-1, SARS-COV-2, MERS-COV), Pneumoviridae virus (e.g. Respiratory syncytial virus, Metapneumovirus) and Paramyxovidirae virus (e.g. Parainfluenza virus, Henipavirus).

47. A kit or kit of parts comprising the antigens, mRNAs or the nucleic acids as defined in claim 32, optionally comprising a liquid vehicle for solubilizing, and, optionally, technical instructions providing information on administration and dosage of the components.

48. A method of treating or preventing a disorder caused by an influenza virus, suitably an influenza A and/or influenza B, wherein the method comprises applying or administering to a subject in need thereof the immunogenic composition according to claim 32, the vaccine according to claim 45 or the kit or kit of parts according to claim 47.

49. A method of eliciting an immune response, wherein the method comprises applying or administering to a subject in need thereof the immunogenic composition according to claim 32, the vaccine according to claim 45 or the kit or kit of parts according to claim 47.

50. The method according to claim 49, wherein the immune response is an adaptative immune response, suitably a protective adaptative immune response against an influenza virus, suitably against an influenza A virus and/or an influenza B virus.

51. The method according to claim 48, wherein the immunogenic composition according to claim 32, the vaccine according to claim 45 or the kit or kit of parts according to claim 47 is administered in an amount effective to induce a T cell response and/or a neutralizing antibody response against influenza A H1N1, influenza A H3N2, influenza B/Yamagata lineage and Influenza B/Victoria lineage.


SEQUENCE LISTING (48 sequences)

SEQ ID NO: 1 - protein, 566 aa, synthetic construct
  REGION 1..566: note = HA from A/Michigan/45/2015 (H1N1).
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHXSNKGVTAACPHAGAKSFYKNLIWLVKKGNSYPKLNQSYINDKGKEVLVLWGIHHPSTTADQQSJYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 2 - protein, 469 aa, synthetic construct
  REGION 1..469: note = Amino acid sequence of NA from A/Michigan/45/2015 (H1N1).
MNPNQKIITIGSICMTIGMANLILQIGNIISIWVSHSIQIGNQSQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVNSDTVGWSWPDGAELPFTIDK

SEQ ID NO: 3 - protein, 566 aa, synthetic construct
  REGION 1..566: note = Amino acid sequence of HA from A/Switzerland/8060/2017 (H3N2).
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSSCYPYDVPDYASLRSLVASSGTLEFNNESFNWTGVKQNGTSSACIRKSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIQSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 4 - protein, 469 aa, synthetic construct
  REGION 1..469: note = Amino acid sequence of NA from A/Switzerland/8060/2017 (H3N2).
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCMAWSSSSCHDGKAWLHVCVTGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKSDSSSSSHCLNPNNEEGGHGVKGWAFDDGNDVWMGRTINETSRLGYETFKVVEGWSNSKSKLQINRQVIVDRGDRSGYSGIFSVEGKSCINRCFYVELIRGRKEETEVLWTSNSIVVFCGTSGTYGTGSWPDGADLNLMHI

SEQ ID NO: 5 - protein, 583 aa, synthetic construct
  REGION 1..583: note = Amino acid sequence of HA from B/Colorado/06/2017.
MKAIIVLLMVVTSSADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEGAPGGPYKIGTSGSCPNITNGNGFFATMAWAVPDKNKTATNPLTIEVPYVCTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDKIAAGTFDAGEFSLPTFDSLNITAASLNDDGLDNHTILLYYSTAASSLAVTLMIAIFVVYMVSRDNVSCSICL

SEQ ID NO: 6 - protein, 466 aa, synthetic construct
  REGION 1..466: note = Amino acid sequence of NA from B/Colorado/06/2017.
MLPSTIQTLTLFLTSGGVLLSLYVSASLSYLLYSDILLKFSPTEITAPTMPLDCANASNVQAVNRSATKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSQTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 7 - protein, 584 aa, synthetic construct
  REGION 1..584: note = Amino acid sequence of HA from B/Phuket/3073/2013.
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 8 - protein, 466 aa, synthetic construct
  REGION 1..466: note = Amino acid sequence of NA from B/Phuket/3073/2013.
MLPSTIQTLTLFLTSGGVLLSLYVSASLSYLLYSDILLKFSRTEVTAPIMPLDCANASNVQAVNRSATKGVTPLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGDEGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 9 - protein, 566 aa, synthetic construct
  REGION 1..566: note = Amino acid sequence of HA from A/Singapore/INFIMH-16-0019/2016 (H3N2).
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRGSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIESIRNETYDHNVYRDEALNNRFQIKGVELKSGYKDWILWISFAISCFLLCVALLGFIMWACQKGNIRCNICI

SEQ ID NO: 10 - protein, 469 aa, synthetic construct
  REGION 1..469: note = Amino acid sequence of NA from A/Singapore/INFIMH-16-0019/2016 (H3N2).
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLIDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKNDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 11 - protein, 564 aa, synthetic construct
  REGION 1..564: note = Amino acid sequence of HA from A/Brisbane/02/2018 (H1N1).
MKAILVVLLYTFTTANADTLCIGYHANNSTDTVDTVLEKNVTVTHSVNLLEDKHNGKLCKLGGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPPTTADQQSLYQNADAYVFVGTSRYSKKFKPEIATRPKVRDREGRMNYYWTLVEPGDKITFEATGNLVVPRYAFTMERNAGSGIIISDTPVHDCNTTCQTAEGAINTSLPFQNVHPVTIGKCPKYVKSTKLRLATGLRNVPSIQSRGLFGAIAGFIEGGWTGMVDGWYGYHHQNEQGSGYAADLKSNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 12 - protein, 469 aa, synthetic construct
  REGION 1..469: note = Amino acid sequence of NA from A/Brisbane/02/2018 (H1N1).
MNPNQKIITIGSTCMTIGMANLILQIGNIISIWVSHSIQIGNQSQIETCNQSVITYENNTWVNQTYVNISNTNFAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSIKQDIVGINEWSGYSGSFVQHPELTGLDCIRPCFWVELIRGRPEENTIWTSGSSISFCGVDSDTVGWSWPDGAELPFTIDK

SEQ ID NO: 13 - protein, 566 aa, synthetic construct
  REGION 1..566: note = Amino acid sequence of HA from A/Kansas/14/2017 (H3N2).
MKTIIALSCILCLVFAQKIPGNDNSTATLCLGHHAVPNGTIVKTITNDRIEVTNATELVQNSSIGEICDSPHQILDGENCTLIDALLGDPQCDGFQNKKWDLFVERNKAYSNCYPYDVPDYASLRSLVASSGTLEFNNESFNWAGVTQNGTSSSCIRGSKSSFFSRLNWLTHLNSKYPALNVTMPNNEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPERQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACMGSIRNGTYDHNVYRDEALNNRFQIKGVELKSGYKDWILWISFAISCFLLCVALLGFIMWACQKGNIRCNICI

SEQ ID NO: 14 - protein, 469 aa, synthetic construct
  REGION 1..469: note = Amino acid sequence of NA from A/Kansas/14/2017 (H3N2).
MNPNQKIITIGSVSLTISTICFFMQIAILITTVTLHFKQYEFNSPPNNQVMLCEPTIIERNITEIVYLTNTTIEREICPKPAEYRNWSKPQCGITGFAPFSKDNSIRLSAGGDIWVTREPYVSCDPDKCYQFALGQGTTINNVHSNNTARDRTPHRTLLMNELGVPFHLGTKQVCIAWSSSSCHDGKAWLHVCITGDDKNATASFIYNGRLVDSVVSWSKDILRTQESECVCINGTCTVVMTDGNATGKADTKILFIEEGKIVHTSKLSGSAQHVEECSCYPRYPGVRCVCRDNWKGSNRPIVDINIKDHSIVSSYVCSGLVGDTPRKTDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 15 - protein, 566 aa, synthetic construct
  REGION 1..566: note = Amino acid sequence of HA from A/South Australia/34/2019 (H3N2).
MKTIIALSYILCLVFAQKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWTGVKQNGTSSACIRGSSSSFFSRLNWLTHLNYTYPALNVTMPNKEQFDKLYIWGVHHPGTDKDQIFLYAQSSGRITVSTKRSQQAVIPNIGFRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMMDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCIALLGFIMWACQKGNIRCNICI

SEQ ID NO: 16 - protein, 469 aa, synthetic construct
  REGION 1..469: note = Amino acid sequence of NA from A/South Australia/34/2019 (H3N2).
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 17 - protein, 582 aa, synthetic construct
  REGION 1..582: note = Amino acid sequence of HA from B/Washington/02/2019.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 18 - protein, 466 aa, synthetic construct
  REGION 1..466: note = Amino acid sequence of NA from B/Washington/02/2019.
MLPSTIQTLTLFLTSGGVLLSLYVSASLSYLLYSDILLKFSPTEITAPTMPLDCANASNVQAVNRSATKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSKTERMGMGLYVKYGGDPWADSDALTFSGVMVSMKEPGWYSFGFEIKDKKCDVPCIGIEMVHDGGKETWHSAATAIYCLMGSGQLLWDTVTGVDMAL

SEQ ID NO: 19 - protein, 564 aa, synthetic construct
  REGION 1..564: note = Amino acid sequence of HA from A/Guangdong- Maonan/SWL1536/2019 (H1N1).
MKAILVVLLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 20 - protein, 469 aa, synthetic construct
  REGION 1..469: note = Amino acid sequence of NA from A/Guangdong- Maonan/SWL1536/2019 (H1N1).
MNPNQKIITIGSICMTIGMANLILQIGNIISIWVSHSIQIGNQSQIETCNKNVITYENNTWVNQTYVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNNILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 21 - protein, 566 aa, synthetic construct
  REGION 1..566: note = Amino acid sequence of HA from A/Hong Kong/2671/2019 (H3N2).
MKTIIALSYILCLVFTQKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWAGVTQNGKSFSCIRGSSSSFFSRLNWLTHLNYTYPALNVTMPNKEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 22 - protein, 469 aa, synthetic construct
  REGION 1..469: note = Amino acid sequence of NA from A/Hong Kong/2671/2019 (H3N2).
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSSYVCSGLVGDTPRKSDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHT

SEQ ID NO: 23 - protein, 566 aa, synthetic construct
  REGION 1..566: note = Amino acid sequence of HA from A/Hawaii/70/2019 (H1N1).
MKAILVVLLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDKGVTAACPHAGAKSFYKNLIWLVKKGNSYPKLNQTYINDKGKEVLVLWGIHHPPTIAAQESLYQNADAYVFVGTSRYSKKFKPEIATRPKVRDQEGRMNYYWTLVEPGDKITFEATGNLVVPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 24 - protein, 469 aa, synthetic construct; identical to SEQ ID NO: 20
  REGION 1..469: note = Amino acid sequence of NA from A/Hawaii/70/2019 (H1N1).

SEQ ID NO: 25 - protein, 566 aa, synthetic construct
  REGION 1..566: note = Amino acid sequence of HA from A/Hong Kong/45/2019 (H3N2).
MKTIIALSYILCLGFTQKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWAGVTQNGKSFSCIRGSSSSFFSRLNWLTHLNYTYPALNVTMPNKEQFDKLYIWGVHHPGTDKDQISLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQNTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAISCFLLCVALLGFIMWACQKGNIRCNICI

SEQ ID NO: 26 - protein, 469 aa, synthetic construct
  REGION 1..469: note = Amino acid sequence of NA from A/Hong Kong/45/2019 (H3N2).
MNPNQKIITIGSVSLTISTICFFMQIAILITTVTLHFKQYEFNSLPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSSYVCSGLVGDTPRKSDSSSSSHCLNPNNEEGGHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGADLNLMHI

SEQ ID NO: 27 - protein, 566 aa, synthetic construct
  REGION 1..566: note = Amino acid sequence of HA from A/Victoria/2570/2019 (H1N1).
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDREGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 28 - protein, 469 aa, synthetic construct
  REGION 1..469: note = Amino acid sequence of NA from A/Victoria/2570/2019 (H1N1).
MNPNQKIITIGSICMTIGTANLILQIGNIISIWVSHSIQIGNQSQIETCNKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIVGWSWPDGAELPFTIDK

SEQ ID NO: 29 - protein, 566 aa, synthetic construct
  REGION 1..566: note = Amino acid sequence of HA from A/Wisconsin/588/2019 (H1N1).
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 30 - protein, 469 aa, synthetic construct; identical to SEQ ID NO: 28
  REGION 1..469: note = Amino acid sequence of NA from A/Wisconsin/588/2019 (H1N1).

SEQ ID NO: 31 - protein, 566 aa, synthetic construct [sequence omitted]

SEQ ID NO: 32 - protein, 469 aa, synthetic construct
  REGION 1..469: note = Amino acid sequence of NA from A/Cambodia/e0826360/2020 (H3N2).
MNPNQKIITIGSVSLTISTICFFMQIAILITTVTLHFKQYEFNSPPNNQVMLCEPTIIERNMTEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 33 - protein, 566 aa, synthetic construct
  REGION 1..566: note = Amino acid sequence of HA from A/Darwin/9/2021 (H3N2).
MKTIIALSNILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPNTDKNQISLFAQSSGRITVSTKRSQQAVIPNIGSRPRIRGIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 34 - protein, 469 aa, synthetic construct
  REGION 1..469: note = Amino acid sequence of NA from A/Darwin/9/2021 (H3N2).
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVSDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 35 - protein, 582 aa, synthetic construct
  REGION 1..582: note = Amino acid sequence of HA from B/Austria/1359417/2021.
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 36 - protein, 466 aa, synthetic construct
  REGION 1..466: note = Amino acid sequence of NA from B/Austria/1359417/2021.
MLPSTIQTLTLFLTSGGVLLSLYVSASLSYLLYSDILLKFSQTEITAPTMPLDCANASNVQAVNRSATKGATLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 37 - protein, 566 aa, synthetic construct
  REGION 1..566: note = Amino acid sequence of HA from A/Darwin/6/2021 (H3N2).
MKTIIALSNILCLVFAQKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRGIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 38 - protein, 469 aa, synthetic construct
  REGION 1..469: note = Amino acid sequence of NA from A/Darwin/6/2021 (H3N2).
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 39 - protein, 566 aa, synthetic construct
  REGION 1..566: note = Amino acid sequence of NA from A/Darwin/6/2021 (H3N2).
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIAARPKVRDRAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 40 - protein, 469 aa, synthetic construct
  REGION 1..469: note = Amino acid sequence of NA from A/Victoria/4897/2022 (H1N1)
MNPNQKIITIGSICMTIGTANLILQIGNIISIWVSHSIQIGNQSQIETCDKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTETDNKFSKKQDIVGINEWSGYSGSFVQHPELTGLNCIRPCFWVELIRGRPEENTIWTSGSSISFCGVDSDIMGWSWPDGAELPFTIDN

SEQ ID NO: 41 - protein, 566 aa, synthetic construct
  REGION 1..566: note = Amino acid sequence of HA from A/Wisconsin/67/2022 (H1N1)
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIATRPKVRDQAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 42 - protein, 469 aa, synthetic construct
  REGION 1..469: note = Amino acid sequence of NA from A/Wisconsin/67/2022 (H1N1)
MNPNQKIITIGSVCMTIGTANLILQIGNIISIWVSHSIQIGNQSQIETCNKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDN

SEQ ID NO: 43 - protein, 566 aa, synthetic construct
  REGION 1..566: note = Amino acid sequence of HA from A/Sydney/5/2021 (H1N1).
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGNFINYEELREQLSSVSSFERFEIFPKTSSWPNHDSDNGVTAACPHAGAKSFYKNLIWLVKKGKSYPKINQTYINDKGKEVLVLWGIHHPPTITDQESLYQNADAYVFVGTSRYSKKFKPEIAARPKVRDQAGRMNYYWTLVEPGDKITFEATGNLVAPRYAFTMEKDAGSGIIISDTPVHDCNTTCQTPEGAINTSLPFQNVHPITIGKCPKYVRSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 44 - protein, 469 aa, synthetic construct
  REGION 1..469: note = Amino acid sequence of NA from A/Sydney/5/2021 (H1N1).
MNPNQKIITIGSICMTIGTANLILQIGNIISIWVSHSIQIGNQSQIETCNKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVSSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDK

SEQ ID NO: 45 - protein, 566 aa, synthetic construct
  REGION 1..566: note = Amino acid sequence of HA from A/Thailand/8/2022 (H3N2).
MKAIIALSNILCLVFAQKIPGNDNSTATLCLGHHAVPNGTIVKTITNDRIEVTNATELVQNSSIGKICNSPHQILDGGNCTLIDALLGDPQCDGFQNKEWDLFVERSRANSSCYPYDVPDYASLRSLVASSGTLEFKNESFNWTGVKQNGTSSACKRGSSSSFFSRLNWLTSLNNIYPAQNVTMPNKEQFDKLYIWGIHHPDTDKNQFSLYAQSSGRITVSTKRSQQAVIPNIGSRPRVRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 46 - protein, 469 aa, synthetic construct
  REGION 1..469: note = Amino acid sequence of NA from A/Thailand/8/2022 (H3N2).
MNPNQKIITIGSVSLTISTIXXFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVHDRTPYRTLLMNELGVPFHLGTKQVCIAWSSSSCHDGKAWLHVCITGDDKNATASFIYNGKLVDSVVSXSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHT

SEQ ID NO: 47 - protein, 566 aa, synthetic construct
  REGION 1..566: note = Amino acid sequence of HA from A/Massachusetts/18/2022 (H3N2).
MKAIIALSNILCLVFAQKIPGNDNSTATLCLGHHAVPNGTIVKTITNDRIEVTNATELVQNSSIGKICNSPHQILDGGNCTLIDALLGDPQCDGFQNKEWDLFVERSRANSSCYPYDVPDYASLRSLVASSGTLEFKNESFNWTGVKQNGTSSACKRGSSSSFFSRLNWLTSLNNIYPAQNVTMPNKEQFDKLYIWGVHHPDTDKNQFSLFAQSSGRITVSTKRSQQAVIPNIGSRPRVRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 48 - protein, 469 aa, synthetic construct
  REGION 1..469: note = Amino acid sequence of NA from A/Massachusetts/18/2022 (H3N2).
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVHDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHT